(19) United States
(12) Patent Application Publication
(10) Pub. No.: US 2012/0304317 A1
(43) Pub. Date: Nov. 29, 2012

(54) TRANSGENIC RODENT EXPRESSING TRUNCATED DISC1

(75) Inventors: Sanbing Shen, Aberdeen (GB); Gernot Riedel, Aberdeen (GB); David St. Clair, Aberdeen (GB)

(73) Assignees: THE UNIVERSITY COURT OF THE UNIVERSITY OF ABERDEEN, Aberdeen (GB); WYETH PHARMACEUTICALS INC., Madison, NJ (US); TMRI LIMITED, Glasgow (GB)

(21) Appl. No.: 13/054,444

(22) PCT Filed: Jul. 16, 2009
(86) PCT No.: PCT/GB2009/001757

§ 371 Date: Jan. 14, 2011

(30) Foreign Application Priority Data
Jul. 16, 2008 (GB) 0813038.7

(51) Int. Cl. A01K 67/027; A01K 67/02; G01N 33/48; C12N 15/873; C12N 5/10; C07H 21/04

(52) U.S. Cl. 800/3; 435/352; 536/23.5; 800/14; 800/21; 800/22

(57) ABSTRACT

The invention provides transgenic rodents, particularly mice, expressing truncated versions of the Disrupted-in-Schizophrenia-1 (DISC1) gene and showing Schizophrenia-related neural and behavioral phenotypes. The rodents of the invention have (1) a plurality of copies of a heterologous truncated Disc1 genomic DNA sequence which includes at least 1 stop codon after exon 8 such as to encode a Disc1 polypeptide truncated before exon 9; (2) 2 copies of endogenous Disc1 genomic DNA sequence encoding full length Disc1 polypeptide. Also provided are related materials and methods.

### TECHNICAL FIELD

The present invention relates generally to transgenic rodents, particularly mice, expressing truncated Disc1 and showing Schizophrenia-related neural and behavioral phenotypes.

### BACKGROUND ART

Schizophrenia

Schizophrenia is a severe mental illness affecting 1% of the world population. The disease is diagnosed by a combination of positive symptoms, negative symptoms and impaired cognitive function. There are no objective tests, nor is there a convincing animal model. The causes of schizophrenia are multi-factorial. Monozygotic twin concordance rates for schizophrenia approach ˜50%. Together with family studies, these data indicate a heritability of ˜85%. Linkage studies suggest significant association with numerous chromosomal regions and some promising candidate genes have emerged but the majority of the genetic risk remains unexplained.

The Disrupted-in-Schizophrenia-1 (DISC1) Gene

One of the most exciting findings in the genetics of schizophrenia is the discovery of a gene termed DISC1 . DISC1 is truncated from intron 8 by a balanced translocation (1;11) in a large Scottish family (Millar et al., 2000), which cosegregates with major mental illness including schizophrenia, depression and bipolar disorders (St Clair et al., 1990; Blackwood et al., 2001). Independent linkage/association studies now implicate DISC1 in schizophrenia, severe affective disorders and autistic spectrum disorders of diverse populations (Devon et al., 2001; Ekelund et al., 2001; Cannon et al., 2005; Hennah et al., 2005; Sachs et al., 2005; Thomson et al., 2005; Zhang et al., 2005; Chen et al., 2007; Hennah et al., 2007; Kilpinen et al., 2008). However, robust evidence for functional variants is still lacking and genetic heterogeneity is likely.

DISC1 is a coiled-coil protein forming developmentally regulated complexes with proteins including PDE4, NDEL1, LIS1 and 14-3-3ε (Brandon et al., 2004), and is involved in nucleus-centrosome association, neuronal proliferation, differentiation and migration. The C-terminus of DISC1 binds NDEL1. DISC1 mutant truncated after exon 8 fails to bind NDEL1, inhibits neurite outgrowth in vitro (Ozeki et al., 2003) and impairs cortical development in vivo (Kamiya et al., 2005). The N-terminus of DISC1 binds all PDE4 isoforms (Murdoch et al., 2007), and PDE4B is independently implicated in schizophrenia and mood disorders (Millar et al., 2005).

How DISC1 truncation results in psychiatric illness is unclear. No truncated DISC1 protein is detected in lymphoblasts from the Scottish family (Millar et al., 2005), while no brains from the translocation carriers have become available for examination. It seems likely that a single copy of the normal DISC1 is insufficient for proper brain development and function. However, if the truncated DISC1 protein is produced from cDNA deletion constructs (Ozeki et al., 2003; Hikida et al., 2007; Pletnikov et al., 2008), it may act in a dominant negative fashion (Kamiya et al., 2005). It is also clear that disruption of DISC1 binding partners alters brain development. For instance, deletion or mutation of PAFAH1B1 encoding LIS1 results in lissencephaly in humans (Reiner et al., 1993; Vallee and Tsai, 2006).

In mice, Pafah1b1^{-/-} embryos die shortly after implantation and Pafah1b1^{+/-} mice display cortical and hippocampal disorganization due to delayed neuronal migration (Assadi et al., 2003). Ywhae encodes 14-3-3ε that binds/stabilizes phosphorylated Ndel1, and Ywhae^{-/-} mutants die at birth with defects similar to Pafah1b1^{+/-} mice (Toyo-oka et al., 2003). Deletion of Lis1 binding partners (Nde1 and Ndel1) either dramatically reduces cerebral cortex (Feng and Walsh, 2004) or is embryonic lethal with neuronal migration defects (Sasaki et al., 2005).

Animal Models

Animal models of Schizophrenia can help to understand the relationship between the biochemical and pathological changes in the brain as well as the behavioural and other neurological symptoms. Furthermore, they can provide a model in which therapeutic strategies can be tested.

Some of the symptoms of schizophrenia such as hallucinations and delusions can not be assessed easily in animals. For other abnormalities such as cognitive dysfunction and affective symptoms, some proxy measures have been developed to test animals. For example, pre-pulse inhibition and latent inhibition are commonly used to detect attention-related deficits, while Porsolt swim tests (PST) and tail suspension tests (TST) can indicate a depression-related phenotype.

Recently reported DISC1 transgenics or mutants exhibit some abnormalities consistent with a schizophrenia phenotype. For example, ENU mutant strain 31 L has a predominant mood disorder-like phenotype with reduced Pde4b activity, while the 100P strain shows profound deficits in pre-pulse inhibition and latent inhibition (Clapcote 2007).

WO03/099995 concerns Disc1 polypeptides, Disc1 nucleic acids, and recombinant Disc1 altered mice. The Disc1 nucleic acid sequence is apparently a cDNA encoding the mouse ortholog to the human DISC1 amino acid sequence. The application discusses generally the possibility of production of a variety of Disc1 deficient mice by utilising the cDNA, and breeding the mice to have alterations in both their alleles. There is no evidence that any such mice are actually produced.

Koike et al 2006 describe a deletion variant in mouse Disc1 specific to the 129S6/SvEv strain of mice that is said to introduce a termination codon at exon 7, abolish production of the full-length protein, and impair working memory performance when transferred to the C57BU6J genetic background. However, a recent publication shows that most of Disc1 gene products detected by Disc1 antibodies in C57BU6 mice are also found in 129S6/SvEv mice (Ishizuka et al 2007). Additionally, neither 129 mice with spontaneous Disc1 truncation (Koike et al 2006, Ishizuka et al 2007), nor DN-DISC1 mice using an ectopic (Hikida et al., 2007) or inducible promoter (Pletnikov et al., 2008) display significant changes in pre-pulse inhibition or some of other schizophrenia-related abnormalities.

Thus it can be seen that novel Disc1 rodent models for schizophrenia or related disorders, particularly those providing combinations of relevant phenotypes not provided by known models, would provide a contribution to the art.

### DISCLOSURE OF THE INVENTION

The present inventors have sought to explore the role of Disc1 in brain development. They have therefore generated Disc1ₜᵣtransgenic mice with a ˜148kb artificial chromosome (BAC) expressing Disc1 exons 1-8.

Using this partial simulation of the human situation, the inventors have provided a combination of disease-relevant phenotypes including a series of novel features not previously reported.

For example the Disc1ₜᵣ transgenic mice of the invention display enlarged lateral ventricles, reduced cerebral cortex, partial agenesis of the corpus callosum, and thinning of layers II/III with reduced neural proliferation at mid-neurogenesis. Parvalbumin GABAergic neurons are reduced in the hippocampus and medial prefrontal cortex, and displaced in the dorsolateral frontal cortex.

In culture, transgenic neurons grow fewer and shorter neurites.

Behaviourally, the inventors have demonstrated that Disc1ₜᵣtransgenic mice are defective in a variety of symptom-related tests. In latent inhibition, the non-pre-exposed Disc1ₜᵣ transgenic mice fail to “freeze” during the tone, shock or post-shock tone periods. In PST and TST, they have longer immobility, with reduced switches from immobile to mobile status. Remarkably, they make fewer stress calls during the TST. This last observation may be a novel indicator of the presence of communication deficits and/or other negative symptoms that resemble those found in schizophrenia.

In various aspects of the invention, there are provided transgenic rodents which include within a plurality of their cells at least 2 copies of a truncated Disc1 genomic DNA sequence encoding at least the first 8 exons of the Disc1 polypeptide. In preferred embodiments the truncated sequence has a GFP coding sequence fused in-frame with the end of exon 8 which includes a translational stop codon followed by a transcriptional termination signal SV40 polyA sequence, such that exon 9 is not expressed. These are expressed against a background of 2 copies of endogenous Disc1 genomic DNA sequence encoding full length Disc1 proteins in heterozygous transgenic animals

In other aspects the rodents include at least 4 copies of the truncated Disc1 genomic DNA sequence against a background of 2 copies of endogenous Disc1 genomic DNA sequence encoding full length Disc1 polypeptides in homozygous transgenic animals.

Other aspects relate to methods for producing such rodents, uses thereof, and materials related to such methods and uses.

Some of these aspects and embodiments will now be described in more detail.

Some of the present results were disclosed after the presently claimed priority date in Shen et al (2008). J Neurosciences 28 (43): 10893-10904.

Thus in one aspect there is provided a transgenic rodent which includes within a plurality of its cells: (1) a plurality of (preferably 2) copies of a heterologous Disc1 genomic DNA sequence which expresses a Disc1 polypeptide truncated after exon 8, and including at least 1 stop codon after or in the final codon of exon 8, such that exon 9 is not expressed; (2) 2 copies of endogenous Disc1 genomic DNA sequence encoding full length Disc1 polypeptide.

As described below, the heterologous Disc1 genomic DNA sequence is preferably truncated, and expresses a Disc1 polypeptide truncated after exon 8, and including at least 1 stop codon after or in the final codon of exon 8, such that exon 9 is not expressed;

In one embodiment the transgenic rodent is heterozygous with respect to the heterologous truncated Disc1 genomic DNA sequences, and there are preferably 2 copies/cell (although 3 is not excluded). Heterozygous rodents having 2 copies are particularly preferred because the 2 copies of the truncated Disc1 and 2 copies of native full length Disc1 can be expressed in an approximately 1:1 ratio (e.g. between 0.8:1 or 0.9:1 and 1.1:1 or 1.2:1). It is understood from the disclosure herein that the truncated Disc1 may act in a dominant negative fashion by binding to other members of the Disc1 complex and thereby reducing normal complex formation, and therefore this ratio (corresponding to the ratio in the Scottish schizophrenic family reported in Millar 2000) is believed to be particularly useful.

In another embodiment the transgenic rodent is homozygous with respect to the heterologous truncated Disc1 genomic DNA sequences, and there are preferably 4 copies/cell (although 6 is not excluded). Although not mimicking so closely the Millar population, nevertheless the disease phenotype of the homozygous rodents may be more pronounced in some respects, and they may therefore also have utility in understanding and modeling the disease.

The rodent may be selected from mice, rats, and guinea pigs. Preferably the rodent is a rat or mouse. Most preferably it is a mouse.

The Heterologous Truncated Disc1 Genomic DNA

The term “heterologous” is used broadly in this aspect to indicate that the truncated Disc1 genomic DNA has been introduced into said cells of the rodent, or an ancestor thereof, using genetic engineering, i.e. by human intervention. In the present case the heterologous truncated is expressed against the background of the full length endogenous equivalent gene. Preferably the truncated gene is from the same species as the transgenic animal.

The 2 or more copies will generally be identical (i.e. introduced by multiple insertions from a single type of construct) and will preferably include at least the first 8 exons and intronic sequences.

The truncated Disc1 genomic DNA will further preferably comprise the native Disc1 genomic promoter e.g. at least 5, 10, 15 or 20 kb thereof, and be operably linked thereto. In such embodiments there will be sufficient sequence for the promoter to be functional i.e. have the ability to initiate transcription of the truncated Disc1 genomic DNA. The level of promoter activity is quantifiable for instance by assessment of the amount of mRNA produced by transcription from the promoter or by assessment of the amount of protein product produced by translation of mRNA produced by transcription from the promoter. The amount of a specific mRNA present in an expression system may be determined for example using specific oligonucleotides which are able to hybridise with the mRNA and which are labelled or may be used in a specific amplification reaction such as the polymerase chain reaction.

The truncated Disc1 genomic DNA. ill include the first 8 exons (although optionally exon 8 may be modified at or around its 3′ end in order to facilitate truncation and\or in-frame fusion as described below). Thus, for example, the final 9, 8, 7, 6, 5, 4, 3, 2 or 1 nucleotides of exon 8 may be modified for this purpose e.g. to introduce a restriction site therein.

Preferably the heterologous truncated Disc1 genomic DNA includes a reporter gene or an epitope tag which is expressed as a fusion with the truncated Disc1 polypeptide, and which includes a stop codon, which facilitates the detection of transgene and transgenic products.

Thus the reporter gene preferably is fused to the end of exon 8.

A preferred reporter may encode EGFP, or beta-gal, or luciferase.

Preferably the heterologous truncated Disc1 genomic DNA includes some or all of intron 8.

Preferably the heterologous truncated Disc1 genomic DNA includes a polyA sequence, not naturally occurring in Disc 1, within intron 8.

Preferably the heterologous truncated Disc1 genomic DNA includes some or all of exon 9 and intron 9, e.g. at least 1, 5, 10, or 15 kb thereof. Since exon 9 is preceded by a stop codon in exon 8 (or a gene fused thereto) and a polyA sequence, it will not be expressed.

In the examples herein a ˜148 kb sequence with entire 5′ intergenic sequences (˜20.8 kb) was employed, up to 16.7 kb of intron 9. The present inventors believe the use of the native genomic contextual sequences of Disc1 has contributed to the excellent phenotype obtainable using the invention. This is shown in FIG. 16.

Some other aspects and embodiments of the invention will now be discussed:

Cells and Tissues

A cell or tissue sample of the transgenic rodent as defined above e.g. which comprises: (1) a plurality of (preferably 2) copies of a heterologous truncated Disc1 genomic DNA sequence as described above; (2) 2 copies of endogenous Disc1 genomic DNA sequence encoding full length Disc1 polypeptide.

Thus the invention also provides a neuron or other somatic cells having these properties from the transgenic rodent, for example in culture.

The invention further provides gametes from the transgenic rodent. These may include:

(1) a plurality of (preferably 2) copies of a heterologous truncated Disc1 genomic DNA sequence as described above; (2) 1 copy of endogenous Disc1 genomic DNA sequence encoding full length Disc1 polypeptide.

Nucleic Acids

The invention also provides modified proteins, RNA and DNA derived from, or for use in the characterization and production of, the transgenic rodents described herein.

Nucleic acids may include a truncated Disc1 genomic DNA sequence encoding a Disc1 polypeptide truncated after exon 8, and including at least 1 stop codon after exon 8 such that exon 9 is not expressed, in the same terms as described above e.g. including a fusion sequence and some or all of intron 8, exon 9, and intron 9.

It will be appreciated that a nucleic acid encoding a fusion polypeptide as described herein will be at least partially synthetic in that it will comprise nucleic acid sequences which are not found together in nature (do not run contiguously) but which have been ligated or otherwise combined artificially.

Nucleic acids may comprise, consist or consist essentially of any of the sequences disclosed herein.

Nucleic acid sequences may be provided and utilised by techniques known in the art (for example, see Sambrook, Fritsch and Maniatis, “Molecular Cloning, A Laboratory Manual”, Cold Spring Harbor Laboratory Press, 1989, and Ausubel at al., Short Protocols in Molecular Biology, John Wiley and Sons, 1992) or later editions of the same. These techniques include (i) the use of the polymerase chain reaction (PCR) to amplify samples of the relevant nucleic acid, e.g. from genomic sources, and RNA.

Nucleic acids may be in the form of vectors e.g. plasmids, cosmids, BAC and YAC vectors.

Phenotypes of Transgenic Rodents

By way of exemplification, in the Examples below the inventors describe Disc1ₜᵣ transgenic mice expressing 2 copies of a Disc1ₜᵣ-EGFP fusion gene in a ˜148 kb native mouse Disc1 genomic environment, which drives transgene expression at the endogenous Disc1 expression sites in the cerebellum, cerebral cortex and hippocampus.

These Disc1ₜᵣtransgenic rodents display an array of schizophrenia-related abnormalities as set out in Table 1, in which they are compared with other DISC1/Disc1 mouse models.

TABLE 1
Phenotypes of DISC1/Disc1 mouse models

| | Koike et | | | Hikida | Pletnikov et | |
|---|---|---|---|---|---|---|
| | al | | | et al | al | Shen et |
| | 129 | Clapcote et al | | DN- | Inducible | al |
| Phenotype | mice | 31L | 100P | DISC1 | DISC1 | Disc1ₜᵣ |
| Brain volume | NS | ↓6% | ↓13% | NS | NS | ↓ in |
| | | | | | | male |
| Lateral ventricles | ? | ? | ? | ↑ | ↑ | ↑ |
| Cerebral cortex | NS | ↓ | ↓ | NS | NS | ↓ |
| Neural proliferation | ? | ? | ? | ? | ? | ↓ |
| Neurite outgrowth | ? | ? | ? | ? | ↓ | ↓ |
| Corpus callosum | ? | ? | ? | ? | ? | ↓ |
| Parvalbumin in MPF cortex | ? | ? | ? | ↓ | ? | ↓ |
| Parvalbumin in | ? | ? | ? | ? | ? | ↓ |
| hippocampus | | | | | | |
| Open field: Horizontal | NS | NS | ↑↑ | ↑ | ↑ in male | NS |
| activity | | | | | | |
| Open field: Vertical activity | NS | NS | ↑ | ↑ | ? | ? |
| Anxiety | NS | NS | NS | NS | NS | ? |
| Aggression | ? | ? | ? | ? | ↑ in male | ? |
| Sociability | ? | ↓ | NS | NS | ↓ in male | ? |
| Spatial learning & memory | ? | NS | NS | NS | ↓ in female | ? |
| Working memory | ↓ | ↓ | ↓↓ | ? | ? | ? |
| Pre-pulse inhibition | NS | ↓ | ↓↓ | ↓ | NS | ? |
| Latent inhibition | ? | ↓↓ | ↓↓ | ? | ? | ↓ |
| Immobility in forced swim | ? | ↑ | NS | ↑ | ? | ↑ |
| test | | | | | | |
| Immobility in tail | ? | ? | ? | ? | ? | ↑ |
| suspension test | | | | | | |
| Stress calls | ? | ? | ? | ? | ? | ↓ in |
| | | | | | | male |
| Social recognition memory | ? | ? | ? | ? | ? | ↓ |
| Abnormal EEG in PF | ? | ? | ? | ? | ? | yes |
| cortex | | | | | | |

?: not known;
NS: no statistical difference;
↓: reduced;
↑: increased.

The changes seen in Disc1ₜᵣ transgenic brains of the invention are consistent with neuropathology in schizophrenia.

For example, examinations on first-episode and unmedicated schizophrenic patients consistently show selective regional deficits in brain volume and ventricular enlargement (Harrison, 1999; Honea et al., 2005; Ross et al., 2006); the latter also appears to be a common feature of Disc1 mutants and transgenic mice reported so far. Morphometric analyses of Disc1ₜᵣ transgenic brains reveal a significant dilation of the lateral ventricles. Although the scale of reduction is not as dramatic as in ENU mutants (Clapcote et al., 2007), quantitative analyses of brain surface areas show a significant reduction in Disc1ₜᵣ transgenic mice (see FIGS. 3 and 11). In contrast to DN-DISC1 mice, compensational changes were not observed (Hikida et al., 2007). Instead, the neuropathology appears to be dosage-related, and most dramatic phenotypes are observed in the M20 female founder (FIGS. 11 and 12) and in some of the M19 homozygotes (FIGS. 8 and 13).

Similar to phenotypes described in most other mouse models, enlarged lateral ventricles and reduced cerebral cortex are observed. Also consistent with the findings in ENU mutants (Clapcote et al., 2007), Disc1ₜᵣtransgenic mice show deficits in conditioning of latent inhibition, and longer immobility in depression-related tests. They also resemble aspects of DN-DISC1 mice, and have reduced parvalbumin neurons in the medial prefrontal cortex (Hikida et al., 2007) and reduced neurite outgrowth in culture (Pletnikov et al., 2008).

Up to 6 months, there was no endophenotype for hippocampal synaptic plasticity (LTP/LTD).

Novel Phenotypes of the Transgenic Rodents of the Invention

In addition to previously characterised phenotypes, Disc1ₜᵣ transgenic mice exhibit a remarkable series of novel phenotypes not previously reported.

One of the most prominent cellular features of schizophrenia that has emerged from post-mortem studies is a consistent reduction of parvalbumin neurons (Lewis et al., 2005). Parvalbumin cells belong to a subgroup of GABAergic inhibitory interneurons, which are vital for neuronal synchronization. Along with the GABA synthesizing enzyme GAD67, parvalbumin expression is consistently reduced in schizophrenic brains. We detected significant reduction of parvalbumin neurons in the medial prefrontal cortex, similar to the findings reported in DN-DISC1 transgenic mice (Hikida et al., 2007). Additionally, parvalbumin neurons also were reduced in the hippocampus of Disc1ₜᵣ transgenic mice. These appear to be anatomically specific, as the total number of parvalbumin cells at the reticular nucleus of the thalamus or the dorsolateral frontal cortex remained unchanged. However, in the frontal cortex, the distribution of parvalbumin-positive cells is altered. In contrast to a more concentrated localization in the inner half of WT littermates, they are more evenly spread throughout the layers in the transgenic cortex. The causes and functional consequences of these abnormalities are yet to be determined. However, dorsolateral frontal cortex-dependent cognitive functions are compromised in schizophrenia (Miller and Cohen, 2001).

Disc1 is critical for cortical neurogenesis, and the reduction of Disc1ₜᵣ brain volume is attributed mainly to the differences in the cerebral cortex. This is paralleled by a reduction in the thickness of cortex in both transgenic males and females. Surprisingly, no significant changes have been detected in the cortex of DN-DISC1 mice (Hikida et al., 2007; Pletnikov et al., 2008), and it is not clear whether this is related to the ectopic promoters used. The reduced cerebral cortex we observe largely results from the thinning of layers II/III. The differences are statistically significant on morphometric analyses of WT and Disc1ₜᵣ transgenic brains. These are precisely the layers altered in schizophrenia (Harrison, 1999).

These cortical layers contain pyramidal neurons, which are involved in inter-hemispheric communication. Indeed a partial agenesis of the corpus callosum is seen in Disc1ₜᵣ transgenic mice. In the rostral brain, the corpus callosum is thinned, and in the caudal brain, it stops crossing the midline before the appearance of the SCO. It is worth noting that some mouse strains (I/LnJ, 129/J and BALB/c) have agenesis of the corpus callosum (Livy and Wahlsten, 1991). However, the background strains (C57BU6J and CBA/Ca) of the Disc1ₜᵣ transgenic mice reported in this study are known to have an intact corpus callosum. Consistent with our findings, a complete agenesis of the corpus callosum is rare in schizophrenia (Motomura, 2002; Chinnasamy et al., 2006; Paul et al., 2007). However, mild alterations in inter-hemispheric callosal connections may be relatively common in schizophrenia and in autism (Innocenti et al., 2003; Miyata et al., 2007). Partial agenesis of the corpus callosum may result from decreased numbers of cortical neurons and/or reduced neurite outgrowth. This is supported by our observation that Disc1ₜᵣ transgenic neurons have fewer and shorter neurites in primary culture, which also echoes earlier studies that truncated DISC1 inhibits neurite outgrowth (Ozeki et al., 2003; Pletnikov et al., 2008).

We have presented evidence that truncated Disc1 selectively reduces proliferation at the outermost cortex during mid-neurogenesis. This corresponds to the peak of Disc1 expression in embryos (Brandon et al., 2004) and the reduced layers II/III we observe in transgenic adults. A more dramatic reduction of cortical neurogenesis is reported in Nde1 null mutants, with reduced proliferation and retarded migration (Feng and Walsh, 2004). Interestingly, the newly identified Disc1 binding partner DBZ (or Su48 or Zfp365) is also a coiled-coil protein (Hattori et al., 2007). It associates with centrosomes and is involved in proliferation (Wang et al., 2006). Ectopic expression of Su48 causes abnormal mitosis, while injection of an anti-Su48 antibody leads to mitotic failure. Su48 associates with Nde1 (Hirohashi et al., 2006), the latter is shown to be vital for cortical development (Feng and Walsh, 2004). Furthermore, Nde1 interacts with Lis1 and Lis1 regulates mitosis in cultured mammalian cells (Faulkner et al., 2000). Our data adds to accumulating evidence that Disc1 complexes play critical roles in the cortical genesis. Alterations in the Disc1 gene lead to failure of normal neuronal proliferation, reduced neurite outgrowth, and decreased/displaced parvalbumin neurons. Consequently, these lead to thinned layers reduced cortical size, enlarged ventricles and behavioral changes, all of which are consistent with schizophrenia-like phenotypes.

Other important novel phenotypes of the Disc1ₜᵣ transgenic mice reported in this study are:
A significant deficit in social recognition memory.
Disc1ₜᵣ mice presented with clearly abnormal EEG activity in prefrontal cortex (reduced power especially in the gamma range) and thus present a robust schizophrenia-like phenotype

Thus in certain aspects and embodiments of the invention the transgenic rodent may be used for experimental purposes in studying schizophrenia, schizoaffective disorder, depression and bipolar disorders. By “experimental” it is meant permissible for use in animal experimentation or testing purposes under prevailing legislation applicable to the research facility where such experimentation occurs.

Thus in certain aspects and embodiments of the invention the transgenic rodent will have one or more, and preferably all, of the phenotypes described in Table 1.

In one embodiment it may display equal to or at least 1, 2, 3, 4, 5 or all 6 of the following novel phenotypes (compared to a corresponding wild-type strain used to generate the transgenic):
thinning of the cortical layers
selective decrease of neural proliferation in the developing cortex at mid-neurogenesis,
partial agenesis of the corpus callosum,
reduced parvalbumin GABAergic neurons in the hippocampus and
displaced parvalbumin cells at the dorsolateral frontal cortex,
increased immobility and reduced vocalization in depression-related tests,

In further embodiments it may display (in addition to equal to or at least 1, 2, 3, 4, 5 or all 6 of the above phenotypes), 1 or preferably both of:
A significant deficit in social recognition memory.
Abnormal EEG activity in prefrontal cortex, an in particular significant decreased power in the gamma frequency band.

(in each case compared to a corresponding wild-type strain used to generate the transgenic).

The invention further provides methods of preparing a transgenic animal model with one or more, and preferably all, of the phenotypes described in Table 1, and preferably equal to or at least 1, 2, 3, 4, 5 or all 6 of the described novel phenotypes, e.g. by:

(a) introducing a nucleic acid as described above into a rodent embryonic stem cell to produce an altered embryonic stem cell;

(b) introducing the altered embryonic stem cell into a rodent blastocyst to produce an altered blastocyst;

(c) introducing the altered blastocyst into a pseudopregnant rodent female to produce a pregnant female rodent;

(d) allowing the pregnant rodent to produce offspring; and

(e) screening the offspring for the introduction of 2 or more copies of the nucleic acid in the genome.

Preferably the transgenic mouse model will display (in addition to equal to or at least 1, 2, 3, 4, 5 or all 6 of the above phenotypes), 1 or preferably both of:
A significant deficit in social recognition memory.
Abnormal EEG activity in prefrontal cortex, and in particular significant decreased power in the gamma frequency band.

In another method, which is preferred, the nucleic acid as described above is injected into the pronucleus of a fertilized rodent egg. This is then implanted into the uterus of a pseudopregnant rodent female to produce a pregnant female rodent, and the process continues as above from step (d).

Such methods are now well within the ability of the skilled person and can be performed in the light of the present disclosure without undue burden.

Also provided are methods of producing an F₁ generation by crossing a founder animal of either sex (F₀ generation) with an animal which is non-transgenic in respect of the proteins discussed herein, and is preferably wild-type). The offspring (F₁ generation) may then be screened and those which carry the transgenes in appropriate dosage resulting in the combinations of phenotypes described above.

Also provided are methods of producing an F₂ generation by crossing 2 F₁ animals of appropriate sex. The offspring (F₂ generation) may then be screened and those which carry the transgenes in appropriate dosage resulting in the combinations of phenotypes described above.

Transgenic Disc1ₜᵣ animals of the invention may be crossed with other genetic models (i.e. Nrg^{+/-}, Ndel1^{+/-}, Pafah1b1^{+/-}, YWHAE^{+/-} or PAC1-overexpressing mice) to produce compound genetic model(s) for use in the methods described herein. Such compound models form a further aspect of the invention.

Also provided is a method of modelling schizophrenia, schizoaffective disorder, depression and bipolar disorders, and in particular schizophrenia, by providing the transgenic organism with the combinations of phenotypes described above and preferably monitoring equal to or at least 1, 2, 3, 4, 5 or all 6 of the following phenotypes under different conditions:
thickness of the cortical layers
neural proliferation in the developing cortex at mid-neurogenesis,
agenesis of the corpus callosum,
parvalbumin GABAergic neurons in the hippocampus and
parvalbumin cells at the dorsolateral frontal cortex,
immobility and vocalization in depression-related tests, plus optionally 1 or both of:
social recognition memory,
EEG activity in prefrontal cortex, and in particular power in the gamma frequency band.

Preferred behavioural phenotypes to model include latent inhibition, immobility and vocalization in conventional depression-related tests as described herein. Other preferred phenotypes are EEG activity.

Methods of Screening

The transgenic rodents described herein may be used in methods of screening or assessing current or potential anti-psychotic and pro-cognitive drugs e.g. by use of otherwise conventional psychopharmacological or neuroanatomical methods.

The methods can serve either as primary screens, in order to identify new inhibitors/modulators of the relevant disorders, or as secondary screens in order to study known inhibitors/modulators in further detail.

Using the transgenic model systems, a compound suspected of having a therapeutic effect in relation to schizophrenia, schizoaffective disorder, depression and bipolar disorders, and in particular schizophrenia, can be administered to the animal, and any effects on the condition (e.g. change in relevant phenotypes or neuroanatomy, and especially improvements in behavioural symptoms, or any other suitable indicator) can be studied. The rodents are thus useful in testing the efficacy of such compounds in a pharmacokinetic context.

For neuroanatomy, generally speaking, a drug to be tested is administered to a control animal or group of animals which are not the transgenic animals of the invention and simultaneously to transgenic animals of the invention. The drug may be continuously administered over a period of time. After administering the drug for a sufficient period of time the control animal(s) along with the transgenic animal(s) are sacrificed. Examination of the brain of the animals is made as described above.

The transgenic rodents described herein may also be used in methods of investigating how truncated Disc1 expression affects other binding partners which may serve as novel drug targets.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### Figures

FIG. 1. The truncated mouse Disc1 transgene and expression. (A) Genomic organization of the mouse Disc1 locus, with an arrow corresponding to the breakpoint in the Scottish family. (B) A BAC clone RP23-236F19 containing ˜148kb mouse Disc1 genomic DNA, starting from the 3′UTR of Tsnax, and ending with 16.7 kb of Disc1 intron 9. The BAC was fused to an EGFP at the end of exon 8 followed by a PolyA. The Nrul fragment (148,730 bp) was purified for microinjection. (C) M19 heterozygous transgenics contained 2 copies of the transgene, as similar intensity of PCR products were obtained from the truncated and endogenous Disc1 genes at 17-30 cycles. (D) Two E17.5 embryos (lanes 1-2) and a transgenic mother (lane 3) were genotyped by PCR with EGFP primers. (E-G) RT-PCR with primers for the transgene (E), the endogenous Disc1 (F) and a housekeeping HPRT (G), showing comparable levels of the truncated (E) and full-length (F) Disc1 mRNA. NC, negative control.

FIG. 2. Comparable temporal and spatial patterns of expression of truncated and full-length Disc1. In situ hybridization was carried out using DIG-labeled sense (S) and antisense (AS) RNA probes on E17.5 (A-F) and 2-month brain sections (G-P). While the Disc1 AS probe hybridized to the endogenous full-length Disc1 transcripts, EGFP AS probe detected the Disc1ₜᵣ-EGFP messages only. Both the endogenous and transgenic transcripts (purple) were detected at the regions of cerebral cortex (ctx) including cingulate (cg) and piriform (pir) cortex, CA1, CA2, CA3 and dentate gyrus of the hippocampus (hip). Bars=500 μm in A-P.

FIG. 3. Enlarged lateral ventricles (LV) and reduced cerebral cortex (Ctx) in Disc1ₜᵣ transgenic (Tg) mice. WT (A) and Tg (B) brain sections were Nissl-stained, imaged at the level where the anterior commissure (AC) crossed the midline, and quantified with AxioVision Rel. 4.5. (C) The LV was significantly (*, p<0.05) enlarged in Tg brains (0.819±0.079 mm², n=11) in comparison to WT littermates (0.568±0.043 mm², n=15). (D-E) Magnified view of the cerebral cortex from WT and Tg representatives showing changes in layers (F) Statistical analyses of the cerebral cortex detected moderate but significant (p<0.05) reduction in Tg mice (1353.6±19.9 μm, n=11) compared with WT littermates (1409.2±10.0 μm, n=15). (G) Layers II/III of the cerebral cortex was thinned (p<0.01) from 347.2±4.5 μm in WT to 302.7±3.4 μm in Tg mice. Bars=1 mm in A-B; Bars=200 μm in D-E.

FIG. 4. Reduced neurogenesis in Disc1ₜᵣ transgenic (Tg) embryos. (A-D) A pulse of BrdU was injected into four E15.5 pregnant females and newborn brains were processed with an anti-BrdU antibody. For each brain, four images were taken from the cerebral cortex at the left and right sides of two consecutive sections with the largest lateral ventricles. Images were arbitrarily divided into 5 layers as shown, and BrdU-positive cells were quantified from each area (400 μm wide×150 μm height). Images A and B were from two WT littermates, C and D from two Tg newborns. (E) Statistical analyses revealed significant reduction of BrdU-positive cells in the arbitrarily assigned layer 1 of Tg mice (140.4±4.9, n=13), compared to their WT littermates (158.6±5.3, n=8). (F) The total number of BrdU-incorporated cells was also significantly reduced in Tg mice compared with that in WT littermates. Bars=200 μm in A-D. * for p<0.05.

FIG. 5. Partial agenesis of the corpus callosum (CC) in 2-month transgenic brains. Coronal sections of WT (A and D) and Tg (B and E) brains were Nissl-stained. (A-B) Images represented average thickness of rostral CC in WT (A) and Tg (B) brains where the AC crossed the midline. (C) Statistical analyses showed significant reduction of the CC in 11 Tg brains in comparison to 15 WT. (D) At the SCO level (arrowed in D-E), a thick layer of the CC crossed the midline in all 15 WT mice, (E) while Tg CC failed to cross the midline in 9 out of 11 cases. Bars=200 μm in A-B, Bars=1 mm in D-E.

FIG. 6. Fewer and shorter neurites in cultured transgenic (Tg) neurons. WT (A) and Tg (B) newborn cortex were dissociated and neurons were cultured for 26 hours in vitro. Images (6-8 fields/mouse) were randomly taken under a 20× objective lens. Neurons (1817 WT and 845 Tg) were quantified for the number (C) and length of neurites with 20 (red), 40 (blue) 60 (green) and 80 μm (yellow) rings (D), respectively. The data were presented as Mean±SEM. * for p<0.05, ** for p<0.01. Bars=50 μm in A-B.

FIG. 7. Parvalbumin (PV) interneurons in the prefrontal cortex of WT and Tg brains. (A) A brain section stained with anti-PV, showing areas of MPFC and DLFC where magnified images (E-H) were taken for counting PV cells. (B) Statistical analyses of PV cells in the MPFC of WT and Tg mice as illustrated in E (WT) and F (Tg). (C) PV-positive cells in 6 arbitrarily assigned layers (1386 μm wide×267 μm height) of the DLFC as illustrated in G and H. (D) Total number of PV cells in the DLFC showing no difference between WT and Tg mice. Bars=1 mm in A; Bars=200 μm in E-H.

FIG. 8. PV interneurons are reduced in the hippocampus of Disc1ₜᵣ transgenic mice. (A-F) Brain sections from 14 WT (A-C), 6 heterozygous (not shown) and 10 homozygous (D-F) transgenic mice were stained with anti-PV. PV-positive cells at the CA1, CA2, CA3 and dentate gyrus (DG) were quantified from 6 comparable images of each mouse brain as shown. (G) Statistical analyses of the mean PV cells in each area of the hippocampus, showing significant reduction of the PV interneurons in the CA1 of the heterozygotes, and in the CA1-CA2-CA3 of the homozygotes. Bars=200 μm in A-F. * for p<0.05, ** for p<0.01.

FIG. 9. DISC1ₜᵣ transgenic mice are defective in conditioning of latent inhibition. (A) The horizontal activity (Mean±SEM) in numbers of beam breaks per second (bb/sec) during the different phases of conditioning. (B) Pooled activity (bb/sec) during the 5×10 sec tone period. (C) Pooled activity (bb/sec) during the 5×2sec shock period. (D) Total number of beam breaks (Mean±SEM) during the 120 sec retention test on the following day. Note that only the npe-WT group showed considerable ‘freezing’ during the tone (B), shock (C) or retention (D) period. * for p<0.05. npe, non-pre-exposed, pe, pre-exposed to tone.

FIG. 10. Increased immobility (A-C) and reduced vocalization (D-F) of Disc1ₜᵣ transgenic mice in depression tests. Disc1ₜᵣ transgenic (Tg) mice and WT littermates were tested individually in 6min PST (A) or TST (B-F). Transgenic mice showed increased immobility in PST (A) and TST (B), with a reduced number of switches from immobile to mobile status in the last 4 min of the TST (C). (D) Disc1ₜᵣ transgenic mice made significantly fewer stress calls. (E) An example of vocalizations (squeaks) recorded by a bat detector showing amplitude and frequency (kHz) of calls during the 6 min TST. (F) Magnified view of the calls. * for p<0.05, ** for p<0.01.

FIG. 11. Severe neuroanatomical phenotypes in the M20 transgenic founder. Nissl-stained coronal sections of a 15-month WT control (A-C) and M20 founder (D-F). In the anterior brain, the lateral ventricles (LV) were dilated 2.26 (D) and 2.98 (E) times, in comparison to that in WT brain (A and B). The cerebral cortex (Ctx) was 16% reduced. In the middle region (C and F), the corpus callosum (CC) in the M20 brain was substantially thinned. (G-H) Magnified view of the cortex from sections A and D showed that the layers II, III and V were affected in the M20 mouse. Bars=1 mm in A-F, 0.1 mm in G-H.

FIG. 12. Reduced parvalbumin interneurons in the hippocampus of M20 transgenic founder. (A-F) Brain sections from M20 transgenic founder (D-F) and a WT littermate (A-C) were stained with anti-PV. PV-positive cells at the CA1, CA2, CA3 and dentate gyrus (DG) were quantified from 6 comparable images of each mouse as shown. (G) Statistical analyses of the mean PV cells in each area of the hippocampus. (H-I) The PV staining in the reticular nucleus of the thalamus is not significantly different between the M20 founder and the WT control. Bars=204 μm in A-F and H-I.

FIG. 13. Morphometric analyses revealed reduced brain volume in Disc1ₜᵣ transgenic males. (A) A representative brain image illustrating measures taken with AxioVision Rel. 4.5 software. (B) Total brain surface (Mean±SEM) including olfactory bulb, cerebral cortex, colliculus and cerebellum was significantly smaller in transgenic males. (C)

Cerebral cortex surface area was significantly reduced in transgenic males. ** for p<0.01. TgF, transgenic female; TgM, transgenic male; WTF, WT female; WTM, WT male. Bar=1 mm in A.

FIG. 14. Design of experimental protocol for assessing sociability and social memory. Animals were given 10 minutes initially to habituate to the box. Sociability was tested by means of placement of stranger 1 into the first chamber, this occurred 15 minutes after habituation period. Social memory was then investigated by quantifying a preference for social novelty 5 minutes after sociability. The original stranger mouse (stranger 1) remained in its cage on one side of apparatus. A new unfamiliar mouse (stranger 2) was placed in the opposite cage.

FIG. 15. Experimental set up for investigating sociability and social memory of Disc1ₜᵣ transgenic mice. The box has three accessible compartments, two of which contain small cages for confinement of stranger mice. These cages are perforated to allow interaction between two mice without the threat of aggressive behaviour.

FIG. 16. Nrul fragment used for the generation of the Disc1ₜᵣ transgenic mice, derived from the BAC clone RP23-236F19 fused with an EGFP reporter. The BAC clones RP23-236F19 sequence can be found from mouse genomic DNA database MM9 at the chromosomal positions of chr8:127557274-127705811 (see website: http://genome.ucsc.edu/cgi-bin/hgc?hgsid=136539000&o=127557273&t=127705811&g=bacEndPairs&i=RP23%2D2 36F19&c=chr8&l=127526354&r=127836790&db=mm9&pix=620

### EXAMPLES

### Methods

BAC clone and validation. The RPCI-23 BAC library was constructed by cloning EcoRI genomic fragments of C57BU6J mice into the pBACe3.6 vector (http://bacpac.chori.org). The RP23-236F19 clone was kindly supplied by Dr de Jong, with end sequences available (AZ705991 and AZ705988). The clone was verified by pulse field gel electrophoresis (PFGE) and polymerase chain reaction (PCR) with primers from MWG-Biotech. The T7 end was defined by a 950bp product with primers BACT7For (5′-CGCAAGATGTGGCGTGTTACGG-3′) and TsnaxRev (5′-GGCTGCTCACAACCTACACACG-3′), and the Sp6 end by a 1099bp band with Disc1In9For (5′-AAGGTAGAACCAGGTGGCTTCC-3′) and BACSp6Rev (5′-CGTGATAGCCGTTGTATTCAGC-3′). The BAC insert was further validated by PCR with primers for the Disc1 promoter (245 bp with PromFor 5′-TATCAACTTCAGCCGCATCCGC-3′ and PromRev 5′-TCATAACCTCGCCTCTGG-3′), exon 2 (626 bp with E2For 5′-GACAATCTGAGAGGCTGACTGG-3′ and E2Rev 5′-GTTGCTCAGTAGGTAGTCCTGC-3′) and intron 5 (545 bp with In5For 5′-AGAGTCTTGTGGTTGGATGGCG-3′ and In5Rev 5′-TGAATACAGCACCAGGCTCTGC-3′).

Modification of RP23-236F19. We carried out homologous recombination as described (Yang et al., 1997), for in-frame fusion of EGFP cDNA to the end of Disc1 exon 8. A homologous recombination cassette was constructed in the pSV1 vector to comprise (1) 1026bp Disc1 intron7-exon8 region with primers In7BamFor (5′-AAAAGGATCCTTGACTAACTACTGTTGCCAGG-3′) and E8NcolRev (5′-GCAGTCCATGGATAGGGCCAGCATCTTGG-3′), (2) 745bp Ncol-EcoRl fragment of EGFP cDNA (gift from Dr Ian Chambers, Edinburgh), (3) 148 bp BgIII-Sall PoIyA from pSG5, (4) 1785bp Disc1 intron 8 (Xhol-EcoRV) with primers InBEcoFor (5′-ATGTGAATTCTAGTGGTGTCAGGCAGGTGTGG-3′) and In8RVRev (5′-TAATGATATCGATGAGGAATACCACAGACGCC-3′), and (5) blunted BamHI fragment of the RecA. The cassette was transformed, into the BAC clone. The first round homologous recombination was screened for a 1076 bp PCR product with primers In7HRFor (5′-ACAGGTGATGTGTGTGGAGTCC-3′), and EGFPRev (5′-ATGCCGTTCTTCTGCTTGTCGG-3′). The second round homologous recombination was confirmed by the appearance of the same 1076 bp PCR product, together with a 2443 bp PCR fragment with primers EGFPFor (5′-TCCTGCTGGAGTTCGTGACC-3′) and In8HRRev (5′-GTCACACAGGAATAAGCCACGG-3′).

PFGE. Both the original and EGFP-modified BAC DNA were digested with Clal, Mlul, Notl, Nrul, Pvul, Sall and Xhol, and run on 1% agarose in 0.5xTBE at 14° C., 150V, 10″-10″ for 18 h and 5″-5″ for 6 h, with Midrange I PFG marker (N3551 S, Biolabs) and Kb ladder (N3232L, Biolabs), to verify the predicted restriction patterns. The modified DNA was diagnosed definitively by the appearance of a 4.5 kb Xhol band instead of 4.9 kb in the un-modified BAC DNA. The 148,730 bp Nrul fragment, with 1335 bp vector sequence at the T7 site and 219 bp vector sequence at the Sp6 site, was purified from PFGE for pronuclear injection.

Generation of Disc1ₜᵣ transgenic mice. All experimental procedures were conducted in accordance with the United Kingdom Animals (Scientific Procedures) Act of 1986 and were approved by the Ethical Review Committee, University of Aberdeen, and the UK Home Office (London). The purified Nrul fragment at ˜5 ng/μl was injected into fertilized eggs superovulated from F1 (CBA/CaCrl:C57BU6JCrl, Charles River UK) mice. Transgenic founders were identified by a 319 bp EGFP product with EGFPFor (5′-ACCATCTTCTTCAAGGACGACG-3′) and EGFPRev (5′-TGCTCAGGTAGTGGTTGTCG-3′), and by a 591 bp fragment with primers 5′-ATAATAAGCGGATGAATGGC-3′ and 5′-CTGCTCACAACCTACACACG-3′. The copy number was determined by semi-quantitative PCR for 17, 21, 25 and 30 cycles, on the ratio of a 517 bp band from the endogenous Disc1 (In13For, 5′-CTACAACACAGAGCCTTGCTGC-3′ and E14Rev, 5′-AGCAGTAGCAGCGGCATTGG-3′), with a 706 bp fragment from the transgene (E8For, 5′-TTGCTGGAAGCCAAGATGCTGG-3′ and EGFPRTR2, 5′-TCACGAACTCCAGCAGGACC-3′). Experiments were carried out on M19 transgenic mice and wildtype (WT) littermates from heterozygote×WT littermate breeding, on the genetic background of 50% CBA/CaCrl and 50% C57BU6JCrl, unless specified otherwise.

RT-PCR. The mRNA was extracted from E17.5 embryonic and adult brains using RNAzol B (Biogenesis). Reverse transcription (RT) was carried out with 1 μg of total RNA using Omniscript kits (QIAGEN) at 37° C. for 1 hour. RT-PCR was performed for 30 cycles with primers for the transgene (696 bp, 5′- TGTGACCTGATGGCACTGGTGG-3′ and 5′-GTTGCCGTCCTCCTTGAAGTCG-3′), and for endogenous Disc1 (363 bp, 5′-TTGCTGGAAGCCAAGATGCTGG-3′ and 5′-CTTCACGCCTATGGCTTCGC-3′), and for the house-keeping gene Hprt (352bp, 5′-CCTGCTGGATTACATTAAAGCACTG-3′ and 5′-GTCAAGGGCATATCCAACAACAAAC-3′).

In situ hybridization was carried out to compare the endogenous and transgene expression. To detect the endogenous Disc1 expression, a 394 bp RT-PCR product comprising exons 12-14 of the mouse Disc1 was amplified and cloned into Xbal-Xhol sites of pBluescript SK⁻ vector with primers Disc1E12XbaFor (ctagtctagaTGCGAAGCCATAGGCGTGAAG) and Disc1E14XhoRev (tatccgctcgagCATCCTGTAGACATCTCCTGAG). The plasmid DNA was linearized with Xbal or Xhol, and DIG-labelled anti-sense or sense probe was transcribed with T3 or T7 RNA polymerase respectively (Roche). The probe for the transgene expression was reversely transcribed from the entire EGFP coding sequence. The hybridization was carried out as described (Nishida et al., 2002).

Morphometric and histological analyses. Adult mice were humanely killed with a lethal dose of sodium pentobarbitone, and brains were dissected; post-fixed in cold paraformaldehyde (4%) for 24 hours and imaged under a Zeiss stereomicroscope with AxioVision Rel. 4.5. After cryoprotection with 30% sucrose in PBS overnight, brains were sectioned coronally on a Vibratome at 40 μm and kept in PBS at 4° C. before use. Newborn brains were freshly dissected, snap-frozen in OCT, processed in 12 μm serial coronal sections on a cryostat (CM1850; Leica Microsystems) and mounted on Polysine slides (VWR). One set of sections from each brain was stained with cresyl violet and imaged using an Axiovert 40CFL microscope for anatomical examination. Images were morphometrically quantified with AxioVision Rel. 4.5 software (Zeiss). Data were analyzed by one-way ANOVA and presented as Mean±SEM. p<0.05 was considered to be statistically significant.

Neuronal culture and neurite outgrowth. Primary neuronal culture was conducted as described previously (Lang, et al., 2006). Briefly, 24-well plates were pre-coated with poly-L-ornithine (Sigma) for 1 hour followed with fibronectin (Invitrogen) for 2 hours. The newborns were genotyped by PCR. Their cortices were individually dissected, trypsinized, and mechanically dissociated into single cell suspension. Cells were seeded in the pre-coated 24-well plates (2×10⁵/well), and cultured in neurobasal medium (Invitrogen) supplemented with 2% B27 (Invitrogen) plus 2 mM glutamine (Sigma) at 37° C. with 5% CO₂. Twelve hours after plating, tissue debris was removed and the medium was renewed.

Cell images were taken randomly around the centre of each well 26 hours after culture, using an Axiovert 40CFL microscope with a 20× objective lens. Cells were quantified with AxioVision Rel. 4.5 software for the number of neurites on individual cells and grouped into one, two, three or more neurites. For the length of neurites, rings with radius at 20, 40, 60 and 80 μm respectively were applied to each cell, and cells were categorized accordingly. Data were analyzed by one-way ANOVA and presented as Mean÷SEM. * for p<0.05, ** for p<0.01.

Birthdating of BrdU labeling. Time-mated pregnant females were injected with a pulse of BrdU (i.p., 50 mg/kg body weight) at E15.5. Newborn brains were processed as described above. Sections were treated with 0.4% pepsin (Sigma-Aldrich) in PBS for 30min at 37° C., denatured with 2N HCl for 30min at 37° C., and neutralized with 0.1 M sodium borate (pH 8.5) for 10min at room temperature. BrdU incorporation assay (BD Biosciences) was executed according to the manufacturers' instructions.

Immunohistochemistry. Sections were processed immunohistochemically as described previously (Lang et al., 2006). The primary antibodies included mouse anti-BrdU (1:200; BD Biosciences) and rabbit anti-parvalbumin (1:1,500; Swant). For immunofluorescent staining, the secondary antibodies were Texas red-conjugated donkey anti-mouse IgG (1:1,000; Invitrogen). For the bright-field staining, the secondary antibody was biotinylated goat anti-rabbit antibody (1:400; Sigma-Aldrich), and sections were developed with Extra-kit (Sigma). Images were taken under an Axiovert 40CFL microscope with a 5x objective lens. BrdU- or Parvalbumin- positive cells were quantified with AxioVision Rel. 4.5 software and analyzed by one-way ANOVA.

Latent inhibition. The latent inhibition procedures were conducted in accordance with the local Animal Care Committee and the EC regulations for animal use in research (86/609/EEC). Eleven WT littermate males and 14 Disc1ₜᵣ transgenic males at 9-10 months old were housed individually under standard conditions (20-21° C., 60-65% relative humidity), with ad libitum access to water and food. They were divided into 4 experimental groups of pre-exposed WT (pe-WT, n=5), non-pre-exposed WT (npe-WT, n=6), pre-exposed transgenic (pe-Tg, n=7) and non-pre-exposed transgenic (npe-Tg, n=7). The tests took place during the light phase of a 12 h light:12 h dark cycle in two square boxes A and B. Both boxes were equipped for tone delivery (10 sec at 70 Db, 1500 Hz) and were enclosed in a sound-attenuating cubicle, while only Box A was designed to deliver 2 sec of 0.2 mA electric shocks through the metallic grid floor. Both were equipped with infrared beams (at 1 cm above floor) to detect horizontal movements.

Before electric shocks, the pre-exposed mice were acclimatized for 2 min to Box A and received 20×10 sec tone with 20 sec intervals on day 1, and 15×10 sec tone with 20 sec intervals on day 2; while non-pre-exposed ones were placed in Box A for the same durations each day with no tone. Then, all mice were given 5 sets of repeated conditioning (10 sec tone+2 sec electric shock+20 sec interval). The session was terminated after a further 40 sec interval. On day 3 animals were tested for retention in Box B, with 2 min habituation, followed by 2 min continuous tone and 2 min post-tone habituation. The horizontal locomotor activity was monitored by the numbers of infrared beam breaks. Data were analyzed statistically using one-way or two-way ANOVA for either repeated or not repeated measures followed by a post-hoc test when required. Differences with a p<0.05 value were considered as significant.

Modified Porsolt swim test (PST) and tail suspension test (TST). PST was conducted by placing individual mice (˜3 month) in a 5 L glass beaker with 3 L fresh tap water (22° C.) for 6 min. A mouse is regarded as immobile when floating motionless or making only adjustments necessary to keep its head above the water. The modified TST was carried out for 6 min, by securing the mouse tail to the edge of a shelf ˜20 cm above a large cage with deep bedding. Mice were considered immobile when they hung passively and completely motionless. Both tests were videotaped and examined by two independent observers. The immobility in the first 2 min, the last 4 min, or the whole 6 min trial was assessed. During the TST, mouse vocalizations also were recorded with a bat detector, and analyzed by BatSound Standard—Sound Analysis version 3.31, for the amplitude, frequency and nature of calls. The number of squeaks was counted in each period. Data were analyzed by one-way ANOVA and presented as Mean±SEM. p<0.05 was considered to be statistically significant.

### Example 1

### Truncated Disc1ₜᵣ EGFP Transgene and Expression

To genetically model the DISC1 truncation (FIG. 1A), we characterized a mouse BAC RP23-236F19 containing Disc1 exons 1-9 with its entire upstream sequences (FIG. 18). To facilitate the identification of the transgene, we fused an EGFP cDNA to the end of exon 8 followed by a SV40 polyA signal. The modified BAC DNA was microinjected into fertilized mouse eggs, and 3 Disc1ₜᵣtransgenic founders (M19, M20 and M22) were generated that contained the EGFP fragment. Most studies were carried out on the M19 heterozygotes and WT littermate controls unless specified otherwise, as the M22 offspring did not express the transgene mRNA, and the M20 female founder failed to transmit the transgene with reduced litter sizes (6.3±0.7 mice/litter, n=6) in comparison to other breeding pairs (8.7±0.5 mice/litter, n=29 litters, p<0.01). The M19 transgenic heterozygotes contained 2 copies of the truncated Disc1 on the background of 2 copies of full length Disc1 (FIG. 1 C), closely mimicked the genetic ratio (1:1) in the Scottish family.

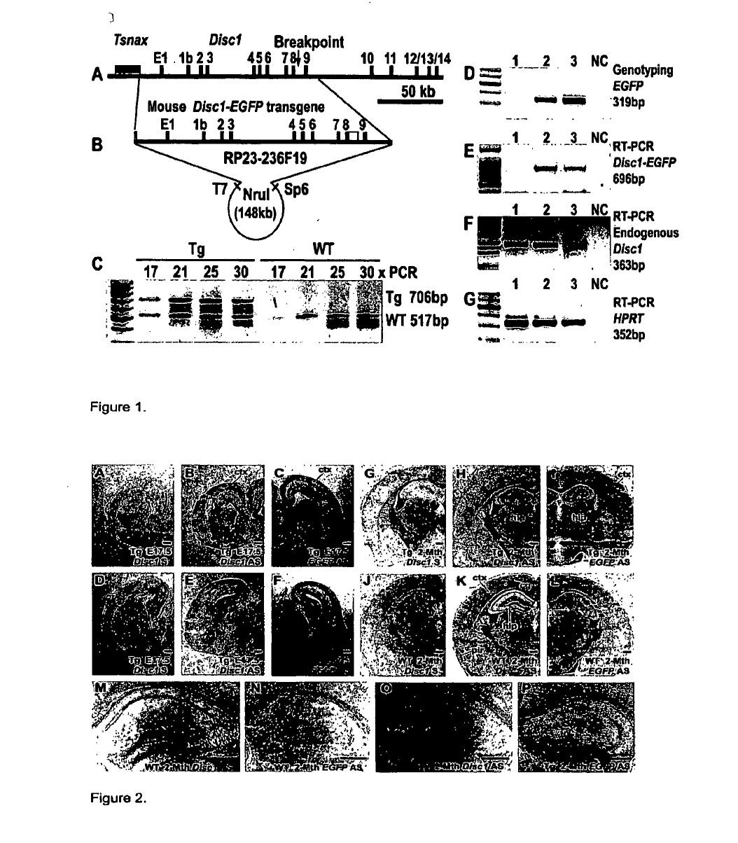

The transgene expression was assessed by RT-PCR and in situ hybridization. RT-PCR suggested that M19 transgenic mice produced comparable levels of endogenous Disc1 and Disc1ₜᵣ-EGFP transcripts in E17.5 (lane 2, FIG. 1E-G) or adult (lane 3, FIG. 1E-G) brains. To determine the spatial and temporal expression patterns of the transgene, we carried out in situ hybridization on E17.5 and 2-month brain sections with antisense Disc1 and EGFP probes. The DIG-labeled Disc1 probe was reversely transcribed from exons 12-14 of the Disc1 which was not present in the transgene, while the EGFP probe was derived from the entire EGFP coding sequence. In E17.5 developing brain, both the Disc1 and EGFP hybridization signals were localized predominantly in the cerebral cortex and hippocampus (FIG. 2A-F). In 2-month brain, the full-length and truncated Disc1 mRNA were found in the cerebellum (not shown), hippocampus and cerebral cortex including cingulate and piriform cortex (FIG. 2G-L). In the hippocampus, both the Disc1 and EGFP probes detected expression in the pyramidal layer of CA1-CA3, and granule layer of the dentate gyrus (FIG. 2M-P). These data suggest that the 148kb BAC expressed Disc1ₜᵣ-EGFP largely in the same anatomical sites as endogenous Disc1.

### Example 2

### Dilated Lateral Ventricles and Reduced Cerebral Cortex in Disc1ₜᵣ Transgenic Mice

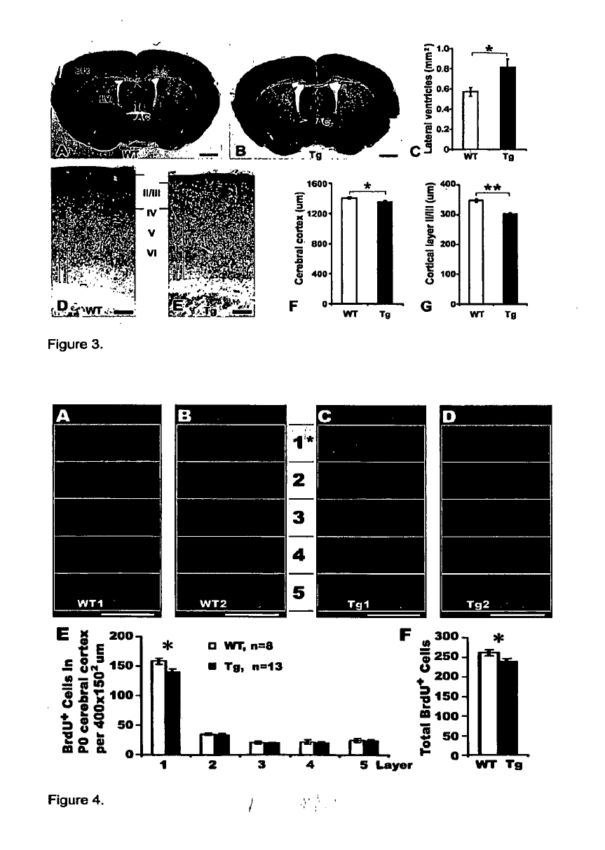

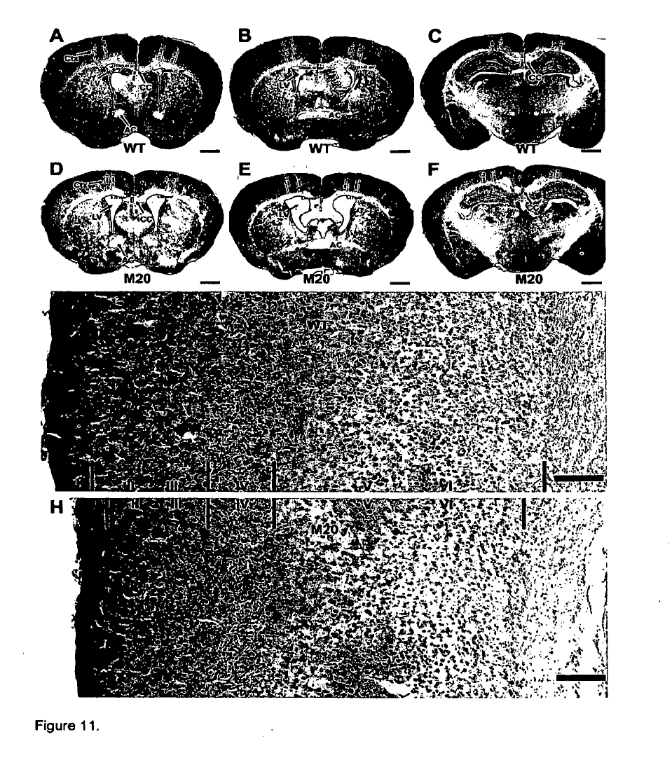

Schizophrenic symptoms usually begin in late adolescence or early adulthood, and neuroanatomic changes in lateral ventricles and cerebral cortex are seen in schizophrenic patients. We analyzed neuroanatomy in sexually mature, 2-month old transgenic mice. Mouse brains were processed histologically from 11 Disc1ₜᵣ transgenics (6 male and 5 female) and 15 WT littermates (8 male and 7 female). Sections were imaged at the plane where the anterior commissure crossed the midline (FIG. 3A-B). Sizes of the cerebral cortex and corpus callosum were quantified. The transgenic lateral ventricles were found to be dilated by ˜44% (p<0.05) (FIG. 3C). In addition, we have detected a subtle (˜4%) but significant (p<0.05) reduction in the thickness of the dorso-lateral frontal cortex (FIG. 3D-F) of transgenic mice. Remarkably, this reduction largely resulted from the thinning of cortical layers which was reduced by ˜17% (FIG. 3G). Severe but consistent neuropathologies were observed in the un-transmittable transgenic founder M20 (see FIG. 11). Compared with a littermate control, the lateral ventricles were enlarged 2.3˜3.0-folds (see FIG. 11A-B, D-E). The frontal cortex was reduced by 16% in thickness. The layers II/III and V, where pyramidal neurons resided, were particularly affected (see FIG. 11G-H). These phenotypes are consistent with mild cytoarchitectural abnormalities reported in schizophrenia (Harrison, 1999; Lewis and Levitt, 2002).

### Example 3

### Reduced Neuronal Proliferation in the Developing Transgenic Brain

In the mouse, cortical neurogenesis starts from E10.5 and is largely completed by E17.5. Cells in the ventricular zone of the dorsolateral telencephalon undergo a maximum of 11 cell divisions, and neurones at different layers are generated in a cell cycle number-dependent manner (Estivill-Torrus et al., 2002). To explore the cellular mechanisms causing thinned layers II/III in Disc1ₜᵣ transgenic mice, we carried out birth dating experiments during mid-neurogenesis.

A pulse of BrdU was injected into pregnant females at E15.5, when layer II/III neurons were formed. Four independent litters of newborn brains were processed with anti-BrdU antibody. BrdU-positive cells were quantified from equally divided areas (400 μm wide×150 μm height) of newborn cortex of 13 transgenic heterozygotes and 8 WT littermates at the lateral ventrical level (FIG. 4A-D).

Transgenic newborns showed a modest but significant reduction of BrdU-labeled cells in the outermost layer (arbitrary layer 1) of the cortex, corresponding to layers II-III in adult brain, while BrdU-positive cells in other layers (2-5) were not significantly different. The total number of BrdU-positive cells was also significantly reduced in transgenic newborns (FIG. 4E, 239.2±6.8, n=13, p<0.05) compared with WT littermates (261.5±7.50, n=8). These data indicate that truncated Disc1 causes reduced neuronal proliferation at mid-neurogenesis, which contributes to subtle alterations in the cytoarchitecture of the cerebral cortex in the Disc1ₜᵣ transgenic mice.

### Example 4

### Partial Agenesis of Corpus Callosum in Disc1ₜᵣ Transgenic Adults

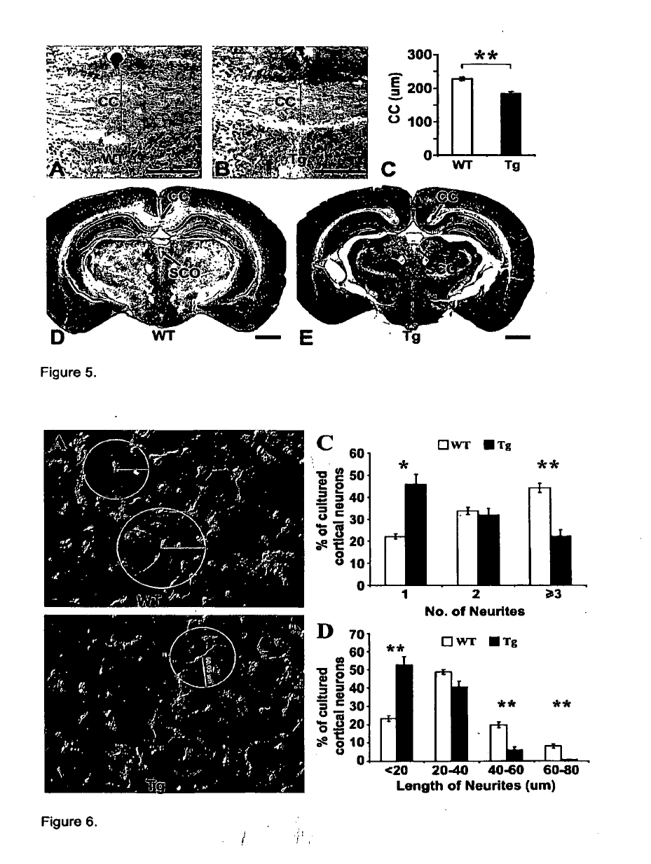

The corpus callosum consists of nerve fibers projecting from cortical neurons to communicate between the two hemispheres. To evaluate the consequence of lamination changes in Disc1ₜᵣ transgenic cortex, we have compared the thickness of the corpus callosum between 11 transgenic (6 male and 5 female) and 15 WT littermates (8 male and 7 female). At the rostral brain where the anterior commissure crosses the midline (FIG. 5A-C), transgenic corpus callosum (184.2±6.1 μm, n=11) was significantly thinner (˜20%) than that in WT littermates (228.3±5.3 μm, n=15, p<0.01). In the caudal brain at the level of the subcommissural organ (SCO), all 15 WT littermates displayed a thick layer of the corpus callosum crossing the midline (FIG. 5D). However, in the majority (9/11) of transgenic brains, the corpus callosum failed to cross the midline above the SCO (FIG. 5E). These data demonstrate clearly a partial agenesis of the corpus callosum both rostrally and caudally in Disc1ₜᵣ transgenic brains.

### Example 5

### Effect of Truncated Disc1 on Neurite Outgrowth in Vitro

Disc1 and its complex members modulate neurite outgrowth (Ozeki et al., 2003; Pletnikov et al., 2008). To further examine the origin of cortical and callosal abnormalities of Disc1ₜᵣ transgenic mice, we cultured primary cortical neurons from two litters of newborn mice. We examined 845 neurons from 4 transgenic and 1817 neurons from 8 WT littermates after 26 hours of culture (FIG. 6). In WT littermate cultures, 44.1% of neurons were found to have 3 or more neurites, and this was reduced to 22.3% in transgenic cultures (FIG. 6C). Meanwhile, the proportion of cells with a single neurite increased from 22.2% to 45.8%. We also measured the length of neurites in 845 transgenic and 1817 WT littermate neurons, and categorized them into 4 groups according to their length (FIG. 6D). Neurons with >40 μm neurites were reduced from 20.5% in WT littermates to 6.9% in transgenic cultures. On the other hand, cells with <20 μm of neurites have risen by 23.6% in transgenic mice. These data suggest that reduced neurite outgrowth may contribute to the neuropathology observed in the Disc1ₜᵣ transgenic mice.

### Example 6

### Parvalbumin Cells in Disc1ₜᵣ Transgenic Prefrontal Cortex

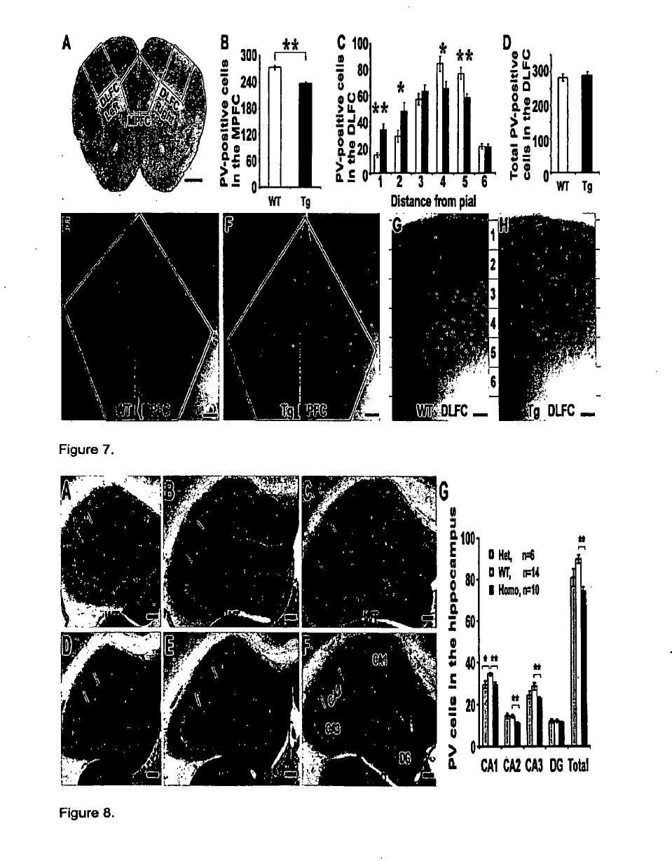

Appropriate neuronal synchronization is crucial for brain function, and a subgroup of GABA inhibitory interneurons, parvalbumin-expressing cells, is reduced in schizophrenic cortex (Lewis et al., 2005). Reduced parvalbumin immunoreactivity also was reported in transgenic mice expressing a dominant negative DISC1 cDNA under the control of a CaMKII promoter (Hikida et al., 2007). We investigated parvalbumin expression in 2-month mice (FIG. 7). Consistent with the DN-DISC1 mice, we found a significant reduction (13%, p<0.01) in the number of parvalbumin cells in the medial prefrontal cortex (MPFC, FIG. 78, E-F) of Disc1ₜᵣ transgenic mice (236.4±2.9, n=11) in comparison to WT littermates (272.2±4.7, n=14).

Additionally, we noticed that there were differences in the patterns of parvalbumin staining at the dorsolateral frontal cortex (DLFC, FIG. 7G-H). Parvalbumin-positive cells were then quantified in arbitrarily divided 6 layers (˜267 μm height each). In WT littermates, most parvalbumin cells were clustered in the inner layers. However, transgenic mice had significantly reduced parvalbumin cells in inner layers (4-5), with a significant increase of parvalbumin cells in outer layers (FIG. 7C). The truncated Disc1 did not seem to affect specification, but lamination, of parvalbumin cells, as the total number of parvalbumin cells in the DLFC was not significantly altered between transgenic (288.3±9.9, n=11, 6 male and 5 female) and WT (281.5±9.6, n=14, 8 male and 6 female) mice (FIG. 7D).

### Example 7

### Reduced Parvalbumin Neurons in Disc1ₜᵣ Hippocampus

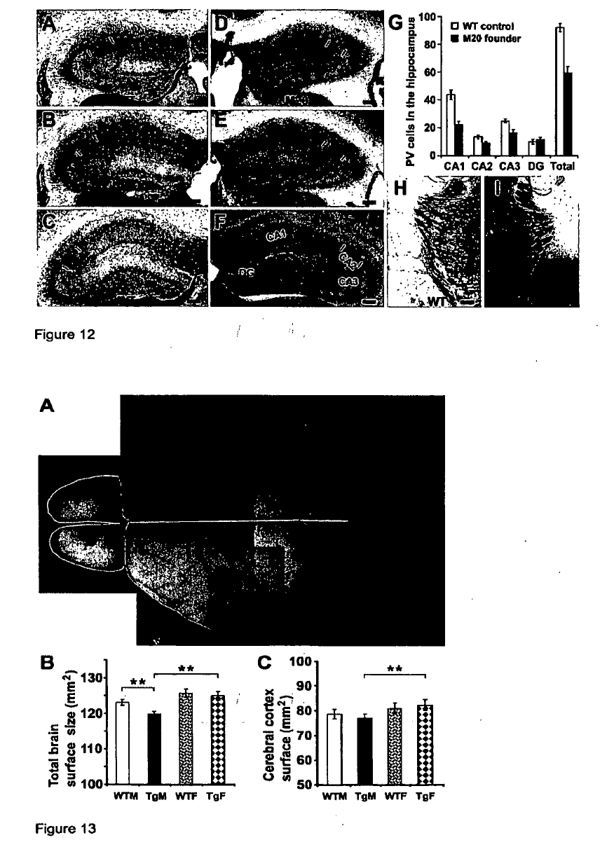

Neuropathological studies have demonstrated deficits of GABAergic interneurons in the hippocampus of post-mortem schizophrenic patients. Glutamate decarboxylase 67 (GAD67) encoding an enzyme synthesizing GABA is strikingly down-regulated in the hippocampus of schizophrenia and bipolar patients (Benes et al 2007). Independently, a profound deficit in the relative density of parvalbumin-immunoreactive neurons was found in all sub-fields of schizophrenic hippocampus (Zhang and Reynolds, 2002). To examine the GABAergic signaling in Disc1ₜᵣ transgenic mice, we have compared parvalbumin neurons in the hippocampus of 14 WT littermates, 6 heterozygous and 10 homozygous transgenic mice. A significant reduction was observed in the CA1 region of the heterozygous mice (p<0.05), with trends of reductions also in the CA3 and total numbers of parvalbumin neurons (FIG. 8G). The reduction of parvalbumin cells was significant statistically in all sub-fields of the homozygous hippocampus with the exception of the dentate gyrus (FIG. 8). Similarly, the un-transmittable transgenic founder M20 displayed dramatic reductions of parvalbumin neurons in the CA1, CA2 and CA3 regions compared with a control mouse (see FIG. 12).

### Example 8

### Defects in Conditioning of Latent Inhibition

Schizophrenic patients often have defects in pre-pulse inhibition (Braff et al., 2001) and latent inhibition (Rascle et al., 2001). Although ENU Disc1 mutants have profound deficits in latent inhibition and pre-pulse inhibition (Clapcote et al., 2007), transgenic mice with ectopic promoters do not show robust changes in pre-pulse inhibition (Hikida et al., 2007; Pletnikov et al., 2008).

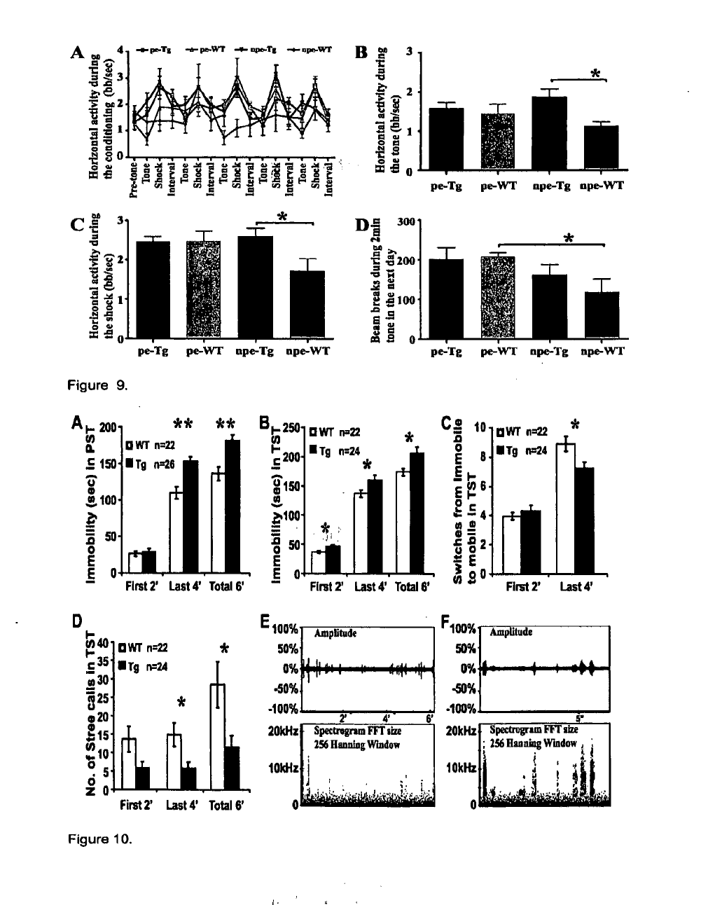

We evaluated Disc1ₜᵣ transgenic mice using a latent inhibition test of fear learning (FIG. 9). When animals were habituated to the test boxes with or without tone delivery prior to the test, there was no difference on horizontal locomotor activity (not shown). During the conditioning phase in the second day, factorial analysis of variance on the horizontal activity revealed a significant genotype/exposure interaction (FIG. 9A). A reliable difference was detected between the non-pre-exposed WT littermate and transgenic groups during the tone periods, suggesting that only the WT littermates acquired predictability of the tone (FIG. 9B). Similarly, the non-pre-exposed WT littermates had significantly reduced activity during the shock (p<0.05), while the non-pre-exposed transgenic mice remained highly active (FIG. 9C).

On the following day, animals were transferred to a different box and given 2 min tone to examine the retention (FIG. 9D). Again, only the non-pre-exposed WT littermates significantly reduced their activity during the 2 min tone, while independent of the pre-exposure, transgenic mice were not significantly different from each other. These data demonstrate that latent inhibition is established in WT littermates, but no conditioning is attained by the Disc1ₜᵣ transgenic mice.

### Example 9

### Increased Immobility in Depression Tests

Schizophrenia is often associated with depressive disorders. In the Scottish schizophrenic family, ˜35% of the carriers develop schizoaffective, bipolar or major . depressive disorders (Blackwood et al., 2001). TST and PST are common behavioral tests for depression-related behavior in animals, and a longer immobility in either of the tests is viewed as increased depressiveness. We first videotaped 26 Disc1ₜᵣ transgenic mice (13 male and 13 female) and 22 WT littermates (12 male and 10 female) in a 6 min PST. Significantly increased immobility was seen in the last 4 min and the total 6 min of the PST in the transgenic group (FIG. 10A).

This was further verified by the TST on an independent cohort of 24 transgenic (13 male and 11 female) and 22 WT littermates (12 male and 10 female) (FIG. 10B). Transgenic mice again were significantly less active throughout the 6 min TST. In addition, a reduced number of switches from immobile to mobile phases were observed in the transgenic group in the last 4 min of the TST (FIG. 10C). Disc1ₜᵣ transgenic mice did not exhibit reduced locomotor activity in the latent inhibition test (FIG. 10) or open field test (not shown). The increased immobility in PST and TST is unlikely due to a general hypoactivity of the Disc1ₜᵣ transgenic mice.

### Example 10

### Reduced Stress Calls

Schizophrenia is associated with social and communication deficits. Mice can produce a variety of social vocalizations, such as mating calls at ultrasonic frequencies beyond human hearing (30-110 KHz; Holy and Guo, 2005), and postpartum/distress calls (0˜30 KHz) audible to humans (Whitney, 1970; Whitney and Nyby, 1983). Under stressful conditions such as TST, mice squeak.

During the TST, variation in individual mouse vocalizations was evident, and the number of squeaks was counted (FIG. 10D). WT littermates squeaked frequently (28.5±6.2, n=22) during the 6 min TST, and WT males (39.7±9.6, n=12) called more than twice as often as WT females (15.0±5.1, n=10, p<0.05). Disc1ₜᵣ transgenic females tended to make fewer calls (10.2±4.0, n=11) compared with WT females. However, transgenic males squeaked 3 times less frequently (12.8±4.7, n=13, p<0.05) than WT littermate males. The reduced calls were particularly prominent in the last 4min of the test; WT males made on average 22.3±4.4 calls and transgenic males only 5.2±2.1 calls (p<0.01).

To examine frequency of the calls during the TST, we recorded vocalizations with a bat detector (FIG. 10E). No ultrasound vocalizations were detected during the tail suspension, and all calls were within the audible range (0-20 kHz, FIG. 10E-F), similar to postpartum/distress calls (Whitney, 1970; Whitney and Nyby, 1983). These data indicate strongly that Disc1ₜᵣ transgenic mice have substantially reduced vocal communication under stress conditions.

### Example 11

### Production of Other Rodents

Thus host cells according to the present invention may be comprised in a transgenic animal which is a rodent. Such animals may be prepared and\or used in analogous manner to those discussed in U.S. Pat. No. 5,912,410 and 5,898,094, or WO02/059150 which disclosures are incorporated herein by cross-reference. Other techniques are described in Ausubel, Current Protocols in Molecular Biology, John Wiley, 2001.

The transgenic animals of the invention all include within a plurality of their cells at least 2 copies of a heterologous truncated Disc1 genomic DNA sequence encoding the first 8 exons of the Disc1 polypeptide as described above.

Since it is possible to produce transgenic organisms of the invention utilizing one or more of the above-described sequences, a general description will be given of the production of transgenic organisms by referring generally to exogenous genetic material. This general description can be adapted by those skilled in the art in order to incorporate the above-described specific DNA sequences into organisms and obtain expression of those sequences utilizing the methods and materials described below.

The exogenous genetic material may be placed in either the male or female pronucleus of the zygote. More preferably, it is placed in the male pronucleus as soon as possible after the sperm enters the egg. In other words, right after the formation of the male pronucleus when the pronuclei are clearly defined and are well separated, each being located near the zygote membrane. The male pronucleus of a fertilized mouse egg is the preferred site for addition of the exogenous genetic material of the present invention.

It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material could then be added to the ovum or the decondensed sperm could be added to the ovum with the exogenous genetic material being added as soon as possible thereafter.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Thus the present invention provides methods in which two or more cloned copies of a heterologous truncated Disc1 genomic DNA sequence encoding the first 8 exons of the Disc1 polypeptide, each sequence encoding a Disc1 polypeptide which is truncated at the appropriate point, are integrated into the genome.

The number of copies of the DNA sequences which are added to the zygote is dependent upon the total amount of exogenous genetic material added and will be the amount which enables the above integration to occur. Theoretically, although two copies are required for the invention, numerous copies will be utilized, for example, 2-20,000 copies of a gene, in order to insure that two or more copies are functional.

Copy number may be determined, for example, by semi-quantitative PCR and/or Southern hybridization.

Following injection, fertilized eggs are implanted in pseudo pregnant females and are grown to term to provide transgenic mice whose cells express proteins related to the pathology of the relevant disease.

### Example 12

### Social Interaction of Disc1ₜᵣ Transgenic Mice

Social recognition paradigms are useful in the understanding of how the brain processes social information and regulates social behaviour, which could lead to the understanding of psychiatric disorders such as schizophrenia, specifically affecting social behaviour. Social withdrawal is one of the most characteristic negative symptoms of schizophrenia.

The presumption in this study was that social memory in mice is established following a brief encounter with a stranger mouse, which allows for collection and storage of information regarding the identity of the stranger (sociability phase). Therefore during the social memory phase (conducted after several minutes interval), the test mouse prefers a novel stranger over the previously encountered subject mouse.

For the study, Disc1ₜᵣ transgenic mice were of mixed gender, maintained on a C57Bl/6×CBA background and of three genotypes. Animals were group-housed except during the social interaction test sessions (3-4 animals/cage). Experiments were conducted with adult mice aged 4-5 months or 8 months and Stranger mice of matched gender (C57BL/6). Stranger mice were housed in a separate room from test subjects. All mice were kept on a 12:12-h light-dark cycle, and the experiments were always conducted during the light phase of the cycle. With exception of the testing times, the mice had free access to food and water.

The social testing apparatus was a three-chambered white Perspex box and each chamber was 20cm×42cm×22cm (length/width/height). Dividing walls were made from clear Perspex, with small rectangular apertures (8 cm in diameter) allowing access into each chamber. One side-chamber contained a stranger mouse which was confined in a cylindrical Perspex cage. The cage still permitted visual, olfactory, auditory, and some tactile contact between the stranger and the test mouse, without the threat of aggressive behaviour. The opposite chamber contained an empty Perspex cage in the case of sociability testing or a new stranger mouse for preference for social novelty/social memory.

Subject trajectories and parameters were recorded by video and Ethovision (Version 3.1, Noldus, Netherlands) which extracted and stored the X-Y coordinates of the subject's position at sample points every 0.08 s. Principal parameters were then analysed using Ethovision software. A target area was defined in the software to determine direct social contacts and was based on the optimal distance for subject mice to sniff at a stranger inside a small cage (4 cm).

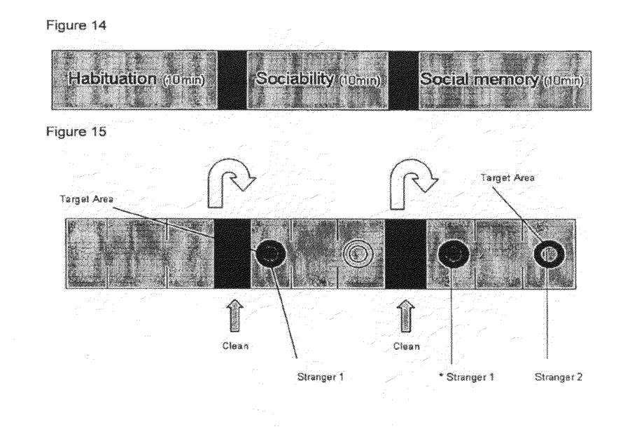

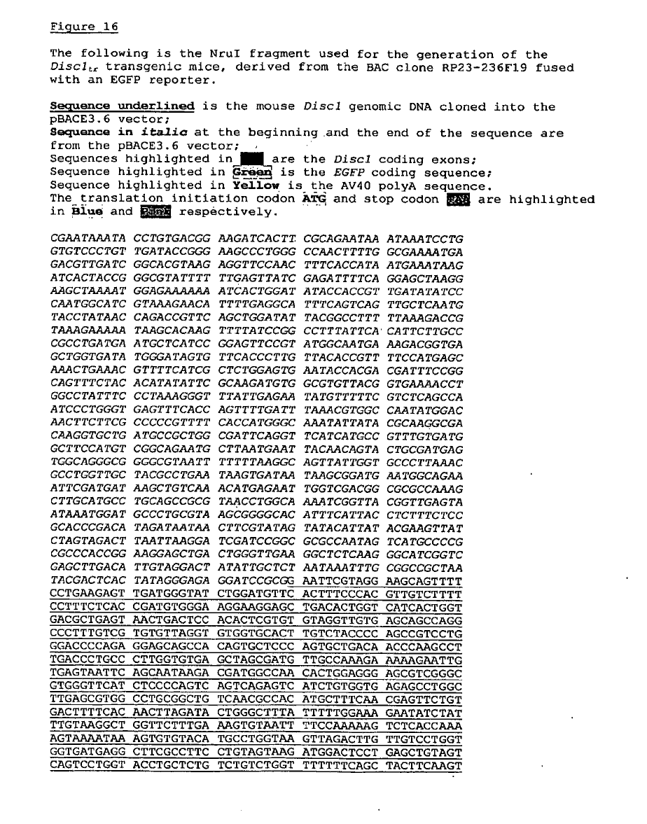

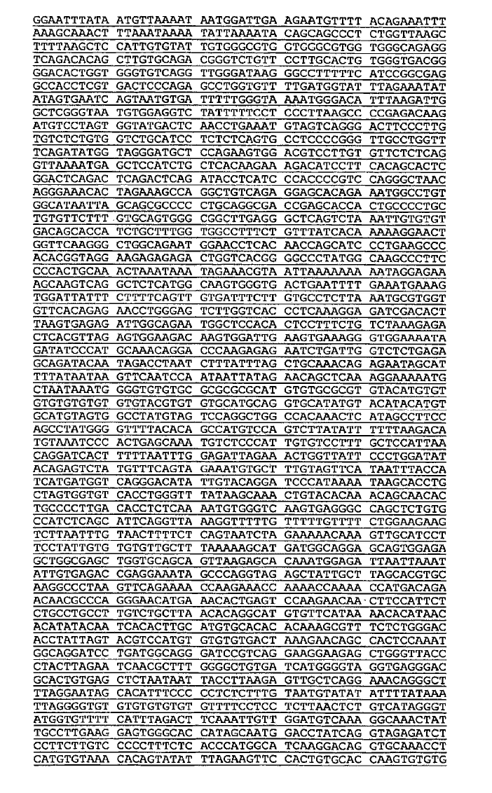

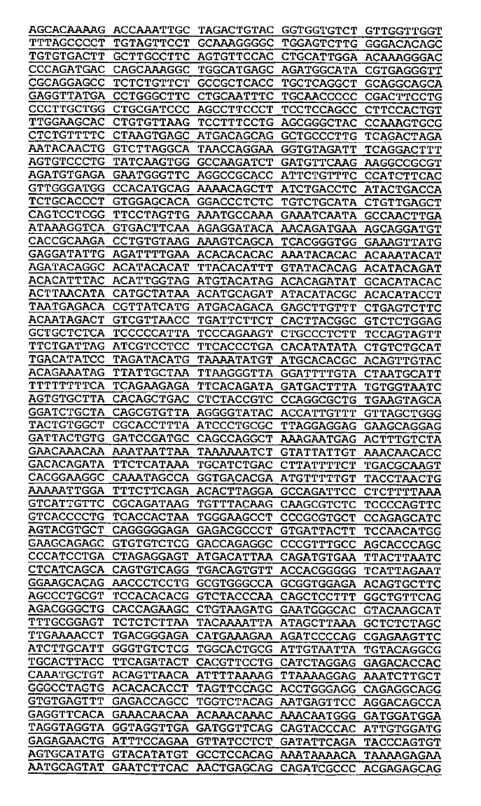

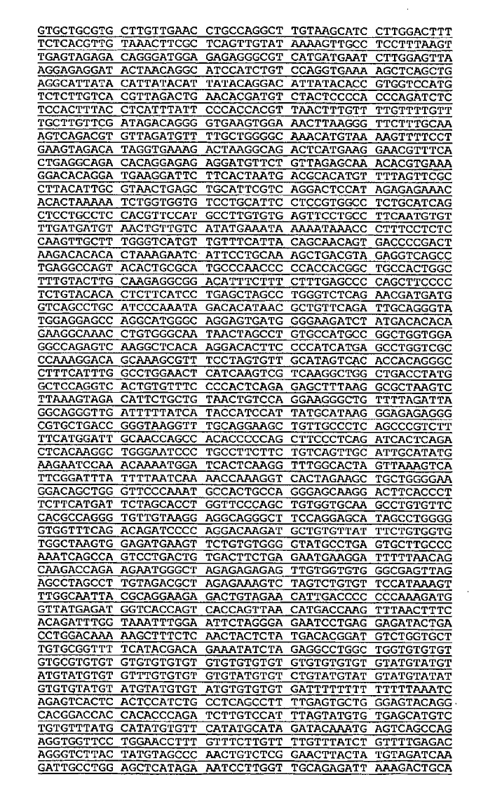

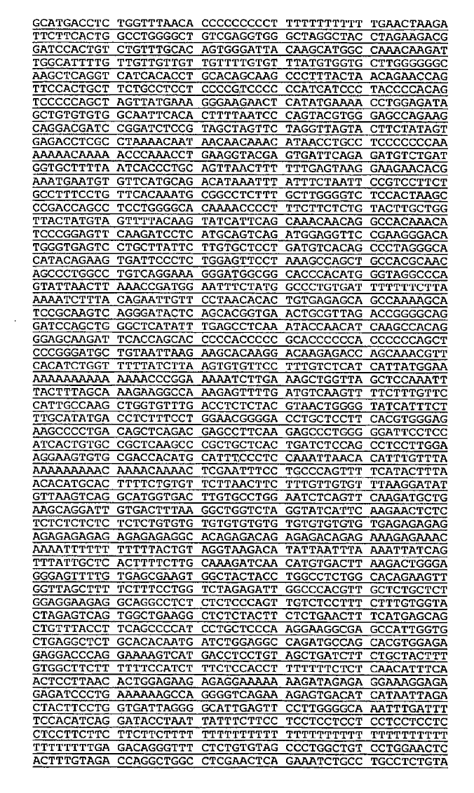

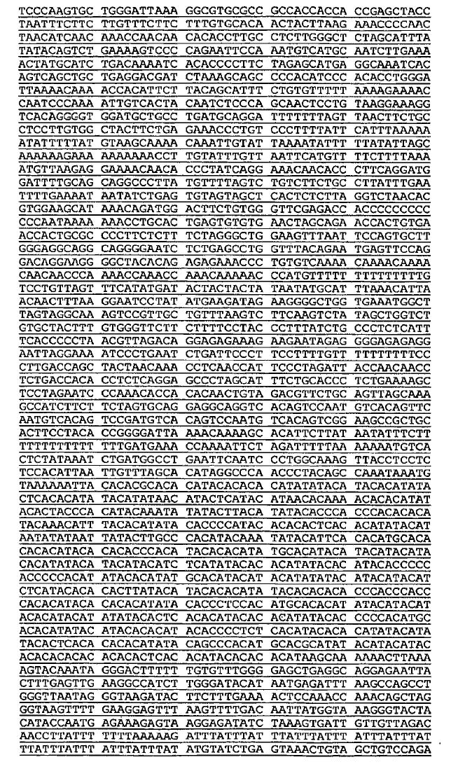

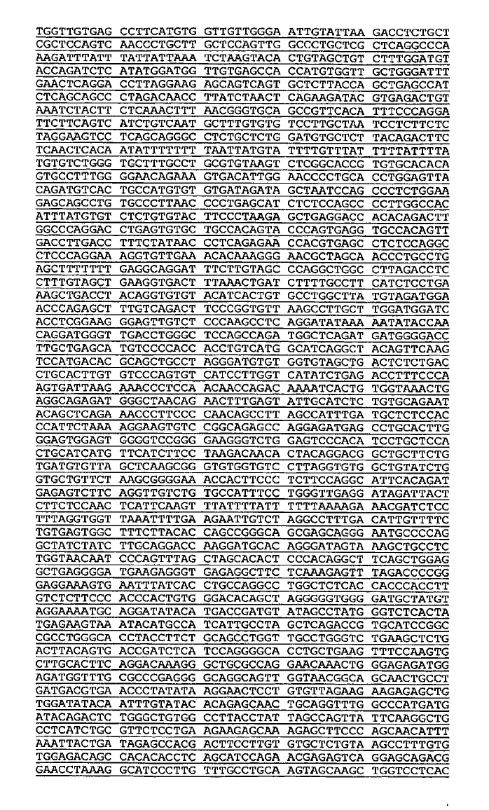

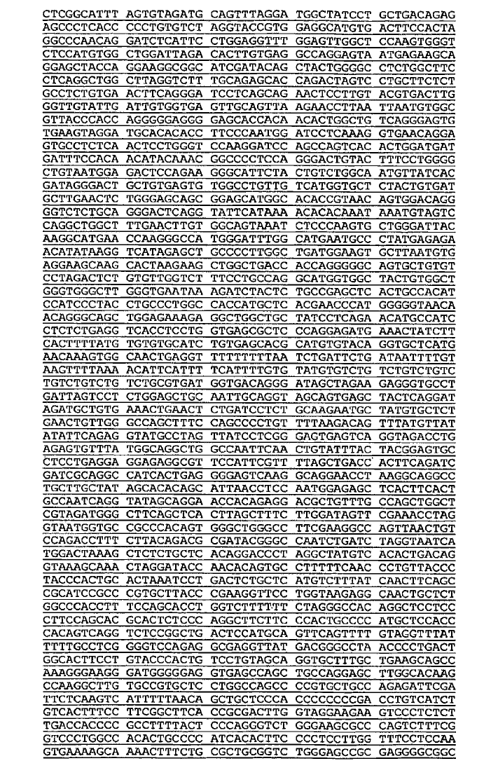

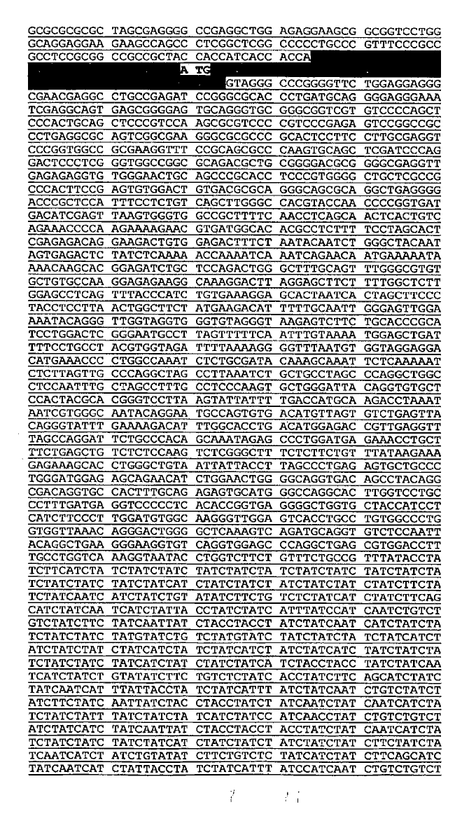

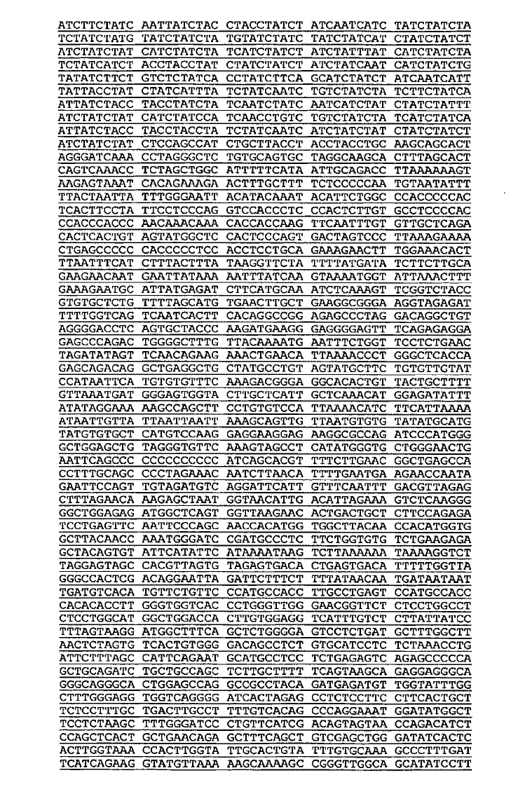

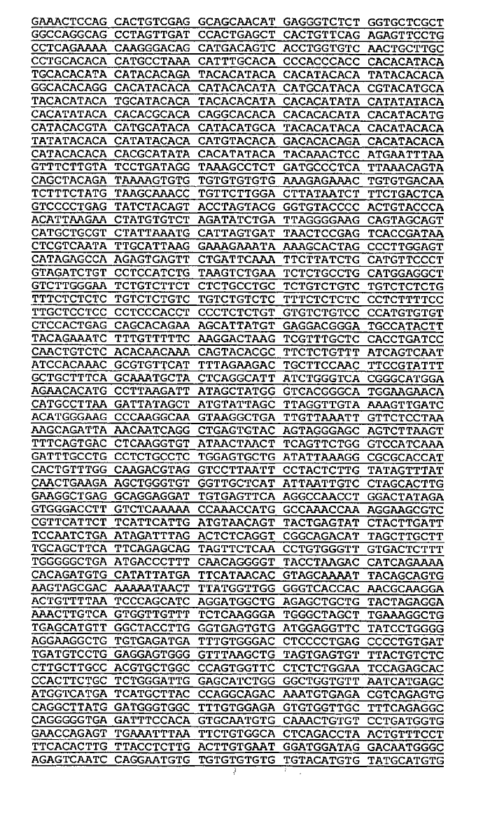

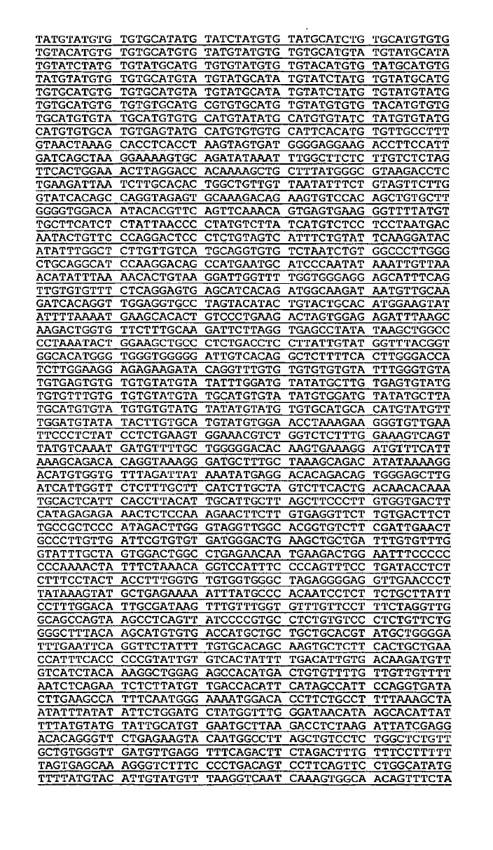

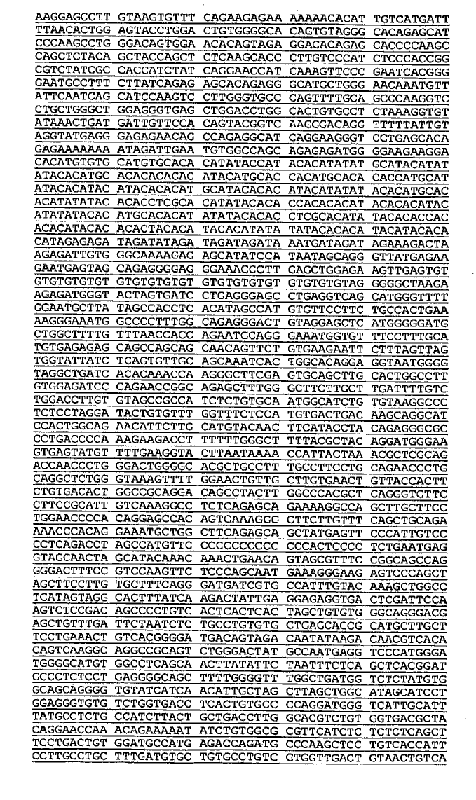

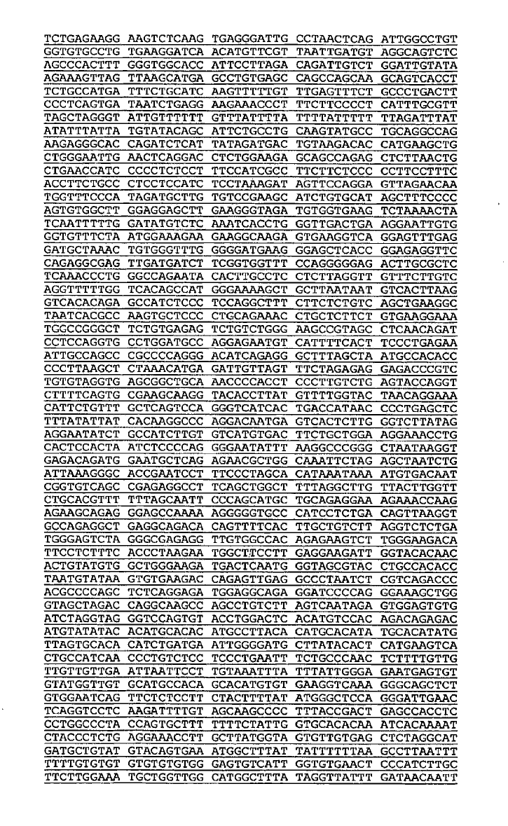

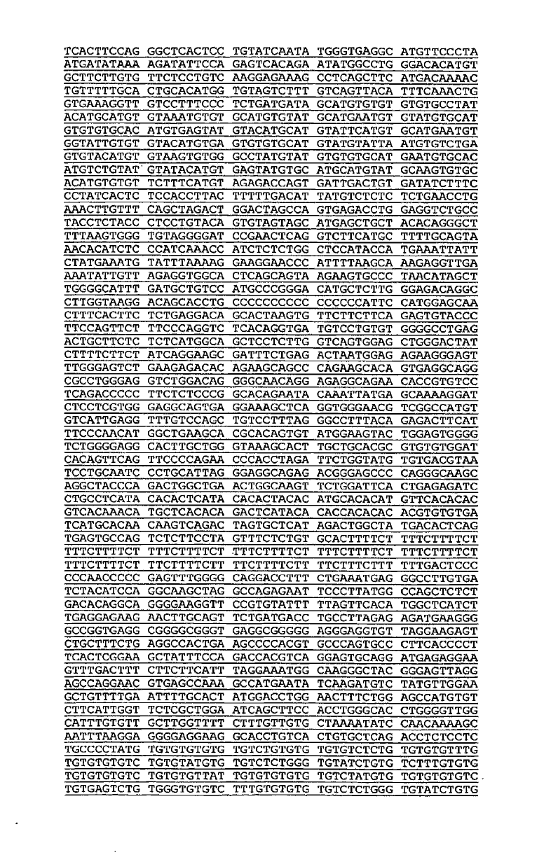

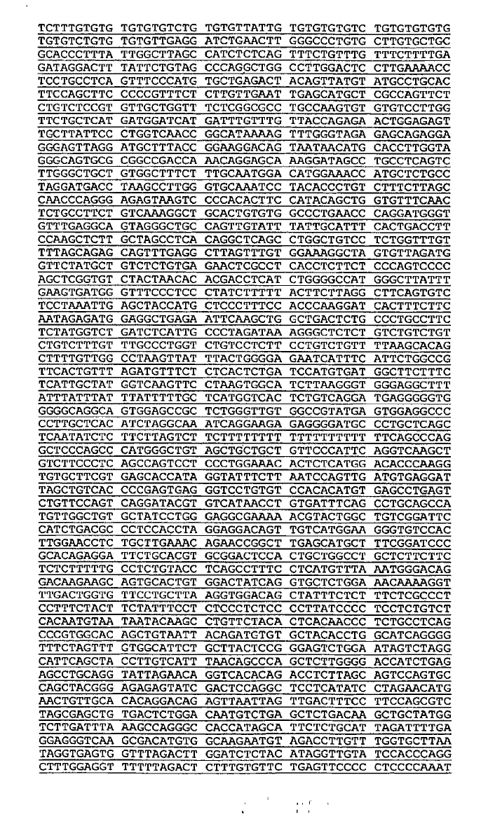

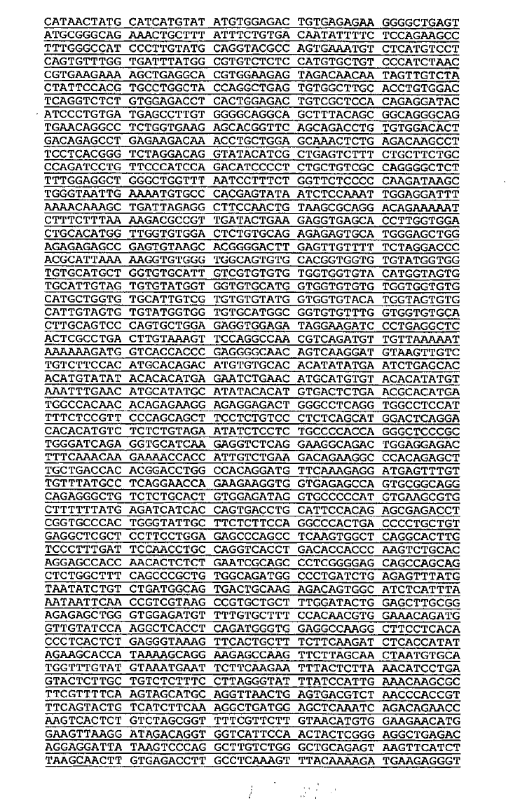

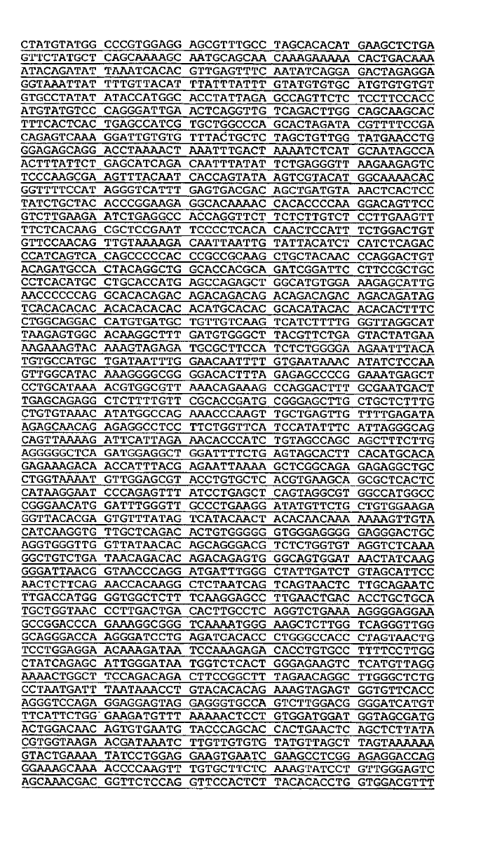

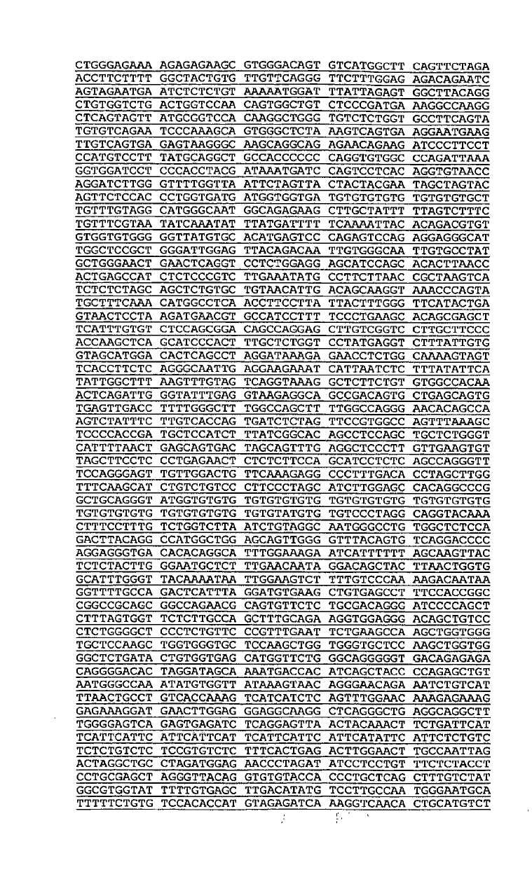

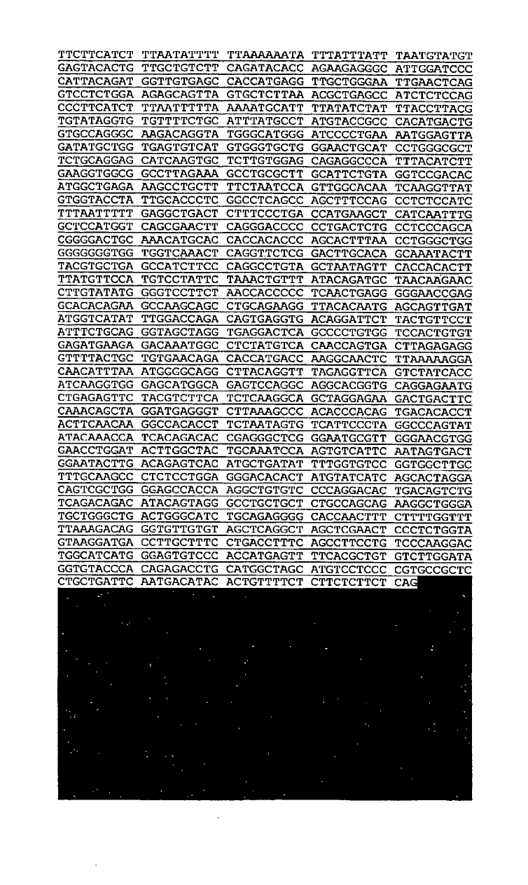

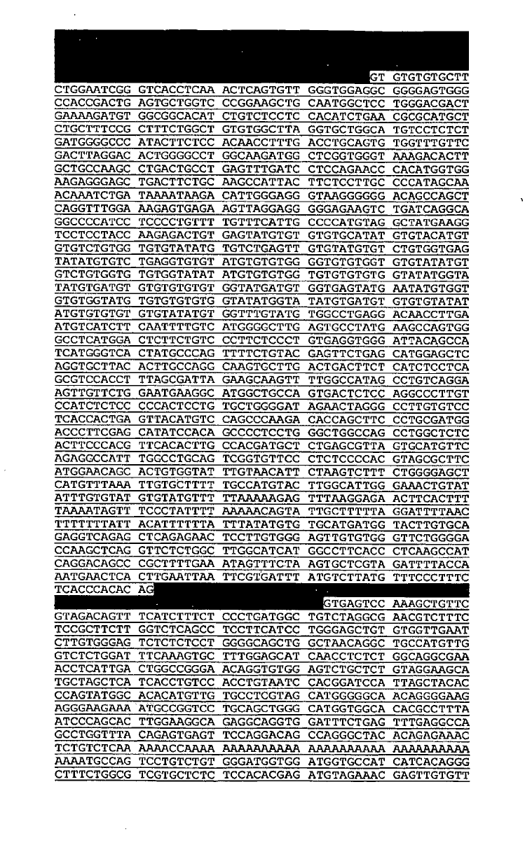

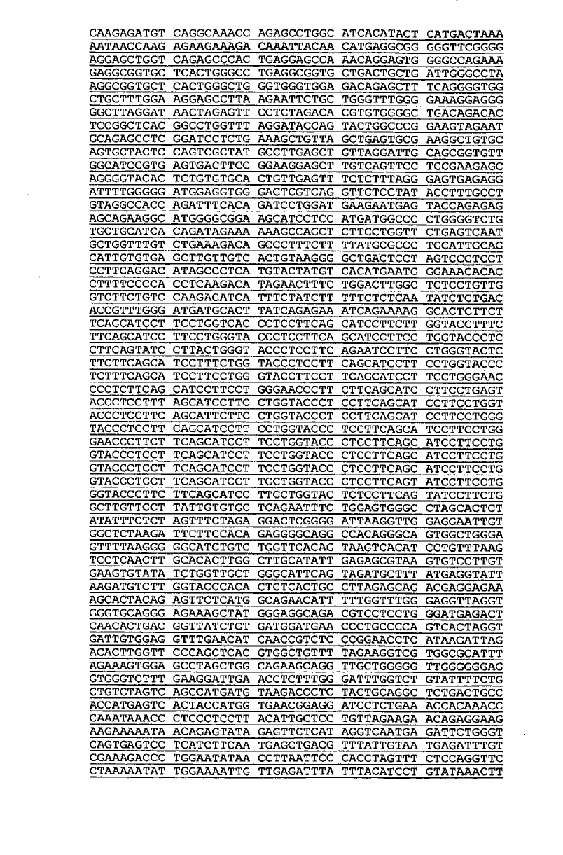

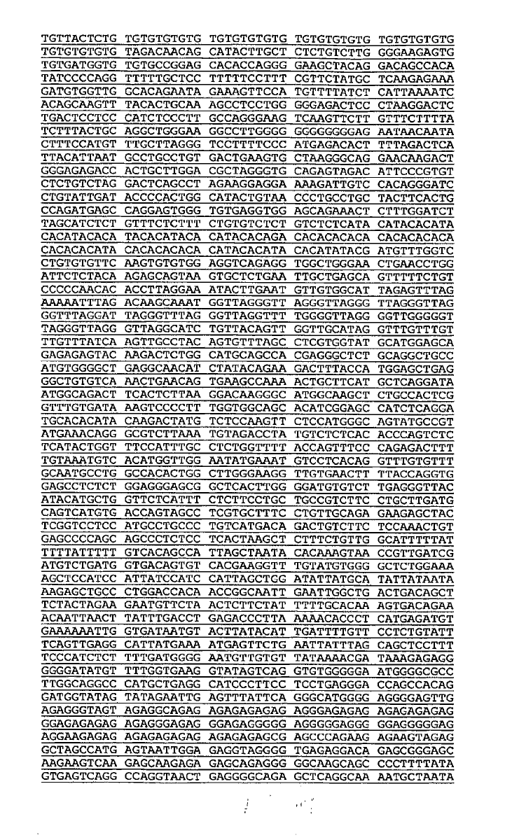

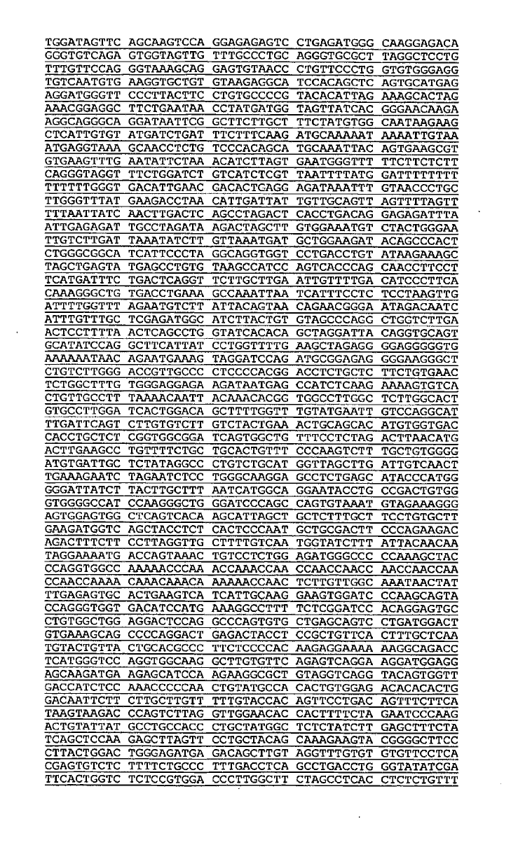

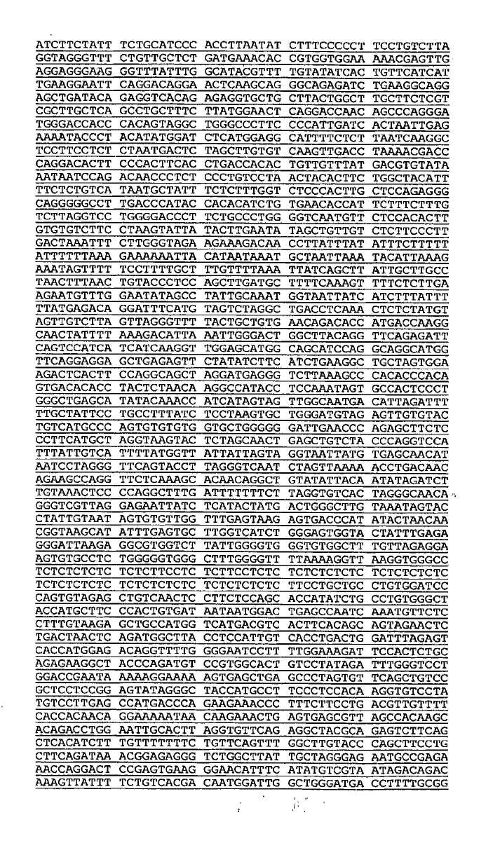

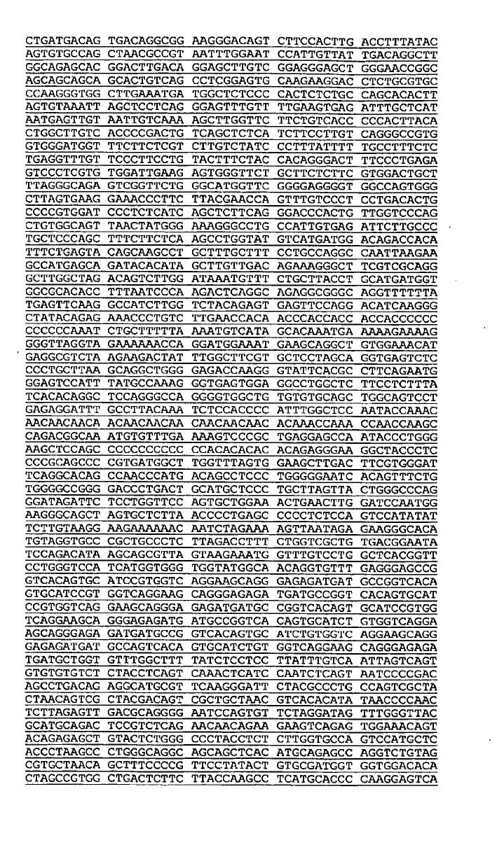

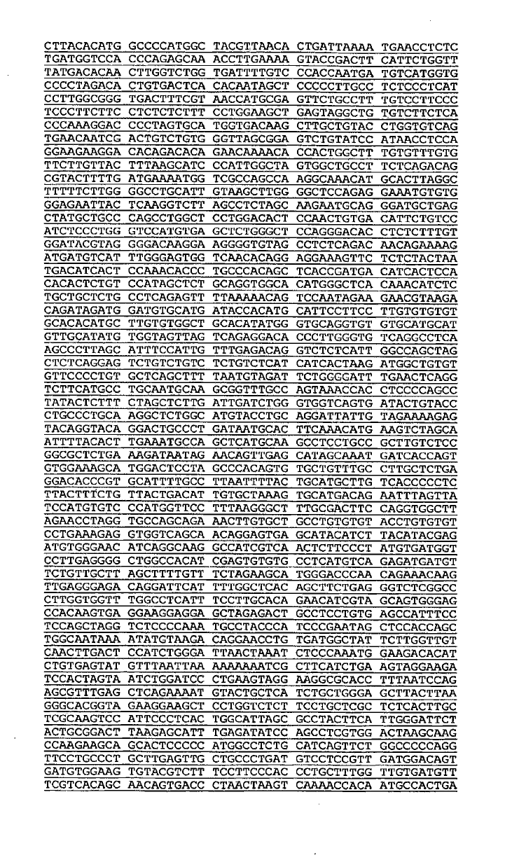

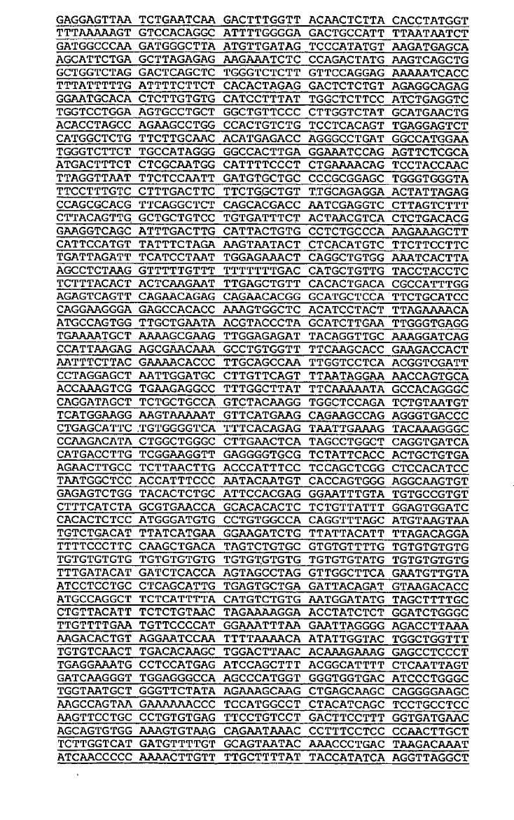

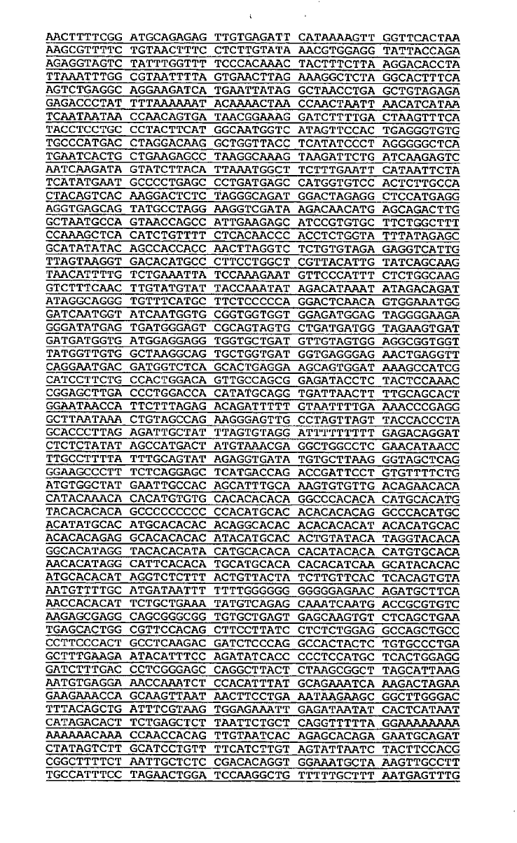

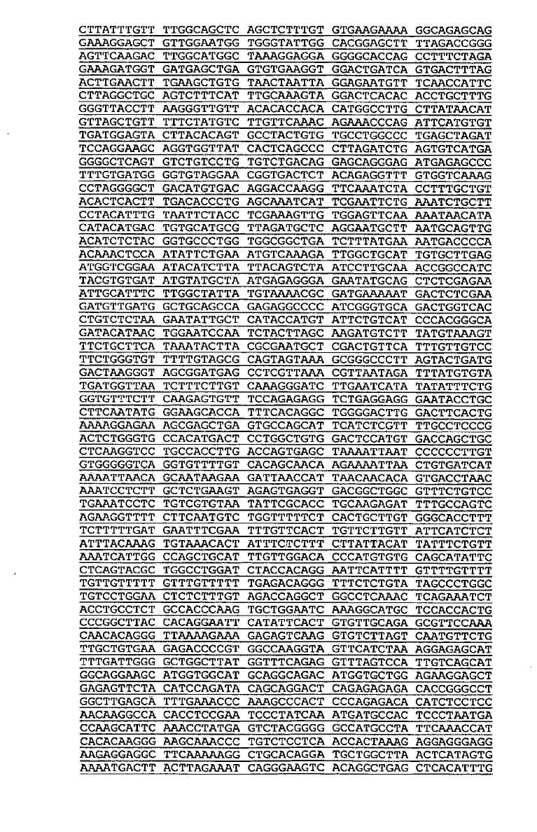

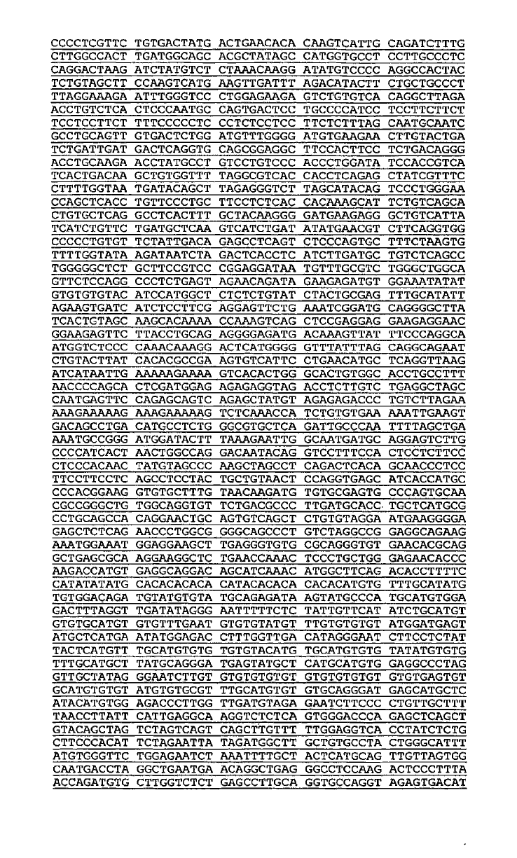

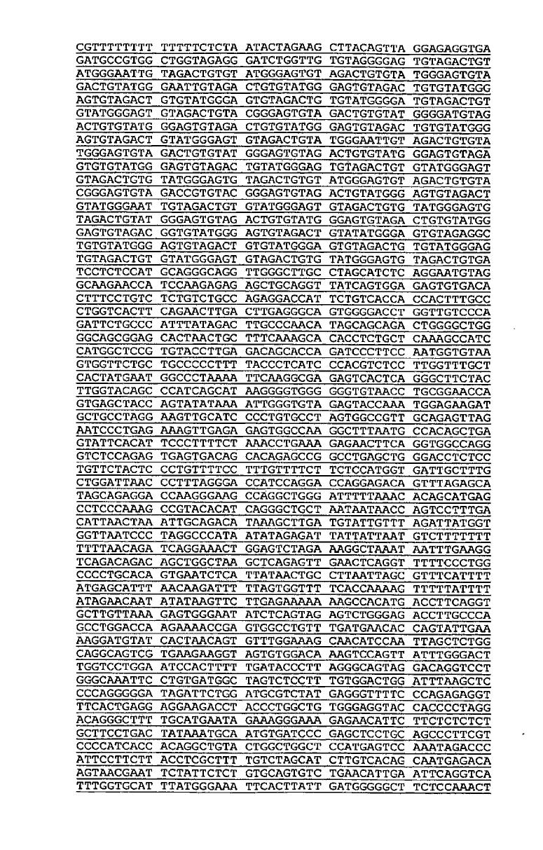

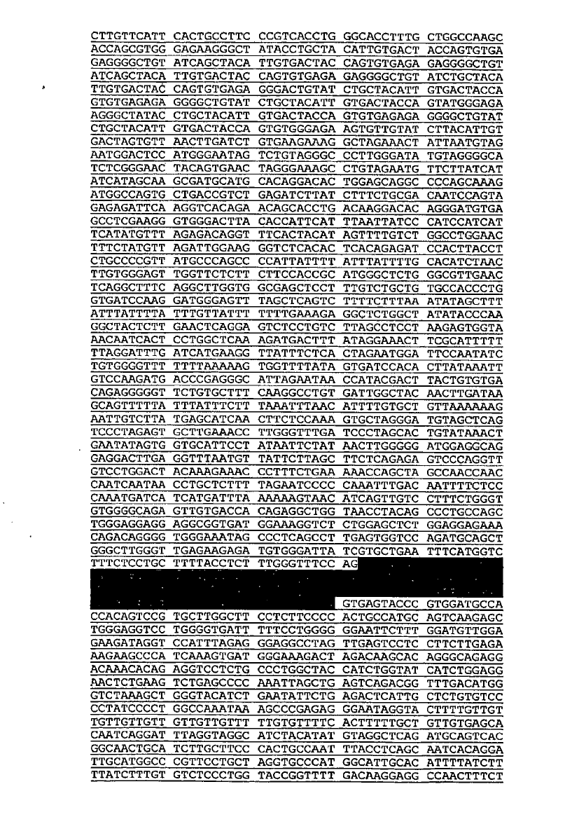

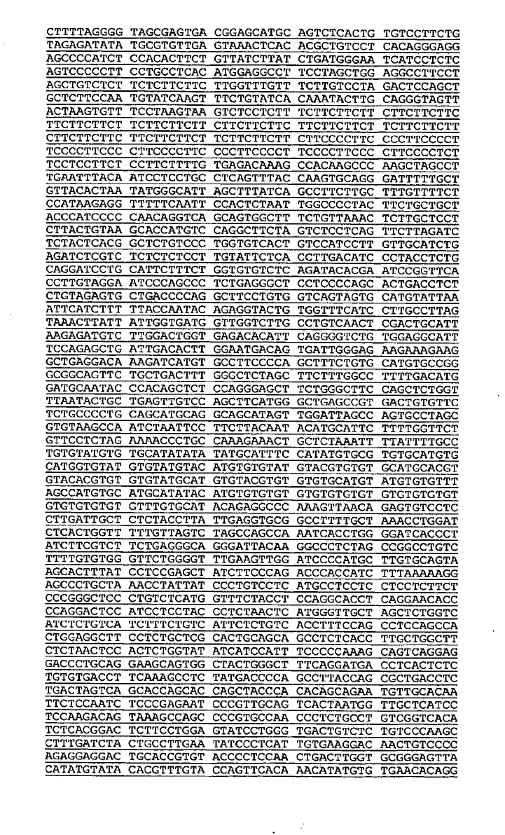

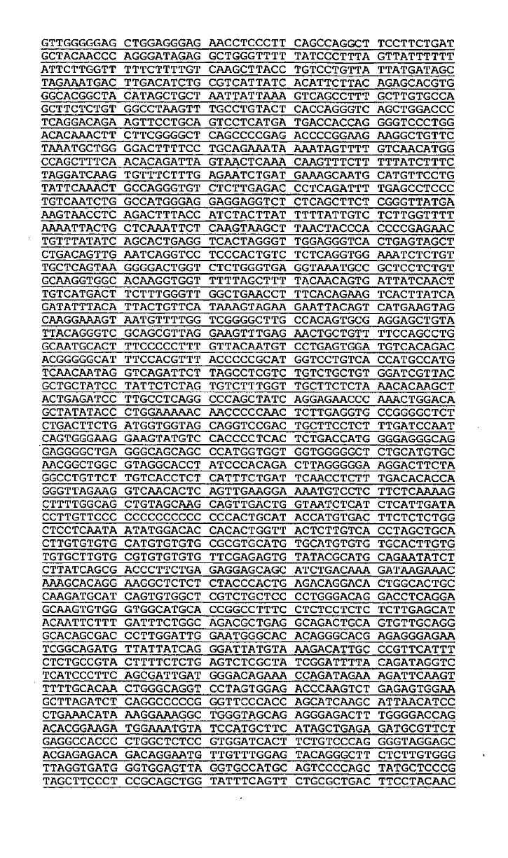

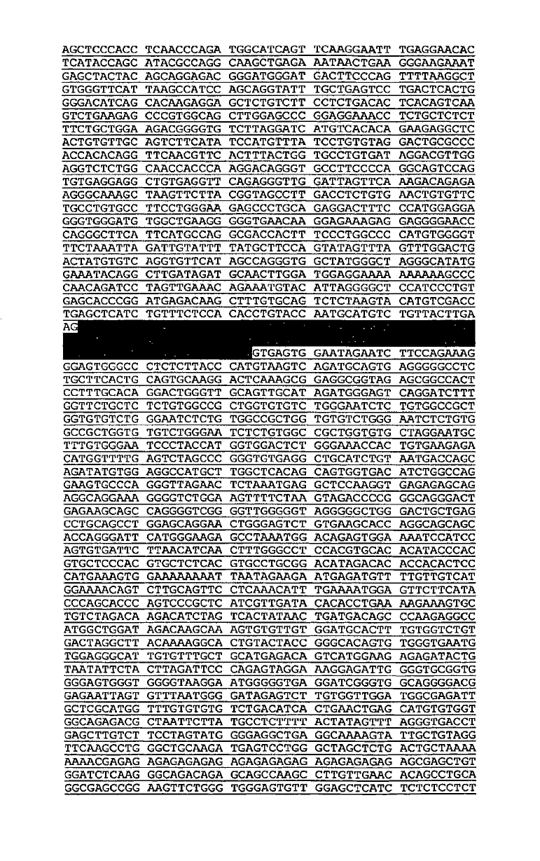

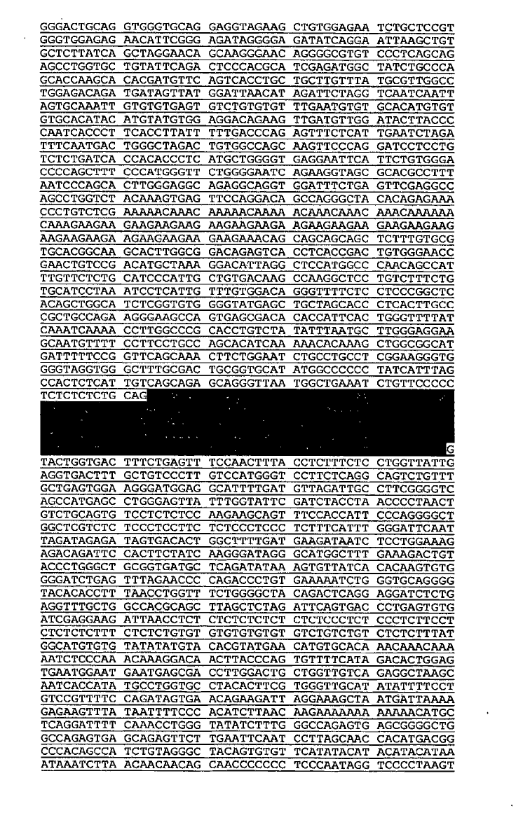

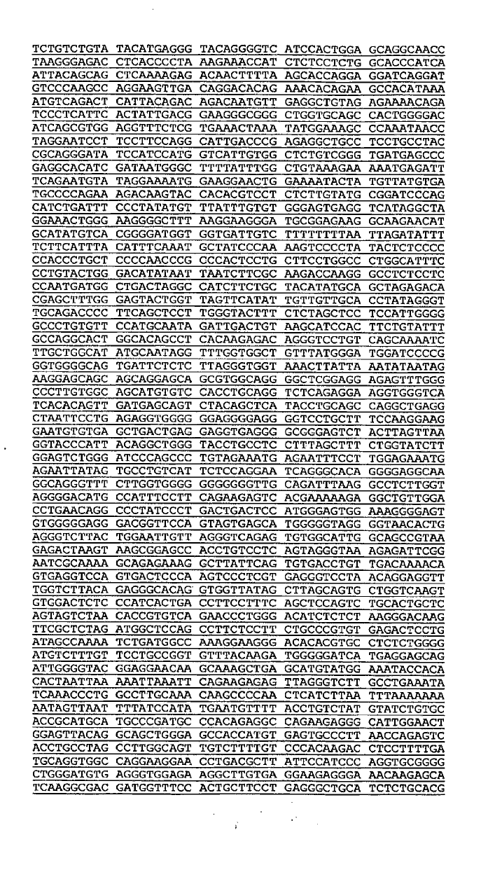

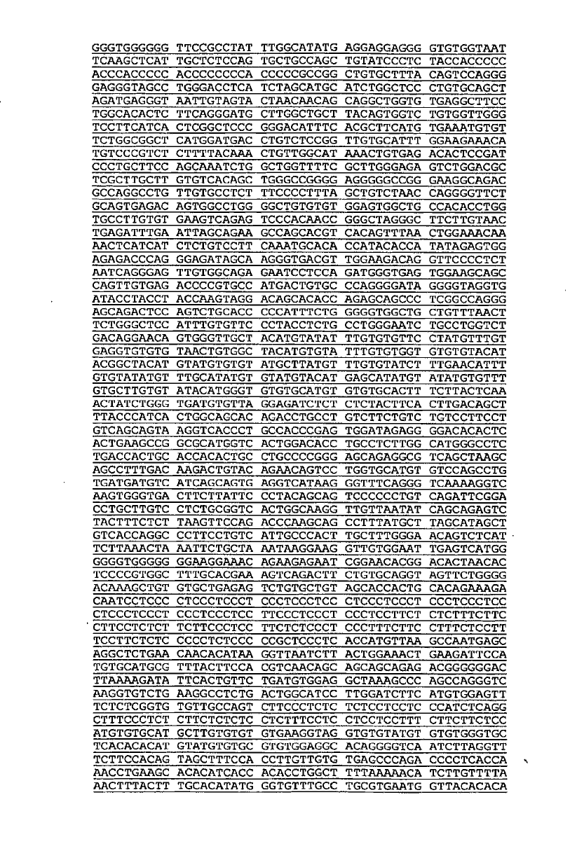

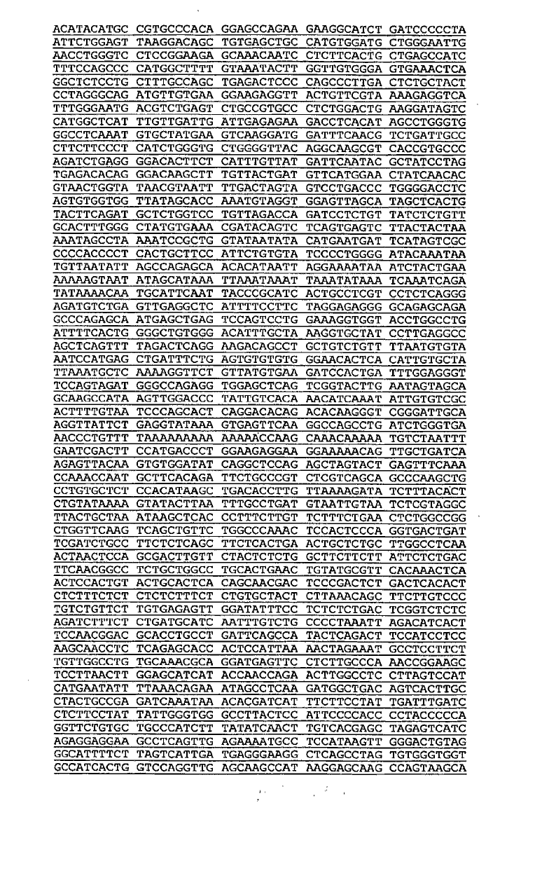

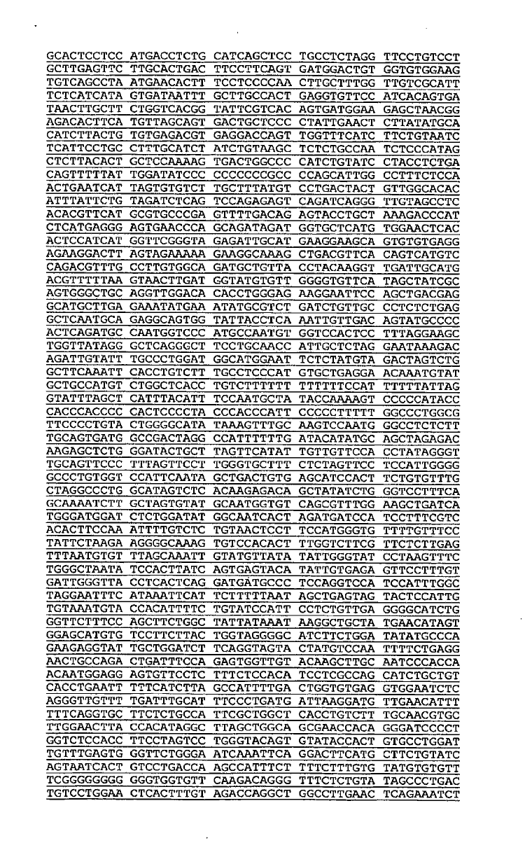

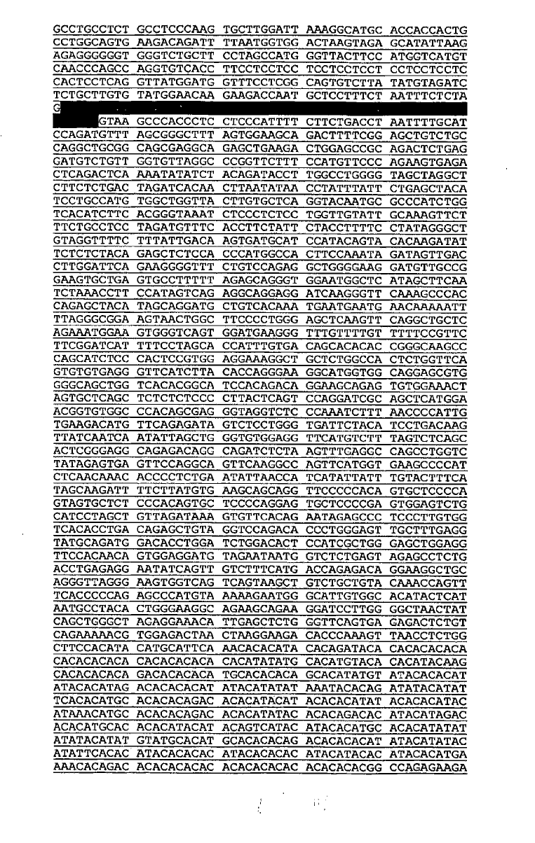

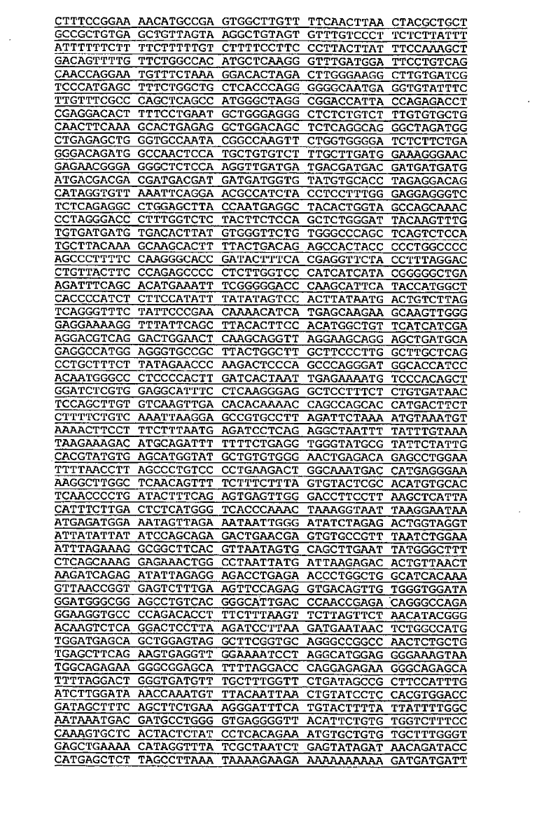

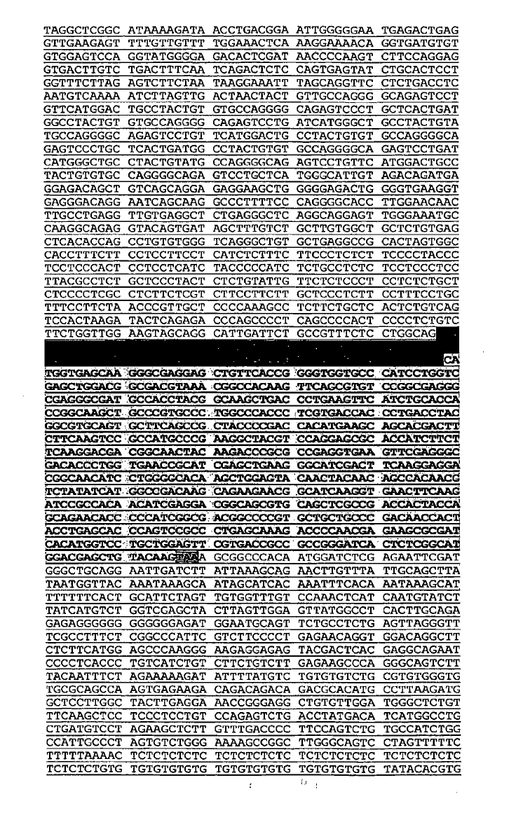

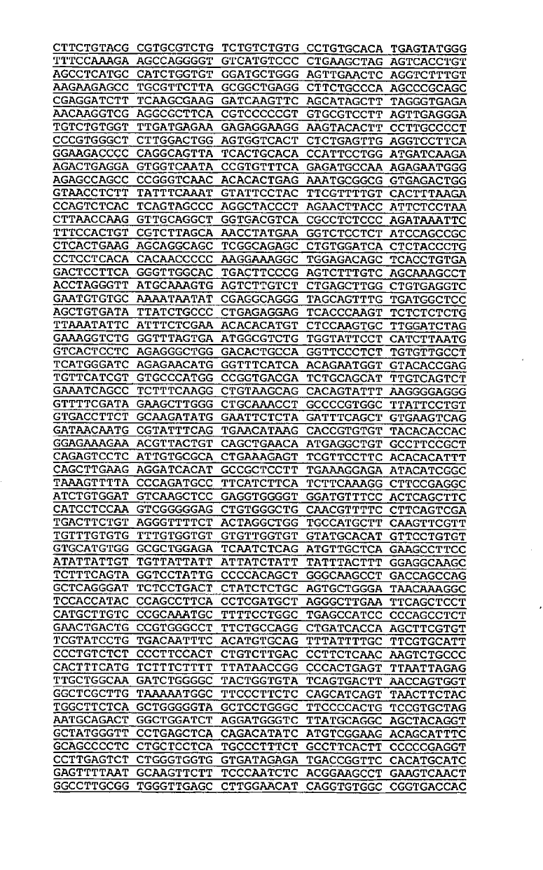

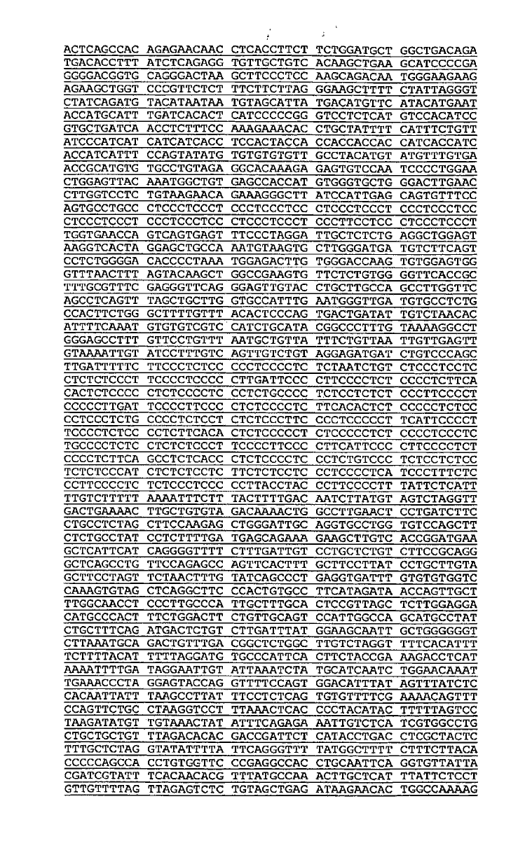

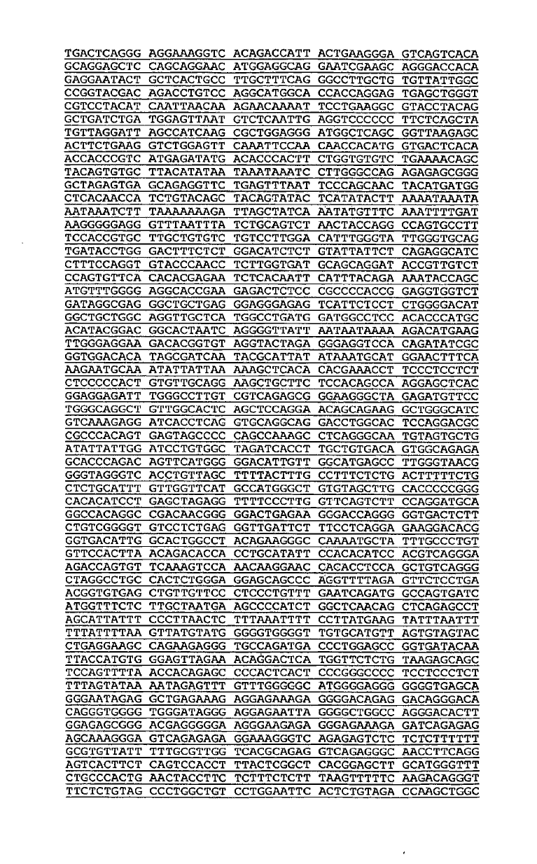

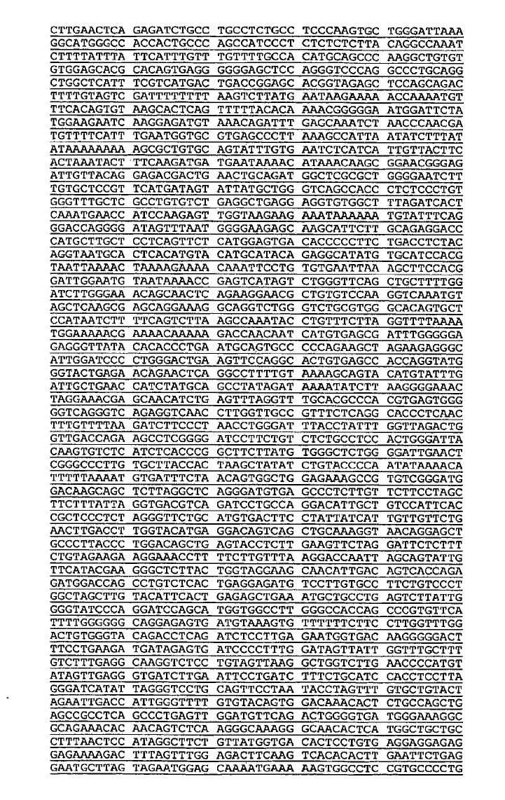

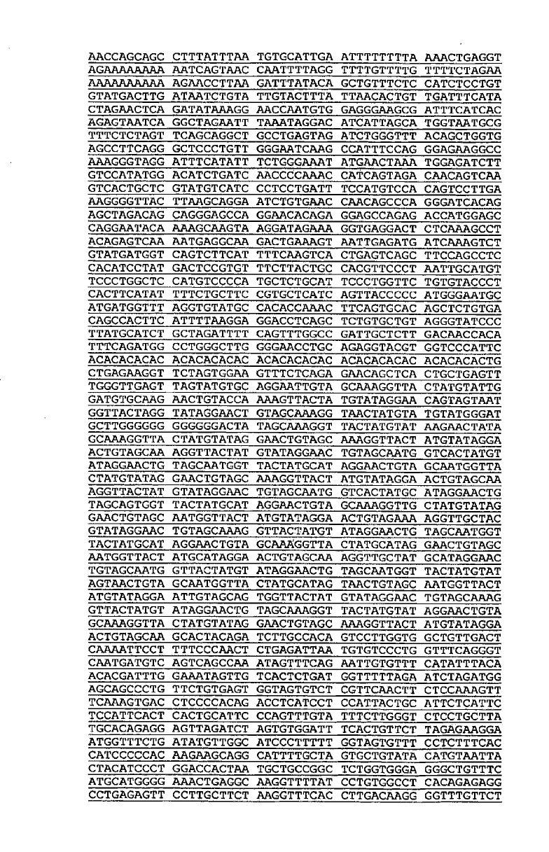

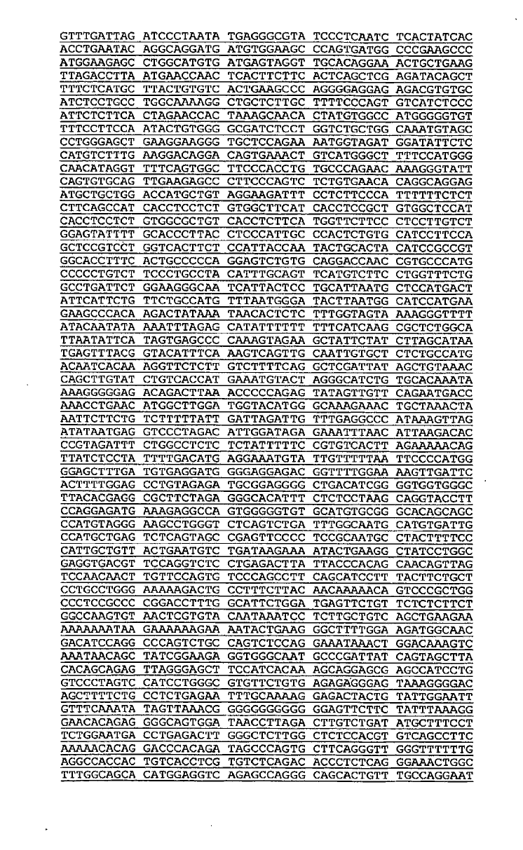

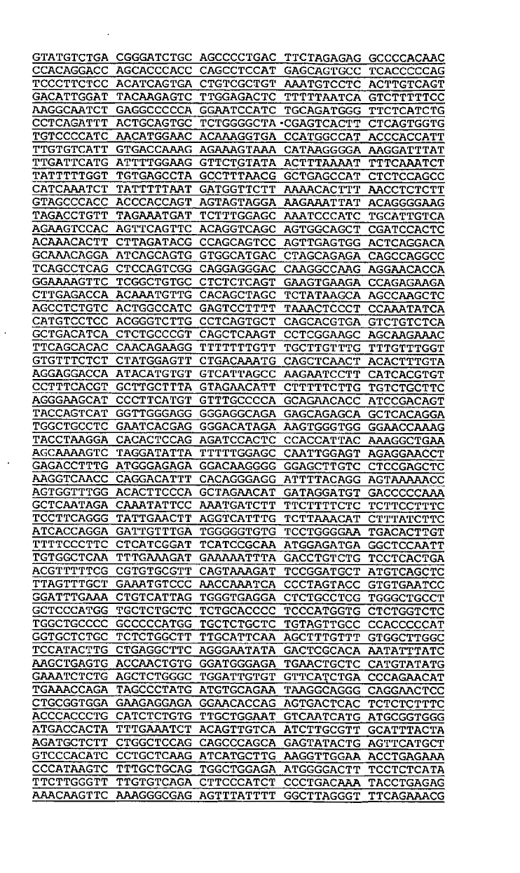

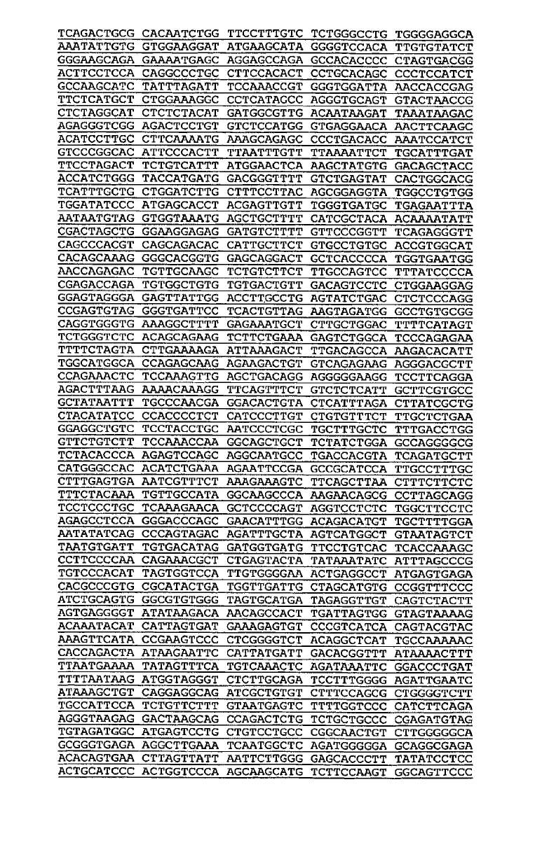

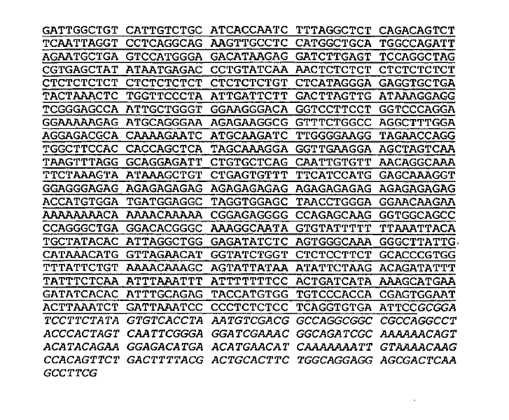

For the social recognition experiment, we used the protocol depicted in FIGS. 14 and 15. All mice were placed into an individual cage for 2 minutes prior to the experimental sessions and during experimentation released into the centre chamber from the same position while facing away from the experimenter. Initially the test mouse was placed into the empty apparatus, allowing the mouse to explore the box including the empty cages for a period of 10 minutes (habituation and object exploration). The mouse was then removed to an empty cage for 5 minutes and the dividers placed in the arena to block entry into the East and West chambers. The test mouse was then reintroduced to the arena (centre chamber only) for a period of 5 minutes (not recorded), before again being removed to an empty cage. Following an inter-trial interval of 5 minutes, stranger 1 (sex depending on the gender of test subject) was placed into one of the cages for the sociability trial and an identical empty Perspex cage placed in the opposite chamber. The test subject was then released into the centre chamber and a 10 minute test to quantify preference for sociability was undertaken. The test subject was then removed to the empty cage for a further interval of 5 minutes. To investigate social memory the original stranger mouse (stranger 1) remained in place and a new unfamiliar mouse (stranger 2) was placed in the opposite cage. Again the test subject was released into the centre chamber and social memory investigated for 10 minutes. Strangers remained in the same geographical location during both phases of the experiment to avoid potential confusion due to smell cues caused by cleaning. The floor and the walls of the arena were thoroughly cleaned with 70% ethanol between subjects.

The following parameters were measured and analysed, using Prism for Windows:
Time spent in compartments during habituation: This would reveal any habituation bias for a compartment.
Time spent in compartments during sociability and social memory trial.
Time spent in immediate vicinity target areas (mouse at 4cm outside edge of small cage): direct interaction zone between the subject mouse and stranger mice.

The advantage of these tests are their short-term nature and the absence of a procedural component, thus allowing us to conduct repeated test sessions without interference from previous experiences.

The studies showed that WT, and Disc1ₜᵣ heterozygous and homozygous mice displayed preference for the unfamiliar mouse over the unoccupied chamber during the sociability phase, and spent significantly longer in the vicinity of the unfamiliar mouse, indicating intact sociability in Disc1ₜᵣ mice.

During social memory, whilst WT mice spent significantly longer in the vicinity of the new unfamiliar mouse, this effect was reduced (in the 3-4 month heterozygous) or absent (in the 3-4 month homozygous, and 8 month heterozygous and homozygous) Disc1ₜᵣ transqenic groups, indicating an impairment of social recognition memory.

Thus, although sociability is intact in Disc1ₜᵣ transgenic mice, there is a significant deficit in social recognition memory.

### Example 13

### EEG Recordings

Schizophrenics often present with sleep abnormalities and altered EEG, especially in the pre-frontal cortex (for review, see Cohrs 2008). It is believed that such abnormalities may play a role in the perceptual disturbances typical for the disease, and may serve as a translational biomarker.

In the present study, Disc1ₜᵣ transgenic mice carrying a truncated Disc1 gene were used to analyse EEG and activity patterns (Shen et al., 2008). These mice may provide a suitable experimental model to study the basis of mental illness and explore potential treatment strategies.

The experiments were performed with transgenic mice heterozygous or homozygous for the Disc1ₜᵣ transgene on the background of two normal copies of the endogenous mouse Disc1 gene; WT littermates were used as controls. These cohorts were placed in a combined PhenoTyper and EEG study (for PhenoTyper report, see TMRC_{—}133_PT_{—}002) and consist of a total of 51 mice aged 5, and 51 mice aged 9, months at EEG recording.

Once recordings were downloaded, they were categorized according to quality (visual inspection) and the best were selected for analysis consisting of:

5 month cohorts

Group 1: WT (n=6);

Group 2: Disc1ₜᵣ Heterozygous (n=6) [termed ‘het’];

Group 3: Disc1ₜᵣ Homozygous (n=6); [termed ‘homo’]

9 month cohorts

Group 1: WT (n=6);

Group 2: Disc1ₜᵣ Heterozygous (n=6) [termed ‘het’];

Group 3: Disc1ₜᵣ Homozygous (n=6); [termed ‘homo’]

Mice were individually housed in standard macrolon cages (82 cm² free space) and kept in a controlled holding environment with a 12-hour day-night cycle (lights on at 7 a.m.). They had free access to water and standard rodent food pellets. A layer of sawdust served as bedding for the cages; no further enrichment was provided to avoid interference with EEG head devices. A Plexiglas divider was used to separate the animals from the food hopper and avoid damage to the head and the electrode assembly. All procedures concerning animal care and treatment were in accordance with international standards on animal welfare and UK Home Office regulations.

For surgery, mice were anesthetized with 3% isoflurane in medical grade oxygen and maintained on 1.5% isoflurane anesthesia during surgery. Epidural gold plated screw electrodes were placed at the following locations to record EEG from prefrontal cortex (2 mm anterior to Bregma/close to midline), left and right hippocampus (2 mm posterior to Bregma/1.5 mm lateral to midline). Reference and ground electrodes were placed at a neutral location above the parietal and occipital cortices. Electrodes were soldered and assembled into a 6-pin adaptor and fixed on to the skull by a mixture of Durelon dental cement and glue. Once the cement dried, the animal was removed from the stereotaxic instrument and injected with 0.5 ml saline (intraperitoneal) and 0.01 μl Temgesic (subcutaneous; analgesic). Further analgesic treatment continued for 2-3 days as required. Following surgery, animals were weighed daily to monitor their recovery. At least 7 days were allowed for recovery before the start of the experiments.

Wireless recording microchips (Neurologger—NewBehavior, Zurich, Switzerland) were used to register EEG. The weight of the microchips in combination with the P10 hearing aid batteries is <3 g (approximately 10% of the body weight) and the physical dimensions are 24×15×5mm. The device contains a built-in accelerometer to record movements. Its weight and size allows placement directly at the head of a mouse (10% body weight). The sample rate is set to ˜200 samples per second (4 channels).

EEG was recorded for 24 hrs in PhenoTyper cages after two days of habituation. Recorded EEG data were downloaded to a PC using a USB connected docking station and data retrieved in hexadecimal format was transformed to a format compatible with our analysis software (SleepSign: Kissei Corp., Japan) by means of EEG_Process (Matlab). EEG recordings were then imported into SleepSign for staging (based on FFT power spectra and activity indicated by accelerometer), and extrapolation of power spectrum values. Spectral characteristics of the EEG were further analyzed for the states of NREM (non-REM sleep), REM (rapid eye movement) or WAKEfulness. Power spectra were normalised to the maximum value of each animal, and averaged per group. Hypnograms were obtained directly from SleepSign.

Statistical comparison was performed with repeated measures or 2-way analysis of variance (ANOVA), followed by planned paired comparison and post-hoc tests, using GraphPad Prim 5.0 (GraphPad Software Inc., San Diego, Calif. USA). Significance was set to the level of P<0.05. For vigilance state analysis, one way ANOVA was performed followed by post hoc tests for comparison between different groups.

EEG-based vigilance stages were recorded as a major phenotype in Disc1ₜᵣ transgenic mice. The results of the study showed that some trends were detected in respect of wake events, wake duration (5 month mice) fewer NREM events (9 month mice). Genotype-specific alterations in different sleep stages were observed specifically in terms of fragmentation due to an overall reduction of events of wakefulness. Despite these fewer events, we obtained a prolongation of wakefulness events that led to normalization of the overall time that animals were awake. A similar yet reciprocal change occurred for NREM sleep, but REM remained unaffected.

EEG power spectrum analyses uncovered a number of significant changes in heterozygous and homozygous animals compared to WT's. Of note are the significant decreases in power for the gamma frequency band, which are reminiscent of the reductions seen in schizophrenia patients (Light et al., 2006).

Table 2 shows the results for 9 month old mice.

TABLE 2
Overall summary of significant genotype (Gen) effect and interactions
(Int. − genotype × frequency) over all frequencies analyzed in this study.
Alpha, beta and gamma rhythms were most strongly affected by
expression of the Disc1ₜᵣ transgene and homozygosity seems
to amplify the effect.

| | | Light | | | | | |
|---|---|---|---|---|---|---|---|
| | | Wake | | REM | | NREM | |
| | | Het | Homo | Het | Homo | Het | Homo |
| PFx | Delta | NS | Int** | NS | NS | NS | Int**; |
| | | | | | | | gen* |
| | Theta | NS | Gen*** | Int* | NS | Gen** | Gen*** |
| | Alpha | Gen*** | Gen*** | Gen*** | Gen* | Gen*** | Gen*** |
| | Beta | Gen*** | Gen*** | Gen** | Gen*** | Gen*** | Gen*** |
| | Gamma | Gen*** | Gen*** | Gen*** | Gen*** | Gen*** | Int***/ |
| | | | | | | | Gen*** |
| RH | Delta | NS | NS | Gen*** | Gen*** | NS | Int* |
| | Theta | NS | Gen* | Int*** | NS | NS | NS |
| | Alpha | NS | NS | Gen*** | Gen** | NS | Gen** |
| | Beta | Gen** | Gen*** | Gen** | Gen*** | NS | NS |
| | Gamma | Gen*** | Gen*** | Gen*** | Gen*** | Gen*** | Gen*** |
| LH | Delta | NS | NS | Gen*** | NS | NS | NS |
| | Theta | NS | Gen* | Int**/ | NS | Gen* | Gen** |
| | | | | gen* | | | |
| | Alpha | NS | Gen* | Gen*** | NS | Gen*** | Gen*** |
| | Beta | Gen** | Gen** | Gen** | NS | Gen*** | Gen*** |
| | Gamma | Gen*** | Gen*** | Gen*** | NS | Gen*** | Gen*** |

### REFERENCES

Assadi A H, Zhang G, Beffert U, McNeil R S, Renfro A L, Niu S, Quattrocchi C C, Antaiffy B A, Sheldon M, Armstrong D D, Wynshaw-Boris A, Herz J, D'Arcangelo G, Clark G D (2003) Interaction of reelin signaling and Lis1 in brain development. Nat Genet 35:270-276.

Benes F M, Lim B, Matzilevich D, Walsh J P, Subburaju S, Minns M (2007) Regulation of the GABA cell phenotype in hippocampus of schizophrenics and bipolars. Proc Natl Acad Sci USA 104:10164-10169.

Blackwood D H, Fordyce A, Walker M T, St Clair D M, Porteous D J, Muir W J (2001) Schizophrenia and affective disorders—cosegregation with a translocation at chromosome 1q42 that directly disrupts brain-expressed genes: clinical and P300 findings in a family. Am J Hum Genet 69:428-433.

Braff D L, Geyer M A, Swerdlow N R (2001) Human studies of prepulse inhibition of startle: normal subjects, patient groups, and pharmacological studies. Psychopharmacology 156:234-258.

Brandon N J, Handford E J, Schurov I, Rain J C, Pelling M, Duran-Jimeniz B, Camargo L M, Oliver K R, Beher D, Shearman M S, Whiting P J (2004) Disrupted in Schizophrenia 1 and Nudel form a neurodevelopmentally regulated protein complex: implications for schizophrenia and other major neurological disorders. Mol Cell Neurosci 25:42-55.

Cannon T D, Hennah W, van Erp T G, Thompson P M, Lonnqvist J, Huttunen M, Gasperoni T, Tuulio-Henriksson A, Pirkola T, Toga A W, Kaprio J, Mazziotta J, Peltonen L (2005) Association of DISC1/TRAX haplotypes with schizophrenia, reduced prefrontal gray matter, and impaired short- and long-term memory. Arch Gen Psychiatry 62:1205-1213.

Chen Q Y, Chen Q, Feng G Y, Lindpaintner K, Wang L J, Chen Z X, Gao Z S, Tang Huang G, He L (2007) Case-control association study of Disrupted-in-Schizophrenia-1 (DISC1) gene and schizophrenia in the Chinese population. J Psychiatr Res 41:428-434.

Chinnasamy D, Rudd R, Velakoulis D (2006) A case of schizophrenia with complete agenesis of the corpus callosum. Australas Psychiatry 14:327-330.

Clapcote S J, Lipina T V, Millar J K, Mackie S, Christie S, Ogawa F, Lerch J P, Trimble K, Uchiyama M, Sakuraba Y, Kaneda H, Shiroishi T, Houslay M D, Henkelman R M, Sled J G, Gondo Y, Porteous D J, Roder J C (2007) Behavioral phenotypes of Disc1 missense mutations in mice. Neuron 54:387-402.

Cohrs S (2008) Sleep disturbances in patients with schizophrenia: impact and effect of antipsychotics. CNS Drugs 22: 939-962.

Devon R S, Anderson S, Teague P W, Burgess P, Kipari T M, Semple C A, Millar J K, Muir W J, Murray V, Pelosi A J, Blackwood D H, Porteous D J (2001) Identification of polymorphisms within Disrupted in Schizophrenia 1 and Disrupted in Schizophrenia 2, and an investigation of their association with schizophrenia and bipolar affective disorder. Psychiatr Genet 11:71-78.

Ekelund J, Hovatta I, Parker A, Paunio T, Varilo T, Martin R, Suhonen J, Ellonen P, Chan G, Sinsheimer JS, Sobel E, Juvonen H, Arajarvi R, Partonen T, Suvisaari J, Lonnqvist J, Meyer J, Peltonen L (2001) Chromosome 1 loci in Finnish schizophrenia families. Hum Mol Genet 10:1611-1617.

Estivill-Torrus G, Pearson H, van Heyningen V, Price D J, Rashbass P (2002) Pax6 is required to regulate the cell cycle and the rate of progression from symmetrical to asymmetrical division in mammalian cortical progenitors. Development 129:455-66.

Faulkner N E, Dujardin D L, Tai C Y, Vaughan K T, O'Connell C B, Wang Y, Vallee R B (2000) A role for the lissencephaly gene LIS1 in mitosis and cytoplasmic dynein function. Nat Cell Biol 2:784-791.

Feng Y, Walsh C A (2004) Mitotic spindle regulation by Nde1 controls cerebral cortical size. Neuron 44:279-293.

Harrison P J (1999) The neuropathology of schizophrenia. A critical review of the data and their interpretation. Brain 122:593-624.

Hattori T, Baba K, Matsuzaki S, Honda A, Miyoshi K, Inoue K, Taniguchi M, Hashimoto H, Shintani N, Baba A, Shimizu S, Yukioka F, Kumamoto N, Yamaguchi A, Tohyama M, Katayama T (2007) A novel DISC1-interacting partner DISC1-Binding Zinc-finger protein: implication in the modulation of DISC1-dependent neurite outgrowth. Mol Psychiatry 12:398-407.

Hennah W, Tuulio-Henriksson A, Paunio T, Ekelund J, Varilo T, Partonen T, Cannon T D, Lonnqvist J, Peltonen L (2005) A haplotype within the DISC1 gene is associated with visual memory functions in families with a high density of schizophrenia. Mol Psychiatry 10:1097-1103.

Hennah W, Thomson P, McQuillin A, Bass N, Loukola A, Anjorin A, Blackwood D, Curtis D, Deary I J, Harris SE, Isometsä ET, Lawrence J, Lönnqvist J, Muir W, Palotie A, Partonen T, Paunio T, Pylkkö E, Robinson M, Soronen P, Suominen K, Suvisaari J, Thirumalai S, Clair D S, Gurling H, Peltonen L, Porteous D (2008) DISC1 association, heterogeneity and interplay in schizophrenia and bipolar disorder. Mol Psychiatry [Epub ahead of print]

Hikida T, Jaaro-Peled H, Seshadri S, Oishi K, Hookway C, Kong S, Wu D, Xue R, Andradé M, Tankou S, Mori S, Gallagher M, Ishizuka K, Pletnikov M, Kida S, Sawa A (2007) Dominant-negative DISC1 transgenic mice display schizophrenia-associated phenotypes detected by measures translatable to humans. Proc Natl Acad Sci USA 104:14501-14506.

Hirohashi Y, Wang Q, Liu Q, Li B, Du X, Zhang H, Furuuchi K, Masuda K, Sato N, Greene M I (2006) Centrosomal proteins Nde1 and Su48 form a complex regulated by phosphorylation. Oncogene 25:6048-6055.

Holy T E, Guo Z (2005) Ultrasonic songs of male mice. PLoS Biol 3:2177-2186.

Honea R, Crow T J, Passingham D, Mackay C E (2005) Regional deficits in brain volume in schizophrenia: a meta-analysis of voxel-based morphometry studies. Am J Psychiatry 162: 2233-2245.

Innocenti G M, Ansermet F, Parnas J (2003) Schizophrenia, neurodevelopment and corpus callosum. Mol Psychiatry 8:261-274.

Ishizuka K, Chen J, Taya S, Li W, Millar J K, Xu Y, Clapcote S J, Hookway C, Morita M, Kamiya A, Tomoda T, Lipska B K, Roder J C, Pletnikov M, Porteous D, Silva A J, Cannon T D, Kaibuchi K, Brandon N J, Weinberger D R, Sawa A (2007) Evidence that many of the DISC1 isoforms in C57BL/6J mice are also expressed in 129S6/SvEv mice. Mol Psychiatry 12:897-899.

Kamiya A, Kubo K, Tomoda T, Takaki M, Youn R, Ozeki Y, Sawamura N, Park U, Kudo C, Okawa M, Ross C A, Hatten M E, Nakajima K, Sawa A (2005) A schizophrenia-associated mutation of DISC1 perturbs cerebral cortex development. Nat Cell Biol 7:1167-1178.

Kilpinen H, Ylisaukko-Oja T, Hennah W, Palo O M, Varilo T, Vanhala R, Nieminen-von Wendt T, von Wendt L, Paunio T, Peltonen L (2008) Association of DISC1 with autism and Asperger syndrome. Mol Psychiatry 13:187-196.

Koike H, Arguello P A, Kvajo M, Karayiorgou M, Gogos J A (2006) Disc1 is mutated in the 129S6/SvEv strain and modulates working memory in mice. Proc Natl Acad Sci USA 103:3693-3697.

Lang B, Song B, Davidson W, MacKenzie A, Smith N, McCaig C D, Harmar A J, Shen S (2006) Expression of the human PAC1 receptor leads to dose-dependent hydrocephalus-related abnormalities in mice. J Clin Invest 116:1924-1934.

Lewis D A, Levitt P (2002) Schizophrenia as a disorder of neurodevelopment. Annu Rev Neurosci 25:409-432.

Lewis D A, Hashimoto T, Volk D W (2005) Cortical inhibitory neurons and schizophrenia. Nat Rev Neurosci 6:312-324.

Light G A, Hsu J L, Hsieh M H, Meyer-Gomes K, Sprock J, Swerdlow N R, Braff D L. (2006) Gamma band oscillations reveal neural network cortical coherence dysfunction in schizophrenia patients. Biol Psychiatry. 60(11)1231-40.

Livy D J, Wahlsten D (1991) Tests of Genetic Allelism between Four Inbred Mouse Strains with Absent Corpus Callosum. J Heredity 82:459-464.

Miller E K, Cohen J D (2001) An integrative theory of prefrontal cortex function. Annu Rev Neurosci 24:167-202.

Millar J K, Wilson-Annan J C, Anderson S, Christie S, Taylor M S, Semple C A, Devon R S, Clair D M, Muir W J, Blackwood D H, Porteous D J (2000) Disruption of two novel genes by a translocation co-segregating with schizophrenia. Hum Mol Genet 9:1415-1423.

Millar J K, Pickard B S, Mackie S, James R, Christie S, Buchanan S R, Malloy M P, Chubb J E, Huston E, Baillie G S, Thomson P A, Hill E V, Brandon N J, Rain J C, Camargo L M, Whiting P J, Houslay M D, Blackwood D H, Muir W J, Porteous D J (2005) DISC1 and PDE4B are interacting genetic factors in schizophrenia that regulate cAMP signaling. Science 310:1187-1191.

Miyata J, Hirao K, Namiki C, Fukuyama H, Okada T, Miki Y, Hayashi T, Murai T (2007) Interfrontal commissural abnormality in schizophrenia: tractography-assisted callosal parcellation. Schizophr Res 97:236-241.

Motomura N H (2002) Monozygotic twin cases of the agenesis of the corpus callosum with schizophrenic disorder. Psychiatry Clin Neurosci 56:199-202.

Murdoch H, Mackie S, Collins D M, Hill E V, Bolger G B, Klussmann E, Porteous D J, Millar J K, Houslay M D (2007) Isoform-selective susceptibility of DISC1/phosphodiesterase-4 complexes to dissociation by elevated intracellular cAMP levels. J Neurosci 27:9513-9524.

Nishida K, Flanagan J G, Nakamoto M (2002) Domain-specific olivocerebellar projection regulated by the EphA-ephrin-A interaction. Development 129:5647-5658

Ozeki Y, Tomoda T, Kleiderlein J, Kamiya A, Bord L, Fujii K, Okawa M, Yamada N, Hatten M E, Snyder S H, Ross C A, Sawa A (2003) Disrupted-in-Schizophrenia-1 (DISC-1): mutant truncation prevents binding to NudE-like (NUDEL) and inhibits neurite outgrowth. Proc Natl Acad Sci USA 100:289-294.

Paul L K, Brown W S, Adolphs R, Tyszka J M, Richards L J, Mukherjee P, Sherr E H (2007) Agenesis of the corpus callosum: genetic, developmental and functional aspects of connectivity. Nat Rev Neurosci 8:287-299.

Pletnikov M V, Ayhan Y, Nikolskaia O, Xu Y, Ovanesov MV, Huang H, Mori S, Moran T H, Ross C A (2008) Inducible expression of mutant human DISC1 in mice is associated with brain and behavioral abnormalities reminiscent of schizophrenia. Mol Psychiatry 13:173-186.

Rascle C, Mazas O, Vaiva G, Toumant M, Raybois O, Goudemand M, Thomas P (2001) Clinical features of latent inhibition in schizophrenia. Schizophr Res 51: 149-161.

Reiner O, Carrozzo R, Shen Y, Wehnert M, Faustinella F, Dobyns W B, Caskey C T, Ledbetter D H (1993) Isolation of a Miller-Dieker lissencephaly gene containing G protein b-subunit-like repeats. Nature 364:717-721.

Ross C A, Margolis R L, Reading S A, Pletnikov M, Coyle J T (2006) Neurobiology of schizophrenia. Neuron 52:139-153.

Sachs N A, Sawa A, Holmes S E, Ross C A, DeLisi L E, Margolis R L (2005) A frameshift mutation in Disrupted in Schizophrenia 1 in an American family with schizophrenia and schizoaffective disorder. Mol Psychiatry 10:758-764.

Sasaki S, Mori D, Toyo-oka K, Chen A, Garrett-Beal L, Muramatsu M, Miyagawa S, Hiraiwa N, Yoshiki A, Wynshaw-Boris A, Hirotsune S (2005) Complete loss of Ndel1 results in neuronal migration defects and early embryonic lethality. Mol Cell Biol 25:7812-7827.

St Clair D, Blackwood D, Muir W, Carothers A, Walker M, Spowart G, Gosden C, Evans H J (1990) Association within a family of a balanced autosomal translocation with major mental illness. Lancet 336:13-16.

Thomson P A, Wray N R, Millar J K, Evans K L, Hellard S L, Condie A, Muir W J, Blackwood D H, Porteous D J (2005) Association between the TRAX/DISC locus and both bipolar disorder and schizophrenia in the Scottish population. Mol Psychiatry 10:657-668.

Toyo-oka K, Shionoya A, Gambello M J, Cardoso C, Leventer R, Ward H L, Ayala R, Tsai L H, Dobyns W, Ledbetter D, Hirotsune S, Wynshaw-Boris A (2003) 14-3-3 epsilon is important for neuronal migration by binding to NUDEL: a molecular explanation for Miller-Dieker syndrome. Nat Genet 34:274-285.

Vallee R B, Tsai J W (2006) The cellular roles of the lissencephaly gene LIS1, and what they tell us about brain development. Genes Dev 20:1384-1393.

Wang Q, Du X, Meinkoth J, Hirohashi Y, Zhang H, Liu Q, Richter M, Greene M I (2006) Characterization of Su48, a centrosome protein essential for cell division. Proc Natl Acad Sci USA 103:6512-6517.

Whitney G (1970) Ontogeny of sonic vocalizations of laboratory mice. Behav Genet 1:269-273.

Whitney G, Nyby J (1983) Sound communication among adults. In: The auditory psychobiology of the mouse (Willott J F, editor). pp 98-129, Springfield (Ill.), C. C. Thomas.

Yang X W, Model P, Heintz N (1997) Homologous recombination based modification in Escherichia coli and germline transmission in Tg mice of a bacterial artificial chromosome. Nat Biotechnol 15:859-865.

Zhang X, Tochigi M, Ohashi J, Maeda K, Kato T, Okazaki Y, Kato N, Tokunaga K, Sawa A, Sasaki T (2005) Association study of the DISC1/TRAX locus with schizophrenia in a Japanese population. Schizophr Res 79:175-180.

Zhang Z J, Reynolds G P (2002) A selective decrease in the relative density of parvalbumin-immunoreactive neurons in the hippocampus in schizophrenia. Schizophr Res 55:1-10.

Nishida K, Flanagan J G, Nakamoto M (2002) Domain-specific olivocerebellar projection regulated by the EphA-ephrin-A interaction. Development 129:5647-565

1. A transgenic rodent which includes within a plurality of its cells:
(1) a plurality of copies of a heterologous truncated Disc1 genomic DNA sequence which includes at least 1 stop codon after exon 8 such as to encode a Disc1 polypeptide truncated before exon 9;
(2) 2 copies of endogenous Disc1 genomic DNA sequence encoding full length Disc1 polypeptide.

2. The transgenic rodent of claim 1 which is heterozygous with respect to the heterologous truncated Disc1 genomic DNA sequences, and wherein there are 2 copies/cell of said heterologous truncated Disc1 genomic DNA.

3. The transgenic rodent of claim 2 wherein the truncated Disc polypeptide and full length Disc 1 polypeptide are expressed in an approximately 1:1 ratio.

4. The transgenic rodent of claim 1 which is homozygous with respect to the heterologous truncated Disc1 genomic DNA sequences, and wherein there are 4 copies/cell of said heterologous truncated Disc1 genomic DNA.

5. The transgenic rodent of claim 1 wherein the final 3, 2 or 1 nucleotides of exon 8 of the heterologous truncated Disc1 genomic DNA sequence are modified.

6. The transgenic rodent of claim 1 wherein the heterologous truncated Disc1 genomic DNA includes a reporter gene or an epitope tag which is expressed as a fusion with the truncated Disc1 polypeptide, wherein the reporter gene is fused at the end of exon 8 and includes the stop codon.

7. The transgenic rodent of claim 6 wherein the reporter gene is EGFP.

8. The transgenic rodent of claim 1 wherein the heterologous truncated Disc1 genomic DNA sequence includes at least the first 9 exons and 8 intronic sequences of Disc1 and optionally a polyA sequence within intron 8

9. The transgenic rodent of claim 8 wherein the heterologous truncated Disc1 genomic DNA includes exon 9 and is truncated within intron 9.

10. The transgenic rodent of claim 1 wherein the heterologous truncated Disc1 genomic DNA sequence includes a functional native Disc1 genomic promoter.

11. Transgenic material obtained from the transgenic rodent of claim 1, wherein the material is selected from the list consisting of: a somatic cell; a tissue sample; a gamete.

12. (canceled)

13. A nucleic acid comprising a truncated Disc1 genomic DNA sequence which includes at least 1 stop codon after exon 8 such as to encode a Disc1 polypeptide truncated before exon 9 and including a reporter gene or an epitope tag which is expressed as a fusion with the truncated Disc1 polypeptide.

14. A nucleic acid as claimed in claim 13 which is a BAC wherein the heterologous truncated Disc1 genomic DNA sequence includes at least the first 9 exons and 8 intronic sequences of Disc1 and optionally a polyA sequence within intron 8; and wherein the heterologous truncated Disc1 genomic DNA optionally includes exon 9 and is truncated within intron 9; and wherein the heterologous truncated Disc1 genomic DNA sequence optionally includes a functional native Disc1 genomic promoter.

15. A nucleic acid as claimed in claim 13 which is a BAC.

16. The transgenic rodent of claim 1 having one or more of the phenotypes described in Table 1.

17. The transgenic rodent of claim 16 having equal to or at least 1, 2, 3, 4, 5 or all 6 of the following phenotypes compared to a wild type rodent: thinning of the cortical layers II/III; selective decrease of neural proliferation in the developing cortex at mid-neurogenesis; partial agenesis of the corpus callosum; reduced parvalbumin GABAergic neurons in the hippocampus and displaced parvalbumin cells at the dorsolateral frontal cortex; increased immobility and reduced vocalization in depression-related tests.

18. The transgenic rodent of claim 16 having one or both of the following phenotypes compared to a wild type rodent: significant deficit in social recognition memory; abnormal EEG activity in prefrontal cortex, which abnormal EEG activity is optionally significant decreased power in the gamma frequency band.

19. A method of preparing a transgenic rodent of claim 17, the method comprising the steps of:
(a) introducing a nucleic acid into the pronucleus of a fertilized rodent egg to produce a modified fertilized rodent egg,
wherein the nucleic acid comprises a truncated Disc1 genomic DNA sequence which includes at least I stop codon after exon 8 such as to encode a Disc1 polypeptide truncated before exon 9 and including a reporter gene or an epitope tag which is expressed as a fusion with the truncated Disc1 polypeptide;
(b) implanting the modified fertilized rodent egg into the uterus of a pseudopregnant rodent female to produce a pregnant female rodent;
(c) allowing the pregnant rodent to produce offspring; and
(d) screening the offspring for the introduction of 2 or more copies of the nucleic acid in the genome.

20. A method as claimed in claim 19 further comprising the step of crossing the offspring with another rodent of the same species.

21. A method of modelling schizophrenia, schizoaffective disorder, depression or bipolar disorder by providing the transgenic rodent of claim 17 and monitoring changes in one or more of the phenotypes of the rodent or in the neuroanatomy of the rodent, which phenotypes are representative of schizophrenia, schizoaffective disorder, depression or bipolar disorder.

22. A method of screening or assessing a compound suspected of having a therapeutic effect in relation to schizophrenia, schizoaffective disorder, depression or bipolar disorder, the method comprising:
(a) providing the transgenic rodent of claim 17;
(b) administering the compound to the rodent;
(c) monitoring changes in one or more of the phenotypes of the rodent or in the neuroanatomy of the rodent in response to the compound so administered;
(d) correlating an improvement in the one or more phenotypes or in the neuroanatomy monitored in (c) with the potential therapeutic effect of the compound.

23. A method as claimed in claim 21 wherein the phenotype monitored is selected from the group consisting of: thinning of the cortical layers II/III; selective decrease of neural proliferation in the developing cortex at mid-neurogenesis; partial agenesis of the corpus callosum; reduced parvalbumin GABAergic neurons in the hippocampus and displaced parvalbumin cells at the dorsolateral frontal cortex; increased immobility; reduced vocalization in depression-related tests; deficit in social recognition memory; abnormal EEG activity in prefrontal cortex, which abnormal EEG activity is optionally decreased power in the gamma frequency band.

24. A transgenic rodent as claimed in claim 1, wherein the transgenic rodent is a mouse.

25. The method of claim 21 wherein the transgenic rodent is a mouse.


SEQUENCE LISTING (35 sequences)

SEQ ID NO: 1 - DNA, 148707 bp, Artificial sequence
  Synthetic sequence NruI fragment used for the generation of the Disc1tr transgenic mice, derived from the BAC clone RP23-236F19 fused with an EGFP reporter
cgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagacttaattaaggatcgatccggcgcgccaatagtcatgccccgcgcccaccggaaggagctgactgggttgaaggctctcaagggcatcggtcgagcttgacattgtaggactatattgctctaataaatttgcggccgctaatacgactcactatagggagaggatccgcggaattcgtaggaagcagttttcctgaagagttgatgggtatctggatgttcactttcccacgttgtcttttcctttctcaccgatgtgggaaggaaggagctgacactggtcatcactggtgacgctgagtaactgactccacactcgtgtgtaggttgtgagcagccaggccctttgtcgtgtgttaggtgtggtgcacttgtctaccccagccgtcctgggaccccagaggagcagccacagtgctcccagtgctgacaacccaagccttgaccctgcccttggtgtgagctagcgatgttgccaaagaaaaagaattgtgagtaattcagcaataagacgatggccaacactggagggagcgtcgggcgtgggttcatctccccagtcagtcagagtcatctgtggtgagagcctggcttgagcgtggcctgcggctgtcaacgccacatgctttcaacgagttctgtgacttttcacaacttagatactgggctttatttttggaaagaatatctatttgtaaggctggttctttgaaagtgtaattttccaaaaagtctcaccaaaagtaaaataaagtgtgtacatgcctggtaagttagacttgttgtcctggtggtgatgaggcttcgccttcctgtagtaagatggactcctgagctgtagtcagtcctggtacctgctctgtctgtctggtttttttcagctacttcaagtggaatttataatgttaaaataatggattgaagaatgttttacagaaatttaaagcaaactttaaataaaatattaaaatacagcagccctctggttaagcttttaagctccattgtgtattgtgggcgtggtgggcgtggtgggcagaggtcagacacagcttgtgcagacgggtctgttccttgcactgtgggtgacggggacactggtgggtgtcaggttgggataagggcctttttcatccggcgaggccacctcgtgactcccagagcctggtgttttgatggtatttagaaatatatagtgaatcagtaatgtgatttttgggtaaaatgggacatttaagattggctcgggtaatgtggaggtctatttttcctcccttaagccccgagacaagatgtcctagtggtatgactcaacctgaaatgtagtcagggacttcccttgtgtctctgtggtctgcatcctctctcagtgcctccccgggttgcctggtttcagatatggtagggatgctccagaagtggacgtccttgtgttctctcaggttaaaatgagctccatctgctcacaagaaagacatccttcacagcactcggactcagactcagactcagatacctcatcccaccccgtccagggctaacagggaaacactagaaagccaggctgtcagaggagcacagaaatggcctgtggcataattagcagcgccccctgcaggcgaccgagcaccactgcccctgctgtgttctttgtgcagtgggcggcttgagggctcagtctaaattgtgtgtgacagcaccatctgctttggtggcctttctgtttatcacaaaaaggaactggttcaagggctggcagaatggaacctcacaaccagcatccctgaagcccacacggtaggaagagagagactggtcacggggccctatggcaagcccttccccactgcaaactaaataaatagaaacgtaattaaaaaaaaataggagaaagcaagtcaggctctcatggcaagtgggtgactgaattttgaaatgaaagtggattatttcttttcagttgtgatttcttgtgcctcttaaatgcgtggtgttcacagagaacctgggagtcttggtcaccctcaaaggagatcgacacttaagtgagagattggcagaatggctccacactcctttctgtctaaagagactcacgttagagtggaagacaagtggattgaagtgaaagggtggaaaatagatatcccatgcaaacaggacccaagagagaatctgattggtctctgagagcagatacaatagacctaatctttatttagctgcaaacagagaatagcattttataataagttcaatccaataattatagaacagctcaaaggaaaaatgctaataaatggggtgtgtgcgcgcgcgcatgtgtgcgcgtgtacatgtgtgtgtgtgtgtgtgtacgtgtgtgcatgcaggtgcatatgtacatacatgtgcatgtagtggcctatgtagtccaggctggccacaaactcatagccttccagcctatggggttttacacagccatgtccagtcttatattttttaagacatgtaaatcccactgagcaaatgtctcccattgtgtcctttgctccattaacaggatcactttttaatttggagattagaaactggttattccctggatatacagagtctatgtttcagtagaaatgtgctttgtagttcataatttaccatcatgatggtcagggacatattgtacaggatcccataaaataagcacctgctagtggtgtcacctgggtttataagcaaactgtacacaaacagcaacactgccccttgacacctctcaaaatgtgggtcaagtgagggccagctctgtgccatctcagcattcaggttaaaggtttttgtttttgttttctggaagaagtcttaatttgtaacttttctcagtaatctagaaaaacaaagttgcatccttcctattgtgtgtgttgctttaaaaagcatgatggcaggagcagtggagagctggcgagctggtgcagcagttaagagcacaaatggagattaattaaatattgtgagaccgaggaaatagcccaggtagagctattgcttagcacgtgcaaggccctaagttcagaaaaccaagaaaccaaaaccaaaaccatgacagaacaacgcccagggaacatgaaacactgagtccaagaacaacttccattctctgcctgccttgtctgcttaacacaggcatgtgttcataaaacacataacacatatacaatcacacttgcatgtgcacacacaaagcgtttctctgggacacctattagtacgtccatgtgtgtgtgactaaagaacagccactccaaatggcaggatcctgatggcagggatccgtcaggaaggaagagctgggttaccctacttagaatcaacgctttggggctgtgatcatggggtaggtgagggacgcactgtgagctctaataattaccttaagagttgctcaggaaacagggctttaggaatagcacatttccccctctctttgtaatgtatatattttataaattaggggtgtgtgtgtgtgtgttttcctcctcttaactctgtcatagggtatggtgttttcatttagacttcaaattgttggatgtcaaaggcaaactattgccttgaaggagtgggcaccatagcaatggacctatcaggtagagatctccttcttgtccccctttctcacccatggcatcaaggacaggtgcaaacctcatgtgtaaacacagtatatttagaagttccactgtgcaccaagtgtgtgagcacaaaagaccaaattgctagactgtacggtggtgtctgttggttggttttagccccttgtagttcctgcaaaggggctggagtcttggggacacagctgtgtgacttgcttgccttcagtgttccacctgcattggaacaaagggaccccagatgaccagcaaaggctggcatgagcagatggcatacgtgagggttcgcaggagcctctctgttctgccgctcacctgctcaggctgcaggcagcagaggttatgacctgggcttcctgcaatttctgcaaccccccgacttcctgcccttgctggctgcgatcccagccttcccttcctccagcccttccactgtttggaagcacctgtgttaagtcctttcctgagcgggctacccaaagtgcgctctgttttcctaagtgagcatgacagcaggctgcccttgtcagactagaaatacaactggtcttaggcataaccaggaaggtgtagatttcaggactttagtgtccctgtatcaagtgggccaagatctgatgttcaagaaggccgcgtagatgtgagagaatgggttcaggccgcaccattctgtttcccatcttcacgttgggatggccacatgcagaaaacagcttatctgacctcatactgaccatctgcaccctgtggagcacaggaccctctctgtctgcatactgttgagctcagtcctcggttcctagttgaaatgccaaagaaatcaatagccaacttgaataaaggtcagtgacttcaaagaggatacaaacagatgaaagcaggatgtcaccgcaagacctgtgtaagaaagtcagcatcacgggtgggaaagttatggaggatattgagattttgaaacacacacacaaatacacacacaaatacatagatacaggcacatacacatttacacatttgtatacacagacatacagatacacatttacacattggtagatgtacatagacacagatatgcacatacacacttaacatacatgctataaacatgcagatatacatacgcacacataccttaatgagacacgttatcatgatgacagacagagcttgtttctgagtcttcacaatagactgtcgttaacctgattcttctcacttacggcgtctctggaggctgctctcatcccccattatcccagaagtctgccctctttccagtagttttctgattagatcgtcctccttcaccctgacacatatatactgtctgcattgacatatcctagatacatgtaaaatatgtatgcacacgcacagttgtacacagaaatagttattgctaattaagggttaggattttgtactaatgcattttttttttcatcagaagagattcacagatagatgactttatgtggtaatcagtgtgcttacacagctgacctctaccgtcccaggcgctgtgaagtagcaggatctgctacagcgtgttaaggggtatacaccattgtttgttagctgggtactgtggctcgcacctttaatccctgcgcttaggaggaggaagcaggaggattactgtggatccgatgccagccaggctaaagaatgagactttgtctagaacaaacaaaaataattaataaaaaatctgtattattgtaaacaacaccgacacagatattctcataaatgcatctgaccttattttcttgacgcaagtcacggaaggccaaatagccaggtgacacgaatgtttttgttacctaactgaaaaattggatttcttcagaacacttaggagccagattccctcttttaaagtcattgttccgcagataagtgtttacaagcaagcgtctctccccagttcgtcacccctgtcaccactaatgggaagcctcccgcgtgctccagagcatcagtacgtgctcagggggagagagacgccctgtgattactttccaacatgggaagcagagcgtgtgtctcggaccagaggccccgtttgccagcacccagccccatcctgactagaggagtatgacattaacagatgtgaattacttaatcctcatcagcacagtgtcaggtgacagtgttaccacgggggtcattagaatggaagcacagaaccctcctggcgtgggccagcggtggagaacagtgcttcagccctgcgttccacacacggtctacccaacagctcctttggctgttcagagacgggctgcaccagaagcctgtaagatggaatgggcacgtacaagcattttgcggagttctctcttaatacaaaattaatagcttaaagctctctagcttgaaaaccttgacgggagacatgaaagaaagatccccagcgagaagttcatcttgcattgggtgtctcgtggcactgcgattgtaattatgtacaggcgtgcacttaccttcagatactcacgttcctgcatctaggaggagacaccaccaaatgctgtacagttaacaattttaaaagttaaaaggagaaatcttgctgggcctagtgacacacaccttagttccagcacctgggaggcagaggcagggtgtgagtttgagaccagcctggtctacagaatgagttccaggacagccagaggttcacagaaacaacaaacaaacaaacaaacaatggggatggatggataggtaggtaggtaggttgagatggttcagcagtacccacattgtggatggagagaactgatttccagaagttatcctctgatattcagatacccagtgtagtgcatatggtacatatgtgcctccacagaaataaaacataaaagagaaaatgcagtatgaatcttcacaactgagcagcagatcgcccacgagagcaggtgctgcgtgcttgttgaacctgccaggcttgtaagcatccttggacttttctcacgttgtaaacttcgctcagttgtataaaagttgcctcctttaagttgagtagagacagggatggagagagggcgtcatgatgaatcttggagttaaggagaggatactaacaggcatccatctgtccaggtgaaaagctcagctgaggcattatacattatacattatacaggacattatacaccgtggtccatgtctcttgtcacgttagactgaacacgatgtctactccccacccagatctctccactttacctcatttattcccaccacgttaactttgttttgttttgtttgcttgttcgatagacaggggtgaagtggaaacttaagggttctttgcaaagtcagacgtgttagatgttttgctggggcaaacatgtaaaagttttcctgaagtagacataggtgaaagactaaggcagactcatgaaggaacgtttcactgaggcagacacaggagagaggatgttctgttagagcaaacacgtgaaaggacacaggatgaaggattcttcactaatgacgcacatgttttagttcgccttacattgcgtaactgagctgcattcgtcaggactccatagagagaaacacactaaaaatctggtggtgtcctgcattcctccgtggcctctgcatcagctcctgcctccacgttccatgccttgtgtgagttcctgccttcaatgtgtttgatgatgtaactgttgtcatatgaaataaaaataaaccctttcctctccaagttgctttgggtcatgttgtttcattacagcaacagtgaccccgactaagacacacactaaagaatcattcctgcaaagctgacgtagaggtcagcctgaggccagtacactgcgcatgcccaacccccaccacggctgccactggctttgtacttgcaagaggcggacatttctttctttgagccccagcttcccctctgtacacactcttcatcctgagctagcctgggtctcagaacgatgatggtcagcctgcatcccaaatagacacataacgctgttcagattgcagggtatggaggagccaggcatgggcaggagtgatggggaagatctatgacacacagaaggcaaacctgtgggcaataactagcctgtgccatgccggctggtggaggccagagtcaaggctcacaaggacacttccccatcatgagcctggtcgcccaaaggacagcaaagcgtttcctagtgttgcatagtcacaccacagggcctttcatttggcctggaactcatcaagtcgtcaaggctggctgacctatggctccaggtcactgtgtttccccactcagagagctttaaggcgctaagtcttaaagtagacattctgctgtaactgtccaggaagggctgttttagattaggcagggttgatttttatcataccatccattatgcataagggagagagggcgtgctgaccgggtaaggtttgcaggaagctgttgccctcagcccgtcttttcatggattgcaaccagccacacccccagcttccctcagatcactcagactcacaaggctgggaatccctgccttcttctgtcagttgcattgcatatgaagaatccaaacaaaatggatcactcaaggtttggcactagttaaagtcattcggatttattttaatcaaaaccaaaggtcactagaagctgctggggaaggacagctgggttcccaaatgccactgccagggagcaaggacttcacccttcttcatgattctagcacctggttcccagctgtggtgcaagcctgtgttccacgccagggtgttgtaaggaggcagggcttccaggagcatagcctgggggtggtttcagacagatccccaggacaagatgctgtgttatttctgtggtgtggctaagtggagatgaagttctgtgtggggtatgcctgagtgcttgcccaaatcagccagtcctgactgtgacttctgagaatgaaggatttttaacagcaagaccagaagaatgggctagagagagagttgtggtgtgggcgagttagagcctagccttgtagacgctagagaaagtctagtctgtgttccataaagtttggcaattacgcaggaagagactgtagaacattgacccccccaaagatggttatgagatggtcaccagtcaccagttaacatgaccaagtttaactttcacagatttggtaaatttggaattctagggagaatcctgaggagatactgacctggacaaaaagctttctcaactactctatgacacggatgtctggtgcttgtgcggttttcatacgacagaaatatctagaggcctggctggtgtgtgtgtgcgtgtgtgtgtgtgtgtgtgtgtgtgtgtgtgtgtgtgtatgtatgtatgtatgtgtgtttgtgtgtgtgtatgtgtctgtatgtatgtatgtatatgtgtgtatgtatgtatgtgtatgtgtgtgtgatttttttttttttaaatcagagtcactcactccatctgcctcagccttttgagtgctgggagtacaggcacggaccaccacacccagatcttgtccatttagtatgtgtgagcatgtctgtgtttatgcatatgtgttcatatgcatagatacaaatgagtcagccagaggtggttcctggaacctttgtttcttgttttgtttatctgttttgagacagggtcttactatgtagcccaactgtctcggaacttactatgtagatcaagattgcctggagctcatagaaatccttggttgcagagattaaagactgcagcatgacctctggtttaacacccccccccttttttttttttgaactaagattcttcactggcctggggctgtcgaggtgggctaggctacctagaagacggatccactgtctgtttgcacagtgggattacaagcatggccaaacaagattggcattttgttgttgttgttgttttgtgtttatgtggtgcttggggggcaagctcaggtcatcacacctgcacagcaagccctttactaacagaaccagttccactgcttctgcctcctccccgtccccccatcatccctaccccacagtcccccagctagttatgaaagggaagaactcatatgaaaacctggagatagctgtgtgtggcaattcacacttttaatcccagtacgtgggagccagaagcaggacgatccggatctccgtagctagttctaggttagtacttctatagtgagacctcgcctaaaacaataacaacaaacataacctgcctcccccccaaaaaaacaaaaacccaaacctgaaggtacgagtgattcagagatgtctgatggtgcttttaatcaccctgcagttaacttttttgagtaaggaagaacacgaaatgaatgtgttcatgcagacataaatttatttctaattccgtccttctgcctttcctgttcacaaatgcggcctctttgcttggggtctccactaagcccgaccagcctcctggggcacaaaacccctttcttctctgtacttgctggttactatgtagttttacaagtatcattcagcaaacaacaggccacaaacatcccggagttcaagatcctcatgcagtcagatggaggttccgaagggacatgggtgagtcctgcttattcttgtgctcctgatgtcacagccctagggcacatacagaagtgattccctctggagttcctaaagccagctgccacgcaacagccctggcctgtcaggaaagggatggcggcacccacatgggtaggcccagtattaacttaaaccgatggaatttctatggccctgtgatttttttcttaaaaatctttacagaattgttcctaacacactgtgagagcagccaaaagcatccgcaagtcagggatactcagcacggtgaactgcgttagaccggggcaggatccagctgggctcatatttgagcctcaaataccaacatcaagccacagggagcaagattcaccagcacccccacccccgcaccccccaccccccagctcccgggatgctgtaattaagaagcacaaggacaagagaccagcaaacgttcacatctggtttttatcttaagtgtgttcctttgtctcatcattatggaaaaaaaaaaaaaaaacccggaaaaatcttgaaagctggttagctccaaatttactttagcaaagaaggccaaagagttttgatgtcaagttttctttgttccattgccaagctggtgtttgacctctctacgtaactggggtatcatttctttgcatatgacctctttcctggaacggggacctgctccttcacgtgggagaagcccctgacagctcagacgagccttcaagagccctgggggattcctccatcactgtgccgctcaagcccgctgctcactgatctccagcctccttggaaggaagtgtgcgaccacatgcatttccctccaaattaacacatttgtttaaaaaaaaaacaaaacaaaactcgaatttcctgcccagttttcatactttaacacatgcacttttctgtgttcttaacttctttgttgtgtttaaggatatgttaagtcaggcatggtgacttgtgcctggaatctcagttcaagatgctgaagcaggattgtgactttaaggctggtctaggtatcattcaagaactctctctctctctctctctgtgtgtgtgtgtgtgtgtgtgtgtgtgagagagagagagagagagagagagaggcacagagacagagagacagagaaagagaaacaaaatttttttttttactgtaggtaagacatattaatttaaaattatcagtttattgctcacttttcttgcaaagatcaacatgtgacttaagactgggagggagttttgtgagcgaagtggctactacctggcctctggcacagaagttggttagcttttctttcctggtctagagattggcccacgttgctctgctctggaggaagaggcaggcctctctctcccagttgtctcctttctttgtggtactagagtcagtggctgaaggctctctacttctctgaactttcatgagcagctgtttaccttcagccccatcctgctcccaaggaaggcgagccattggtgctgaggctctgcacacaatgatctggaggccagatgccagcacgtggagagaggacccaggaaaagtcatgacctcctgtagctgatcttctgctactttgtggcttcttttttccatctttctccacctttttttctctcaacatttcaactccttaacactggagaagagaggaaaaaaagatagagaggaaaggagagagatccctgaaaaaagccaggggtcagaaagagtgacatcataattagactacttcctggtgattaggggcattgagttccttggggcaaatttgattttccacatcaggatacctaattatttcttcctcctcctcctcctcctcctcctccttcttcttcttcttttttttttttttttttttttttttttttttttttttttttgagacagggtttctctgtgtagccctggctgtcctggaactcactttgtagaccaggctggcctcgaactcagaaatctgcctgcctctgtatcccaagtgctgggattaaaggcgtgcgccgccaccaccaccgagctacctaatttcttcttgtttcttctttgtgcacaactacttaagaaaccccaactaacatcaacaaaccaacaacacaccttgcctcttgggctctagcatttatatacagtctgaaaagtccccagaattccaaatgtcatgcaatcttgaaaactatgcatctgacaaaatcacaccccttctagagcatgaggcaaatcacagtcagctgctgaggacgatctaaagcagccccacatcccacacctgggattaaaacaaaaccacattcttacagcatttctgtgtttttaaaagaaaaccaatcccaaaattgtcactacaatctcccagcaactcctgtaaggaaaggtcacaggggtggatgctgcctgatgcaggatttttttagttaacttctgcctccttgtggctacttctgagaaaccctgtcccttttattcatttaaaaaatatttttatgtaagcaaaacaaattgtattaaaatatttttatattagcaaaaaagaaaaaaaaaaccttgtatttgttaattcatgttttcttttaaaatgttaagaggaaaacaacaccctatcaggaaacaacacccttcaggatggattttgcagcaggcccttatgttttagtctgtcttctgccttatttgaattttgaaaataatatctgagtgtagtagctcactctcttaggtctaacacgtggaagcataaacagatggacttctgtgggttcgagaccaccccccccccccaataaaaaaacctgcactgagtgtgtgaactagcagaaccactgtgaaccactgcgccccttctctttctagggctggaagtttaattccagtgcttgggaggcaggcaggggaatctctgagcctggtttacagaatgagttccaggacaggaaggggctacacagagagaaaccctgtgtcaaaacaaaacaaaacaacaacccaaaaccaaaccaaacaaaaacccatgttttttttttttttgtcctgttagtttcatatgatactactactataatatgcatttaaacattaacaactttaaggaatcctatatgaagatagaaggggctggtgaaatggcttagtaggcaaagtccgttgctgtttaagtcttcaagtctatagctggtctgtgctactttgtgggttcttcttttcctaccctttatctgccctctcatttcaccccctaacgttagacaggagagaaagaagaatagaggggagagaggaattaggaaaatccctgaatctgattcccttccttttgttttttttttcccttgaccagctactaacaaacctcaaccattccctagattaccaacaacctctgaccacacctctcaggagccctagcatttctgcaccctctgaaaagctcctagaatcccaaacaccacacaactgtagacgttctgcagttagcaaagccatcttcttctagtgcaggaggcaggtcacagtccaatgtcacagttcaatgtcacagtccgatgtcacagtccaatgtcacagtcggaagccgctgcacttcctacaccggggattaaaacaaaagcacattcttataatatttctttttttttttttttgatgaaaccaaaattctagatttttaaaaaaatgtcactctataaatctgatggcctgaattcaatccctggcaaagttacctcctctccacattaattgtttagcacataggcccaaccctacagccaaataaatgtaaaaaattacacacgcacacatacacacacatatatacatacacatatactcacacatatacatataacatactcatacataacacaaaacacacatatacactacccacatacaaatatatacttacatatacacccacccacacacatacaaacatttacacatatacaccccatacacacactcacacatatacataatatataattatacttgcccacatacaaatatacattcacacatgcacacacacatacacacacccacatacacacatatgcacatacatacatacatacacatatacatacatacatctcatatacacacatatacacatacacccccacccccacatatacacatatgcacatacatacatatatacatacatacatctcatacacacacttatacatacacacatatacacacacacccacccacccacacatacacacacatatacaccctccacatgcacacatatacatacatacacatacatatatacactcacacatacacacatatacacccccacatgcacacatatacatacacacatacacccctctcacatacacacatatacatatacactcacacacacatatacagcccacatgcacgcatatacatacatacacacacacacacacactcacacatacacacacataagcaaaaaacttaaaagtacaaatagggacttttttgtgtttggggagctgaggcaggagaattactttgagttgaaggccatcttgggatacataatgagatttaagccagcctgggttaatagggtaagatacttctttgaaaactccaaaccaaacagctagggtaagttttgaaggagtttaagttttgacaattatggtaaagggtactacataccaatgagaaagagtaaggagatatctaaagtgattgttgttagacaaccttatttttttaaaaagatttatttatttatttatttatttatttatttatttatttatttatttatatgtatctgagtaaactgtagctgtccagatggttgtgagccttcatgtggttgttgggaattgtattaagacctctgctcgctccagtcaaccctgcttgctccagttggccctgctcgctcaggcccaaagatttatttattattaaatctaagtacactgtagctgtctttggatgtaccagatctcatatggatggttgtgagccaccatgtggttgctgggatttgaactcaggaccttaggaagagcagtcagtgctcttaccagctgagccatctcagcagccctagacaaccttatctaactcagaagatacgtgagactgtaaatctacttctcaaactttaacgggtgcagccgttcacatttcccaggattcttcagtcatctgtcaatgctttgtgtgtccttgctaatcctcttctctaggaagtcctcagcagggcctctgctctggatgtgctcttacagacttctcaactcacaatattttttttaattatgtattttgtttatttttattttatgtgtctgggtgctttgcctgcgtgtaagtctcggcaccgtgtgcacacagtgcctttggggaacagaaagtgacattggaacccctgcacctggagttacagatgtcactgccatgtgtgtgatagatagctaatccagccctctggaagagcagcctgtgcccttaacccctgagcatctctccagccccttggccacatttatgtgtctctgtgtacttccctaagagctgaggaccacacagacttggcccaggacctgagtgtgctgccacagtacccagtgaggtgccacagttgaccttgacctttctataaccctcagagaaccacgtgagcctctccaggcctcccaggaaaggtgttgaaacacaaagggaacgctagcaaccctgcctgagctttttttgaggcaggatttcttgtagcccaggctggccttagacctcctttgtagctgaaggtgactttaaactgatcttttgccttcatctcctgaaagctgacctacaggtgtgtacatcactgtgcctggcttatgtagatggaacccagagctttgtcagacttcccggtgttaagccttgcttggatggatcacctcggaagggagttgtctcccaagcctcaggatataaaaatataccaacaggatgggttgacctgggctccagccagatggctcagatgatggggaccttgctgagcatgtccccaccacctgtcatggcatcaggctacagttcaagtccatgacacgcagctgcctagggatgtgtggtgtagctgactctctgacctgcacttgtgtcccagtgtcatccttggtcatatctgagacctttcccaagtgattaagaaaccctccaacaaccagacaaaatcactgtggtaaactgaggcagagatgggctaacagaactttgagtattgcatctctgtgcagaatacagctcagaaacccttccccaacagccttagccatttgatgctctccacccattctaaaaggaagtgtccggcagagccaggagatgagcctgcacttgggagtggagtggggtccggggaagggtctggagtcccacatcctgctccactgcatcatgttcatcttcctaagacaacactacaggacggctgcttctgtgatgtgttagctcaagcgggtgtggtgtccttaggtgtggctgtatctggtgctgttctaagcggggaaaccacttccctcttccaggcattcacagatgagagtcttcaggttgtctgtgccatttcctgggttgaggatagattactcttctccaactcattcaagtttattttattttttaaaagaaacgatctcctttaggtggttaaattttgaagaattgtctaggcctttgacattgttttctgtgagtggctttcttacaccagccgggcagcgagcagggaatgccccaggctatctatcttgcaggaccaaggatgcacagggatagtaaagctgcctctggtaacaatcccagtttagctagcacactcccacaggcttcagctggaggctgaggggatgaagagggtgagaggcttctcaaagagtttagaccccgggaggaaagtgaatttatcacctgccaggcctggctctcaccacccaccttgtctcttcccacccactgtgggacacagctagggggtggggatgctatgtaggaaaatgcaggatatacatgaccgatgtatagcctatgggtctcactatgagaagtaaatacatgccatcattgcctagctcagaccgtgcatccggccgcctgggcacctaccttctgcagcctggttgcctgggtctgaagctctgacttacagtgaccgatctcatccaggggcacctgctgaagtttccaagtgcttgcacttcaggacaaagggctgcgccaggaacaaactgggagagatggagatggtttgcgcccgaggggcaggcagttggtaacggcagcaactgcctgatgacgtgaaccctatataaggaactcctgtgttagaagaagagagctgtggatatacaatttgtatacacagagcaactgcaggtttggcccatgatgatacagactctgggctgtggccttacctattagccagttattcaaggctgcctcatctgcgttctcctgaagaagagcaaagagcttcccagcaacatttaaattactgatagagccacgacttccttgtgtgctctgtaagcctttgtgtggagacagccacacacctcagcatccagaacgagagtcaggagcagacggaacctaaaggcatcccttgtttgcctgcaagtagcaagctggtcctcacctcggcatttagtgtagatgcagtttaggatggctatcctgctgacagagagccctcaccccctgtgtctaggtaccgtggaggcatgtgacttccactaggcccaacaggatctcattcctggaggtttggagttggctccaagtgggtctccatgtggctggattagacacttgtgaggccaggagtaatgagaagcaggagctaccaggaaggcggcatcgatacagctactggggcctctggcttcctcaggctggcttaggtctttgcagagcaccagactagtcctgcttctctgcctctgtgaacttcagggatcctcagcagaactccttgtacgtgacttgggttgtattgattgtggtgagttgcagttaagaaccttaattaatgtggcgttacccaccagggggaggggagcaccacaacactggctgtcagggagtgtgaagtaggatgcacacaccttcccaatggatcctcaaaggtgaacaggagtgcctctcaactcctgggtccaaggatccagccagtcacactggatgatgatttccacaacatacaaacggcccctccagggactgtactttcctggggctgtaatggagactccagaagggcattctactgtctggcaatgttatcacgatagggactgctgtgagtgtggcctgttgtcatggtgctctactgtgatgcttgaactctgggagcagcggagcatggcacaccgtaacagtggacaggggtctctgcagggactcaggtattcataaaacacacaaataaatgtagtccaggctggctttgaacttgtggcagtaaatctcccaagtgctgggattacaaggcatgaaccaagggccatgggatttggcatgaatgccctatgagagaacatataaggtcatagagctgccccttggctgatggaagtgcttaatgtgaggaagcaagcactaagaagctggctgaccaccagggggcagtgctgtgtcctagactctgtgttggtctttcctgccaggcatggtggctactgtggctgggtgggcttgggtgaataaagatctactctgccgagctcactgccacatccatccctacctgccctggccaccatgctcacgaacccatgggggtaacaacagggcagctggagaaagaggctggctgctatcctcagaacatgccatcctctctgaggtcacctcctggtgagcgctcccaggagatgaaactatcttcacttttatgtgtgtgcatctgtgagcacgcatgtgtacaggtgctcatgaacaaagtggcaactgaggtttttttttaatctgattctgataattttgtaagttttaaaacattcattttcattttgtgtatgtgtctgtctgtctgtctgtctgtctgtctgcgtgatggtgacagggatagctagaagagggtgcctgattagtcctctggagctgcaattgcaggtagcagtgagctactcaggatagatgctgtgaaactgaactctgatcctctgcaagaatgctatgtgctctgaactgttgggccagctttccagcccctgttttaagacagtttatgttatatattcagaggtatgcctagttatcctcgggagtgagtcaggtagacctgagagtgtttatggcaggctggccaattcaactgtatttactacggagtgcctcctgaggaggagaggcgttccattcgttttagctgaccacttcagatcgatcgcaggccatcactgaggggagtcaaggcaggaacctaaggcaggcctgcttgctatagcacacagcattaacctccaatggagagctcacttcactgccaatcaggtatagcaggaaccacagaggacgctgtttgccagctggctcgtagatgggcttcagctcacttagctttcttggatagttcgaaacctaggtaatggtgccgcccacagtgggctgggccttcgaaggccagttaactgtccagacctttcttacagacgcgatacgggccaatctgatctaggtaatcatggactaaagctctctgctcacaggaccctaggctatgtcacactgacaggtaaagcaaactaggataccaacacagtgcctttttcaaccctgttaccctacccactgcactaaatcctgactctgctcatgtctttatcaacttcagccgcatccgcccgtgcttacccgaaggttcctggtaagaggcaactgctctggcccacctttccagcacctggtcttttttctagggccacaggctcctcccttccagcacgcactctcccaggcttcttcccactgccccatgctccacccacagtcaggtctccggctgactccatgcagttcagttttgtaggtttatttttgcctcggggtccagaggcgaggttatgacgggcctaacccctgactggcacttcctgtacccactgtcctgtagcaggtgctttgctgaagcagccaaagggaagggatgggggaggtgagccagctgccaggagcttggcacaagccaaggcttgtgccgtgctcctggccagccccgtgctgccagagattcgattctcaagtcatttttaacagctgctcccaccccccccgacctgtcatctgtcactttccttcggcttcaccgcgacttggtaggaagaagtccctctcttgaccaccccgccttttactcccagggtctgggaagcgcccagtctttcggtccctggccacactgccccatcacacttcccctccttggtttcctccaagtgaaaagcaaaactttctgcgctgcggtctgggagccgcgaggggcggcgcgcgcgcgctagcgaggggccgaggctggagaggaagcggcggtcctgggcaggaggaagaagccagccctcggctcggccccctgcccgtttcccgccgcctccgcggccgccgctaccaccatcaccaccacagtcaccgccgtccggggcgcagccggccgcacgatgcagggcgggggtccccggggcgctccgatccacagtccgagccacggcgcaggtagggcccggggttctggaggagggcgaacgaggcctgccgagatccgggcgcaccctgatgcaggggagggaaatcgaggcagtgagcggggagtgcagggtgcgggcggtcgtgtccccagctcccactgcagctcccgtccaagcgcgtccccgtcccgagagtccggccgccctgaggcgcagtcggcgaagggcgcgcccgcactccttccttgcgaggtcccggtggccgcgaaggtttccgcagcgcccaagtgcagctcgatcccaggactccctcgggtggccggcgcagacgctgcggggacgcggggcgaggttgagagaggtgtgggaactgcagcccgcacctcccgtggggctgctcgccgcccacttccgagtgtggactgtgacgcgcagggcagcgcaggctgaggggacccgctccatttcctctgtcagcttgggccacgtaccaaccccggtgatgacatcgagttaagtgggtggccgcttttcaacctcagcaactcactgtcagaaaccccaagaaaagaacgtgatggcacacgcctcttttcctagcactcgagagacaggaagactgtggagactttctaatacaatctgggctacaatagtgagactctatctcaaaaaccaaaatcaaatcagaacaatgaaaaataaaacaagcacggagatctgctccagactgggctttgcagtttgggcgtgtgctgtgccaaggagagaaggcaaaggacttaggagcttcttttggctcttggagcctcagtttacccatctgtgaaaggagcactaatcactagcttccctacctccttaactggcttctatgaagacatttttgcaattgggagttggaaaatacagggttggtaggtgggtgtagggtaagagtcttctgcacccgcatcctggactcgggaatgccttagtttttcaatttgtaaaatggagctgattttcctgcctacgtggtagattttaaaaggggtttaatgtggtaggaggacatgaaacccctggccaaatctctgcgatacaaagcaaattctcaaaaatctcttagttgcccaggctagccttaaatctgctgcctagcccaggctggcctccaatttgctagcctttgcctcccaagtgctgggattacaggtgtgctccactacgcacgggtccttaagtattattttgaccatgcaagacctaaataatcgtgggcaatacaggaatgccagtgtgacatgttagtgtctgagttacagggtatttgaaaagacatttggcacctgacatggagaccgttgaggtttagccaggattctgcccacagcaaatagagccctggatgagaaacctgctttctgagctgtctctccaagtctcgggctttctcttctgtttataagaaagagaaagcacctgggctgtaattattaccttagccctgagagtgctgccctgggatggagagcagaacatctggaactggggcaggtgacagcctacaggcgacaggtgccactttgcagagagtgcatgggccaggcacttggtcctgccctttgatgaggtccccctcacaccggtgaggggctggtgctaccatcctcatcttcccttggatgtggcaagggttggagtcacctgcctgtggccctggtggttaaacagggactggggctcaaagtcagatgcaggtgtctccaattacaggctgaagggaaggtgtcaggtggagcccaggctgagcgtggacctttgcctggtcaaaggtaatacctggtcttctgtttctgccgtttatacctatcttcatctatctatctatctatctatctatctatctatctatctatctatctatctatctatctatcatctatctatctatctatctatctatcttctatctatcaatcatctatctgtatatcttctgtctctatcatctatcttcagcatctatcaatcatctattacctatctatcatttatccatcaatctgtctgtctatcttctatcaattatctacctacctatctatcaatcatctatctatctatctatctatgtatctgtctatgtatctatctatctatctatcatctatctatctatctatcatctatctatcatctatctatcatctatctatctatctatctatctatcatctatctatctatcatctacctacctatctatcaatcatctatctgtatatcttctgtctctatcacctatcttcagcatctatctatcaatcatttattacctatctatcatttatctatcaatctgtctatctatcttctatcaattatctacctacctatctatcaatctatcaatcatctatctatctatttatctatctatcatctatccatcaacctatctgtctgtctatctatcatctatcaattatctacctacctacctatctatcaatcatctatctatctatctatctatcatctatctatctatctatctatcttctatctatcaatcatctatctgtatatcttctgtctctatcatctatcttcagcatctatcaatcatctattacctatctatcatttatccatcaatctgtctgtctatcttctatcaattatctacctacctatctatcaatcatctatctatctatctatctatgtatctatctatgtatctatctatctatcatctatctatctatctatctatcatctatctatcatctatctatctatttatcatctatctatctatcatctacctacctatctatctatctatctatcaatcatctatctgtatatcttctgtctctatcacctatcttcagcatctatctatcaatcatttattacctatctatcatttatctatcaatctgtctatctatcttctatcaattatctacctacctatctatcaatctatcaatcatctatctatctatttatctatctatcatctatccatcaacctgtctgtctatctatcatctatcaattatctacctacctacctatctatcaatcatctatctatctatctatctatctatctatctccagccatctgcttacctacctacctgcaagcagcactagggatcaaacctagggctctgtgcagtgctaggcaagcactttagcactcagtcaaacctctagctggcatttttcataattgcagaccttaaaaaagtaagagtaaatcacagaaagaactttgcttttctcccccaatgtaatatttttactaattatttgggaattacatacaaatacattctggcccacccccactcacttcctattcctcccaggtccaccctcccactcttgtgcctccccacccacccacccaacaaacaaacaccaccaagttcaatttgtgttgctcagacactcactgtagtatggctccactcccagtgactagtcccttaaagaaaactgagccccccaccccctccacctcctgcagaaagaactttggaaacactttaatttcatctttactttataaggttctattttatgatatcttcttgcagaagaacaatgaattataaaaatttatcaagtaaaatggtattaaactttgaaagaatgcattatgagatcttcatgcaaatctcaaagttcggtctaccgtgtgctctgttttagcatgtgaacttgctgaaggcgggaaggtagagatttttggtcagtcaatcacttcacaggccggagagccctaggacaggctgtaggggacctcagtgctacccaagatgaagggaggggagtttcagagaggagagcccagactggggctttgttacaaaatgaatttctggttcctctgaactagatatagttcaacagaagaaactgaacattaaaaccctgggctcaccagagcagacaggctgaggctgctatgcctgtagtatgcttctgtgttgtatccataattcatgtgtgtttcaaagacgggaggcacactgttactgcttttgttaaatgatgggagtggtacttgctcattgctcaaacatggagatatttatataggaaaaagccagcttcctgtgtccattaaaacatcttcattaaaaataattgttattaattaattaaagcagttgttaatgtgtgtatatgcatgtatgtgtgctcatgtccaaggaggaaggagaaggcgccagatcccatggggctggagctgtagggtgttcaaagtagcctcatatgggtgctgggaactgaattcagcccccccccccccatcagcacgttttcttgaacggctgagccacctttgcagcccctagaaacaatcttaacattttgaatgaagaaccaatagaattccagttgtagatgtcaggattcattgtttcaatttgacgttagagctttagaacaaagagctaatggtaacattgacattagaaagtctcaagggggctggagagatggctcagtggttaagaacactgactgctcttccagagatcctgagttcaattcccagcaaccacatggtggcttacaaccacatggtggcttacaaccaaatgggatccgatgccctcttctggtgtgtctgaagagagctacagtgtattcatattcataaaataagtcttaaaaaataaaaggtcttaggagtagccacgttagtgtagagtgacactgagtgacatttttggttagggccactcgacaggaattagattctttcttttataacaatgataataattgatgtcacatgttctgttcccatgccaccttgcctgagtccatgccacccacacaccttgggtggtcaccctgggttgggaacggttctctcctggcctctcctggcatggctggaccacttgtggaggtcatttgtctcttattatcctttagtaaggatggctttcagctctggggagtcctctgatgctttggcttaactctagtgtcactgtggggacagcctctgtgcatcctctctaaacctgattctttagccattcagaatgcatgcctcctctgagagtcagagcccccagctgcagatctgctgccagctcttgctttttcagtaagcagaggagggcagggcagggcactggagccaggccgcctacagatgagatgttggtatttggctttgggaggtggtcaggggatcactagagcctctccttccttcactgcttctcctttgctgacttgccttttgtcacagcccaggaaatggatatggcttcctctaagctttgggatccctgttcatcgacagtagtaaccagacatctccagctcactgctgaacagagctttcagctgtcgagctgggatatcactcacttggtaaaccacttggtattgcactgtatttgtgcaaagccctttgattcatcagaaggtatgttaaaaagcaaaagccgggttggcagcatatccttgaaactccagcactgtcgaggcagcaacatgagggtctctggtgctcgctggccaggcagcctagttgatccactgagctcactgttcagagagttcctgcctcagaaaacaagggacagcatgacagtcacctggtgtcaactgcttgccctgcacacacatgcctaaacatttgcacacccacccacccacacatacatgcacacatacatacacagatacacatacacacatacacatatacacacaggcacacaggcacatacacacatacacatacatgcatacacgtacatgcatacacatacatgcatacacatacacacatacacacatatacatatatacacacatatacacacacgcacacaggcacacacacacacatacacatacatgcatacacgtacatgcatacacatacatgcatacacatacacacatacacatatatacacacatatacacacatgtacacagacacacagacacatacacacatacacacacacgcatatacacatatacatacaaactccatgaatttaagtttcttgtatcctgataggtaaagcctctgatgccctcattaaacagtacagctacagataaaagtgtgtgtgtgtgtgaaagagaaactgtgtgacaatctttctatgtaagcaaacctgttcttggacttataatctttctgactcagtcccctgagtatctacagtacctagtacgggtgtaccccactgtacccaacattaagaactatgtgtctagatatctgattaggggaagcagtagcagtcatgctgcgtctattaaatgcattagtgattaactccgagtcaccgataactcgtcaatattgcattaaggaaagaaataaaagcactagcccttggagtcatagagccaagagtgagttctgattcaaattcttatctgcatgttccctgtagatctgtcctccatctgtaagtctgaatctctgcctgcatggaggctgtcttgggaatctgtcttctctctgcctgctctgtctgtctgtctctctgtttctctctctgtctctgtctgtctgtctctttctctctccctcttttccttgctcctcccctcccacctccctctctgtgtgtctgtccccatgtgtgtctccactgagcagcacagaaagcattatgtgaggacgggatgccatactttacagaaatctttgtttttcaaggactaagtcgtttgctccacctgatcccaactgtctcacacaacaaacagtacacgcttctctgtttatcagtcaatatccacaaacgcgtgttcattttagaagactgcttccaacttccgtatttgctgctttcagcaaatgctactcaggcattatctgggtcacgggcatggaagaacacatgccttaagattatagctatgggtcacgggcatggaagaacacatgccttaagattatagctatgtattagcttaggttgtaaaagttgatcacatgggaagcccaaggcaagtaaggctgattgttaaattgttctcctaaaagcagattaaacaatcaggctgagtgtacagtagggagcagtcttaagttttcagtgacctcaaggtgtataactaacttcagttctgggtccatcaaagatttgcctgcctctgcctctggagtgctgatattaaaggcgcgcaccatcactgtttggcaagacgtaggtccttaattcctactcttgtatagtttatcaactgaagaagctgggtgtggttgctcatattaattgtcctagcacttggaaggctgaggcaggaggattgtgagttcaaggccaacctggactatagagtgggaccttgtctcaaaaaccaaaccatggccaaaccaaaggaagcgtccgttcattcttcattcattgatgtaacagttactgagtatctacttgatttccaatctgaatagatttagactctcaggtcggcagacattagcttgctttgcagcttcattcagagcagtagttctcaacctgtgggttgtgactcttttgggggctgaatgaccctttcaacaggggttacctaagaccatcagaaaacacagatgtgcatattatgattcataacacgtagcaaaattacagcagtgaagtagcgacaaaaataactttatggttgggggtcaccacaacgcaaggaactgttttaatcccagcatcaggatggctgagagctgctgtactagaggaaaacttgtcagtggttgttttctcaagggatgggctagcttgaaaggctgtgagcatgttggctaccttgggtgagtgtgatggaggttctatcctggggaggaaggctgtgtgagatgatttgtgggacctcccctgagcccctgtgattgatgtcctggaggagtggggtttaagctgtagtgagtgtttactgtctccttgcttgccacgtgctggcccagtggttcctctctggaatccagagcacccacttctgctctgggattggagcatctggggctggtgttaatcatgagcatggtcatgatcatgcttacccaggcagacaaatgtgagacgtcagagtgcaggcttatggatgggtggctttgtggagagtgtggttgctttcagaggccagggggtgagatttccacagtgcaatgtgcaaactgtgtcctgatggtggaaccagagttgaaatttaattctgtggcactcagacctaactgtttcctttcacacttgttacctcttgacttgtgaatggatggataggacaatgggcagagtcaatccaggaatgtgtgtgtgtgtgtgtacatgtgtatgcatgtgtatgtatgtgtgtgcatatgtatctatgtgtatgcatctgtgcatgtgtgtgtacatgtgtgtgcatgtgtatgtatgtgtgtgcatgtatgtatgcatatgtatctatgtgtatgcatgtgtgtatgtgtgtacatgtgtatgcatgtgtatgtatgtgtgtgcatgtatgtatgcatatgtatctatgtgtatgcatgtgtgcatgtgtgtgcatgtatgtatgcatatgtatctatgtgtatgtatgtgtgcatgtgtgtgtgcatgcgtgtgcatgtgtatgtgtgtacatgtgtgtgcatgtgtatgcatgtgtgcatgtatatgcatgtgtatctatgtgtatgcatgtgtgcatgtgagtatgcatgtgtgtgcattcacatgtgttgcctttgtaactaaagcacctcacctaagtagtgatggggaggaagaccttccattgatcagctaaggaaaagtgcagatataaatttggcttctcttgtctctagttcactggaaacttaggaccacaaaagctgctttatgggcgtaagacctctgaagattaatcttgcacactggctgttgttaatatttctgtagttcttggtatcacagccaggtagagtgcaaagacagaagtgtccacagctgtgcttggggtggacaatacacgttcagttcaaacagtgagtgaagggttttatgttgcttcatctctattaacccctatgtcttatcatgtctcctcctaatgacaatactgttcccaggactccctctgtagtcatttctgtattcaaggatacatatttggctcttgttgtcatgcaggtgtgtctaatctgtggcccttgggctgcaggcatccaaggacagccatgaatgcatcccaatataaattgttaaacatatttaaaacactgtaaggattggttttggtgggaggagcatttcagttgtgtgtttctcaggagtgagcatcacagatggcaagataatgttgcaagatcacaggttggaggtgcctagtacatactgtactgcacatggaagtatattttaaaatgaagcacactgtccctgaagactagtggagagatttaagcaagactggtgttctttgcaagattcttaggtgagcctatataagctggcccctaaatactggaagctgccctctgacctccttattgtatggtttacggtggcacatgggtgggtgggggattgtcacaggctcttttcacttgggaccatcttggaaggagagaagatacaggtttgtgtgtgtgtgtatttgggtgtatgtgagtgtgtgtgtatgtatatttggatgtatatgcttgtgagtgtatgtgtgtttgtgtgtgtatgtatgcatgtgtatatgtggatgtatatgcttatgcatgtgtatgtgtgtatgtatatgtatgtgtgcatgcacatgtatgtttggatgtatatacttgtgcatgtatgtggaacctaaagaagggtgttgaattccctctatcctctgaagtggaaacgtctggtctctttggaaagtcagttatgtcaaatgatgttttgctgggggacacaagtgaaaggatgtttcattaaagcagacacaggtaaagggatgctttgctaaagcagacatataaaaggacatgtggtgtttagattataaatatgaggacacagacagtgggagcttgatcattggttctctttgcttcatcttgctagtcttcactgacaacacaaatgcactcattcaccttacattgcattgcttagcttcccttgtggtgacttcatagagagaaactctccaaagaacttcttgtgaggttcttgtgacttcttgccgctcccatagacttgggtaggttggcacggtgtcttcgattgaactgcccttgttgattcgtgtgtgatgggactgaagctgctgatttgtgtttggtatttgctagtggactggcctgagaacaatgaagactggaatttccccccccaaaactatttctaaacaggtccatttccccagtttcctgatacctctctttcctactacctttggtgtgtggtgggctagaggggaggttgaacccttataaagtatgctgagaaaaatttatgcccacaatcctcttctgcttattcctttggacattgcgataagtttgtttggtgtttgttcctttctaggttggcagccagtaagcctcagttatccccgtgcctctgtgtccctctgttctggggctttacaagcatgtgtgaccatgctgctgctgcacgtatgctggggatttgaattcaggttctattttgtgcacagcaagtgctcttcactgctgaaccatttcacccccgtattgtgtcactattttgacattgtgacaagatgttgtcatctacaaaggctggagagccacatgactgtgttttgttgttgttttaatctcagaatctcttatgttgaccacattcatagccattccaggtgatacttgaagccatttcaatgggaaaatggacaccttctgccttttaaagctaatatttatatattctggatgctatggtttgggataacataagcacattattttatgtatgtattgcatgtgaatgcttaagacctctaagattatcgaggacacagggttctgagaagtacaatggccttagctgtcctctggctctgttgctgtgggttgatgttgaggtttcagacttctagactttgtttccttttttagtgagcaaagggtctttcccctgacagtccttcagttcctggcatatgttttatgtacattgtatgtttaaggtcaatcaaagtggcaacagtttctaaaggagccttgtaagtgtttcagaagagaaaaaaacacattgtcatgattttaacactggagtacctggactgtggggcacagtgtagggcacagagcatcccaagcctgggacagtggaacacagtagaggacacagagcaccccaagccagctctacagctaccagctctcaagcacccttgtcccatctcccaccggcgtctatcgccaccatctatcaggaaccatcaaagttcccgaatcacggggaatgcctttcttatcagagagcacagagggcatgctgggaacaaatgttattcaatcagcatccaagtccttgggtgcccagttttgcagcccaaggtcctgctgggctggagggtgagctggacctggcactgtgcctctaaaggtgtataaactgatgattgttccacagtacggtcaagggacaggtttttattgtaggtatgagggagagaacagccagaggcatcaggaagggtcctgagcacagagaaaaaaaatagattgaatgtggccagcagagagatggggaagaaggacacatgtgtgcatgtgcacacatataccatacacatatatgcatacatatatacacatgcacacacacacatacatgcaccacatgcacacaccatgcatatacacatacatacacacatgcatacacacatacatatatacacatgcacacatatatacacacctcgcacatatacacaccacacacatacacacatacatatatacacatgcacacatatatacacacctcgcacatatacacaccacacacatacacacactacacatacacatatatatacacacatacatacacacatagagagatagatatagatagatagataaatgatagatagaaagactaagagattgtgggcaaaagagagcatatccataatagcagggttatgagaagaatgagtagcagaggggagggaaacccttgagctggagaagttgagtgtgtgtgtgtgtgtgtgtgtgtgtgtgtgtgtgtgtgtgtagggggctaagaagagatgggtactagtgatcctgagggagcctgaggtcagcatgggttttggaatgcttatagccacctcacatagccatgtgttccttctgccactgaaaagggaaatggcccctttggcagagggactgtaggagctcatgggggatgctggcttttgtttaaccaccagaatgcagggaaatggtgtttcctttgcatgtgagagagcagccagcagcaacagttctgtgaagaattctttagttagtggtattatctcagtgttgcagcaaatcactggcacaggaggtaatggggtaggctgatcacacaaaccaagggcttcgagtgcagcttgcactggccttgtggagatcccagaaccggcagagctttgggcttcttgcttgattttgtctggaccttgtgtagccgccatctctgtgcaatggcatctgtgtaaggccctctcctaggatactgtgtttggtttctccatgtgactgacaagcaggcatccactggcagaacattcttgcatgtacaacttcatacctacagagggcgccctgaccccaaagaagaccttttttgggcttttacgctacaggatgggaagtgagtatgttttgaaggtacttaataaaaccattactaaacgctcgcagaccaaccctgggactggggcacgctgcctttgccttcctgcagaaccctgcaggctctgggtaaagttttggaactgttgcttgtgaactgttaccacttctgtgacactggccgcaggacagcctacttggcccacgctcagggtgttccttccgcattgtcaaaggcctctcagagcagaaaaggccagcttgcttcctggaaccccacaggagccacagtcaaagggcttcttgtttcagctgcagaaaacccacaggaaatgctggcttcagagcagctatgagttcccattgtcccctcagacctagccatgttccccccccccccccactcccctctgaatgaggtagcaactagcatacaaacaaactgaacagtagcgtttccggcagccaggggactttccgtccaagttctcccagcaatgaaagggaagagtcccagctagcttccttgtgctttcagggatgatcgtgccatttgtacaaagctggcctcatagtaggcactttatcaagactattgaggagaggtgactcgattccaagtctccgacagcccctgtcactcactcactagctgtgtgggcagggacgagctgtttgattctaatctctgcctgtgtgctgagcaccgcatgcttgcttcctgaaactgtcacggggatgacagtagacaatataagacaacgtcacacagtcaaggcaggccgcagtctgggactatgccaatgaggtcccatgggatggggcatgtggcctcagcaacttatattctaatttctcagctcacggatgccctctcctgaggggcagcttttggggtttggctgatggtctctatgtggcagcaggggtgtatcatcaacattgctagcttagctggcatagcatcctggagggtgtgtctggtgacctcactgtgccccaggatgggtcattgcatttatgcctctgccatcttactgctgaccttggcacgtctgtggtgacgctacaggaaccaaacagaaaaatatctgtggcgcgttcatctctctctcagcttcctgactgtggatgccatgagaccagatgcccaagctcctgtcaccattccttgcctgctttgatgtgctgtgcctgtcctggttgactgtaactgtcatctgagaaggaagtctcaagtgagggattgcctaactcagattggcctgtggtgtgcctgtgaaggatcaacatgttcgttaattgatgtaggcagtctcagcccactttgggtggcaccattccttagacagattgtctggattgtataagaaagttagttaagcatgagcctgtgagccagccagcaagcagtcaccttctgccatgatttctgcatcaagtttttgtttgagtttctgccctgacttccctcagtgataatctgaggaagaaaccctttcttcccctcatttgcgtttagctagggtattgttttttgtttattttattttatttttttagatttatatatttattatgtatacagcattctgcctgcaagtatgcctgcaggccagaagagggcaccagatctcattatagatgactgtaagacaccatgaagctgctgggaattgaactcaggacctctggaagagcagccagagctcttaactgctgaaccatccccctctcctttccatcgccttcttctcccccttcctttcaccttctgccctcctccatctcctaaagatagttccaggagttagaacaatggtttcccatagatgcttgtgtccgaagcatctgtgcatagctttccccagtgtggcttggaggagcttgaagggtagatgtggtgaagtctaaaactatcaatttttggatatgtctcaaatcacctgggttgactgaaggaattgtgggtgtttctaatggaaagaagaaggcaagagtgaaggtcaggagtttgaggatgctaaactgtgggtttgggggatgaagggagctcaccggagaggttccagaggcgagttgatgatcttcggtggtttccagggggagacttgcgctctcaaaccctgggccagaatacacttgcctcctcttaggttgtttcttgtcaggtttttggtcacagccatgggaaaagctgcttaataatgtcacttaaggtcacacagagccatctccctccaggctttcttctctgtcagctgaaggctaatcacgccaagtgctcccctgcagaaacctgctcttctgtgaaggaaatggccgggcttctgtgagagtctgtctgggaagccgtagcctcaacagatcctccaggtgcctggatgccaggagaatgtcattttcacttccctgagaaattgccagcccgccccagggacatcagagggctttagctaatgccacacccccttaagctctaaacatgagattgttagtttctagagaggagacccgtctgtgtaggtgagcggctgcaaaccccacctcccttgtctgagtaccaggtcttttcagtgcgaagcaaggtacaccttatgttttggtactaacaggaaacattctgtttgctcagtccagggtcatcactgaccataacccctgagctctttatattatcacaaggcccaggacaatgagtcactcttgggtcttatagaggaatatctgccatcttgtgtcatgtgacttctgctggaaggaaacctgcactccactaatctccccaggggaatatttaaggcccgggctaataaggtgagacagatggaatgctcagagaacgctggcaaattctagagctaatctgattaaagggcaccgaatcctttccctagcacataaataaaatgtgacaatcggtgtcagccgagaggccttcagctggcttttaggcttgttacttggttctgcacgttttttagcaattcccagcatgctgcagaggaaagaaaccaagagaagcagagggagccaaaaagggggtgcccatcctctgacagttaaggtgccagaggctgaggcagacacagttttcacttgctgtcttaggtctctgatgggagtctagggcgagaggttgtggccacagagaagtcttgggaagacattcctctttcaccctaagaatggcttccttgaggaagattggtacacaacactgtatgtggctgggaagatgactcaatgggtagcgtacctgccacacctaatgtataagtgtgaagaccagagttgaggccctaatctcgtcagacccacgccccagctctcaggagatggaggcagaggatccccagggaaagctgggtagctagaccaggcaagccagcctgtcttagtcaatagagtggagtgtgatctaggtagggtccagtgtacctggactcacatgtccacagacagagacatgtatatacacatgcacacatgccttacacatgcacatatgcacatatgttagtgcacacatctgatgaattggggatgcttatacactcatgaagtcactgccatcaaccctgtctcctccctgaatttctgcccaactcttttgttgttgttgttgaattaattccttgtaaatttatttattgggagaatgagtgtgtatggttgtgcatgccacagcacatgtgtgaaggtcaaagggcagctctgtggaatcagttctctccttctacttttatatgggctccagggattgaactcaggtcctcaagattttgtagcaagcccctttaccgactgagccacctccctggccctaccagtgctttttttctattggtgcacacaaatcacaaaatctaccctctgaggaaaccttgcttatggtagtgttgtgagctctaggcatgatgctgtatgtacagtgaaatggctttattattttttaagccttaatttttttgtgtgtgtgtgtgtgggagtgtcattggtgtgaactcccatcttgcttcttggaaatgctggttggcatggctttataggttatttgataacaatttcacttccagggctcactcctgtatcaatatgggtgaggcatgttccctaatgatataaaagatattccagagtcacagaatatggcctgggacacatgtgcttcttgtgttctcctgtcaaggagaaagcctcagcttcatgacaaaactgtttttgcactgcacatggtgtagtctttgtcagttacatttcaaactggtgaaaggttgtcctttccctctgatgatagcatgtgtgtgtgtgcctatacatgcatgtgtaaatgtgtgcatgtgtatgcatgaatgtgtatgtgcatgtgtgtgcacatgtgagtatgtacatgcatgtattcatgtgcatgaatgtggtattgtgtgtacatgtgagtgtgtgcatgtatgtattaatgtgtctgagtgtacatgtgtaagtgtgggcctatgtatgtgtgtgcatgaatgtgcacatgtctgtatgtatacatgtgagtatgtgcatgcatgtatgcaagtgtgcacatgtgtgttctttcatgtagagaccagtgattgactgtgatatctttccctatcactctccaccttactttttgacattatgtctctctctgaacctgaaacttgtttcagctagactggactagccagtgagacctggaggtctgcctacctctaccctcctgtacagtgtagtagcatgagctgctacacagggcttttaagtgggtgtaggggatccgaactcaggtcttcatgcttttgcagtaaacacatctcccatcaaaccatctctctggctccataccatgaaattattctatgaaatgtatttaaaaggaaggaacccattttaagcaaagaggttgaaaatattgttagaggtggcactcagcagtaagaagtgccctaacatagcttggggcatttgatgctgtccatgcccgggacatgctcttgggagacaggccttggtaaggacagcacctgccccccccccccccccattccatggagcaactttcacttctctgaggacagcactaagtgttcttcttcagagtgtacccttccagttctttcccaggtctcacaggtgatgtcctgtgtggggcctgagactgcttctctctcatggcagctcctcttggtcagtggagctgggactatcttttcttctatcaggaagcgatttctgagactaatggagagaagggagtttgggagtctgaagagacacagaagcagcccagaagcacagtgaggcaggcgcctgggaggtctggacaggggcaacaggagaggcagaacaccgtgtcctcagacccccttctctcccggcacagaatacaaattatgagcaaaaggatctcctcgtgggaggcagtgaggaaagctcaggtgggaacgtcggccatgtgtcattgaggtttgtccagctgtcctttagggcctttacagagacttcatttcccaacatggctgaagcacgcacagtgtatggaagtactggagtggggtctggggaggcacttgctgggtaaagcacttgctgcacgcgtgtgtggatcacagttcagttccccagaacccacctagattctggtatgtgtgacgtaatcctgcaatccctgcattagggaggcagagacgggagccccagggcaagcaggctacccagactggctgaactggcaagttctggattcactgagagatcctgcctcatacacactcatacacactacacatgcacacatgttcacacacgtcacaaacatgctcacacagactcatacacaccacacacacgtgtgtgatcatgcacaacaagtcagactagtgctcatagactggctatgacactcagtgagtgccagtctcttcctagtttctctgtgcacttttcttttcttttcttttcttttcttttcttttcttttcttttcttttcttttcttttcttttcttttcttttctttcttttcttttcttttcttttctttcttttttgactccccccaacccccgagtttggggcaggacctttctgaaatgagggccttgtgatctacatccaggcaagctaggccagagaattcccttatggccagctctctgacacaggcaggggaaggttccgtgtatttttagttcacatggctcatcttgaggagaagaacttgcagttctgatgacctgccttagagagatgaaggggccggtgaggcggggcgggtgaggcgggggagggaggtgttaggaagagtctgctttctgaggccactgaagccccacgtgcccagtgcccttcaccccttcactcggaagctatttccagaccacgtcaggagtgcaggatgagaggaagtttgactttcttcttcatttaggaaatggcaagggctacgggagttaggagccaggaacgtgagccaaagccatgaatatcaagatgtctatgttggaagctgttttgaattttgcactatggacctggaactttctggagccatgtgtcttcattggttctcgctggaatcagcttccacctgggcacctggggttggcatttgtgttgcttggttttctttgttgtgctaaaatatccaacaaaagcaatttaaggaggggaggaaggcacctgtcactgtgctcagacctctcctctgcccctatgtgtgtgtgtgtgtctgtgtgtgtgtctctgtgtgtgtttgtgtgtgtgtctgtgtatgtgtgtctctgggtgtatctgtgtctttgtgtgtgtgtgtgtctgtgtgttattgtgtgtgtgtgtctatgtgtgtgtgtgtctgtgagtctgtgggtgtgtctttgtgtgtgtgtctctgggtgtatctgtgtctttgtgtgtgtgtgtctgtgtgttattgtgtgtgtgtctgtgtgtgtgtgtgtctgtgtgtgttgaggatctgaacttgggccctgtgcttgtgctgcgcaccctttattggcttagccatctctcagtttctgtttgtttcttttgagataggactttattctgtagcccaggctggccttggactccttgaaaacctcctgcctcagtttcccatgtgctgagactacagttatgtatgcctgcacttccagcttcccccgtttctcttgttgaattgagcatgctcgccagttctctgtctccgtgttgctggtttctcggcgcctgccaagtgtgtgtccttggttctgctcatgatggatcatgatttgtttgttaccagagaactggagagttgcttattccctggtcaaccggcataaaagtttgggtagagagcagaggagggagttaggatgctttaccggaaggacagtaataacatgcaccttggtagggcagtgcgcggccgaccaaacaggagcaaaggatagcctgcctcagtcttgggctgctgtggctttctttgcaatggacatggaaaccatgctctgcctaggatgacctaagccttgggtgcaaatcctacaccctgtctttcttagccaacccagggagagtaagtccccacacttccatacagctggtgtttcaactctgccttctgtcaaaggctgcactgtgtggccctgaacccaggatgggtgtttgaggcagtagggctgccagttgtatttattgcatttcactgaccttccaagctcttgctagcctcacaggctcagcctggctgtcctctggtttgttttagcagagcagtttgaggcttagtttgtggaaaggctagtgttagatggttctatgctgtctctgtgagaactcgcctcacctcttctcccagtccccagctcggtgtctactaacacacgacctcatctggggccatgggcttatttgaagtgatgggtttccctccctatctttttacttcttaggcttcagtgtctcctaaattgagctaccatgctccctttccacccaaggatcactttcttcaatagagatggaggctgagaattcaagctggctgactctgccctgccttctctatggtctgatctcattgccctagataaagggctctctgtctgtctgtctgtctttgtttgccctggtctgtcctcttcctgtctgttttaagcacagcttttgttggcctaagttatttactggggagaatcatttcattctggccgttcactgtttagatgtttctctcactctgatccatgtgatggcttctttctcattgctatggtcaagttcctaagtggcatcttaagggtgggaggctttatttatttatttatttttgctcatggtcactctgtcaggatgagggggtgggggcaggcagtggagccgctctgggttgtggccgtatgagtggaggcccccttgctcacatctaggcaaatcaggaagagaggggatgccctgctcagctcaatatctcttcttagtcttcttttttttttttttttttttcagcccaggctcccagcccatgggctgtagctgctgctgttcccattcaggtcaagctgtcttccctcagccagtcctccctggaaacactctcatggacacccaaggtgtgcttcgtgagcaccataggtatttcttaatccagttgatgtgaggattagctgtcaccccgagtgagggtcctgtgtccacacatgtgagcctgagtctgttccagtcaggatacgtgtcataacctgtgatttcagcctgcagccatgttggctgtgctatcctgggaggcgaaaaacgtactggctgtcggattccatctgacgccctccacctaggaggacagttgtcatggaagggtgtccacttggaacctctgcttgaaacagaaccggcttgagcatgctttcggatcccgcacagaggattctgcacgtgcggactccactgctggcctgctcttcttctctctttttgcctctgtacctcagcctttcctcatgtttaaatgggacaggacaagaagcagtgcactgtggactatcaggtgctctggaaacaaaaggtttgactggtgttcctgcttaaggtggacagctatttctctttctcgccctcctttctacttctatttcctctccctctccccttatcccctcctctgtctcacaatgtaataatacaagcctgttctacactcacaaccctctgcctcagcccgtggcacagctgtaattacagatgtgtgctacacctggcatcaggggtttctagtttgtggcattctgcttactccgggagtctggaatagtctaggcattcagctaccttgtcatttaacagcccagctcttggggaccatctgagagcctgcaggtattagaacaggtcacacagacctcttagcagtccagtgccagctacgggagagagtatcgactccaggctcctcatatcctagaacatgaactgttgcacacaggacagagttaattagttgactttccttccagcgtctagcgagctgtgactctggacaatgtctgagctctgacaagctgctatggtcttgatttaaagccagggccaccatagcattctctgcattagattttgaggagggtcaagcgacatgtggcaagaatgtagaccttgtttggtgcttaataggtgagtggtttagacttggatctctacataggttgtatccacccaggctttggaggttttttagactctttgtgttctgagttccccctccccaaatcataactatgcatcatgtatatgtggagactgtgagagaaggggctgagtatgcgggcagaaactgctttatttctgtgacaatattttctccagaagcctttgggccatcccttgtatgcaggtacgccagtgaaatgtctcatgtcctcagtgtttggtgatttatggcgtgtctctccatgtgctgtcccatctaaccgtgaagaaaagctgaggcacgtggaagagtagacaacaatagttgtctactattccacgtgcctggctaccaggctgagtgtggcttgcacctgtggactcaggtctctgtggagacctcactggagactgtcgctccacagaggatacatccctgtgatgagccttgtggggcaggcagctttacagcggcagggcagtgaacaggcctctggtgaagagcacggttcagcagacctgtgtggacactgacagagcctgagaagacaaacctgctggagcaaactctgagacaagccttcctcacgggtctaggacaggtatacatcgctgagtctttctgcttctgcccagatcctgttcccatccagacatcccctctgctgtcgccaggggctcttttggaggctgggctggtttaatcctttctggttctcccccaagataagctgggtaattgaaaatgtgcccacgagtataatctccaaattggaggatttaaaacaaagctgattagaggcttccaactgtaagcgcaggacagaaaaatctttctttaaaagacgccgttgatactgaagaggtgagcaccttggtggactgcacatggttggtgtggactctgtgcagagagagtgcatgggagctggagagagagccgagtgtaagcacggggacttgagttgtttttctaggacccacgcattaaaaaggtgtgggtggcagtgtgcacggtggtgtgtatggtggtgtgcatgctggtgtgcattgtcgtgtgtgtggtggtgtacatggtagtgtgcattgtagtgtgtatggtggtgtgcatggtggtgtgtgtggtggtgtgcatgctggtgtgcattgtcgtgtgtgtatggtggtgtacatggtagtgtgcattgtagtgtgtatggtggtgtgcatggcggtgtgtttggtggtgtgcacttgcagtcccagtgctggagaggtggagataggaagatccctgaggctcactcgcctgacttgtaaagttccaggccaacgtcagatgttgttaaaaataaaaaagatggtcaccacccgaggggcaacagtcaaggatgtaagttgtctgtcttccacatgcacagacatgtgtgcacacatatatgaatctgagcacacatgtatatacacacatgagaatctgaacatgcatgtgtacacatatgtaaatttgaacatgcatatgcatatacacatgtgactctgaacgcacatgatggccacaacacagagaaggagaggagactgggcctcaggtggcctccattttctccgttcccagcagcttcctctgtccctctcagcatggactcaggacacacatgtctctctgtagaatatctcctctgccccaccagggctcccgctgggatcagaggtgcatcaagaggtctcaggaaggcagactggaggagactttcaaacaagaaaaccaccattgtctgaagacagaaggcccacagagcttgctgaccacacggacctggccacaggatgttcaaagaggatgagtttgttgtttatgcctcaggaaccagaagaaggtggtgagagccagtgcggcaggcagagggctgtctctgcactgtggagataggtgcccccatgtgaagcgtgcttttttatgagatcatcaccagtgacctgcattccacagagcgagacctcggtgcccactgggtattgcttctcttccaggcccactgacccctgctgtgaggctcgctccttcctggagagcccagcctcaagtggctcaggcacttgtccctttgattccaacctgccaggtcacctgacaccacccaagtctgcacaggagccaccaacactctctgaatcgcagccctcggggagcagccagcagctctggctttcagcccgctgtggcagatggccctgatctgagagtttatgtaatatctgtctgatggcagtgactgcaagagacagtggcatctcatttaaataattcaaccgtcgtaagccgtgctgctttggatactggagcttgcggagagagctgggtggagatgttttgtgctttccacaacgtggaaacagatggttgtatccaaggctcacctcagatgggtggaggccaaggcttcctcacaccctcactctgagggtaaagttcactgctttcttcaagatctcaccatatagaagcaccataaaagcaggaagagccaagttcttagcaactaatgtgcatggtttgtatgtaaatgaattcttcaagaatttactcttaaacatcctgagtactcttgctgtctctttccttagggtatttatccattgaaacaagcgcttcgttttcaagtagcatgcaggttaactgagtgacgtctaacccaccgtttcagtactgtcatcttcaaaggctgatggagctcaaatcagacagaaccaagtcactctgtctagcggttttcgttcttgtaacatgtggaagaacatggaagttaaggatagacaggtggtcattccaactactcgggaggctgagacaggaggattataagtcccaggcttgtctgggctgcagagtaagttcatcttaagcaacttgtgagaccttgcctcaaagtttacaaaagatgaagagggtctatgtatggcccgtggaggagcgtttgcctagcacacatgaagctctgagttctatgctcagcaaaagcaatgcagcaacaaagaaaaacactgacaaaatacagatattaaatcacacgttgagtttcaatatcaggagactagaggaggtaaattattttgttacatttatttatttgtatgtgtgcatgtgtgtgtgtgcctatatataccatggcacctattagagccagttctctccttccaccatgtatgtcccagggattgaactcaggttgtcagacttggcagcaagcactttcactcactgagccatcgtgctggcccagcactagatacgttttccgacagagtcaaaggattgtgtgtttactgctctagctgttggtatgaacctgggagagcaggacctaaaactaaatttgactaaaatctcatgcaatagccaactttattctgagcatcagacaatttatattctgagggttaagaagagtctcccaagcgaagtttacaatcaccagtataagtcgtacatggcaaaacacggttttccatagggtcatttgagtgacgacagctgatgtaaactcactcctatctgctacacccggaagaggcacaaaaccacaccccaaggacagttccgtcttgaagaatctgaggccaccaggttcttctcttgtctccttgaagttttctcacaagcgctccgaattcccctcacacaactccatttctggactgtgttccaacagttgtaaaagacaattaattgtattacatctcatctcagacccatcagtcacagcccccacccgccgcaagctgctacaacccaggactgtacagatgccactacaggctggcaccacgcagatcggattccttccgctgccctcacatgcctgcaccatgagccagagctggcatgtggaaagagcattgaaccccccaggcacacagacagacagacagacagacagacagacagatagtcacacacacacacacacacacatgcacacgcacatacacacacactttcctggcaggaccatgtgatgctgttgtcaagtcatcttttgggttaggcattaagagtggcacaaggctttgatgtgggcttacgttctgagtactatgaaaagaaagtacaaagtagagatgcgcttccatctctggggaagaatttacatgtgccatgctgataatttggaacaattttgtgaataaacatatctccaagttggcatacaaaggggcggggacactttagagagccccggaaatgagctcctgcataaaacgtggcgttaaacagaaagccaggactttgcgaatgacttgagcagaggctcttttgttcgcaccgatgcgggagcttgctgctctttgctgtgtaaacatatggccagaaacccaagttgctgagttgttttgagataagagcaacagagaggcctccttctggttcatccatatttcattagggcagcagttaaaagattcattagaaacacccatctgtagccagcagctttcttgagggggctcagatggaggctggattttctgagtagcacttcacatgcacagagaaagacaaccatttacgagaattaaaagctcggcagagagaggctgcctggtaaaatgttggagcgtacctgtgctcacgtgaagcagcgctcactccataaggaatcccagagtttatcctgagctcagtaggcgtggccatggcccgggaacatggatttgggttgccctgaaggatatgttctgctgtggaagaggttacacgagtgtttatagtcatacaactacacaacaaaaaaagttgtacatcaaggtgttgctcagacactgtggggggtgggagggggagggactgcaggtgggttggttataacacagcagggacgtctctggtgtaggtctcaaaggctgtctgataacagacacagacagagtgggcagtggataactatcaaggggattaacggtaacccaggatgatttgggctattgatctgtagcattccaactcttcagaaccacaaggctctaatcagtcagtaactcttgcagaatcttgaccatggggtggctctttcaaggagccttgaactgacacctgctgcatgctggtaacccttgactgacacttgcctcaggtctgaaaaggggaggaagccggacccagaaaggcgggtcaaaatgggaagctcttggtcagggttgggcagggaccaagggatcctgagatcacaccctgggccaccctagtaactgtcctggaggaacaaagataatccaaagagacacctgtgccttttccttggctatcagagcattgggataatggtctcactgggagaagtctcatgttaggaaaactggcttccagacagacttccggctttagaacaggcttgggctctgcctaatgatttaataaacctgtacacacagaaagtagagtggtgttcaccagggtccagaggaggagtaggagggtgccagtcttggacggggatcatgtttcattctgggaagatgtttaaaaactcctgtggatggatggtagcgatgactggacaacagtgtgaatgtacccagcaccactgaactcagctcttatacgtggtaagaacgataaatcttgttgtgtgtatgttagcttagtaaaaaagtactgaaaatatcctggaggaagtgaatcgaagcctcggagaggaccagggaaagcaaaaccccaagtttgtgcttctcaaagtatcctgttgggagtcagcaaacgacggttctccaggttccactcttacacacctggtggacgtttctgggagaaaagagagaagcgtgggacagtgtcatggcttcagttctagaaccttcttttggctactgtgttgttcagggttctttggagagacagaatcagtagaatgaatctctctgtaaaaatggatttattagagtggcttacaggctgtggtctgactggtccaacagtggctgtctcccgatgaaaggccaaggctcagtagttatgcggtccacaaggctgggtgtctctggtgccttcagtatgtgtcagaatcccaaagcagtgggctctaaagtcagtgaaggaatgaagttgtcagtgagagtaagggcaagcaggcagagaacagaagatcccttcctccatgtcctttatgcaggctgccacccccccaggtgtggcccagattaaaggtggatcctcccacctacgataaatgatccagtcctcacaggtgtaaccaggatcttgggttttggttaattctagttactactacgaatagctagtacagttctccaccctggtgatgatggtggtgatgtgtgtgtgtgtgtgtgcttgtttgtaggcatgggcaatggcagagaagcttgctatttttagtctttctgtttcgtaatatcaaatatttatgatttttcaaaattacacagacgtgtgtggtgtgggggttatgtgcacatgagtcccagagtccagaggagggcattggctccgctgggattggagttacagacaattgtgggcaattgtgcctatgctgggaactgaactcaggtcctctggaggagcatccagcacacttaaccactgagccatctctcccgtcttgaaatatgccttcttaaccgctaagtcatctctctagcagctctgtgctgtaacattgacagcaaggtaaacccagtatgctttcaaacatggcctcaaccttccttattactttgggttcatactgagtaactcctaagatgaacgtgccatccttttccctgaagcacagcgagcttcatttgtgtctccagcggacagccaggagcttgtcggtccttgcttcccaccaagctcagcatcccactttgctctggtcctatgaggtctttattgtggtagcatggacactcagcctaggataaagagaacctctggcaaaagtagttcaccttctcagggcaattgaggaagaaatcattaatctctttatattcatattggctttaagtttgtagtcaggtaaaggctcttctgtgtggccacaaactcagattgggtatttgaggtaagaggcagccgacagtgctgagcagtgtgagttgaccttttgggctttggccagcttttggccagggaacacagccaagtctatttcttgtcaccagtgatctctagttccgtggccagtttaaagctccccaccgatgctccatctttatcggcacagcctccagctgctctgggtcattttaactgagcagtgactagcagtttgaggctcccttgttgaagtgttagcttcctccctgagaactctctcttccagcatcctctcagccagggtttccagggagttgttggactgttcaaagaggccctttgacacctagcttggtttcaagcatctgtctgtcccttccctagcatcttggagccacaggcccggctgcagggtatggtgtgtgtgtgtgtgtgtgtgtgtgtgtgtgtgtgtgtgtgtgtgtgtgtgtgtgtgtgtgtatgtgtgtccctaggcaggtacaaactttcctttgtctggtcttaatctgtaggcaatgggcctgtggctctccagacttacaggccatggctggagcagttggggtttacagtgtcaggaccccaggagggtgacacacaggcatttggaaagaatcattttttagcaagttactctctacttgggaatgctctttgaacaataggacagctacttaactggtggcatttgggttacaaaataattggaagtcttttgtcccaaaagacaataaggttttgccagactcatttaggatgtgaagctgtgagcctttccaccggccggccgcagcggccagaacgcagtgttctctgcgacagggatccccagctctttagtggttctcttgccagctttgcagaaggtggagggacagctgtccctctggggctccctctgttcccgtttgaattctgaagccaagctggtgggtgctccaagctggtgggtgctccaagctggtgggtgctccaagctggtggggctctgatactgtggtgagcatggttctgggcagggggtgacagagagacaggggacactaggatagcaaaatgaccacatcagctaccccagagctgtaatgggccaaatatgtggttataaagtaacagggaacagaaatctgtcatttaactgcctgtcaccaaagtcatcatctcagtttggaacaaagagaaaggagaaaggatgaacttggagggaggcaaggctcagggctgaggcaggctttggggagtcagagtgagatctcaggagttaactacaaacttctgattcattcattcattcattcattcattcattcattcattcatattcattctctgtctctctgtctctccgtgtctctttcactgagacttggaacttgccaattagactaggctgcctagatggagaaccctagatatcctcctgtttctctacctcctgcgagctagggttacaggtgtgtaccaccctgctcagctttgtctatggcgtggtatttttgtgagcttgacatatgtccttgccaatgggaatgcatttttctgtgtccacaccatgtagagatcaaaggtcaacactgcatgtctttcttcatctttaatattttttaaaaaatatttatttatttaatgtatgtgagtacactgttgctgtcttcagatacaccagaagagggcattggatccccattacagatggttgtgagccaccatgaggttgctgggaattgaactcaggtcctctggaagagcagttagtgctcttaaacgctgagccatctctccagcccttcatctttaatttttaaaaatgcattttatatctatttaccttacgtgtataggtgtgttttctgcatttatgcctatgtaccgcccacatgactggtgccagggcaagacaggtatgggcatgggatcccctgaaaatggagttagatatgctggtgagtgtcatgtgggtgctgggaactgcatcctgggcgcttctgcaggagcatcaagtgctcttgtggagcagaggcccatttacatcttgaaggtggcggccttagaaagcctgcgcttgcattctgtaggtccgacacatggctgagaaagcctgcttttctaatccagttggcacaatcaaggttatgtggtacctattgcaccctcggcctcagccagctttccagcctctccatctttaatttttgaggctgactctttccctgaccatgaagctcatcaatttggctccatggtcagcgaacttcagggacccccctgactctgcctcccagcacggggactgcaaacatgcaccaccacacccagcactttaacctgggctgggggggggtggtggtcaaactcaggttctcggacttgcacagcaaatactttacgtgctgagccatcttcccaggcctgtagctaatagttcaccacacttttatgttccatgtcctattctaaactgtttatacagatgctaacaagaaccttgtatatggggtccttctaaccaccccctcaactgagggggaaccgaggcacacagaagccaagcagcctgcagaaggttacacaatgagcagttgatatggtcatatttggaccagacagtgaggtgacaggattcttactgttcctatttctgcagggtagctaggtgaggactcagcccctgtggtccactgtgtgagatgaagagacaaatggcctctatgtcacaaccagtgacttagagagggttttactgctgtgaacagacaccatgaccaaggcaactcttaaaaaggacaacatttaaatggggcaggcttacaggtttagaggttcagtctatcaccatcaaggtgggagcatggcagagtccaggcaggcacggtgcaggagaatgctgagagttctacgtcttcatctcaaggcagctaggagaagactgacttccaaacagctaggatgagggtcttaaagcccacacccacagtgacacacctacttcaacaaggccacaccttctaatagtgtcattccctaggcccagtatatacaaaccatcacagacaccgagggctcgggaatgcgttgggaacgtgggaacctggatacttggctactgcaaatccaagtgtcattcaatagtgactggaatacttgacagagtcacatgctgatattttggtgtccggtggcttgctttgcaagccctctcctggagggacacactatgtatcatcagcactaggacagtcgctggggagccaccaaggctgtgtccccaggacactgacagtctgtcagacagacatacagtagggcctgctgctctgccagcagaaggctgggatgctgggctgactgggcatctgcagaggggcaccaactttcttttggtttttaaagacagggtgttgtgtagctcaggctagctcgaactccctctggtagtaaggatgaccttgctttcctgacctttcagccttcctgtcccaaggactggcatcatgggagtgtcccaccatgagttttcacgctgtgtcttggataggtgtacccacagagacctgcatggctagcatgtcctccccgtgccgctcctgctgattcaatgacatacactgttttctcttctcttctcagacagtgggcatggcttaccgcctgcagtagcccctcagaggcggcggctgacacggagaccaggctacatgagaagcacagcgggttctgggatcgggttcctctctccagcagtgggcatgccacacccgagctcagcagggctgacaggccagcagtcccaacactcacagtccaaggctggacagtgcggacttgaccctgggagccactgccaagcctcactggtgggcaagccttttctcaagagctcccttgtccctgctgtggcctctgagggccacctgcacccagcccagcgctctatgagaaaaagaccagtgcactttgcggttcattccaagaatgacagcagacaatctgagaggctgactgggtcatttaagcctggggacagtgggttttggcaagaattattatcttcagacagctttaagtctctggctcctagccttgatgcaccctggaacaagggatcaaggggcctgaagactgtgaaacctctggcatcaccggcgttgaatggccccgctgatatcgcatcccttcccggcttccaagacaccttcacttccagcttcagcttcatccaactctcccttggtgctgctggagaacgcggagaagcagaaggttgcctgccatccagagaggccgaacctctgcatcagaggccccaagagatggcagctgaagcatctagctcagacaggccccacggggatcctcggcatctctggaccttcagtcttcacgctgctccaggcttggcggacttggctcaggtgacaaggagcagcagcaggcaatcagaatgtggcacggtctcctcctcctcctcggacactggcttctcttcccaggatgcatcctccgctggtgggcggggcgaccagggcggcggctgggccgatgcccatggatggcatacattgctcagggaatgggagcccatgctgcaggactacctactgagcaaccgcaggcagctggaggtgtgtgtgcttctggaatcgggtcacctcaaactcagtgttgggtggaggcggggagtgggccaccgactgagtgctggtcccggaagctgcaatggctcctgggacgactgaaaagatgtggcggcacatctgtctcctccacatctgaacgcgcatgctctgctttccgctttctggctgtgtggcttaggtgctggcatgtcctctctgatggggcccatacttctccacaacctttgacctgcagtgtggtttgttcgacttaggacactggggcctggcaagatggctcggtgggtaaagacacttgctgccaagcctgactgcctgagtttgatcctccagaacccacatggtggaagagggagctgacttctgcaagccattacttctccttgccccatagcaaacaaatctgataaaataagacattgggagggtaaggggggacagccagctcaggtttggaaagagtgagaagttaggagggggagaagtctgatcaggcaggccccatcctcccctgttttgtttcattgccccatgtaggctatgaaggtcctcctaccaagagactgtgagtatgtgtgtgtgcatatgtgtacatgtgtgtctgtggtgtgtatatgtgtctgagttgtgtatgtgtctgtggtgagtatatgtgtctgaggtgtgtatgtgtgtggggtgtgtggtgtgtatatgtgtctgtggtgtgtggtatatatgtgtgtggtgtgtgtgtggtatatggtatatgtgatgtgtgtgtgtgtggtatgatgtggtgagtatgaatatgtggtgtgtggtatgtgtgtgtgtggtatatggtatatgtgatgtgtgtgtatatatgtgtgtgtgtgtatatgtggtttgtatgtggcctgaggacaaccttgaatgtcatcttcaattttgtcatggggcttgagtgcctatgaagccagtgggcctcatggactcttctgtcccttctccctgtgaggtgggattacagccatcatgggtcactatgcccagttttctgtacgagttctgagcatggagctcaggtgcttacacttgccaggcaagtgcttgactgacttctcatctcctcagcgtccacctttagcgattagaagcaagttttggccatagcctgtcaggaagttgttctggaatgaaggcatggctgccagtgactctccaggcccttgtccatctctcccccactcctgtgctggggatagaactagggccttgtgtcctcaccactgagttacatgtccagcccaagacaccagcttccctgcgatggacccttcgagcatatccacagcccctcctgggctggccagcctggctctcacttcccacgttcacacttgccacgatgctctgagcgttagtgcatgttcagaggccatttggcctgcagtcggtgttccctctccccacgtagcgcttcatggaacagcactgtggtatttgtaacattctaagtctttctggggagctcatgtttaaattgtgctttttgccatgtacttggcattgggaaactgtatatttgtgtatgtgtatgtttttaaaaagagtttaaggagaacttcacttttaaaatagtttccctattttaaaaacagtattgctttttaggattttaactttttttattacattttttatttatatgtgtgcatgatggtacttgtgcagaggtcagagctcagagaactccttgtgggagttgtgtgggttctggggaccaagctcaggttctctggcttggcatcatggccttcaccctcaagccatcaggacagcccgcttttgaaatagtttctaagtgctcgtagattttaccaaatgaactcacttgaattaattcgtgatttatgtcttatgtttccctttctcacccacacaggtcacttccttaattttaaagcttcagaaatgtcaagaaaaagtggtcgaggatggcgattacgatactggtgagtccaaagctgttcgtagacagtttcatctttctccctgatggctgtctaggcgaacgtctttctccgcttcttggtctcagcctccttcatcctgggagctgtgtggttgaatcttgtgggagtctctctcctggggcagctggctaacaggctgccatgttggtctctggatttcaaagtgctttggagcatcaacctctctggcaggcgaaacctcattgactggccgggaacaggtgtggagtctgctctgtaggaagcatgctagctcatcacctgtccacctgtaatccacggatccattagctacacccagtatggcacacatgttgtgcctcgtagcatgggggcaacaggggaagagggaagaaaatgccggtcctgcagctgggcatggtggcacacgcctttaatcccagcacttggaaggcagaggcaggtggatttctgagtttgaggccagcctggtttacagagtgagttccaggacagccagggctacacagagaaactctgtctcaaaaaaccaaaaaaaaaaaaaaaaaaaaaaaaaaaaaaaaaaaaaatgccagtcctgtctgtgggatggtggatggtgccatcatcacagggctttctggcgtcgtgctctctccacacgagatgtagaaacgagttgtgttcaagagatgtcaggcaaaccagagcctggcatcacatactcatgactaaaaataaccaagagaagaaagacaaattacaacatgaggcgggggttcggggaggagctggtcagagcccactgaggagccaaacaggagtggggccagaaagaggcggtgctcactgggcctgaggcggtgctgactgctgattgggcctaaggcggtgctcactgggctgggtgggtggagacagagctttcaggggtggctgctttggaaggagccttaagaattctgctgggtttggggaaaggagggggcttaggataactagagttcctctagacacgtgtggggctgacagacactccggctcacggcctggtttaggataccagtactggcccggaagtagaatgcagagcctcggatcctctgaaagctgttagctgagtgcgaaggctgtgcagtgctactccagtcgctatgccttgagctgttaggattgcagcggtgttggcatccgtgagtgacttccggaaggagcttgtcagttcctccgaagagcaggggtacactctgtgtgcactgttgagtttctctttagggagtgagaggattttgggggatggaggtgggactcgtcaggttctcctatacctttgcctgtaggccaccagatttcacagatcctggatgaagaatgagtaccagagagagcagaaggcatggggcggaagcatcctccatgatggcccctggggtctgtgctgcatcacagatagaaaaaagccagctcttcctggttctgagtcaatgctggtttgtctgaaagacagccctttcttttatgcgccctgcattgcagcattgtgtgagcttgttgtcactgtaaggggctgactcctagtccctcctccttcaggacatagccctcatgtactatgtcacatgaatgggaaacacaccttttccccacctcaagacatagaactttctggacttggctctcctgttggtcttctgtccaagacatcatttctatctttttctctcaatatctctgacaccgtttgggatgatgcacttatcagagaaatcagaaaaggcactcttcttcagcatccttcctggtcaccctccttcagcatccttcttggtacctttcttcagcatccttcctgggtaccctccttcagcatccttcctggtaccctccttcagtatccttactgggtaccctccttcagaatccttcctgggtactcttcttcagcatcctttctggtaccctccttcagcatccttcctggtaccctctttcagcatccttcctgggtaccttccttcagcatccttcctgggaacccctcttcagcatccttcctgggaacccttcttcagcatccttcctgagtaccctcctttagcatccttcctggtaccctccttcagcatccttcctggtaccctccttcagcattcttcctggtaccctccttcagcatccttcctgggtaccctccttcagcatccttcctggtaccctccttcagcatccttcctgggaacccttcttcagcatccttcctggtaccctccttcagcatccttcctggtaccctccttcagcatccttcctggtaccctccttcagcatccttcctggtaccctccttcagcatccttcctggtaccctccttcagcatccttcctggtaccctccttcagcatccttcctggtaccctccttcagtatccttcctgggtacccttcttcagcatccttcctggtactctccttcagtatccttctggcttgttccttattgtgtgctcagaatttctggagtgggcctagcactctatatttctctagtttctagaggactcggggattaaggttggaggaattgtggctctaagattcttccacagaggggcaggccacagggcagtggctgggagttttaagggggcatctgtctggttcacagtaagtcacatcctgtttaagtcctcaacttgcacacttggcttgcatattgagagcgtaagtgtccttgtgaagtgtatatctggttgctgggcattcagtagatgctttatgaggtattaagatgtcttggtacccacactctcactgccttagagcagacgaggagaaagcactacagagttctcatggcagaacatttttggtttgggaggttaggtgggtgcagggagaaagctatgggaggcagacgtcctcctgggatgagactcaacactgacggttatctgtgatggatgaaccctgccccagtcactaggtgattgtggaggtttgaacatcaaccgtctcccggaacctcataagattagacacttggttcccagctcacgtggctgttttagaaggtcgtggcgcatttagaaagtggagcctagctggcagaagcaggttgctgggggttggggggaggtgggtctttgaaggattgaacctctttgggatttggtctgtattttctgctgtctagtcagccatgatgtaagaccctctactgcaggctctgactgccaccatgagtcactaccatggtgaacggaggatcctctgaaaccacaaacccaaataaaccctccctccttacattgctcctgttagaagaacagaggaagaagaaaaataacagagtatagagttctcataggtcaatgagattctgggtcagtgagtcctcatcttcaatgagctgacgtttattgtaatgagatttgtcgaaagaccctggaatataaccttaattcccacctagtttctccaggttcctaaaaatattggaaaattgttgagatttatttacatcctgtataaactttgttactctgtgtgtgtgtgtgtgtgtgtgtgtgtgtgtgtgtgtgtgtgtgtgtgtgtgtagacaacagcatacttgctctctgtcttggggaagagtgtgtgatggtgtgtgccggagcacaccaggggaagctacaggacagccacatatccccaggtttttgctcctttttcctttcgttctatgctcaagagaaagatgtggttggcacagaatagaaagttccatgttttatctcattaaaatcacagcaagtttacactgcaaagcctcctgggggagactccctaaggactctgactcctcccatctcccttgccagggaagtcaagttcttgtttcttttatctttactgcaggctgggaaggccttggggggggggggagaataacaatactttccatgtttgcttagggtccttttcccatgagacacttttagactcattacattaatgcctgcctgtgactgaagtgctaagggcaggaacaagactgggagagaccactgcttggacgctagggtgcagagtagacattcccgtgtctctgtctaggactcagcctagaaggaggaaaagattgtccacagggatcctgtattgataccccactggcatactgtaaccctgcctgctacttcactgccagatgagccaggagtgggtgtgaggtggagcagaaactctttggatcttagcatctctgtttctctttctgtgtctctgtctctcatacatacacatacacatacacatacacatacacatacacagacacacacacacacacacacacacacacatacacacacacacatacacatacacatatacgatgtttggtcctgtgtgttcaagtgtgtggaggtcagaggtggctgggaactgaacctggattctctacaagagcagtaagtgctctgaattgctgagcagtttttctgtcccccaacacaccttaggaaatacttgaatgttgtggcattagagtttagaaaaatttagacaagcaaatggttagggttagggttagggttagggttagggtttaggattagggtttagggttaggttttggggttaggggttgggggttagggttagggttaggcatctgttacagttggttgcataggtttgtttgtttgtttatcaagttgcctacagtgtttagcctcgtggtatgcatggagcagagagagtacaagactctggcatgcagccacgagggctctgcaggctgccatgtggggctgaggcaacatctatacagaagactttaccatggagctgagggctgtgtcaaactgaacagtgaagccaaaactgcttcatgctcaggataatggcagacttcactcttaaggacaagggcatggcaagctctgccactcggtttgtgataaagtcccctttggtggcagcacatcggagccatctcaggatgcacacatacaagactatgtctccaagttctccatgggcagtatgccgtatgaaacagggcgtcttaaatgtagacctatgtctctcacacccagtctctcatactggtttccatttgcctctggttttaccagtttcccagagacttttgtaaatgtcacatggttggaatatgaaatgtcctcacaggtttgtgtttgcaatgcctggccacactggcttgggaaggttgtgaacttttaccaggtggagcctctctggagggagcggctcacttggggatgtgtcttgagggttacatacatgctggttctcatttctcttcctgctgccgtcttcctgcttgatgcagtcatgtgaccagtagcctcgtgctttcctgttgcagagaagagctactcggtcctccatgcctgccctgtcatgacagactgtcttctccaaactgtgagccccagcagccctctcctcactaagctctttctgttggcatttttatttttatttttgtcacagccattagctaatacacaaagtaaccgttgatcgatgtctgatggtgacagtgtcacgaaggtttgtatgtggggctctggaaaagctccatccattatccatccattagctggatattatgcatattataataaagagctgccctggaccacaaccggcaattgaattggctgactgacagcttctactagaagaatgttctaactcttctatttttgcacaaagtgacagaaacaattaacttatttgacctgagacccttaaaaacaccctcatgagatgtgaaaaaattggtgataatgtacttatacattgattttgttcctctgtatttcagttgaggcattatgaaaatgagttctgaattatttagcagctccttttcccatctcttttgatggggaatgttgtgttataaaacgataaagagaggggggatatgttttggtgaaggtatagtcaggtgtgggggaatggggcgccttggcaggcccatgctgaggcatcccttcctcctgagggaccagccacaggatggtatagtatagaattgagtttattcagggcatggggaggggagttgagagggtagtagaggcagagagagagagagagggagagagagagagagagggagagagagagagggagagggagagggggagggggagggggagggggagaggaagagagagagagagagagagagagcgagcccagaagagaagtagaggctagccatgagtaattggagaggtaggggtgagaggacagagcgggagcaagaagtcaagagcaagagagagcagagggggcaagcagccccttttatagtgagtcaggccaggtaactgaggggcagagctcaggcaaaatgctaatatggatagttcagcaagtccaggagagagtcctgagatgggcaaggagacagggtgtcagagtggtagttgtttgccctgcagggtgcgcttaggctcctgtttgttccagggtaaagcaggagtgtaaccctgttccctggtgtgggaggtgtcaatgtgaaggtgctgtgtaagaggcatccacagctcagtgcatgagaggatgggttcccttacttcctgtgccccgtacacattagaaagcactagaaacggaggcttctgaataacctatgatggtagttatcacgggaacaagaaggcagggcaggataattcggcttcttgctttctatgtggcaataagaagctcattgtgtatgatctgatttctttcaagatgcaaaaataaaattgtaaatgaggtaaagcaacctctgtcccacagcatgcaaattacagtgaagcgtgtgaagtttgaatattctaaacatcttagtgaatgggtttttcttctcttcagggtaggtttctggatctgtcatctcgttaattttatggattttttttttttttgggtgacattgaacgacactgaggagataaatttgtaaccctgcttgggtttatgaagacctaacattgattattgttgcagttagttttagtttttaattatcaacttgactcagcctagactcacctgacaggagagatttaattgagagattgcctagataagactagcttgtggaaatgtctactgggaattgtcttgattaaatatcttgttaaatgatgctggaagatacagcccactctgggcggcatcattccctaggcaggtggtcctgacctgtataagaaagctagctgagtatgagcctgtgtaagccatccagtcacccagcaaccttccttcatgatttctgactcaggttcttgcttgaattgttttgacatcccttcacaaagggctgtgacctgaaagccaaattaatcatttcctctcctaagttgattttggtttagaatgtcttattacagtaacagaacgggaatagacaatcatttgtttgctcgagatggcatcttactgtgtagcccaggctggtcttgaactccttttaactcagcctggtatcacacagctaggattacaggtgcagtgcatatccaggcttcattatcctggttttgaagctagaggggagggggtgaaaaaataacagaatgaaagtaggatccagatgcggagaggggaagggctctgtcttgggaccgttgcccctccccacggacctctgctcttctgtgaactctggctttgtgggaggagaagataatgagccatctcaagaaaagtgtcactgttgcctttaaaacaattacaaacacggtggccttggctcttggcactgtgccttggatcactggacagcttttggtttgtatgaattgtccaggcatttgattcagtcttgtgtcttgtctactgaaactgcagcacatgtggtgaccacctgctctcggtggcggatcagtggctgtttcctctagacttaacatgacttgaagcctgttttctgctgcactgtttcccaagtctttgctgtggggatgtgattgctctataggccctgtctgcatggttagcttgattgtcaacttgaaagaatctagaatctcctgggcaaggagcctctgagcatacccatgggggattatcttacttgctttaatcatggcaggaatacctgccgactgtgggtggggccatccaagggctgggatcccagccagtgtaaatgtagaaagggagtggagtggctcagtcacaagcattagctgctctttgcttcctgtgcttgaagatggtcagctacctctcactcccaatgctgcgacttcccagaagacagactttcttccttaggttgcttttgtcaatggtatctttattacaacaataggaaaatgaccagtaaactgtcctctggagatgggcccccaaagctacccaggtggccaaaaacccaaaccaaaccaaccaaccaaccaaccaaccaaccaaccaaaacaaacaaacaaaaaaccaactcttgttggcaaataactatttgagagtgcactgaagtcatcattgcaaggaagtggatcccaagcagtaccagggtggtgacatccatgaaaggccttttctcggatccacaggagtgcctgtggctggaggactccaggcccagtgtgctgagcagtcctgatggactgtgaaagcagccccaggactgagactacctccgctgttcactttgctcaatgtactgttactgcacgcccttctccccacaagaggaaaaaaggcagacctcatgggtccaggtggcaaggcttgtgttcagagtcaggaaggatggaggagcaagatgaagagcatccaagaaggcgctgtaggtcaggtacagtggttgaccatctccaaacccccaactgtatgccacactgtggagacacacactggacaattcttcttgcttgtttttgtaccacagttcctgacagtttcttcataagtaagacccagtcttaggttggaacaccacttttctagaatcccaagactgtattatgcctgccaccctgctatggctctctatcttgagctttctatcagctccaagagcttagttcctgctacagcaaagaagtacggggcttcccttactggactgggagatgagacagcttgtaggtttgtgtgtgttcctcacgagtgtctcttttctgccctttgacctcagcctgacctgggtatatcgattcactggtctctccgtggacccttggcttctagcctcacctctctgtttatcttctatttctgcatcccaccttaatatctttcccccttcctgtcttaggtagggtttctgttgctctgatgaaacaccgtggtggaaaaacgagttgaggagggaagggtttatttggcatacgttttgtatatcactgttcatcattgaaggaattcaggacaggaactcaagcagggcagagatctgaaggcaggagctgatacagaggtcacagagaggtgctgcttactggcttgcttctcgtcgcttgctcagcctgctttcttatggaactcaggaccaacagcccagggatgggaccacccacagtaggctgggcccttccccattgatcactaattgagaaaataccctacatatggatctcatggaggcattttctcttaatcaaggctccttcctctctaatgactctagcttgtgtcaagttgacctaaaacgacccaggacacttcccacttcacctgaccacactgttgtttatgacgtgtataaataatccagacaaccctctccctgtcctaactacacttctggctacattttctctgtcataatgctatttctctttggtctcccacttgctccagagggcagggggccttgacccataccacacatctgtgaacaccattctttctttgtcttaggtcctggggacccttctgccctggggtcaatgttctccacacttgtgtgtcttcctaagtattatacttgaatatagctgttgtctcttcccttgactaaatttcttgggtagaagaaagacaaccttatttatatttctttttattttttaaagaaaaaattacataataaatgctaattaaatacattaaagaaatagtttttccttttgctttgttttaaattatcagcttattgcttgcctaactttaactgtaccctccagcttgatgcttttcaaagttttctcttgaagaatgtttggaatatagcctattgcaaatggtaattatcatctttatttttatgagacaggatttcatgtagtctaggctgacctcaaactctctatgtagttgtcttagttagggttttactgctgtgaacagacaccatgaccaaggcaactattttaaagacattaaattgggactggcttacaggttcagagattcagtccatcatcatcaaggttggagcatggcagcatccaggcaggcatggttcaggaggagctgagagttctatatcttcatctgaaggctgctagtggaagactcacttccaggcagctaggatgagggtcttaaagcccacacccacagtgacacacctactctaacaaggccatacctccaaatagtgccactccctgggctgagcatatacaaaccatcatagtagttggcaatgacattagatttttgctattcctgcctttatctcctaagtgctgggatgtagagttgtgtactgtcatgcccagtgtgtgtggtgctggggggattgaacccagagcttctcccttcatgctaggtaagtactctagcaactgagctgtctacccaggtccatttattgtcattttatggttattattagtaggtaattatgtgagcaacataatcctagggttcagtaccttagggtcaatctagttaaaaacctgacaacagaagccaggttctcaaagcacaacaggctgtatattacaatatagatcttgtaaactccccaggctttgatttttttcttaggtgtcactagggcaacagggtcgttaggagaattatctcatactatgactgggcttgtaaatagtacctattgtaatagtgtgttggtttgagtaagagtgacccatatactaacaacggtaagcatatttgagtgcttggtcatctgggagtggtactatttgagagggattaagaggcgtggtcttattggggtgggtgtggctttgttagaggaagtgtgcctctgggggtgggctttggggttttaaaaggttaaggtgggcctctctctctctctcttcctctcttcctctctctctctctctctctctctctctctctctctctctctctctctctctctcttcctgctgcctgtggatcccagtgtagagctgtcaactccttctccagcaccatatctgcctgtgggctaccatgcttcccactgtgataataatggactgagccaatcaaatgttctcctttgtaagagctgccatggtcatgacgtcacttcacagcagtagaactctgactaactcagatggcttacctccattgtcacctgactggatttagagtcaccatggagacaggttttggggaatccttttggaaagattccactctgcagagaaggctacccagatgtccgtggcactgtcctatagatttgggtcctggaccgaataaaaaggaaaaagtgagctgagccctagtgttcagctgtccgctcctccggagtatagggctaccatgccttccctccacaaggtgtcctatgtccttgagccatgacccagaagaaaccctttcttcctgacgttgttttcaccacaacaggaaaaataacaagaaactgagtgagcgttagccacaagcacagacctggaattgcacttaggtgttcagaggctacgcagagtcttcagctcacatctttgtttttttctgttcagtttggcttgtacccagcttcctgcttcagataaacggagagggtctggcttattgctagggagaatgccgagaaaccaggactccgagtgaagggaacatttcatatgtcgtaatagacagacaaagttattttctgtcacgacaatggattggctgggatgaccttttgcggctgatgacagtgacaggcggaagggacagtcttccacttgacctttatacagtgtgccagctaacgccgtaatttggaatccattgttattgacaggcttggcagagcacggacttgacaggagcttgtcggagggagctgggaaccggcagcagcagcagcactgtcagcctcggagtgcaagaaggacctctgcgtgcccaagggtggcttgaaatgatggctctccccactctctgccagcacacttagtgtaaattagctcctcagggagtttgttttgaagtgagatttgctcataatgagttgtaattgtcaaaagcttggttcttctgtcacccccacttacactggcttgtcaccccgactgtcagctctcatcttccttgtcagggccgtggtgggatggtttcttctcgtcttgtctatccctttatttttgcctttctctgaggtttgttcccttcctgtactttctaccacagggactttccctgagagtccctcgtgtggattgaagagtgggttctgcttctcttcgtggactgctttagggcagagtcggttctgggcatggttcggggaggggtggccagtgggcttagtgaaggaaacccttcttacgaaccagtttgtccctcctgacactgccccgtggatccctctcatcagctcttcagggacccactgttggtcccagctgtggcagttaactatgggaaagggcctgccattgtgagattcttgccctgctcccagctttcttctcaagcctggtatgtcatgatggacagaccacatttctgagtacagcaagcctgctttgctttcctgccaggccaattaagaagccatgagcagatacacatagcttgttgacagaaagggcttcgtcgcagggcttggctagacagtcttggataaatgtttctgcttacctgcatgatggtggcgcacacctttaatcccaagactcaggcagaggcgggcaggttttttatgagttcaaggccatcttggtctacagagtgagttccaggacatcaagggctatacagagaaaccctgtcttgaaccacaacccaccaccaccaccccccccccccaaatctgctttttaaaatgtcatagcacaaatgaaaaagaaaaggggttaggtagaaaaaaccaggatggaaatgaagcaggctgtggaaacatgaggcgtctaagaagactatttggcttcgtgctcctagcaggtgagtctcccctgcttaagcaggctggggagaccaagggtattcacgccttcagaatgggagtccatttatgccaaagggtgagtggaggcctggctcttcctctttatcacacaggctccagggccaggggtggctgtgtgtgcagctggcagtcctgagaggatttgccttacaaatctccaccccatttggctccaataccaaacaacaacaacaacaacaacaacaacaacaacacaaaccaaaccaaccaagccagacggcaaatgtgtttgaaaagtcccgctgaggagccaataccctgggaagctccagcccccccccccccacacacacacagagggaaggctaccctccccgcagccccgtgatggcttggtttagtggaagcttgacttcgtgggattcaggcacagccaacccatgacagcctccctgggggaatcacagtttctgtggggccggggaccctgactgcatgctccctgcttagttactgggcccagggatagattctcctggttccagtgctggaaactgaacttggatccaatggaagggcagctagtgctcttaacccctgagccccctctccagtccatatattcttgtaaggaagaaaaaacaatctagaaaagttaatagagaagggcacatgtaggtgcccgctgccctcttagacctttctggtcgctgtgacggaatatccagacataagcagcgttagtaagaaatggtttgtcctggctcacggttcctgggtccatcatggtgggtggtatggcaacaggtgtttgagggagccggtcacagtgcatccgtggtcaggaagcagggagagatgatgccggtcacagtgcatccgtggtcaggaagcagggagagatgatgccggtcacagtgcatccgtggtcaggaagcagggagagatgatgccggtcacagtgcatccgtggtcaggaagcagggagagatgatgccggtcacagtgcatctgtggtcaggaagcagggagagatgatgccggtcacagtgcatctgtggtcaggaagcagggagagatgatgccagtcacagtgcatctgtggtcaggaagcagggagagatgatgctggtgtttggcttttatctcctccttatttgtcaattagtcagtgtgtgtgtctctacctcagtcaaactcatccaatctcagtaatccccgacagcctgacagaggcatgcgttcaagggattctacgccctgccagtcgctactaacagtcgctacgacagtcgctgctaacgtcacacatataaccccaactcttagagttgacgcaggggaatccagtgttctaggatagtttgggttacgcatgcagactccgtctcagaacaacagaagaagtcagagtggaaacagtacagagagctgtactctgggccctacctctcttggtgccagtccatgctcaccctaagccctgggcaggcagcagctcacatgcagagccaggtctgtagcgtgctaacagctttccccgttcctatactgtgcgatggtggtggacacactagccgtggctgactcttcttaccaagcctcatgcaccccaaggagtcacttacacatggccccatggctacgttaacactgattaaaatgaacctctctgatggtccacccagagcaaaccttgaaaagtaccgacttcattctggtttatgacacaacttggtctggtgattttgtcccaccaatgatgtcatggtgcccctagacactgtgactcacacaatagctcccccttgcctctccctcatccttggcgggtgactttcgtaaccatgcgagttctgcctttgtccttccctcccttcttcctctctctttcctggaagctgagtaggctgtgtcttctcacccaaaggacccctagtgcatggtgacaagcttgctgtacctggtgtcagtgaacaatcgactgtctgtgggttagcggagtctgtatccataacctccaggaagaaggacacagacacagaacaaaacaccactggctttgtgtttgtgttcttgttactttaagcatcccattggctagtggctgccttctcagacagcgtacttttgatgaaaatggtcgccagccaaggcaaacatgcacttaggctttttcttggggcctgcattgtaagcttggggctccagaggaaatgtgtgggagaattactcaaggtcttagcctctagcaagaatgcagggatgctgagctatgctgcccagcctggctcctggacactccaactgtgacattctgtccatctccctgggtccatgtgagctctgggctccagggacacctctctttgtggatacgtaggggacaaggaaggggtgtagcctctcagacaacagaaaagatgatgtcatttgggagtggtcaacacaggaggaaagttctctctactaatgacatcactccaaacaccctgcccacagctcaccgatgacatcactccacacactctgtccatagctctgcaggtggcacatgggctcacaaacatctctgctgctctgcctcagagttttaaaaacagtccaatagaagaacgtaagacagatagatggatgtgcatgataccacatgcattccttccttgtgtgtgtgcacacatgcttgtgtggctgcacatatgggtgcaggtgtgtgcatgcatgttgcatatgtggtagttagtcagaggacacccttgggtgtcaggcctcaagcccttagcatttccattgtttgagacaggtctctcattggccagctagctctcaggagtctgtctgtctctgtctcatcatcactaagatggctgtgtgttcccctgtgctcagcttttaatgtagattctggggatttgaactcaggtcttcatgcctgcaatgcaagcggtttgccagtaaaccacctccccagcctatactctttctagctcttgattgatctgggtggtcagtgatactgtaccctgccctgcaaggctctggcatgtacctgcaggattattgtagaaaagagtacaggtacaggactgccctgataatgcacttcaaacatgaagtctagcaattttacacttgaaatgccagctcatgcaagcctcctgccgcttgtctccggcgctctgaaagataatagaacagttgagcatagcaaatgatcaccagtgtggaaagcatggactcctagcccacagtgtgctgtttgccttgctctgaggacacccgtgcattttgccttaattttactgcatgcttgtcaccccctcttactttctgttactgacattgtgctaaagtgcatgacagaatttagttatccatgtgtcccatggttcctttaagggctttgcgacttccaggtggcttagaacctaggtgccagcagaaacttgtgctgcctgtgtgtacctgtgtgtcctgaaagaggtggtcagcaacaggagtgagcatacatcttacatacgagatgtgggaacatcaggcaaggccatcgtcaactcttccctatgtgatggtccttgaggggctggccacatcgagtgtgtgcctcatgtcagagatgatgttctgttgcttagcttttgtttctagaagcatgggacccaacagaaacaagttgagggagacaggattcattttggctcacagcttctgagggtctcggcccttggtggtttggcctcatttccttgcacagaacatcgtagcagtgggagccacaagtgaggaaggaggagctagagactgcctcctgtgagccatttcctccagctaggtctccccaaatgcctacccatcccgaatagctccaccagctggcaataaaatatgtaagacaggaacctgtgatggctattcttggttgtcaacttgactccatctgggattaactaaatctcccaaatggaagacacatctgtgagtatgtttaattaaaaaaaaatcgcttcatctgaagtaggaagatccactagtaatctggatccctgaagtaggaaggcgcacctttaatccagagcgtttgagctcagaaaatgtactgctcatctgctgggagcttacttaagggcacggtagaaggaagctcctggtctcttcctgctcgctctcacttgctcgcaagtccattccctcactggcattagcgcctacttcattgggattctactgcggacttaagagcatttgagatatccagcctcgtggactaagcaagccaagaagcagcactcccccatggcctctgcatcagttctggcccccaggttcctgccctgcttgagttgctgccctgatgtcctccgttgatggacagtgatgtggaagtgtacgtctttccttcccaccctgctttggttgtgatgtttcgtcacagcaacagtgaccctaactaagtcaaaaccacaatgccactgagaggagttaatctgaatcaagactttggttacaactcttacacctatggttttaaaaagtgtccacaggcattttggggagactgccattttaataatctgatggcccaagatgggcttaatgttgatagtcccatatgtaagatgagcaagcattctgagcttagagagaagaaatctcccagactatgaagtcagctggctggtctaggactcagctctgggtctcttgttccaggagaaaaatcacctttatttttgattttcttctcacactagaggactctctgtagaggcagagggaatgcacactcttgtgtgcatcctttattggctcttccatctgaggtctggtcctggaagtgcctgctggctgttccccttggtctatgcatgaactgacacctagccagaagcctggccactgtctgtcctcacagttgaggagtctcatggctctgttcttgcaacacatgagaccagggcctgatggccatggaatgggtcttcttgccatagggggccacttgaggaaatccagagttctcgcaatgactttctctcgcaatggcattttccctctgaaaacagtcctaccaacttaggttaatttctccaattgatgtgctgccccgcggagctgggtgggtattcctttgtcctttgacttcttctggctgtttgcagaggaactattagagccagcgcacgttcaggctctcagcacgaccaatcgaggtccttagtctttcttacagttggctgctgtcctgtgatttctactaacgtcactctgacacggaaggtcagcatttgacttgcattactgtgcctctgcccaaagaaagcttcattccatgttatttctagaaagtaatactctcacatgtcttcttccttctgattagatttcatcctaattggagaaactcaggctgtggaaatcacttaagcctctaaggtttttgttttttttttgaccatgctgttgtacctacctctctttacactactcaagaatttgagctgttcacactgacacgccatttggagagtcagttcagaacagagcagaacacgggcatgctccattctgcatcccaggaagggagagccacaccaaagtggctcacatcctactttagaaaacaatgccagtggttgctgaataacgtaccctagcatcttgaattgggtgaggtgaaaatgctaaaagcgaagttggagagattacaggttgcaaaggatcagccattaagagagcgaacaaagcctgtggttttcaagcaccgaagaccactaatttcttacgaaaacacccttgcagccaattggtcctcaacggtcgattcctaggagctaattggatgccttgttcagtttaataggaaaaccagtgcaaccaaagtcgtgaagaggcctttggcttatttcaaaaatagccacagggccaggatagcttctgctgccagtctacaaggtggctccagatctgtaatgttcatggaaggaagtaaaaatgttcatgaagcagaagccagagggtgacccctgagcattctgtggggtcatttcacagagtaattgaaagtacaaagggcccaagacatactggctgggccttgaactcatagcctggctcaggtgatcacatgaccttgtcggaaggttgaggggtgcgtctattcaccactgctgtgaagaacttgcctcttaacttgacccatttcctccagctcggctccacatcctaatggctccaccatttcccaatacaatgtcaccagtgggaggcaagtgtgagagtctggtacactctgcattccacgagggaatttgtatgtgccgtgtctttcatctagcgtgaaccagcacacactctctgttatttggagtggatccacactctccatgggatgtgcctgtggccacaggtttagcatgtaagtaatgtctgacatttatcatgaaggaagatctgttattacattttagacaggattttcccttccaagctgacatagtctgtgcgtgtgttttgtgtgtgtgtgtgtgtgtgtgtgtgtgtgtgtgtgtgtgtgtgtgtgtatgtgtgtgtgtgtttgatacatgatctcaccaagtagcctaggttggcttcagaatgttgtaatcctcctgcctcagcattgtgagtgctgagattacagatgtaagacaccatgccaggcttctcattttacatgtctgtgaatggatatgtagcttttgcctgttacatttctctgtaactagaaaaggaacctatctctggatctgggcttgttttgaatgttccccatggaaatttaagaattaggggagaccttaaaaagacactgtaggaatccaattttaaaacaatattggtactggctggttttgtgtcaacttgacacaagctggacttaacacaaagaaaggagcctcccttgaggaaatgcctccatgagatccagctttacggcattttctcaattagtgatcaagggttggagggccaagcccatggtgggtggtgacatccctgggctggtaatgctgggttctataagaaagcaagctgagcaagccaggggaagcaagccagtaagaaaaaaccctccatggcctctacatcagctcctgcctccaagttcctgccctgtgtgagttcctgtcctgacttcctttggtgatgaacagcagtgtggaaagtgtaagcagaataaaccctttcctccccaacttgcttcttggtcatgatgttttgtgcagtaatacaaaccctgactaagacaaatatcaacccccaaaacttgttttgcttttattaccatatcaaggttaggctaacttttcggatgcagagagttgtgagattcataaaagttggttcactaaaagcgttttctgtaactttcctcttgtataaacgtggaggtattaccagaagaggtagtctatttggttttcccacaaactactttcttaaggacacctattaaatttggcgtaattttagtgaacttagaaaggctctaggcactttcaagtctgaggcaggaagatcatgaattataggctaacctgagctgtagagagagaccctattttaaaaaatacaaaactaaccaactaattaacatcataatcaataataaccaacagtgataacggaaaggatcttttgactaagtttcatacctcctgccctacttcatggcaatggtcatagttccactgagggtgtgtgcccatgacctaggacaaggctggttacctcatatccctagggggctcatgaatcactgctgaagagcctaaggcaaagtaagattctgatcaagagtcaatcaagatagtatcttacattaaatggcttctttgaattcataattctatcatatgaatgcccctgagccctgatgagccatggtgtccactcttgccactacagtcacaaggactctctagggcagatggactagaggctccatgaggaggtgagcagtatgcctaggaaggtcgataagacaacatgagcagacttggctaatgccagtaaccagccattgaagagcatccgtgtgcttctggctttccaaagctcacatctgttttctcacaacccacctctggtatttatagagcgcatatatacagccaccaccaacttaggtctctgtgtagagaggtcattgttagtaaggtgacacatgcccttcctggctcgttacattgtatcagcaagtaacattttgtctgaaattatccaaagaatgttcccatttctctggcaaggtctttcaacttgtatgtattaccaaatatagacataaatatagacagatataggcagggtgtttcatgcttctcccccaggactcaacagtggaaatgggatcaatggtatcaatggtgcggtggtggtggagatggagtaggggaagagggatatgagtgatgggagtcgcagtagtgctgatgatggtagaagtgatgatgatggtgatggaggaggtggtgctgatgttgtagtggaggcggtggttatggttgtggctaaggcagtgctggtgatggtgagggagaactgaggttcaggaatgacgatggtctcagcactgaggaagcagtggataaagccatcgcatccttctgccactggacagttgccagcggagatacctctactccaaaccggagcttgaccctggaccacatatgcaggtgattaacttttgcagcactggaataaccattctttagagacagatttttgtaattttgaaaacccgagggcttaataaactgtagccagaagggagttgcctagttagttaccaccctagcacccttagagattgctatttagtgtaggatttttttttgagacaggatctctctatatagccatgactatgtaaacgaggctggcctcgaacataaccttgccttttatttgcagtatagaggtgatatgtgcttaagggtagctcagggaagccctttctcaggagctcatgaccagaccgattcctgtgttttctgatgtggctatgaattgccacagcatttgcaaagtgtgttgacagaacacacatacaaacacacatgtgtgcacacacacaggcccacacacatgcacatgtacacacacagcccccccccccacatgcacacacacacaggcccacatgcacatatgcacatgcacacacacaggcacacacacacacatacacatgcacacacacagaggcacacacacatacatgcacactgtatacataggtacacaggcacataggtacacacatacatgcacacacacatacacacatgtgcacaaacacataggcattcacacatgcatgcacacacacatcaagcatacacacatgcacacataggtctctttactgttactatcttgttcactcacagtgtaaatgttttgcatgataatttttttgggggggggggagaacagatgcttcaaaccacacattctgctgaaatatgtcagagcaaatcaatgaccgcgtgtcaagagcgaggcagcgggcggtgtgctgagtgagcaagtgtctcagctgaatgagcactggcgttccacagcttccttatcctctctggaggccagctgccccttcccactgcctcaagacgatctcccaggccactactctgtgccctgagctttgaagaatacatttccagatatcaccccctccatgctcactggagggatctttgaccctcgggagccaggcttactctaagcggcttagcattaagaatgtgaggaaaccaaatctccacatttatgcagaaatcaaagactagaagaagaaaccagcaagttaataacttcctgaaataagaagcggcttgggactttacagctgatttcgtaagtggagaaattgagataatatcactcataatcatagacacttctgagctcttaattctgctcaggtttttaggaaaaaaaaaaaaaacaaaccaaccacagttgtaatcacagagcacagagaatgcagatctatagtcttgcatcctgttttcatcttgtagtattaatctacttccacgcggcttttctaattgctctccgacacaggtggaaatgctaaagttgcctttgccatttcctagaactggatccaaggctgtttttgctttaatgagtttgcttatttgttttggcagctcagctctttgtgtgaagaaaaggcagagcaggaaaggagctgttggaatggtgggtattggcacggagcttttagaccgggagttcaagacttggcatggctaaaggaggaggggcaccagcctttctagagaaagatggtgatgagctgagtgtgaaggtggactgatcagtgactttagacttgaactttgaagctgtgtaactaattaggagaatgtttcaaccattccttaggctgcagtctttcatttgcaaagtaggactcacacacctgctttggggttaccttaagggttgttacacaccacacatggccttgcttataacatgttagctgtttttctatgtcttgttcaaacagaaacccagattcatgtgttgatggagtacttacacagtgcctactgtgtgcctggccctgagctagattccaggaagcaggtggttatcactcagccccttagatctgagtgtcatgaggggctcagtgtctgtcctgtgtctgacaggagcagggagatgagagccctttgtgatggggtgtaggaacggtgactctacagaggtttgtggtcaaagcctaggggctgacatgtgacaggaccaaggttcaaatctacctttgctgtacactcactttgacaccctgagcaaatcattcgaattctgaaatctgcttcctacatttgtaattctacctcgaaagttgtggagttcaaaaataacatacatacatgactgtgcatgcgttagatgctcaggaatgcttaatgcagttgacatctctacggtgccctggtggcggctgatctttatgaaaatgaccccaacaaactccaatattctgaaatgtcaaagattggctgcattgtgcttgagatggtcggaaatacatcttattacagtctaatccttgcaaaccggccatctacgtgtgatatgtatgctaatgagagggagaatatgcagctctcgagaaattgcatttcttggctattatgtaaaacgcgatgaaaaatgactctcgaagatgttgatggctgcagccagagaggccccatcgggtgcagactggtcacctgtctctaagaatattgctcataccatgtattctgtcatcccacgggcagatacataactggaatccaatctacttagcaagatgtctttatgtaaagtttctgcttcataaatacttacgcgaatgctcgactgttcatttgttgtccttctgggtgtttttgtagcgcagtagtaaagcgggcccttagtactgatggactaagggtagcggatgagcctcgttaaacgttaatagatttatgtgtatgatggttaatctttcttgtcaaagggatcttgaatcatatatatttctgggtgtttcttcaagagtgtttccagagaggtctgaggagggaatacctgccttcaatatgggaagcaccatttcacaggctggggacttggacttcactgaaaaggagaaagcgagctgagtgccagcattcatctcgttttgcctcccgactctgggtgccacatgactcctggctgtggactccatgtgaccagctgcctcaaggtcctgccaccttgaccagtgagctaaaattaatccccccttgtgtgggggtcaggtgttttgtcacagcaacaagaaaattaactgtgatcataaaattaacagcaataagaagattaaccattaacaacacagtgacctaacaaatcctcttgctctgaagtagagtgaggtgacggctggcgtttctgtcctgaaatcctctgtcgtgtaatattcgcacctgcaagagattttgccagtcagaaggttttcttcaatgtctggtttttctcactgcttgtgggcaccttttctttttgatgaatttcgaatttgttcacttgttcttgttattcatctctatttacaaagtgtaaacactatttctctttcttattacattatttctgttaaatcattggccagctgcatttgttggacacccatgtgtgcagcatattcctcagtacgctggcctggatctaccacaggaattcattttgttttgtttttgttgtttttgtttgttttttgagacagggtttctctgtatagccctggctgtcctggaactctctttgtagaccaggctggcctcaaactcagaaatctacctgcctctgccacccaagtgctggaatcaaaggcatgctccaccactgcccggcttaccacaggaattcatattcactgtgttgcagagcgttccaaacaacacagggttaaaagaaagagagtcaaggtgtcttagtcaatgttctgttgctgtgaagagaccccgtggccaaggtagttcatctaaaggagagcattttgattggggctggcttatggtttcagaggtttagtccattgtcagcatggcaggaagcatggtggcatgcaggcagacatggtgctggagaaggagctgagagttctacatccagatacagcaggactcagagagagacaccgggcctggcttgagcatttgaaacccaaagcccactcccagagacacatctcctccaacaaggccacacctccgaatccctatcaaatgatgccactccctaatgaccaagcattcaaacctatgagtctacgggggccatgcctattcaaaccatcacacaagggaagcaaaccctgtctcctcaaccactaaagaggagggaggaagaggaggcttcaaaaaggctgcacaggatgctggcttaactcatagtgaaaatgacttacttagaaatcagggaagtcacaggctgagctcacatttgcccctcgttctgtgactatgactgaacacacaagtcattgcagatctttgcttggccacttgatggcagcacgctatagccatggtgcctccttgccctccaggactaagatctatgtctctaaacaaggatatgtccccaggccactactctgtagcttccaagtcatgaagttgatttagacatacttctgctgccctttaggaaagaatttgggtccctggagaagagtctgtgtcacaggcttagaacctgtctcactcccaatgccagtgactcctgccccatcctccttcttcttcctccttcttttccccctccctctcctccttctctttagcaatgcaatcgcctgcagttgtgactctggatgtttggggatgtgaagaacttgtactgatctgattgatgactcaggtgcagcggaggcttccacttcctctgacagggacctgcaagaacctatgcctgtcctgtcccaccctggatatccaccgtcatcactgacaagctgtggttttaggcgtcaccacctcagagctatcgtttccttttggtaatgatacagcttagagggtcttagcatacagtccctgggaaccagctcacctgttccctgcttcctctcaccacaaagcattctgtcagcactgtgctcaggcctcactttgctacaaggggatgaagagggctgtcattatcatctgttctgatgctcaagtcatctgatatatgaacgtcttcaggtggccccctgtgttctattgacagagcctcagtctcccagtgctttctaagtgttttggtataagataatctagactcacctcatcttgatgctgtctcagcctgggggctctgcttccgtcccggaggataatgtttgcgtctgggctggcagttctccaggccctctgagtagaacagatagaagagatgtggaaatatatgtgtgtgtacatccatggctctctctgtatctactgcgagtttgcatattagaagtgatcatctccttcgaggagttctgaaatcggatgcaggggcttatcactgtagcaagcacaaaaccaaagtcagctccgaggaggaagaggaacggaagagttcttacctgcagaggggagatgacaaagttatttcccaggcaatggtctccccaaacaaaggactcatgggggtttatttagcaggcagaatctgtacttatcacacgccgaagtgtcattcctgaacatgctcaggttaagatcataattgaaaaagaaaagtcacactgggcactgtggcacctgcctttaaccccagcactcgatggagagagaggtagacctcttgtctgaggctagccaatgagttccagagcagtcagagctatgtagagagaccctgtcttagaaaaagaaaaagaaagaaaaagtctcaaaccatctgtgtgaaaaattgaagtgacagcctgacatgcctctgggcgtgctcagattgcccaattttagctgaaaatgccgggatggatactttaaagaattggcaatgatgcaggagtcttgccccatcactaactggccaggacaatacaggtcctttccactcctcttccctcccacaactatgtagcccaagctagcctcagactcacagcaaccctccttccttcctcagcctcctactgctgtaactccaggtgagcatcaccatgccccacggaaggtgtgctttgtaacaagatgtgtgcgagtgcccagtgcaacgccgggctgtggcaggtgttctgacgcccttgatgcacctgctcatgcgcctgcagccacaggaactgcagtgtcagctctgtgtaggaatgaaggggagagctctcagaaccctggcggggcagccctgtctaggccggaggcagaagaaatggaaatggaggaagcttgagggtgtgcgcagggtgtgaacacgcaggctgagcgcaaggaaggctctgaaccaaactccctgctgggagaacacccaagaccatgtgaggcaggacagcatcaaacatggcttcagacaccttttccatatatatgcacacacacacatacacacacacacatgtgtttgcatatgtgtggacagatgtatgtgtatgcagagataagtatgcccatgcatgtggagactttaggttgatatagggaatttttctctattgttcatatctgcatgtgtgtgcatgtgtgtttgaatgtgtgtatgtttgtgtgtgtatggatgagtatgctcatgaatatggagacctttggttgacatagggaatcttcctctattactcatgtttgcatgtgtgtgtgtacatgtgcatgtgtgtatatgtgtgtttgcatgcttatgcagggatgagtatgctcatgcatgtggaggccctaggttgctatagggaatcttgtgtgtgtgtgtgtgtgtgtgtgtgtgagtgtgcatgtgtgtatgtgtgcgtttgcatgtgtgtgcagggatgagcatgctcatacatgtggagacccttggttgatgtagagaatcttcccctgttgcttttaaccttattcattgaggcaaggtctctcagtgggacccagagctcagctgtacagctagtctagtcagtcagcttgtttttggaggtcacctatctctgcttcccacattctagaattatagatggcttgctgtgcctactgggcatttatgtgggttctggagaatctaaattttgctactcatgcagttgttagtggcaatgacctaggctgaatgaacaggctgagggcctccaagactccctttaaccagatgtgcttggtctctgagccttgcaggtgccaggtagagtgacatcgtttttttttttttctctaatactagaagcttacagttaggagaggtgagatgccgtggctggtagagggatctggttgtgtaggggagtgtagactgtatgggaattgtagactgtgtatgggagtgtagactgtgtatgggagtgtagactgtatgggaattgtagactgtgtatgggagtgtagactgtgtatgggagtgtagactgtgtatgggagtgtagactgtgtatggggatgtagactgtgtatgggagtgtagactgtacgggagtgtagactgtgtatggggatgtagactgtgtatgggagtgtagactgtgtatgggagtgtagactgtgtatgggagtgtagactgtatgggagtgtagactgtatgggaattgtagactgtgtatgggagtgtagactgtgtatgggagtgtagactgtgtatgggagtgtagagtgtgtatgggagtgtagactgtatgggagtgtagactgtgtatgggagtgtagactgtgtatgggagtgtagactgtgtatgggagtgtagactgtgtacgggagtgtagaccgtgtacgggagtgtagactgtatgggagtgtagactgtatgggaattgtagactgtgtatgggagtgtagactgtgtatgggagtgtagactgtatgggagtgtagactgtgtatgggagtgtagactgtgtatgggagtgtagacggtgtatgggagtgtagactgtatatgggagtgtagaggctgtgtatgggagtgtagactgtgtatgggagtgtagactgtgtatgggagtgtagactgtgtatgggagtgtagactgtgtatgggagtgtagactgtgatcctctccatgcagggcaggttgggcttgcctagcatctcaggaatgtaggcaagaaccatccaagagagagctgcaggttatcagtggagagtgtgacactttcctgtctctgtctgccagaggaccattctgtcaccaccactttgccctggtcacttcagaacttgacttgagggcagtggggacctggttgtcccagattctgcccatttatagacttgcccaacatagcagcagactggggctggggcagcggagcactaactgctttcaaagcacacctctgctcaaagccatccatggctccgtgtaccttgagacagcaccagatcccttccaatggtgtaagtggttctgctgcccccttttaccctcatcccacgtctccttggtttgctcactatgaatggccctaaaattcaaggcgagagtcactcagggcttctacttggtacagcccatcagcataaggggtggggggtgtaacctgcggaaccagtgagctaccagtatataaaattgggtgtagagtaccaaatggagaagatgctgcctaggaagttgcatcccctgtgcctagtggccgttgcagagttagaatccctgagaaagttgagagagtggccaaggctttaatgccacagctgagtattcacattcccttttctaaacctgaaagagaacttcaggtggccagggtctccagagtgagtgacagcacagagccggcctgagctgggacctctcctgttctactccctgttttcctttgttttcttctccatggtgattgctttgctggattaaccctttagggaccatccaggaccaggagacagtttagagcatagcagaggaccaagggaagccaggctgggatttttaaacacagcatgagcctcccaaagccgtacacatcagggctgctaataataaccagtcctttgacattaactaaattgcagacataaagcttgatgtattgtttagattatggtggttaatccctaggcccataatatagagattattattaatgtctttttttttttaacagatcaggaaactggagtctagaaaggctaaataatttgaaggtcagacagacagctggctaagctcagagttgaactcaggtttttccctggcccctgcacagtgaatctcattataactgccttaattagcgtttcattttatgagcatttaacaagatttttagtggttttcaccaaaagtttttattttatagaacaatatataagttcttgagaaaaaaagccacatgaccttcaggtgcttgttaaagagtgggaatatctcagtagagtctgggagaccttgcccagcctggaccaagaaaaccgagtggcctgtttgatgaacaccagtattgaaaaggatgtatcactaacagtgtttggaaagcaacatccaattagctctggcaggcagtcgtgaagaaggtagtgtggacaaagtccagttatttgggacttggtcctggaatccactttttgatacccttagggcagtaggacaggtcctgggcaaattcctgtgatggctagtctcctttgtggactggatttaagctccccagggggatagattctggatgcgtctatgagggttttcccagagaggtttcactgaggaggaagacctaccctggctgtgggaggtaccacccctaggacagggcttttgcatgaatagaaagggaaagagaacattcttctctctctgcttcctgactataaatgcaatgtgatcccgagctcctgcagcccttcgtccccatcaccacaggctgtactggctggctccatgagtccaaatagacccattccttcttacctcgcttttgtctagcatcttgtcacagcaatgagacaagtaacgaattctattctctgtgcagtgtctgaacattgaattcaggtcatttggtgcatttatgggaaattcacttattgatgggggcttctccaaactcttgttcattcactgccttcccgtcacctgggcacctttgctggccaagcaccagcgtgggagaagggctatacctgctacattgtgactaccagtgtgagaggggctgtatcagctacattgtgactaccagtgtgagagaggggctgtatcagctacattgtgactaccagtgtgagagaggggctgtatctgctacattgtgactaccagtgtgagagggactgtatctgctacattgtgactaccagtgtgagagaggggctgtatctgctacattgtgactaccagtatgggagaagggctatacctgctacattgtgactaccagtgtgagagaggggctgtatctgctacattgtgactaccagtgtgggagaagtgttgtatcttacattgtgactagtgttaacttgatctgtgaagaaaggctagaaactattaatgtagaatggactccatgggaatagtctgtagggcccttgggatatgtaggggcatctcgggaactacagtgaactagggaaagcctgtagaatgttcttatcatatcatagcaagcgatgcatgcacaggacactggagcaggccccagcaaagatggccagtgctgaccgtctgagatcttatctttctgcgacaatccagtagagagattcaaggtcacagaacagcacctgacaaggacacagggatgtgagcctcgaagggtgggacttacaccattcatttaattatcccatccatcattcatatgtttagagacaggtttcactacatagttttgtctggcctggaactttctatgttagattggaagggtctcacactcacagagatccacttacctctgccccgttatgcccagccccattattttatttattttgcacatctaacttgtgggagttggttctcttcttccaccgcatgggctctgggcgttgaactcaggctttcaggcttggtggcgagctcctttgtctgctgtgccaccctggtgatccaaggatgggagtttagctcagtcttttctttaaatatagctttatttattttatttgttatttttttgaaagaggctctggctatatacccaaggctactcttgaactcaggagtctcctgtcttagcctcctaagagtggtaaacaatcactcctggctcaaagatgactttataggaaacttcgcatttttttaggatttgatcatgaaggttatttctcactagaatggattccaatatctgtggggtttttttaaaaagtggttttatagtgatccacacttataaattgtccaagatgacccgagggcattagaataaccatacgacttactgtgtgacagagggggttctgtgctttcaaggcctgtgattggctacaacttgataagcagtttttatttatttctttaaatttaacattttgtgctgttaaaaaagaattgtcttatgagcatcaacttctccaaagtgctagggatgtagctcagtccctagagtgcttgaaaccttgggtttgatccctagcactgtataaactgaatatagtggtgcattcctataattctataacttgggggatggaggcaggaggacttgaggtttaatgttattcttagcttctcagagagtcccaggttgtcctggactacaaagaaaccctttctgaaaaaccagctagccaaccaaccaatcaataacctgctcttttagaatcccccaaatttgacaattttctcccaaatgatcatcatgatttaaaaaagtaacatcagttgtcctttctgggtgtggggcagagttgtgaccacagaggctggtaacctacagccctgccagctgggaggaggaggcggtgatggaaaggtctctggagctctggaggagaaacagacaggggtgggaaatagccctcagccttgagtggtccagatgcagctgggcttgggttgagaagagatgtgggattatcgtgctgaatttcatggtctttctcctgcttttacctctttgggtttccagcagagacattgagacagaggttggaagaactggaacaggagaaaggccgcctgtcctgggctctgccttcacagcaacctgctcttcgcagcttcttgggttacctggcagcacagatacaggtggccttgcatggagccacccaaaggtgagtacccgtggatgccaccacagtccgtgcttggcttcctcttccccactgccatgcagtcaagagctgggaggtcctggggtgatttttcctggggggaattctttggatgttggagaagataggtccatttagagggaggcctagttgagtcctccttcttgagaaagaagcccatcaaagtgatgggaaagactagacaagcacagggcagaggacaaacacagaggtcctctgccctggctaccatctggtatcatctggaggaactctgaagtctgagccccaaattagctgagtcagacggtttgacatgggtctaaagctgggtacatctgaatattctgagactcattgctctgtgtcccctatcccctggccaaataaagcccgagagggaataggtacttttgttgttgttgttgttgttgttgtttttgtgttttcactttttgctgttgtgagcacaatcaggatttaggtaggcatctacatatgtaggctcagatgcagtcacggcaactgcatcttgcttcccactgccaatttacctcagcaatcacaggattgcatggcccgttcctgctaggtgcccatggcattgcacattttatcttttatctttgtgtctccctggtaccggttttgacaaggaggccaactttctcttttaggggtagcgagtgacggagcatgcagtctcactgtgtccttctgtagagatatatgcgtgttgagtaaactcacacgctgtcctcacagggaggagccccatctccacacttctgttatcttatctgatgggaatcatcctctcagtcccccttcctgcctcacatggaggccttcctagctggaggccttcctagctgtctcttctcttcttcttggtttgtttcttgtcctagactccagctgctcttccaatgtatcaagtttctgtatcacaaatacttgcagggtagttactaagtgtttcctaagtaagtctcctctttcttcttcttcttcttcttcttcttcttcttcttcttcttcttcttcttcttcttcttcttcttcttcttcttcttcttcttcttcttcttcttcttcttcttccccttccccttccccttccccttccccttccccttccccttccccttccccttccccttcccctcttcctccttctccttcttttgtgagacaaagccacaagcccaagctagccttgaatttacaatcctcctgcctcagtttaccaagtgcagggatttttgctgttacactaatatgggcattagctttatcagccttcttgctttgttttctccataagaggtttttcaattccactctaattggcccctacttctgctgctacccatcccccaacaggtcagcagtggctttctgttaaactcttgctcctcttactgtaagcaccatgtccaggcttctagtctcctcagttcttagatctctactcacggctctgtccctggtgtcactgtccatccttgttgcatctgagatctcgtctctctctccttgtattctcaccttgacatccctacctctgcaggatcctgcattctttctggtgtgtctcagatacacgaatccggttcaccttgtaggaatcccagccctctgagggctcctccccagcactgacctctctgtagagtgctgaccccaggcttcctgtggtcagtagtgcatgtattaaattcatctttttaccaatacagaggtactgtggtttcatccttgccttagtaaacttattattggtgatggttggtcttgcctgtcaactcgactgcattaagagatgtcttggactggtgagacacattcaggggtctgtggaggcatttccagagctgattgacacttggaatgacagtgattgggagaagaaagaaggctgaggacaaagatcatgtgccttccccagctttctgtgcatgtgccgggcggcagttctgctgactttgggctctagcttctttggccttttgacatggatgcaatacccacagctctccagggagcttctgggcttccagctctggtttaatactgctgagttgtccagcttcatgggctgagccgtgactgtgttctctgcccctgcagcatgcaggcagcatagttggattagccagtgcctagcgtgtaagccaatctaattccttcttacaatacatgcattcttttggttctgttcctctagaaaaccctgccaaagaaactgctctaaattttattttgcctgtgtatgtgtgcatatatatatgcatttccatatgtgcgtgtgcatgtgcatggtgtatgtgtatgtacatgtgtgtatgtacgtgtgtgcatgcacgtgtacacgtgtgtgtatgcatgtgtacgtgtgtgtgcatgtatgtgtgtttagccatgtgcatgcatatacatgtgtgtgtgtgtgtgtgtgtgtgtgtgtgtgtgtgtgtgtttgtgcatacagaggcccaaagttaacagagtgtcctccttgattgctctctaccttattgaggtgcggccttttgctaaacctggatctcactggtttttgttagtctagccagccaaatcacctggggatcaccctatcttcgtcttctgagggcagggattacaaggccctctagccggcctgtcttttgtgtgggttctggggtttgaagttggatccccatgcttgtgcagtaagcactttatcctccgagctatcttcccagacccaccatctttaaaaaggagccctgctaaacctattatccctgtcctcatgcctcctcctcctcttctcccgggctccctgtctcatggtttctacctccaggcacctcaggaacaccccaggactccatcctcctaccctctaactcatgggttgctagctctggtcatctctgtcatctttctgtcattctctgtcacctttccagcctccagccactggaggcttcctctgctcgcactgcagcagcctctcaccttgctggcttctctaactccactctggtatatcatccatttcccccaaagcagtcaggaggaccctgcaggaagcagtggctactgggctttcaggatgacctcactctctgtgtgaccttcaaagcctctatgaccccagccttaccagcgctgacctctgactagtcagcaccagcaccagctacccacacagcagaatgttgcacaattctccaatctcccgagaatcccgttgcagtcactaatggttgctcatcctccaagacagtaaagccagccccgtgccaaccctctgcctgtcggtcacatctcacggactcttcctggagtatcctgggtgactgtctctgtcccaagcctttgatctactgccttgaatatccctcattgtgaaggacaactgtccccagaggaggactgcaccgtgtacccctccaactgacttggtgcgggagttacatatgtatacacgtttgtaccagttcacaaacatatgtgtgaacacagggttgggggagctggagggagaacctcccttcagccaggcttccttctgatgctacaacccagggatagaggctgggtttttatccctttagttattttttattcttggtttttcttttgtcaagcttacctgtcctgttattatgatagctagaaatgacttgacatctgcgtcattatcacattcttacagagcacgtgggcacggctacatagctgctaattattaaagtcagcctttgcttgtgccagcttctctgtggcctaagtttgcctgtactcaccagggtcagctggaccctcaggacagaagttcctgcagtcctcatgatgaccaccaggggtccctggacacaaacttcttcggggctcagccccgagaccccggaagaaggctgttctaaatgctggggacttttcctgcagaaataaaatagttttgtcaacatggccagctttcaacacagattagtaactcaaacaagtttctttttatctttctaggatcaagtgtttctttgagaatctgatgaaagcaatgcatgttcctgtattcaaactgccagggtgtctcttgagaccctcagattttgagcctccctgtcaatctggccatgggaggaggaggtctctcagcttctcgggttatgaaagtaacctcagactttaccatctacttatttttattgtctcttggttttaaaattactgctcaaattctcaagtaagcttaactacccaccccgagaactgtttatatcagcactgaggtcactagggttggagggtcactgagtagctctgacagttgaatcaggtcctcccactgtctctcaggtggaaatctctgttgctcagtaaggggactggtctctgggtgaggtaaatgccgctcctctgtgcaaggtggcacaaggtggtttttagcttttacaacagtgattatcaacttgtcatgacttctttgggttggctgaacctttcacagaagtcacttatcagatatttacattactgttcataaagtagaagaattacagtcatgaagtagcaaggaaagtaatgttttggtcggggcttgccacagtgcgaggagctgtattacagggtcgcagcgttaggaagtttgagaactgctgttttccagcctggcaatgcactttccccctttgttacaatgtcctgagtggatgtcacagacacgggggcatttccacgtttacccccgcatggtcctgtcaccatgccatgtcaacaataggtcagattcttagcctcgtctgtctgctgtggatcgttacgctgctatcctattctctagtgtctttggttgcttctctaaacacaagctactgagatccttgcctcaggcccagctatcaggagaacccaaactggacagctatataccctggaaaaacaacccccaactcttgaggtgccggggctctctgacttctgatggtggtagcaggtccgactgcttcctctttgatccaatcagtgggaaggaagtatgtccacccctcactctgaccatggggagggcaggaggggctgagggcagcagcccatggtggtggtgggggctctgcatgtgcaacggctggcgtaggcacctatcccacagacttagggggaaggacttctaggcctgttcttgtcacctctcatttctgattcaacctctttgacacaccagggttagaaggtcaacactcagttgaaggaaaatgtcctcttctcaaaagcttttggcagctgtagcaagcagttgactggtaatctcatctcattgataccttgttccccccccccccccccactgcataccatgtgacttctctctggctcctcaataatatggacaccacactggttactcttgtcacctagctgcacttgtgtgtgcatgtgtgtgcgcgtgcatgtgcatgtgtgtgcacttgtgtgtgcttgtgcgtgtgtgtgttcgagagtgtatacgcatgcagaatatctcttatcagcgacccttctgagaggagcagcatctgacaaagataagaaacaaagcacaggaaggctctctctacccactgagacaggacactggcactgccaagatgcatcagtgtggctcgtctgctcccctgggacaggacctcaggagcaagtgtgggtggcatgcaccggcctttcctctcctctctcttgagcatacaattctttgatttctggcagacgctgaggcagactgcagtgttgcagggcacagcgacccttggattggaatgggcacacagggcacgagagggagaatcggcagatgttattatcagggattatgtaaagacattgcccgttcatttctctgccgtacttttctctgagtctcgctatcggattttacagataggtctcatcccttcagcgattgatgggacagaaaccagatagaaagattcaagtttttgcacaactgggcaggtcctagtggagacccaagtctgagagtggaagcttagatctcaggcccccgggttcccaccagcatcaagcattaacatccctgaaacataaaggaaaggctgggtagcagagggagactttggggaccagacacggaagatggaaatgtatccatgcttcatagctgagagatgcgttctgaggccacccctggctctccgtggatcacttctgtcccaggggtaggagcacgagagacagacaggaatgttgtttggagtacagggcttctcttgtgggttaggtgatgggtggagttaggtgccatgcagtccccagctatgctcccgtagcttccctccgcagctggtatttcagttctgcgctgacttcctacaacagctcccacctcaacccagatggcatcagttcaaggaatttgaggaacactcataccagcatacgccaggcaagctgagaaataactgaagggaagaaatgagctactacagcaggagacgggatgggatgacttcccagttttaaggctgtgggttcattaagccatccagcaggtatttgctgagtcctgactcactggggacatcagcacaagaggagctctgtcttcctctgacactcacagtcaagtctgaagagcccgtggcagcttggagcccggaggaaacctctgctctctttctgctggaagacggggtgtcttaggatcatgtcacacagaagaggctcactgtgttgcagtcttcatatccatgtttatcctgtgtaggactgcgcccaccacacaggttcaacgttcactttactggtgcctgtgataggacgttggaggtctctggcaaccacccaaggacagggtgccttccccaggcagtccagtgtgaggaggctgtgaggttcagagggttggattagttcaaagacagagaagggcaaagctaagttcttacggtagccttgacctctgtgaactgtgttctgcctgtgccttcctgggaagagccctgcagaggactttcccatggaggagggtgggatgtggctgaagggggtgaacaaggagaaagaggaggggaacccagggcttcattcatgccaggcgaccactttccctggccccatgtggggtttctaaattagattgtattttatgcttccagtatagtttagtttggactgactatgtgtcaggtgttcatagccagggtggctatgggctagggcatatggaaatacaggcttgatagatgcaacttggatggaggaaaaaaaaaagccccaacagatcctagttgaaacagaaatgtacattaggggctccatccctgtgagcacccggatgagacaagctttgtgcagtctctaagtacatgtcgacctgagctcatctgtttctccacacctgtaccaatgcatgtctgttacttgaagggccggcagcgatgatccagaagccccacttgaaggacagctgaggactaccgcccaggatagcctgcctgcatccatcaccaggagggactggcttattcgagagaaacagcgattgcaggtgagtggaatagaatcttccagaaagggagtgggccctctcttacccatgtaagtcagatgcagtgagggggcctctgcttcactgcagtgcaaggactcaaagcggaggcggtagagcggccactcctttgcacaggactgggttgcagttgcatagatgggagtcaggatctttggttctgctctctgtggccgctggtgtgtctgggaatctctgtggccgctggtgtgtctgggaatctctgtggccgctggtgtgtctgggaatctctgtggccgctggtgtgtctgggaatctctgtggccgctggtgtgctaggaatgctttgtgggaatccctaccatggtggactctgggaaaccactgtgaagagacatggttttgagtctagcccgggtgtgaggctgcatctgtaatgaccagcagatatgtggaggccatgcttggctcacagcagtggtgacatctggccaggaagtgcccagggttagaactctaaatgaggctccaaggtgagagagcagaggcaggaaaggggtctggaagttttctaagtagaccccgggcagggactgagaagcagccaggggtcggggttgggggtagggggctgggactgctgagcctgcagcctggagcaggaactgggagtctgtgaagcaccaggcagcagcaccagggattcatgggaagagcctaaatggacagagtggaaaatccatccagtgtgattcttaacatcaactttgggcctccacgtgcacacatacccacgtgctcccacgtgctctcacgtgcctgcggacatagacacaccacactcccatgaaagtggaaaaaaaattaatagaagaatgagatgttttgttgtcatggaaaacagtcttgcagttcctcaaacatttgaaaatggagttcttcatacccagcacccagtcccgctcatcgttgatacacacctgaaaagaaagtgctgtctagacaagacatctagtcactataactgatgacagcccaagaggccatggctggatagacaagcaaagtgtgttgtggatgcactttgtggtctgtgactaggcttacaaaaggcactgtactaccgggcacagtgtgggtgaatgtggagggcattgtgtttgctgcatgagacagtcatggaagagagatactgtaatattctacttagattcccagagtaggaaaggagattggggtgcggtggggagtgggtggggtaaggaatgggggtgaggatcgggtggcaggggacggagaattagtgtttaatggggatagagtcttgtggttggatggcgagattgctcgcatggtttgtgtgtgtctgacatcactgaactgagcatgtgtggtggcagagacgctaattcttatgcctcttttactatagtttagggtgacctgagcttgtcttcctagtatggggaggctgaggcaaaagtattgctgtaggttcaagcctgggctgcaagatgagtcctgggctagctctgactgctaaaaaaaacgagagagagagagagagagagagagagagagagagagcgagctgtggatctcaagggcagacagagcagccaagccttgttgaacacagcctgcaggcgagccggaagttctgggtgggagtgttggagctcatctctctcctctgggactgcaggtgggtgcaggaggtagaagctgtggagaatctgctccgtgggtggagagaacattcgggagataggggagatatcaggaattaagctgtgctcttatcagctaggaacagcaagggaacaggggcgtgtccctcagcagagcctggtgctgtattcagactcccacgcatcgagatggctatctgcccagcaccaagcacacgatgttcagtcacctgctgcttgtttatgcgttggcctggagacagatgatagttatggattaacatagattctaggtcaatcaattagtgcaaattgtgtgtgagtgtctgtgtgtttgaatgtgtgcacatgtgtgtgcacatacatgtatgtggaggacagaagttgatgttggatacttaccccaatcacccttcaccttatttttgacccagagtttctcattgaatctagatttcaatgactgggctagactgtggccagcaagttcccaggatcctcctgtctctgatcaccacaccctcatgctggggtgaggaattcattctgtgggaccccagctttcccatgggttctggggaatcagaaggtagcgcacgcctttaatcccagcacttgggaggcagaggcaggtggatttctgagttcgaggccagcctggtctacaaagtgagttccaggacagccagggctacacagagaaaccctgtctcgaaaaacaaacaaaaacaaaaacaaacaaacaaacaaaaaacaaagaagaagaagaagaagaagaagaagaagaagaagaagaagaagaagaagaagaagaagaagaagaagaagaaacagcagcagcagctctttgtgcgtgcacggcaagcacttggcggacagagtcacctcaccgactgtgggaaccgaactgtccgacatgctaaaggacattaggctccatggcccaacagccatttgttctctgcatcccattgctgtgacaagccaaggctcctgtctttctgtgcatcctaaatcctcattgtttgtggacagggttttctcctcccggctcacagctggcatctcggtgtggggtatgagctgctagcaccctcacttgcccgctgccagaagggaagccagtgagcgacacaccattcactgggttttatcaaatcaaaaccttggcccgcacctgtctatatttaatgcttgggaggaagcaatgttttccttcctgccagcacatcaaaaacacaaagctggcggcatgatttttccggttcagcaaacttctggaatctgcctgcctcggaagggtggggtaggtgggctttgcgactgcggtgcatatggcccccctatcatttagccactctcattgtcagcagagcagggttaatggctgaaatctgttccccctctctctctgcagaaggaaatcgaagctctccaagcacggatgtctgcgctggaggcaaaggaaaaacggctgagccaagagttggaggagcaggaggtgctgctccggtggccgggctgtgacctgatggcactggtggcccagatgtccccaggccagctgcaggaggtcagcaaggccttgggagagaccctgacctctgccaaccaggctcccttccaggtggagccacctgagaccctcaggaggtactggtgactttctgagtttccaactttacctctttctcctggttattgaggtgactttgctgtcccttgtccatgggtccttctcaggcagtctgtttgctgagtggaagggatggaggcattttgatgttagattgccttcggggtcagccatgagcctgggagttatttggtattcgatctacctaacccctaactgtctgcagtgtcctctctccaagaagcagtttccaccattcccaggggctggctcgtctctccctccttctctccctccctctttcatttgggattcaattagatagagatagtgacactggcttttgatgaagataatctcctggaaagagacagattccacttctatcaagggatagggcatggctttgaaagactgtaccctgggctgcggtgatgctcagatataaagtgttatcacacaagtgtggggatctgagtttagaaccccagaccctgtgaaaaatctgggtgcaggggtacacacctttaacctggtttctggggctacagactcaggaggatctctgaggtttgctggccacgcagcttagctctagattcagtgaccctgagtgtgatcgaggaagattaacctctctctctctctctctccctctccctcttcctctctctctttctctctgtgtgtgtgtgtgtgtctgtctgtctctctttatggcatgtgtgtatatatgtacacgtatgaacatgtgcacaaacaaacaaaaatctcccaaacaaaggacaacttacccagtgttttcatagacactggagtgaatggaatgaatgagcgaccttggactgctggttgtcagaggctaagcaatcaccatatgcctggtgcctacacttcgtgggttgcatatattttcctgtccgttttccagatagtgaacagaagattaggaaagctaatgattaaaagagaagtttataattttcccacatcttaacaagaaaaaaaaaaaacatgctcaggattttcaaacctgggtatatctttgggccagagtgagcggggctggccagagtgagcagagttcttgaattcaatccttagcaaccacatgacggcccacagccatctgtagggctacagtgtgttcatatacatacatacataaataaatcttaacaacaacagcaaccccccctcccaataggtcccctaagttctgtctgtatacatgagggtacaggggtcatccactggagcaggcaacctaagggagacctcacccctaaagaaaccatctctcctctggcacccatcaattacagcagctcaaaagagacaacttttaagcaccaggaggatcaggatgtcccaagccaggaagttgacaggacacagaaacacagaagccacataaaatgtcagactcattacagacagacaatgttgaggctgtagagaaaacagatccctcattcactattgacggaagggcgggctggtgcagccactggggacatcagcgtggaggtttctcgtgaaactaaatatggaaagcccaaataacctaggaatccttccttccaggcattgacccgagaggctgcctcctgcctaccgcagggatatccatccatggtcattgtggctctgtcgggtgatgagcccgaggcacatcgataatgggcttttatttggctgtaaagaaaaatgagatttcagaatgtataggaaaatggaaggaactggaaaatactatgttatgtgatgccccagaaagacaagtaccacacgtcctctcttgtatgcggatcccagcatctgatttccctatatgtttatttgtgtgggagtgaggtcataggctaggaaactgggaaggggctttaaggaagggatgcggagaaggcaagaacatgcatatgtcacggggatggtggtgattgtcttttttttaattagatattttcttcatttacatttcaaatgctatcccaaaagtcccctatactctccccccaccctgctccccaacccgcccactcctgcttcctggccctggcatttccctgtactgggacatataattaatcttcgcaagaccaagggcctctcctcccaatgatggctgactaggccatcttctgctacatatgcagctagagacacgagctttgggagtactggttagttcatattgttgttgcacctatagggttgcagaccccttcagctccttgggtactttctctagctcctccattgggggccctgtgttccatgcaatagattgactgtaagcatccacttctgtatttgccaggcactggcacagcctcacaagagacagggtcctgtcagcaaaatcttgctggcatatgcaataggtttggtggctgtttatgggatggatccccgggtggggcagtgattctctcttagggtggtaaacttattaaatataatagaaggagcagcagcaggagcagcgtggcaggggctcggaggagagtttgggcccttgtggcagcatgtgtccacctgcaggtctcagaggaaggtgggtcatcacacagttgatgagcagtctacagctcatacctgcagccaggctgaggctaattcctgagaggtggggggaggggaggggtcctgctttccaaggaaggaatgtgtgagctgactgaggaggtgaggggcgggagtctacttagttaaggtacccattacaggctgggtacctgcctcctttagctttctggtatcttggagtctgggatcccagccctgtagaaatgagaatttccttggagaaatgagaattatagtgcctgtcattctccaggaatcagggcacaggggaggcaaggcagggtttcttggtgggggggggggttgcagatttaaggcctcttggtaggggacatgccatttccttcagaagagtcacgaaaaagaggctgttggacctgaacaggccctatccctgactgactccatgggagtggaaaggggagtgtgggggagggacggttccagtagtgagcatgggggtaggggtaacactgagggtcttactggaattgttagggtcagagtgtggcattggcagccgtaagagactaagtaagcggagccacctgtcctcagtagggtaaagagattcggaatcgcaaaagcagagaaaggcttattcagtgtgacctgttgacaaaacagtgaggtccagtgactcccaagtccctcgtgagggtcctaacaggaggtttggtcttacagagggcacaggtggttatagcttagcagtgctggtcaagtgtggactctcccatcactgaccttcctttcagctccagtctgcactgctcagtagtctaacaccgtgtcagaaccctgggacatctctctaagggacaagttcgctctagatggctccagccttctccttctgcccgtgtgagactcctgatagccaaaatctgatggccaaaggaagggacacacgtgcctctctggggatgtctttgttcctgccggtgtttacaagatgggggatcatgaggagcagattggggtacggaggaacaagcaaagctgagcatgtatggaaataccacacactaattaaaaattaaattcagaagagagttagggtcttgcctgaaatatcaaaccctggccttgcaaacaagccccaactcatcttaatttaaaaaaaaatagttaattttatccatatgaatgttttacctgtctatgtatctgtgcaccgcatgcatgcccgatgcccacagaggccagaagagggcattggaactggagttacaggcagctgggagccaccatgtgagtgcccttaaccagagtcacctgcctagccttggcagttgtcttttgtcccacaagacctccttttgatgcaggtggccaggaaggaacctgacgcttattccatcccaggtgcggggctgggatgtgagggtggagaaggcttgtgaggaagagggaaacaagagcatcaaggcgacgatggtttccactgcttcctgagggctgcatctctgcacggggtggggggttccgcctatttggcatatgaggaggaggggtgtggtaattcaagctcattgctctccagtgctgccagctgtatccctctaccacccccacccacccccaccccccccacccccgccggctgtgctttacagtccaggggagggtagcctgggacctcatctagcatgcatctggctccctgtgcagctagatgagggtaattgtagtactaacaacagcaggctggtgtgaggcttcctggcacactcttcagggatgcttggctgcttacagtggtctgtggttgggtccttcatcactcggctcccgggacatttcacgcttcatgtgaaatgtgttctggcggctcatggatgacctgtctccggttgtgcatttggaagaaacatgtcccgtctcttttacaaactgttggcataaactgtgagacactccgatccctgcttccagcaaatctggctggttttcgcttgggagagtctggacgctcgcttgcttgtgtcacagctgggccggggagggggccgggaaggcagacgccaggcctgttgtgcctctttcccctttagctgtctaaccaggggttctgcagtgagacagtggcctggggctgtgtgtggagtggctgccacacctggtgccttgtgtgaagtcagagtcccacaaccgggctagggcttcttgtaactgagatttgaattagcagaagccagcacgtcacagtttaactggaaacaaaactcatcatctctgtccttcaaatgcacaccatacaccatatagagtggagagacccagggagatagcaagggtgacgttggaagacaggttcccctctaatcagggagttgtggcagagaatcctccagatgggtgagtggaagcagccagttgtgagaccccgtgccatgactgtgcccaggggataggggtaggtgatacctacctaccaagtaggacagcacaccagagcagccctcggccagggagcagactccagtctgcacccccatttctgggggtggctgctgtttaacttctgggctccatttgtgttccctacctctgcctgggaatctgcctggtctgacaggaacagtgggttgctacatgtatatttgtgtgttcctatgtttgtgaggtgtgtgtaactgtggctacatgtgtatttgtgtggtgtgtgtacatacggctacatgtatgtgtgtatgcttatgtttgtgtatctttgaacatttgtgtatatgtttgcatatgtgtatgtacatgagcatatgtatatgtgtttgtgcttgtgtatacatgggtgtgtgcatgtgtgtgcactttcttactcaaactatctgggtgatgtgttaggagatctctctctacttcacttgacagctttacccatcactggcagcacagacctgcctgtcttctgtctgtccttcctgtcagcagtaaggtcaccctgccacccgagtggatagaggggacacactcactgaagccggcgcatggtcactggacacctgcctcttggcatgggcctctgaccactgcaccacactgcctgccccgggagcagaggcgtcagctaagcagcctttgacaagactgtacagaacagtcctggtgcatgtgtccagcctgtgatgatgtcatcagcagtgaggtcataagggtttcagggtcaaaaggtcaagtgggtgacttcttattccctacagcagtcccccctgtcagattcggacctgcttgtcctctgcggtcactggcaaggttgttaatatcagcagagtctactttctcttaagttccagacccaagcagcctttatgcttagcatagctgtcaccaggcccttcctgtcattgcccacttgctttgggaacagtctcattcttaaactaaattctgctaaataaggaaggttgtggaattgagtcatggggggtgggggggaaggaaacagaagagaatcggaacacggacactaacactccccgtggctttgcacgaaagtcagacttctgtgcaggtagttctggggacaaagctgtgtgctgagagtctgtgctgtagcaccactgcacagaaagacaatcctcccctccctccctccctccctccctccctccctccctccctccctccctccctccctccctccttccctccctccctccttctctctttcttccttcctctcttcttccctccttctctccctccctttcttcctttctcctttccttctctccccctctcccccgctccctcaccatgttaagccaatgagcaggctctgaacaacacataaggttaatcttactggaaactgaagattccatgtgcatgcgtttacttccacgtcaacagcagcagcagagacggggggacttaaaagatattcactgttctgatgtggaggctaaagcccagccagggtcaaggtgtctgaaggcctctgactggcatccttggatcttcatgtggagtttctctcggtgtgttgccagtcttccctctctctcctcctcccatctcaggctttccctctcttctctctcctctttcctcctcctcctttcttcttctccatgtgtgcatgcttgtgtgtgtgaaggtaggtgtgtatgtgtgtgggtgctcacacacatgtatgtgtgcgtgtggaggcacaggggtcaatcttaggtttcttccacagtagctttccaccttgttgtgtgagcccagacccctcaccaaacctgaagcacacatcaccacacctggcttttaaaaacatcttgttttaaactttactttgcacatatgggtgtttgcctgcgtgaatggttacacacaacatacatgccgtgcccacaggagccagaagaaggcatctgatccccctaattctggagttaaggacagctgtgagctgccatgtggatgctgggaattgaacctgggtcctccggaagagcaaacaatcctcttcactgctgagccatctttccagccccatggcttttgtaaatacttggttgtgggagtgaaactcaggctctcctgctttgccagctgagactccccagcccttgactctgctactcctagggcagatgttgtgaaggaagaggttactgttcgtaaaagaggtcatttgggaatgacgtctgagtctgccgtgccctctggactgaaggatagtccatggctcatttgttgattgattgagagaagacctcacatagcctgggtgggcctcaaatgtgctatgaagtcaaggatggatttcaacgtctgattgcccttcttccctcatctgggtgctggggttacaggcaagcgtcaccgtgcccagatctgaggggacacttctcatttgttatgattcaatacgctatcctagtgagacacagggacaagctttgttactgatgttcatggaactatcaacacgtaactggtataacgtaattttgactagtagtcctgaccctggggacctcagtgtggtggttatagcaccaaatgtaggtggagttagcatagctcactgtacttcagatgctctggtcctgttagaccagatcctctgttatctctgttgcactttgggctatgtgaaacgatacagtctcagtgagtcttactactaaaaatagcctaaaatccgctggtataatatacatgaatgattcatagtcgcccccacccctcactgcttccattctgtgtatcccctggggatacaaataatgttaatattagccagagcaacacataattaggaaaataaatctactgaaaaaaagtaatatagcataaattaaataaattaaatataaatcaaatcagatataaaacaatgcattcaattacccgcatcactgcctcgtcctctcagggagatgtctgagttgaggctcattttccttctaggagaggggcagagcagagcccagagcaatgagctgagtccagtcctggaaaggtggtacctggcctgattttcactggggctgtgggacatttgctaaaggtgctatccttgaggccagctcagttttagactcaggaagacagcctgctgtctgttttaatgtgtaaatccatgagctgatttctgagtgtgtgtgggaacactcacattgtgctattaaatgctcaaaaggttctgttatgtgaagatccactgatttggagggttccagtagatgggccagaggtggagctcagtcggtacttgaatagtagcagcaagccataagttggaccctattgtcacaaacatcaaatattgtgtcgcacttttgtaatcccagcactcaggacacagacacaagggtcgggattgcaaggttattctgaggtataaagtgagttcaaggccagcctgatctgggtgaaaccctgttttaaaaaaaaaaaaaaccaagcaaacaaaaatgtctaatttgaatcgacttccatgaccctggaagaggaaggaaaaacagttgctgatcaagagttacaagtgtggatatcaggctccagagctagtactgagtttcaaaccaaaccaatgcttcacagattctgcccgtctcgtcagcagcccaagctgcctgtgctctccacataagctgacaccttgttaaaagatatctttacactctgtataaaagtatacttaatttgcctgatgtaattgtaatctcgtaggcttactgctaaataagctcaccctttcttgttctttctgaactctggccggctggttcaagtcagctgttctggcccaaactccactcccaggtgactgattcgatctgccttctctcagcttctcactgaactgctctgcttggcctcaaactaactccagcgacttgttctactctctggcttcttcttattctctgacttcaacggcctctgctggcctgcactgaactgtatgcgttcacaaactcaactccactgtactgcactcacagcaacgactcccgactctgactcacactctctttctctctctctttctctgtgctactcttaaacagcttcttgtccctgtctgttcttgtgagagttggatatttcctctctctgactcggtctctcagatctttctctgatgcatcaatttgtctgcccctaaattagacatcacttccaacggacgcacctgcctgattcagccatactcagacttccatcctccaagcaacctctcagagcaccactccattaaaactagaaatgcctccttcttgttggcctgtgcaaacgcaggatgagttcctcttgcccaaaccggaagctccttaacttggagcatcataccaaccagaacttggcctccttagtccatcatgaatattttaaacagaaatagcctcaagatggctgacagtcacttgcctactgccgagatcaaataaacacgatcatttcttcctattgatttgatcctcttcctattattgggtgggccttactccattccccacccctacccccaggttctgtgctgcccatctttatatcaacttgtcacgagctagagtcatcagaggaggaagcctcagttgagaaaatgcctccataagttgggactgtagggcattttcttagtcattgatgagggaaggctcagcctagtgtgggtggtgccatcactggtccaggttgagcaagccataaggagcaagccagtaagcagcactcctccatgacctctgcatcagctcctgcctctaggttcctgtcctgcttgagttcttgcactgacttccttcagtgatggactgtggtgtggaagtgtcagcctaatgaacactttcctccccaacttgctttggttgtcgcatttctcatcatagtgataatttgcttgccactgaggtgttccatcacagtgataacttgcttctggtcacggtattcgtcacagtgatggaagagctaacggagacacttcatgttagcagtgactgctcccctattgaactcttatatgcacatcttactgtgtgagacgtgaggaccagttggtttcatcttctgtaatctcattcctgcctttgcatctatctgtaagctctctgccaatctcccatagctcttacactgctccaaaagtgactggccccatctgtatcctacctctgacagtttttattggatatccccccccccgccccagcattggcctttctccaactgaatcattagtgtgtcttgctttatgtcctgactactgttggcacacatttattctgtagatctcagtccagagagtcagatcagggttgtagcctcacacgttcatgcgtgcccgagttttgacagagtacctgctaaagacccatctcatgagggagtgaacccagcagatagatggtgctcatgtggaactcacactccatcatggttcgggtagagattgcatgaaggaagcagtgtgtgaggagaaggacttagtagaaaaagaaggcaaagctgacgttcacagtcatgtccagacgtttgccttgtggcagatgctgttacctacaaggttgattgcatgacgtttttaagtaacttgatggtatgtgttggggtgttcatagctatcgcagtgggctgcaggttggacacacctgggagaaggaattccagctgacgaggcatgcttgagaaatatgaaatatgcgtctgatctgttgccctctctgaggctcaatgcagaggcagtggtattacctcaaattgttgacagtatgccccactcagatgccaatggtcccatgccaatgtggtccactcctttaggaagctggttatagggctcagggcttcctgcaaccattgctctaggaataaagacagattgtatttgccctggatggcatggaattctctatgtagactagtctggcttcaaattcacctgtctttgcctcccatgtgctgaggaacaaatgtatgctgccatgtctggctcacctgtcttttttttttttccattttttattaggtatttagctcatttacatttccaatgctataccaaaagtcccccatacccacccacccccactcccctacccacccattccccctttttggccctggcgttcccctgtactggggcatataaagtttgcaagtccaatgggcctctctttgcagtgatggccgactaggccattttttgatacatatgcagctagagacaagagctctgggatactgcttagttcatattgttgttccacctatagggttgcagttccctttagttccttgggtgctttctctagttcctccattgggggccctgtggtccattcaatagctgactgtgagcatccacttctgtgtttgctaggccctggcatagtctcacaagagacagctatatctgggtcctttcagcaaaatcttgctagtgtatgcaatggtgtcagcgtttggaagctgatcatgggatggatctctggatatggcaatcactagatgatccatcctttcgtcacacttccaaattttgtctctgtaactccttccatgggtgttttgtttcctattctaagaaggggcaaagtgtccacactttggtcttcgttctcttgagtttaatgtgtttagcaaattgtatgttatatattgggtatcctaagtttctgggctaatatccacttatcagtgagtacatattgtgagagttcctttgtgattgggttacctcactcaggatgatgccctccaggtccatccatttggctaggaatttcataaattcattctttttaatagctgagtagtactccattgtgtaaatgtaccacattttctgtatccattcctctgttgaggggcatctgggttctttccagcttctggctattataaataaggctgctatgaacatagtggagcatgtgtccttcttactggtaggggcatcttctggatatatgcccagaagaggtattgctggatcttcaggtagtactatgtccaattttctgaggaactgccagactgatttccagagtggttgtacaagcttgcaatcccaccaacaatggaggagtgttcctctttctccacatcctcgccagcatctgctgtcacctgaatttttcatcttagccattttgactggtgtgaggtggaatctcagggttgttttgatttgcatttccctgatgattaaggatgttgaacattttttcaggtgcttctctgccattcgctggctcacctgtctttgcaacgtgcttggaacttaccacataggcttagctggcagcgaaccacagggatcccctggtctccaccttcctagtcctgggtacagtgtataccactgtgcctggattgtttgagtgggttctgggaatcaaattcaggacttcatgcttctgtatcagtaatcactgtcctgaccaagccatttcttttctttgtgtatgtgtgtttcgggggggggggtggtgttcaagacagggtttctctgtatagccctgactgtcctggaactcactttgtagaccaggctggccttgaactcagaaatctgcctgcctctgcctcccaagtgcttggattaaaggcatgcaccaccactgcctggcagtgaagacagattttaatggtggactaagtagagcatattaagagaggggggtgggtctgcttcctagccatgggttacttccatggtcatgtcaacccagccaggtgtcaccttcctcctcctcctcctcctcctcctcctccactcctcaggttatggatggtttcctcggcagtgtcttatatgtagatctctgcttgtgtatggaacaagaagaccaatgctcctttctaatttctctagcctccgggaaaggacaaaatcactgaacctggctgtcagagaactcactgctcaggtaagcccaccctcctcccattttcttctgacctaattttgcatccagatgtttagcgggctttagtggaagcagacttttcggagctgtctgccaggctgcggcagcgaggcagagctgaagactggagccgcagactctgaggatgtctgttggtgttaggcccggttctttccatgttcccagaagtgagactcagactcaaaatatatctacagataccttggcctggggtagctaggctcttctctgactagatcacaacttaatataacctatttattctgagctacatcctgccatgtggctggttacttgtgctcaggtacaatgcgcccatctggtcacatcttcacgggtaaatctccctctcctggttgtattgcaaagttctttctgcctcctagatgtttcaccttctattctaccttttcctatagggctgtaggttttctttattgacaagtgatgcatccatacagtacacaagatattctctctacagagctctccacccatggccacttccaaatagatagttgaccttggattcagaaggggtttctgtccagaggctggggaaggatgttgccggaagtgctgagtgcctttttagagcagggtggaatggctcatagcttcaatctaaaccttccatagtcagaggcaggaggatcaagggttcaaagcccaccagagctacatagcaggatgctgtcacaaatgaatgaatgaacaaaaattttagggcggaagtaactggcttcccctgggagctcaagttcaggctgctcagaaatggaagtgggtcagtggatgaagggtttgttttgtttttccgttcttcggatcattttcctagcaccatttgtgacagcacacaccgggcaagcccagcatctcccactccgtggaggaaaggctgctctggccactctggttcagtgtgtgagggttcatcttacaccagggaaggcatggtggcaggagcgtggggcagctggtcacacggcatccacagacaggaagcagagtgtggaaactagtgctcagctctctctccccttactcagtccaggatcgcagctcatggaacggtgtggcccacagcgagggtaggtctcccaaatctttaaccccattgtgaagacatgttcagagatagtctcctgggtgattctacatcctgacaagttatcaatcaatattagctgggtgtggaggttcatgtctttagtctcagcactcgggaggcagagacaggcagatctctaagtttgaggccagcctggtctatagagtgagttccaggcagttcaaggccagttcatggtgaagccccatctcaacaaacacccctctgaatattaaccatcatattatttgtactttcatagcaagattttcttatgtgaagcagcaggttcccccacagtgctccccagtagtgctctcccacagtgctccccaggagtgctccccgagtggagtctgcatcctagctgttagataaagtgttcacagaatagagccctcccttgtggtcacacctgacagagctgtaggtccagacaccctgggagttgctttgaggtatgcagatggacacctggatctggacactccatcgctgggagctggaggttccacaacagtggaggatgtagaataatggtctctgagtagagcctctgacctgagaggaatatcagttgtctttcatgaccagagacaggaaggctgcagggttagggaagtggtcagtcagtaagctgtctgctgtacaaaccagtttcacccccagagcccatgtaaaaagaatgggcattgtggcacatactcataatgcctacactgggaaggcagaagcagaaggatccttggggctaactatcagctgggctagaggaaacattgagctctgggttcagtgagagactctgtcagaaaaacgtggagactaactaaggaagacacccaaagttaacctctggcttccacatacatgcattcaaacacacatacacagatacacacacacacacacacacacacacacacacacacatatatgcacatgtacacacatacaagcacacacacagacacacacatgcacacacagcacatatgtatacacacatatacacatagacacacacatatacatatataaatacacagatatacatattcacacatgcacacacagacacacatacatacacacatatacacacatacataaacatgcacacacagacacacatatacacacagacacatacatagacacacatgcacacacatacatacagtcatacatacacatgcacacatatatatatacatatgtatgcacatgcacacacagacacacacatatacatatacatattcacacatacacacacatacacacacatacatacacatacacatgaaaacacagacacacacacacacacacacacacacacacggccagagaagactttccggaaaacatgccgagtggcttgttttcaacttaactacgctgctgccgctgtgagctgttagtaaggctgtagtgtttgtcccttctcttatttattttttcttttctttttgtcttttccttcccttacttatttccaaagctgacagttttgttctggccacatgctcaagggtttgatggattcctgtcagcaaccaggaatgtttctaaaggacactagacttgggaaggcttgtgatcgtcccatgagctttctggctgctcacccaggggggcaatgaggtgtatttcttgtttcgcccagctcagccatgggctaggcggaccattaccagagacctcgaggacacttttcctgaatgctgggagggctctctgtctttgtgtgctgcaacttcaaagcactgagaggctggacagctctcaggcagggctagatggctgagagctgggtgccaatacggccaagttctggtggggatctcttctgagggacagatggccaactccatgctgtgtctttgcttgatggaaagggaacgagaacgggagggctctccaaggttgatgatgacgatgacgatgatgatgatgacgacgacgatgacgatgatgatggtgtatgtgcacctagaggacagcataggtgttaaattcaggaacgccatctacctcctttgggaggagggtctctcagaggcctggagcttaccaatgaggctacactggtagccagcaaaccctagggaccctttggtctctacttctccagctctgggattacaagtttgtgtgatgatgtgacacttatgtgggttctgtgggcccagctcagtctccatgcttacaaagcaagcacttttactgacagagccactaccccctggccccagcccttttccaagggcaccgatactttcacgaggttctacctttaggacctgttacttcccagagccccctcttggtcccatcatcatacgggggctgaagatttcagcacatgaaatttcgggggacccaagcattcataccatggctcaccccatctcttccatatttatatagtccacttataatgactgtcttagtcagggtttctattcccgaacaaaacatcatgagcaagaagcaagttggggaggaaaaggtttattcagcttacacttccacatggctgttcatcatcgaaggacgtcaggactggaactcaagcaggttaggaagcaggagctgatgcagaggccatggagggtgccgcttactggcttgcttcccttggcttgctcagcctgctttcttatagaacccaagactcccagcccagggatggcaccatccacaatgggccctccccacttgatcactaattgagaaaatgtcccacagctggatctcgtggaggcatttcctcaagggaggctcctttctctgtgataactccagcttgtgtcaagttgacacacaaaaccagccagcaccatgacttctcttttctgtcaaattaaggagccgtgccttagattctaaaatgtaaatgtaaaacttcctttctttaatgagatcctcagaggctaattttatttgtaaataagaaagacatgcagatttttttctgaggtgggtatgcgtattctattgcacgtatgtgagcatggtatgctgtgtgggaactgagacagagcctggaattttaaccttagccctgtcccctgaagactggcaaatgaccatgagggaaaaggcttggctcaacagttttctttctttagtgtactcgcacatgtgcactcaacccctgatactttcagagtgagttgggaccttccttaagctcattacatttcttgactctcatgggtcacccaaactaaaggtaattaaggaataaatgagatggaaatagttagaaataattgggatatctagagactggtaggtattatattatatccagcagagactgaacgagtgtgccgtttaatctggaaatttagaaaggcggcttcacgttaatagtgcagcttgaattatgggctttctcagcaaaggagaaactggcctaattatgattaagagacactgttaactaagatcagagatattagaggagacctgagaaccctggctggcatcacaaagttaaccggtgagtctttgaagttccagaggtgacagttgtgggtggataggatgggcggagcctgtcacgggcattgacccaaccgagacagggccagaggaaggtgccccagacacctttctttaagttcttagttctaacatacgggacaagtctcaggactccttaagatccttaagatgaataactctggccatgtggatgagcagctggagtaggcttcggtgcagggccggccaactctgctgtgagcttcagaagtgaggttggaaaatcctaggcatggaggggaaagtaatggcagagaagggcggagcattttaggacccaggagagaagggcagagcattttaggactgggtgatgtttgctttggttctgatagccgcttccatttgatcttggataaaccaaatgtttacaattaactgtatcctccacgtggaccgatagctttcagcttctgaaagggatttcatgtacttttattattttggcaataaatgacgatgcctggggtgaggggttacattctgtgtggtctttcccaaagtgctcactactctatcctcacagaaatgtgctgtgtgctttgggtgagctgaaaacataggtttatcgctaatctgagtatagataacagatacccatgagctcttagccttaaataaaagaagaaaaaaaaaaagatgatgatttaggctcggcataaaagataacctgacggaattgggggaatgagactgaggttgaagagttttgttgttttggaaactcaaaggaaaacaggtgatgtgtgtggagtccaggtatggggagacactcgataaccccaagtcttccaggaggtgacttgtctgactttcaatcagactctccagtgagtatctgcactcctggtttcttagagtcttctaataaggaaatttagcaggttcctctgacctcaatgtcaaaaatcttagttgactaactactgttgccaggggcagagtcctgttcatggactgcctactgtgtgccaggggcagagtccctgctcactgatggcctactgtgtgccaggggcagagtcctgatcatgggctgcctactgtatgccaggggcagagtcctgttcatggactgcctactgtgtgccaggggcagagtccctgctcactgatggcctactgtgtgccaggggcagagtcctgatcatgggctgcctactgtatgccaggggcagagtcctgttcatggactgcctactgtgtgccaggggcagagtcctgctcatgggcattgtagacagatgaggagacagctgtcagcaggagaggaagctgggggagactggggtgaaggtgagggacaggaatcagcaaggcccttttcccaggggcaccttggaacaacttgcctgaggttgtgaggctctgagggctcaggcaggagttgggaaatgccaaggcagaggtacagtgatagctttgtctgcttgtggctgctctgtgagctcacaccagcctgtgtgggtcagggctgtgctgaggccgcactagtggccacctttcttcctccttcctcatctctttcttccctctcttcccctaccctcctcccactcctcctcatctacccccatctctgcctctctcctccctccttacgcctctgctccctactctctgtattgttctctccctcctctctgctctcccctcgcctcttctcgtcttccttcttgctccctcttcctttcctgctttccttctaacccgttgctccccaaagcctcttctgctcactctgtcagtccactaagatactcagagacccagcccctcagccccactcccctctgtcttctggttggaagtagcaggcattgattctgccgtttctcctggcaggtgtgctcaggtgagaagctgtgcagctctctgaggaggagactcagtgacctcgacaccaggctgcctgccttgctggaagccaagatgctggccctatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggcccacaatggatctcgagaattcgatgggctgcaggaattgatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgagctacttagttggagttatggcctcacttgcagagagaggggggggggggagatggaatgcagttctgcctctgagttagggtttcgcctttctcggcccattcgtcttcccctgagaacaggtggacaggcttctcttcatggagcccaagggaagaggagagtacgactcacgaggcagaatcccctcaccctgtcatctgtcttctgtcttgagaagcccagggcagtctttacaatttctagaaaaagatattttatgtctgtgtgtctgcgtgtgggtgtgcgcagccaagtgagaagacagacagacagacgcacatgccttaagatggctccttggctacttgaggaaaccgggaggctgtgttggatgggctctgtttcaagctcctccctcctgtccagagtctgacctatgacatcatggcctgctgatgtcctagaagctcttgtttgaccccttccagtctgtgccatctggccattgccctagtgtctgggaaaagccggcttgggcagtcctagtttttctttttaaaactctctctctctctctctctctctctctctctctctctctctctctctgtgtgtgtgtgtgtgtgtgtgtgtgtgtgtgtgtatacacgtgcttctgtacgcgtgcgtctgtctgtctgtgcctgtgcacatgagtatgggtttccaaagaagccaggggtgtcatgtcccctgaagctagagtcacctgtagcctcatgccatctggtgtggatgctgggagttgaactcaggtctttgtaagaagagcctgcgttcttagcggctgaggcttctgcccaagcccgcagccgaggatctttcaagcgaaggatcaagttcagcatagctttagggtgagaaacaaggtcgaggcgcttcacgtcccccgtgtgcgtccttagttgagggatgtctgtggtttgatgagaagagaggaaggaagtacacttccttgcccctcccgtgggctcttggactggagtggtcactctctgagttgaggtccttcaggaagacccccaggcagttatcactgcacaccattcctggatgatcaagaagactgaggagtggtcaataccgtgtttcagagatgccaaagagaatgggagagccagccccgggtcaacacacactgagaaatgcggcggtgagactgggtaacctctttatttcaaatgtattcctacttcgttttgtcactttaagaccagtctcactcagtagcccaggctaccctagaacttaccattctcctaacttaaccaaggttgcaggctggtgacgtcacgcctctcccagataaattctttccactgtcgtcttagcaaacctatgaaggtctcctctatccagccgcctcactgaagagcaggcagctcggcagagcctgtggatcactctaccctgcctcctcacacacaacccccaaggaaaggctggagacagctcacctgtgagactccttcagggttggcactgacttcccgagtctttgtcagcaaagcctacctagggttatgcaaagtgagtcttgtctctgagcttggctgtgaggtcgaatgtgtgcaaaataatatcgaggcagggtagcagtttgtgatggctccagctgtgatattatctgcccctgagaggagtcacccaagttctctctctgttaaatattcatttctcgaaacacacatgtctccaagtgcttggatctaggaaaggtctgggtttagtgaatggcgtctgtggtattcctcatcttaatggtcactcctcagagggctgggacactgccaggttccctcttgtgttgccttcatgggatcagagaacatgggtttcatcaacagaatggtgtacaccgagtgttcatcgtgtgcccatggccggtgacgatctgcagcatttgtcagtctgaaatcagcctctttcaaggctgtaagcagcacagtatttaaggggaggggttttcgatagaagcttgggctgcaaacctgccccgtggcttattcctgtgtgaccttctgcaagatatggaattctctagatttcagctgtgaagtcaggataacaatgcgtatttcagtgaacataagcaccgtgtgttacacaccacggagaaagaaacgttactgtcagctgaacaatgaggctgtgccttccgctcagagtcctcattgtgcgcactgaaagagttcgttccttcacacacatttcagcttgaagaggatcacatgccgctcctttgaaaggagaatacatcggctaaagttttacccagatgccttcatcttcatcttcaaaggcttccgaggcatctgtggatgtcaagctccgaggtggggtggatgtttccactcagcttccatcctccaagtcgggggagctgtgggctgcaacgttttccttcagtcgatgacttctgtagggttttctactaggctggtgccatgcttcaagttcgtttgtttgtgtgtttgtggtgtgtgttggtgtgtatgcacatgttcctgtgtgtgcatgtgggcgctggagatcaatctcagatgttgctcagaagccttccatattattgttgttattattattatctatttatttactttggaggcaagctctttcagtaggtcctattgccccacagctgggcaagcctgaccagccaggctcagggattctcctgactctatctctgcagtgctgggataacaaaggctccaccatacccagccttcacctcgatgctagggcttgaattcagctcctcatgcttgtcccgcaaatgcttttcctggctgagccatcccccagcctctgaactgactgccgtgggcctttctgccaggctgatcaccaagcttcgtgttcgtatcctgtgacaatttcacatgtgcagtttattttgcttcgtgcattccctgtctctcccttccactctgtcttgacccttctcaacaagtctgccccactttcatgtctttcttttttataaccggcccactgagtttaattagagttgctggcaagatctggggctactggtgtatcagtgacttaaccagtggtggctcgcttgtaaaaatggcttcccttctccagcatcagttaacttctactggcttctcagctgggggtagctcctgggcttccccactgtccgtgctagaatgcagactggctggatctaggatgggtcttatgcaggcagctacaggtgctatgggttcctgagctcacagacatatcatgtcggaagacagcatttcgcagcccctcctgctcctcatgccctttctgccttcacttcccccgaggtccttgagtctctgggtggtggtgatagagatgaccggttccacatgcatcgagttttaatgcaagttctttcccaatctcacggaagcctgaagtcaactggccttgcggtgggttgagccttggaacatcaggtgtggccggtgaccacactcagccacagagaacaacctcaccttcttctggatgctggctgacagatgacacctttatctcagaggtgttgctgtcacaagctgaagcatccccgaggggacggtgcagggactaagcttccctccaagcagacaatgggaagaagagaagctggtcccgttctctttcttcttagggaagcttttctattagggtctatcagatgtacataataatgtagcattatgacatgttcatacatgaataccatgcatttgatcacactcatcccccgggtcctctcatgtccacatccgtgctgatcaacctctttccaaagaaacacctgctattttcatttctgttatcccatcatcatcatcacctccactaccaccaccaccaccatcaccatcaccatcatttccagtatatgtgtgtgtgttgcctacatgtatgtttgtgaaccgcatgtgtgcctgtagaggcacaaagagagtgtccaatcccctggaactggagttacaaatggctgtgagccaccatgtgggtgctgggacttgaaccttggtcctctgtaagaacagaaagggcttatccattgagcagtgtttccagtgcctgccctccctccctccctccctccctccctccctccctccctccctccctccctccctccctccctccctccctcccttcctccctccctcccttggtgaaccagtcagtgagtttccctaggattgctctctgaggctggagtaaggtcactaggagctgccaaatgtaagtgcttgggatgatgtcttcagtcctctggggacacccctaaatggagacttgtgggaccaagtgtggagtgggtttaactttagtacaagctggccgaagtgttctctgtggggttcaccgctttgcgtttcgagggttcagggagttgtacctgcttgccagccttggttcagcctcagtttagctgcttggtgccatttgaatgggttgatgtgcctctgccacttctgggcttttgtttacactcccagtgactgatattgtctaacacattttcaaatgtgtgtcgtccatctgcatacggccctttgtaaaaggcctgggagcctttgttcctgtttaatgctgttatttctgttaattgttgagttgtaaaattgtatcctttgtcagttgtctgtaggagatgatctgtcccagcttgatttttcttccctctccccctcccctctctaatctgtctccctcctcctctctcccttcccctcccccttgattccccttcccctctcccctcttcacactctccccctctcccctccctctgcccctctcctctctcccttcccctcccccttgattccccttcccctctcccctcttcacactctccccctctcccctccctctgcccctctcctctctcccttcccctcccccttcattccccttcccctctcccctcttcacactctccccctctccccctctcccctccctctgcccctctcctctctcccttccccttccccttcattccccttcccctctcccctcttcagcctctcaccctctcccctccctctgtccctctcctctcctctctcccatctctctcctcttctctcctccctcccctcatccctttctcccttcccctctctccctcccccttacctacccttcccctttattctcattttgtctttttaaaatttctttacttttgacaatcttatgtagtctaggttgactgaaaacttgctgtgtagacaaaactggccttgaactcctgatcttcctgcctctagcttccaagagctgggattgcaggtgcctggtgtccagcttctctgcctatcctcttttgatgagcagaaagaagcttgtcaccggatgaagctcattcatcaggggttttctttgattgtcctgctctgtcttccgcagggctcagcctgttccagagccagttcactttgcttccttatcctgcttgtagcttcctagttctaactttgtatcagccctgaggtgatttgtgtgtggtccaaagtgtagctcaggcttcccactgtgccttcatagataaccagttgctttggcaacctcccttgcccattgctttgcactccgttagctcttggaggacatgcccactttctggacttctgttgcagtccattggccagcatgcctatctgctttcagatgactctgtcttgatttatggaagcaattgctggggggtcttaaatgcagactgtttgacggctctggcttgtctaggttttcacattttcttttacatttttaggatgtgcccattcacttctaccgaaagacctcataaaattttgataggaattgtattaaatctatgcatcaatctggaacaaattgaaaccctaggagtaccaggttttccagtggacatttatagtttatctccacaattatttaagccttatttcctctcagtgtgttttcgaaaacagtttccagttctgcctaaggtcctttaaactcacccctacatactttttagtcctaagatatgttgtaaactatatttcagagaaattgtctcatcgtggcctgctgctgctgtttagacacacgaccgattctcatacctgacctcgctactctttgctctaggtatattttattcagggttttatggcttttctttcttacacccccagccacctgtggttcccgaggccacctgcaattcaggtgttattacgatcgtatttcacaacacgtttatgccaaacttgctcatttattctcctgttgttttagttagagtctctgtagctgagataagaacactggccaaaagtgactcagggaggaaaggtcacagaccattactgaagggagtcagtcacagcaggagctccagcaggaacatggaggcaggaatcgaagcagggaccacagaggaatactgctcactgccttgctttcagggccttgctgtgttattggcccggtacgacagacctgtccaggcatggcaccaccaggagtgagctgggtcgtcctacatcaattaacaaagaacaaaattcctgaaggcgtacctacaggctgatctgatggagttaatgtctcaattgaggtccccccttctcagctatgttaggattagccatcaagcgctggagggatggctcagcggttaagagcacttctgaaggtctggagttcaaattccaacaaccacatggtgactcacaaccacccgtcatgagatatgacacccacttctggtgtgtctgaaaacagctacagtgtgcttacatataataaataaatccttgggccagagagagcggggctagagtgagcagaggttctgagtttaattcccagcaactacatgatggctcacaaccatctgtacagctacagtatactcatatacttaaaataaataaataaatctttaaaaaaagattagctatcaaatatgtttcaaattttgataagggggagggtttaatttatctgcagtctaactaccaggccagtgcctttccaccgtgcttgctgtgtctgtccttggacatttgggtattgggtgcagtgatacctgggactttctctggacatctctgtattattctcagaggcatcctttccaggtgtacccaacctcttggtgatgcagcaggataccgttgtctccagtgttcacacacgagaatctcacaattcatttacagaaaataccagcatgtttggggaggcaccgaagagactctcccgccccaccggaggtggtctgataggcgagggctgctgagggagggagagtcattctcctctggggacatggctgctggcaggttgctcatggcctgatggatggcctccacacccatgcacatacggacggcactaatcaggggttattaataataaaaagacatgaagttgggaggaagacacggtgtaggtactagagggaggtccacagatatcgcggtggacacatagcgatcaatacgcattatataaatgcatggaactttcaaagaatgcaaatattattaaaaagctcacacacgaaaccttccctcctctctcccccactgtgttgcaggaagctgcttctccacagccaaggagctcacggaggagatttgggccttgtcgtcagagcgggaagggctagagatgttcctgggcaggctgttggcactcagctccaggaacagcagaaggctgggcatcgtcaaagaggatcacctcaggtgcaggcaggacctggcactccaggacgccgcccacagtgagtagcccccagccaaagcctcagggcaatgtagtgctgatattattggatcctgtggctagatcaccttgctgtgacagtggcagagagcacccagacagttcatgggggacattgttggcatgagccttgggtaacggggtagggtcacctgttagcttttactttgcctttctctgactttttctgctctgcatttgttggttcatgccatgggctgtgtagcttgcacccccgggcacacatcctgagctagaggttttcccttggttcagtcttccaggatgcaggccacaggccgacaacgggggactgagaagggaccagggggtgactcttctgtcggggtgtcctctgagggttgattctttcctcaggagaaggacacgggtgacattggcactggcctacagaagggccaaaatgctatttgccctgtgttccacttaacagacaccacctgcatattccacacatccacgtcagggaagaccagtgttcaaagtccaaacaaggaaccacacctccagctgtcagggctaggcctgccactctgggaggagcagcccaggttttagagttctcctgaacggtgtgagctgttgttccctccctgtttgaatcagatggccagtgatcatggtttctcttgctaatgaagccccatctggctcaacagctcagagcctagcattatttcccttaactctttaaattttccttatgaagtatttaattttttattttaagttatgtatgggggtggggttgtgcatgttagtgtagtacctgaggaagccagaagagggtgccagatgaccctggagccggtgatacaattaccatgtgggagttagaaacaggactcatggttctctgtaagagcagctccagttttaaccacagagccccactcactcccgggcccctcctccctcttttagtataaaatagagtttgtttgggggcatggggagggggggtgagcagggaatagaggctgagaaagaggagaaagaggggacagaggacagggacacagggtggggtgggatagggaggagaattaggggctggccagggacacttggagagcgggacgagggggaagggaagagagggagaaagagatcagagagagcaaagggagtcagagagaggaaagggtcagagagtctctctcttttttgcgtgttatttttgcgttggtcacgcagaggtcagagggcaaccttcaggagtcacttctcagtccacctttactcggctcacggagcttgcatgggtttctgcccactgaactaccttctctttctctttaagtttttcaagacagggtttctctgtagccctggctgtcctggaattcactctgtagaccaagctggccttgaactcagagatctgcctgcctctgcctcccaagtgctgggattaaaggcatgggccaccactgcccagccatccctctctctcttacaggccaaatcttttatttattcatttgtttgttttgccacatgcagcccaaggctgtgtgtggagcacgcacagtgaggggggagctccagggtcccaggccctgcaggctggctcatttcgtcatgactgaccggagcacggtagagctccagcagacttttgtagtcgattttttttaagtcttatgaataagaaaaaccaaaatgtttcacagtgtaagcactcagtttttacacaaaacgggggaatggattctatggaagaatcaaggagatgtaaacagatttgagcaaatctaacccaacgatgttttcatttgaatggtgcgtgagcccttaaagccattaatatctttatataaaaaaaaagcgctgtgcagtatttgtgaatctcatcattgttacttcactaaatactttcaagatgatgaataaaacataaacaagcggaacgggagattgttacaggagacgactgaactgcagatggctcgcgctggggaatctttgtgctccgttcatgatagtattatgctgggtcagccaccctctccctgtgggtttgctcgcctgtgtctgaggctgaggaggtgtggctttagatcactcaaatgaaccatccaagagttggtaagaagaaataaaaaatgtatttcagggaccaggggatagtttaatggggaagagcaagcattcttgcagaggacccatgcttgctcctcagttctcatggagtgacacccccttctgacctctacaggtaatgcactcacatgtacatgcatacagaggcatatgtgcatccacgtaattaaaactaaaagaaaacaaattcctgtgtgaattaaagcttccacggattggaatgtaataaaaccgagtcatagtctgggttcagctgcttttggatcttgggaaacagcaactcagaaggaacgctgtgtccaaggtcaaatgtagctcaagcgagcaggaaaggcaggtctgggtctgcgtgggcacagtgctccataatcttttcagtcttaagccaaatacctgtttcttaggttttaaaatggaaaaacgaaaacaaaaagaccaacaatcatgtgagcgatttgggggagagggttatacacaccctgaatgcagtgcccccagaagctagaagagggcattggatcccctgggactgaagttccaggcactgtgagccaccaggtatgggtactgagaacagaactcaggccttttgtaaaagcagtacatgtatttgattgctgaaccatctatgcagcctatagataaaatatcttaaggggaaactaggaaacgagcaacatctgagtttaggtttgcacgcccacgtgagtgggggtcagggtcagaggtcaaccttggttgccgtttctcaggcaccctcaactttgttttaagatcttccctaacctgggatttacctatttggttagactggttgaccagaagcctcggggatccttctgtctctgcctccactgggattacaagtgtctcatctcacccggcttcttatgtgggctctggggattgaactcgggcccttgtgcttaccactaagctatatctgtaccccaatataaaacatttttaaaatgtgatttctaacagtggctggagaaagccgtgtcgggatggacaagcagctcttaggctcagggatgtgagccctcttgttcttcctagcttctttattaggtgacgtcagatcctgccaggacattgctgtccattcaccgctccctctagggttctgcatgtgacttcctattatcattgttgttctgaacttgaccttggtacatgaggacagtcagctgcaaaggtaacaggagctgcccttaccctggacagctgagtacctcttgaagttctaggattctctttctgtagaagaaggaaaccttttcttgtttaaggaccaattagcagtattgttcatacgaagggctcttactggtaggaagcaacattgacagtcaccagagatggaccagcctgtctcactgaggagatgtccttgtgccttctgtccctggctagcttgtacattcactgagagctgaaatgctgcctgagtcttattggggtatcccaggatccagcatggtggccttgggccaccagcccgtgttcattttggggggcaggagagtgatgtaaagtgttttttcttccttggtttggactgtgggtacagacctcagatctccttgagaatggtgacaagggggactttcctgaagatgatagagtgatcccctttggatagttattggtttgctttgtctttgaggcaaggtctcctgtagttaaggctggtcttgaaccccatgtatagttgagggtgatcttgaattcctgatctttctgcatccacctccttagggatcatattagggtcctgcagttcctaatacctagtttgtgctgtactagaattgaccattgggttttgtgtacagtggacaaacactctgccagctgagccgcctcagccctgagttggatgttcagactggggtgatgggaaaggcgcagaaacacaacagtctcaagggcaaagggcaacactcatggctgctgcctttaactccataggcttctgttatggtgacactcctgtgaggaggagaggagaaaagactttagtttggagacttcaagtcacacacttgaattctgaggaatgcttagtagaatggagcaaaatgaaaaagtggcctccgtgcccctgaaccagcagcctttatttaatgtgcattgaattttttttaaaactgaggtagaaaaaaaaaatcagtaaccaattttaggttttgttttgttttctagaaaaaaaaaaaaagaaccttaagatttatacagctgtttctccatctcctgtgtatgacttgataatctgtattgtactttattaacactgttgatttcatactagaactcagatataaaggaaccaatgtggagggaagcgatttcatcacagagtaatcaggctagaatttaaataggacatcattagcatggtaatgcgtttctctagttcagcaggctgcctgagtagatctgggtttacagctggtgagccttcagggctccctgttgggaatcaagccatttccagggagaaggccaaagggtaggatttcatatttctgggaaatatgaactaaatggagatcttgtccatatggacatctgatcaaccccaaaccatcagtagacaacagtcaagtcactgctcgtatgtcatccctcctgatttccatgtccacagtccttgaaaggggttacttaagcaggaatctgtgaaccaacagcccagggatcacagagctagacagcagggagccaggaacacagaggagccagagaccatggagccaggaatacaaaagcaagtaaggatagaaaggtgaggactctcaaagcctacagagtcaaaatgaggcaagactgaaagtaattgagatgatcaaagtctgtatgatggtcagtcttcattttcaagtcactgagtcagcttccagcctccacatcctatgactccgtgtttcttactgccacgttccctaattgcatgttccctggctccatgtccccatgctctgcattccctggttctgtgtaccctcacttcatattttctgcttccgtgctcatcagttacccccatgggaatgcatgatggtttaggtgtatgccacaccaaacttcagtgcacagctctgtgacagccacttcattttaaggaggacctcagctctgtgctgtagggtatcccttatgcatctgctagattttcagtttggccgattgctcttgacaaccacatttcagatggcctgggcttggggaacctgcagaggtacgtggtcccattcacacacacacacacacacacacacacacacacacacacacacacacactgctgagaaggttctagtggaagtttctcagagaacagctcactgctgagtttgggttgagttagtatgtgcaggaattgtagcaaaggttactatgtattggatgtgcaagaactgtaccaaaagttactatgtataggaacagtagtaatggttactaggtataggaactgtagcaaaggtaactatgtatgtatgggatgcttggggggggggggactatagcaaaggttactatgtataagaactatagcaaaggttactatgtataggaactgtagcaaaggttactatgtataggaactgtagcaaaggttactatgtataggaactgtagcaatggtcactatgtataggaactgtagcaatggttactatgcataggaactgtagcaatggttactatgtataggaactgtagcaaaggttactatgtataggaactgtagcaaaggttactatgtataggaactgtagcaatggtcactatgcataggaactgtagcagtggttactatgcataggaactgtagcaaaggttgctatgtataggaactgtagcaatggttactatgtataggaactgtagaaaaggttgctacgtataggaactgtagcaaaggttactatgtataggaactgtagcaatggttactatgcataggaactgtagcaaaggttactatgcataggaactgtagcaatggttactatgcataggaactgtagcaaaggttgctatgcataggaactgtagcaatggttactatgtataggaactgtagcaatggttactatgtatagtaactgtagcaatggttactatgcatagtaactgtagcaatggttactatgtataggaattgtagcagtggttactatgtataggaactgtagcaaaggttactatgtataggaactgtagcaaaggttactatgtataggaactgtagcaaaggttactatgtataggaactgtagcaaaggttactatgtataggaactgtagcaagcactacagatcttgccacagtccttggtggctgttgactcaaaattccttttcccaactctgagattaatgtgtccctggtttcagggtcaatgatgtcagtcagccaaatagtttcagaattgtgtttcatatttacaacacgatttggaaatagttgtcactctgatggtttttagaatctagatggagcagccctgttctgtgagtggtagtgtctcgttcaacttctccaaagtttcaaagtgacctccccacagacctcatcctccattactgcattctcattctccattcactcactgcattcccagtttgtatttcttgggtctcctgcttatgcacagaggagttagatctagtgtggatttcactgttcttagagaaggaatggtttctgatatgttggcatccctttttggtagtgtttcctctttcaccatcccccacaagaagcaggcattttgctagtgctgtatacatgtaattactacatccctggaccactaatgctgccggctctggtgggagggctgtttcatgcatggggaaactgaggcaaggttttatcctgtggcctcacagagaggcctgagagttccttgcttctaaggtttcaccttgacaaggggtttgttctgtttgattagatccctaatatgagggcgtatccctcaatctcactatcacacctgaatacaggcaggatgatgtggaagcccagtgatggcccgaagcccatggaagagcctggcatgtgatgagtaggttgcacaggaaactgctgaagttagaccttaatgaaccaactcacttcttcactcagctcgagatacagcttttctcatgcttactgtgtcactgaagcccaggggaggagagacgtgtgcatctcctgcctggcaaaaggctgctcttgcttttcccagtgtcatctcccattctcttcactagaaccactaaagcaacactatgtggccatgggggtgttttccttccaatactgtggggcgatctcctggtctgctggcaaatgtagccctgggagctgaaggaagggtgctccagaaaatggtagatggatattctccatgtctttgaaggacaggacagtgaaactgtcatgggcttttccatgggcaacataggttttcagtggcttcccacctgtgcccagaacaaagggtattcagtgtgcagttgaagagcccttcccagtctctgtgaacacaggcaggagatgctgctggaccatgctgtaggaagatttcctcttcccattttttctctcttcagccatcacctcctctgtggcttcatcacctccgctgtggctccatcacctcctctgtggcgctgtcacctcttcatggttcttccctccttgtctggagtattttgcacccttacctcccattgcccactctgtgcatccttccagctccgtcctggtcacttctccattaccaatactgcactacatccgccgtggcacctttcactgcccccaggagtctgtgcaggaccaaccgtgcccatgccccctgtcttccctgcctacatttgcagttcatgtcttcctggtttctggcctgattctggaagggcaatcattactcctgcattaatgctccatgactattcattctgttctgccatgtttaatgggatacttaatggcatccatgaagaagcccacaagactataaataacactctctttggtagtaaaagggttttatacaatataaaatttagagcatatttttttttcatcaagcgctctggcattaatattcatagtgagccccaaagtagaagctattctatcttagcataatgagtttacggtacatttcaaagtcagttgcaattgtgctctctgccatgacaatcacaaaggttctcttgtcttttcaggctcgattatagctgtaaaccagcttgtatctgtcaccatgaaatgtactagggcatctgtgcacaaataaaagggggagacagacttaaacccccagagtatagttgttcagaatgaccaaacctgaacatggcttggatggtacatgggcaaagaaactgctaaactaaattcttctgtctttttattgattagattgtttgaggcccataaagttagatataatgaggtccctagacattggatagagaaatttaacattaagacacccgtagatttctggcctctctctatttttccgtgtcacttagaaaaacagttatctcctattttgacatgaggaaatgtattgtttttaattccccatggggagctttgatgtgaggatggggaggagacggttttggaaaagttgattcacttttggagcctgtagagatgcggaggggctgacatcggggtggtgggcttacacgaggcgcttctagagggcacatttctctcctaagcaggtaccttccaggagatgaaagaggccagtgggggtgtgcatgtgcgggcacagcagcccatgtagggaagcctgggtctcagtctgatttggcaatgcatgtgattgccatgctgagtctcagtagccgagttcccctccgcaatgcctacttttcccattgctgttactgaatgtctgataagaaaatactgaaggctatcctggcgaggtgacgttccaggtctcctgagacttattacccacagcaacagttagtccaacaacttgttccagtgtcccagccttcagcatcctttacttctgctcctgcctgggaaaaagactgcctttcttacaacaaaaacagtcccgctggccctccgccccggacctttggcattctggatgagttctgttctctcttctggccaagtgtaactcgtgtacaataaatcctcttgctgtcagctgaagaaaaaaaaataagaaaaaagaaaatactgaagggcttttggaagatggcaacgacatccaggcccagtctgccagtctccaggaaataaactggacaaagtcaaataacagctatcggaagaggtgggcaatgcccgattatcagtagcttacacagcagagttagggagcttccatcacaaagcaggagcgagccatcctggtccctagtccatcctgggcgtgttctgtgagagagggagtaaaggggacagcttttctgcctctgagaatttgcaaaaggagactactgtattggaattgtttcaaatatagttaaacgggggggggggggagttcttctatttaaagggaacacagaggggcagtggataaccttagacttgtctgatatgctttccttctggaatgacctgagacttgggctcttggctctccacgtgtcagccttcaaaaacacaggacccacagatagcccagtgcttcagggttgggttttttgaggccaccactgtcacctcgtgtctcagacaccctctcagggaaactggctttggcagcacatggaggtcagagccagggcagcactgtttgccaggaatgtatgtctgacgggatctgcagcccctgacttctagagaggccccacaacccacaggaccagcacccacccagcctccatgagcagtgcctcacccccagtcccttctccacatcagtgactgtcgctgtaaatgtcctcacttgtcagtgacattggattacaagagtcttggagactctttttaatcagtctttttccaaggcaatctgaggcccccaggaatccatctgcagatgggttctcatctgcctcagatttactgcagtgctctggggctacgagtcacttctcagtggtgtgtccccatcaacatggaacacaaaggtgaccatggccatacccaccattttgtgtcattgtgaccaaagagaaagtaaacataaggggaaaggatttatttgattcatgattttggaaggttctgtataactttaaaattttcaaatcttatttttggttgtgagcctagcctttaacggctgagccatctctccagcccatcaaatcttatttttaatgatggttcttaaaacactttaacctctcttgtagcccaccacccaccagtagtagtaggaaagaaattatacaggggaagtagacctgtttagaaatgattctttggagcaaatcccatctgcattgtcaagaagtccacagttcagttcacaggtcagcagtggcagctcgatccactcacaaacacttcttagatacgccagcagtccagttgagtggactcaggacagcaaacaggaatcagcagtggtggcatgacctagcagagacagccaggcctcagcctcagctccagtcggcaggagggaccaaggccaagaggaacaccaggaaaagttctcggctgtgcctctctcagtgaagtgaagaccagagaagacttgagaccaacaaatgttgcacagctagctctataagcaagccaagctcagcctctgtcactggccatcgagtcctttttaaactccctccaaatatcacatgtcctccacgggtcttgcctcagtgctcagcacgtgagtctgtctcagctgacatcactctgcccgtcagctcaagtcctcggaagcagcaagaaacttcagcacaccaacagaaggtttttttgtttgcttgtttgtttgtttggtgtgtttctctctatggagttctgacaaatgcagctcaactacactttgtaaggaggaccaatacatgtgtgtcattagccaagaatccttcatcacgtgtcctttcacgtgcttgctttagtagaacattctttttcttgtgtctgcttcagggaagcatcccttcatgtgtttgccccagcagaacaccatccgacagttaccagtcatggttgggagggggaggcagagagcagagcagctcacaggatggctgcctcgaatcacgaggggacatagaaagtgggtggggaaccaaagtacctaaggacacactccagagatccactcccaccattacaaaggctgaaagcaaaagtctaggatattatttttggagccaattggagtagaggaacctgagacctttgatgggagagaggacaaggggggagcttgtcctccgagctcaaggtcaacccaggacatttcacagggaggattttacaggagtaaaaaccagtggtttggacacttcccagctagaacatgataggatgtgacccccaaagctcaatagacaaatattccaaatgatcttttcttttctctcttcctttctccttcagggtattgaacttaggtcatttgtcttaaacatctttatcttcatcaccaggagattgtttgatgggggtgtgtcctggggaatgacacttgtttttcccttcctcatcggattcatccgcaaatggagatgaggctccaatttgtggctcaatttgaaagatgaaaaatttagacctgtctgtcctcactgaacgtttttcgcgtgtgcgttcagtaaagattccggatgctatgtcagctcttagtttgctgaaatgtcccaaccaaatcaccctagtaccgtgtgaatccggatttgaaactgtcattagtgggtgaggactctgcctcgtgggctgcctgctcccatggtgctctgctctctgcacccctcccatggtgctctggtctctggctgccccgcccccatggtgctctgctctgtagttgccccacccccatggtgctctgctctctggcttttgcattcaaagctttgtttgtggcttggctccatacttgctgaggcttcagggaatatagactcgcacaaatatttatcaagctgagtgaccaactgtgggatgggagatgaactgctccatgtatatggaaatctctgagctctgggctggattgtgtgttcatctgacccagaacattgaaaccagatagccctatgatgtgcagaataaggcagggcaggaactccctgcggtggagaagaggagaggaacaccagagtgactcactctctctttcacccaccctgcatctctgtgttgctggaatgtcaatcatgatgcggtgggatgaccactatttgaaatctacagttgtcaatcttgcgttgcatttactaagatgctcttctggctccagcagcccagcagagtatactgagttcatgctgtcccacatccctgctcaagatcatgcttgaaggttggaaacctgagaaacccataagtctttgctgcagtggctggagaatggggactttcctctcatattcttgggttttgtgtcagacttcccatctccctgacaaatacctgagagaaacaagttcaaagggcgagagtttattttggcttagggtttcagaaacgtcagactgcgcacaatctggttcctttgtctctgggcctgtggggaggcaaaatattgtggtggaaggatatgaagcataggggtccacattgtgtatctgggaagcagagaaaatgagcaggagccagagccacaccccctagtgacggacttcctccacaggccctgccttccacactcctgcacagcccctccatctgccaagcatctatttagatttccaaaccgtgggtggattaaaccaccgagttctcatgctctggaaaggccctcatagccagggtgcagtgtactaaccgctctaggcatctctctacatgatggcgttgacaataagattaaataagacagagggtcggagactcctgtgtctccatgggtgaggaacaaacttcaagcacatccttgccttcaaaatgaaagcagagcccctgacaccaaatccatctgtcccggcacattcccacttttaatttgttttaaaattcttgcatttgatttcctagacttctgtcatttatggaactcaaagctatgtggacagctaccaccatctgggtaccatgatggacgggttttgtctgagtatcactggcacgtcatttgctgctggatcttgctttccttacagcggaggtatggcctgtggtggatatcccatgagcacctacgagttgtttgggtgatgctgagaatttaaataatgtaggtggtaaatgagctgcttttcatcgctacaacaaaatattcgactagctgggaaggagaggatgtcttttgttcccggtttcagagggttcagcccacgtcagcagacaccattgcttctgtgcctgtgcaccgtggcatcacagcaaaggggcacggtggagcaggactgctcaccccatggtgaatggaaccagagactgttgcaagctctgtcttctttgccagtcctttatccccacgagaccagatgtggctgtgtgtgactgttgacagtcctcctggaaggagggagtagggagagttattggaccttgcctgagtatctgacctctcccaggccgagtgtaggggtgattcctcactgttagaagtagatgggcctgtgcggcaggtgggtgaaaggcttttgagaaatgctcttgctggacttttcatagttctgggtctcacagcagaagtcttctgaaagagtctggcatcccagagaattttctagtacttgaaaagaattaaagactttgacagccaaagacacatttggcatggcaccagagcaagagaagactgtgtcagagaagagggacgcttccagaaactctccaaagttgagctgacaggagggggaaggtccttcaggaagactttaagaaaacaaaggttcagtttctgtctctcattgcttcgtgccgctataattttgcccaacgaggacactgtactcatttagacttatcgctgctacatatccccacccctctcatcccttgtctgtgtttctttgctctgaaggaggctgtctcctacctgcaatccctcgctgctttgctctttgacctgggttctgtctttccaaaccaaggcagctgcttctatctggagccaggggcgtctacacccaagagtccagcaggcaatgcctgaccacgtatcagatgcttcatgggccacacatctgaaaagaattccgagccgcatccattgcctttgcctttgagtgaaatcgtttctaaagaaagtcttcagcttaactttcttctctttctacaaatgttgccataggcaagcccaaagaacagcgccttagcaggtcctccctgctcaaagaacagctccccagtaggtcctctctggcttcctcagagcctccagggacccagcgaacatttggacagacatgttgcttttggaaatatatcagcccagtagacagatttgctaagtcatggctgtaatagtcttaatgtgatttgtgacataggatggtgatgttcctgtcactcaccaaagcccttccccaacagaaacgctctgagtactatataaatatcatttagcccgtgtcccacattagtggtccattgtggggaaactgaggcctatgagtgagacacgcccgtgcgcatactgatggttgattgctagcatgtgccggtttcccatctgcagtgggcgtgtgggtagtgcatgatagaggttgtcagtctacttagtgaggggtatataagacaaacagccacttgattagtgggtagtaaaagacaaatacatcattagtgatgaaagagtgtcccgtcatcacagtacgtacaaagttcataccgaagtcccctcggggtctacaggctcattgccaaaaaccaccagactaataagaattccattatgattgacacggtttataaaactttttaatgaaaatatagtttcatgtcaaactcagataaattcggaccctgatttttaataagatggtagggtctcttgcagatcctttggggagattgaatcataaagctgtcaggaggcagatcgctgtgtctttccagcgctggggtctttgccattccatctgttctttgtaatgagtcttttggtccccatcttcagaagggtaagaggactaagcagccagactctgtctgctgccccgagatgtagtgtagatggcatgagtcctgctgtcctgcccggcaactgtcttgggggcagcgggtgagaaggcttgaaatcaatggctcagatgggggagcaggcgagaacacagtgaacttagttattaattcttggggagcaccctttatatcctccactgcatcccactggtcccaagcaagcatgtcttccaagtggcagttcccgattggctgtcattgtctgcatcaccaatctttaggctctcagacagtcttcaattaggtcctcaggcagaagttgcctccatggctgcatggccagattagaatgctgagtccatgggagacataagaggatcttgagttccaggctagcgtgagctatataatgagaccctgtatcaaaactctctctctctctctctctctctctctctctctctctctctctctgtctcatagggagaggtgctgatactaaactctggttccctaattgattcttgacttagttgataaaggaggtcgggagccaattgctgggtggaagggacaggtccttcctggtcccaggaggaaaaagagatgcagggaaagagaaggcggtttctggccaggctttggaaggagacgcacaaaagaatcatgcaagatcttggggaaggtagaaccaggtggcttccaccaccagctcatagcaaaggaggttgaaggaagctagtcaataagtttagggcaggagattctgtgctcagcaattgtgttaacaggcaaattctaaagtaataaagctgtctgagtgtttttcatccatggagcaaaggtggagggagagagagagagagagagagagagagagagagagagagagagagaccatgtggatgatggaggctaggtggagctaacctgggaggaacaagaaaaaaaaaacaaaaacaaaaacggagaggggccagagcaagggtggcagccccagggctgaggacacgggcaaaggcaatagtgtatttttttaaattacatgctatacacattaggctgggagatatctcagtgggcaaagggcttattgcataaacatggttagaacatggtatctggtctctccttctgcacccgtggtttattctgtaaaacaaagcagtattataaatattctaagacagatattttatttctcaaatttaaatttatttttttccactgatcataaaagcatgaagatatcacacatttgcagagtaccatgtggtgtcccaccacgagtggaatacttaaatctgattaaatccccctctctcctcaggtgtgaattccgcggatccttctatagtgtcacctaaatgtcgacggccaggcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcg

SEQ ID NO: 2 - DNA, 22 bp, Artificial sequence
  Synthetic sequence Primer BACT7For
cgcaagatgtggcgtgttacgg

SEQ ID NO: 3 - DNA, 22 bp, Artificial sequence
  Synthetic sequence Primer TsnaxRev
ggctgctcacaacctacacacg

SEQ ID NO: 4 - DNA, 22 bp, Artificial sequence
  Synthetic sequence Primer Disc1In9For
aaggtagaaccaggtggcttcc

SEQ ID NO: 5 - DNA, 22 bp, Artificial sequence
  Synthetic sequence Primer BACSp6Rev
cgtgatagccgttgtattcagc

SEQ ID NO: 6 - DNA, 22 bp, Artificial sequence
  Synthetic sequence Primer PromFor
tatcaacttcagccgcatccgc

SEQ ID NO: 7 - DNA, 18 bp, Artificial sequence
  Synthetic sequence Primer PromRev
tcataacctcgcctctgg

SEQ ID NO: 8 - DNA, 22 bp, Artificial sequence
  Synthetic sequence Primer E2For
gacaatctgagaggctgactgg

SEQ ID NO: 9 - DNA, 22 bp, Artificial sequence
  Synthetic sequence Primer E2Rev
gttgctcagtaggtagtcctgc

SEQ ID NO: 10 - DNA, 22 bp, Artificial sequence
  Synthetic sequence Primer In5For
agagtcttgtggttggatggcg

SEQ ID NO: 11 - DNA, 22 bp, Artificial sequence
  Synthetic sequence Primer In5Rev
tgaatacagcaccaggctctgc

SEQ ID NO: 12 - DNA, 32 bp, Artificial sequence
  Synthetic sequence Primer In7BamFor
aaaaggatccttgactaactactgttgccagg

SEQ ID NO: 13 - DNA, 29 bp, Artificial sequence
  Synthetic sequence Primer E8NcoIRev
gcagtccatggatagggccagcatcttgg

SEQ ID NO: 14 - DNA, 32 bp, Artificial sequence
  Synthetic sequence Primer In8EcoFor
atgtgaattctagtggtgtcaggcaggtgtgg

SEQ ID NO: 15 - DNA, 32 bp, Artificial sequence
  Synthetic sequence Primer In8RVRev
taatgatatcgatgaggaataccacagacgcc

SEQ ID NO: 16 - DNA, 22 bp, Artificial sequence
  Synthetic sequence Primer In7HRFor
acaggtgatgtgtgtggagtcc

SEQ ID NO: 17 - DNA, 22 bp, Artificial sequence
  Synthetic sequence Primer EGFPRev
atgccgttcttctgcttgtcgg

SEQ ID NO: 18 - DNA, 20 bp, Artificial sequence
  Synthetic sequence Primer EGFPFor
tcctgctggagttcgtgacc

SEQ ID NO: 19 - DNA, 22 bp, Artificial sequence
  Synthetic sequence Primer In8HRRev
gtcacacaggaataagccacgg

SEQ ID NO: 20 - DNA, 22 bp, Artificial sequence
  Synthetic sequence Primer EGFPFor
accatcttcttcaaggacgacg

SEQ ID NO: 21 - DNA, 20 bp, Artificial sequence
  Synthetic sequence Primer EGFPRev
tgctcaggtagtggttgtcg

SEQ ID NO: 22 - DNA, 20 bp, Artificial sequence
  Synthetic sequence Primer
ataataagcggatgaatggc

SEQ ID NO: 23 - DNA, 20 bp, Artificial sequence
  Synthetic sequence Primer
ctgctcacaacctacacacg

SEQ ID NO: 24 - DNA, 22 bp, Artificial sequence
  Synthetic sequence Primer In13For
ctacaacacagagccttgctgc

SEQ ID NO: 25 - DNA, 20 bp, Artificial sequence
  Synthetic sequence Primer E14Rev
agcagtagcagcggcattgg

SEQ ID NO: 26 - DNA, 22 bp, Artificial sequence
  Synthetic sequence Primer E8For
ttgctggaagccaagatgctgg

SEQ ID NO: 27 - DNA, 20 bp, Artificial sequence
  Synthetic sequence Primer EGFPRTR2
tcacgaactccagcaggacc

SEQ ID NO: 28 - DNA, 22 bp, Artificial sequence
  Synthetic sequence Primer
tgtgacctgatggcactggtgg

SEQ ID NO: 29 - DNA, 22 bp, Artificial sequence
  Synthetic sequence Primer
gttgccgtcctccttgaagtcg

SEQ ID NO: 30 - DNA, 22 bp, Artificial sequence
  Synthetic sequence Primer
ttgctggaagccaagatgctgg

SEQ ID NO: 31 - DNA, 20 bp, Artificial sequence
  Synthetic sequence Primer
cttcacgcctatggcttcgc

SEQ ID NO: 32 - DNA, 25 bp, Artificial sequence
  Synthetic sequence Primer
cctgctggattacattaaagcactg

SEQ ID NO: 33 - DNA, 25 bp, Artificial sequence
  Synthetic sequence Primer
gtcaagggcatatccaacaacaaac

SEQ ID NO: 34 - DNA, 31 bp, Artificial sequence
  Synthetic sequence Primer Disc1E12XbaFor
ctagtctagatgcgaagccataggcgtgaag

SEQ ID NO: 35 - DNA, 34 bp, Artificial sequence
  Synthetic sequence Primer Disc1E14XhoRev
tatccgctcgagcatcctgtagacatctcctgag